SEQUENCE LISTING (220 sequences)

SEQ ID NO: 1 - protein, 118 aa, synthetic construct
KCYFLRKAKAKQMPVEMSRPAVPLLNSNNEKMSDPNMEANSHYGHNDDVRNHAMKPINDNKEPLNSDVQYTEVQVSSAESHKDLGKKDTETVYSEVRKAVPDAVESRYSRTEGSLDGT

SEQ ID NO: 2 - protein, 95 aa, synthetic construct
MAEAITYADLRFVKAPLKKSISSRLGQDPGADDDGEITYENVQVPAVLGVPSSLASSVLGDKAAVKSEQPTASWRAVTSPAVGRILPCRTTCLRY

SEQ ID NO: 3 - protein, 105 aa, synthetic construct
LSRKAGRKPASDPARSPSDSDSQEPTYHNVPAWEELQPVYTNANPRGENVVYSEVRIIQEKKKHAVASDPRHLRNKGSPIIYSEVKVASTPVSGSLFLASSAPHR

SEQ ID NO: 4 - protein, 86 aa, synthetic construct
HKISGESSATNEPRGASRPNPQEFTYSSPTPDMEELQPVYVNVGSVDVDVVYSQVWSMQQPESSANIRTLLENKDSQVIYSSVKKS

SEQ ID NO: 5 - protein, 113 aa, synthetic construct
WRMMKYQQKAAGMSPEQVLQPLEGDLCYADLTLQLAGTSPQKATTKLSSAQVDQVEVEYVTMASLPKEDISYASLTLGAEDQEPTYCNMGHLSSHLPGRGPEEPTEYSTISRP

SEQ ID NO: 6 - protein, 182 aa, synthetic construct
YRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNV

SEQ ID NO: 7 - protein, 373 aa, synthetic construct
LRKKHLQMARECGAGTFVNFASLERDGKLPYNWRRSIFAFLTLLPSCLWTDYLLAFYINPWSKNGLKKRKLTNPVQLDDFDAYIKDMAKDSDYKFSLQFEELKLIGLDIPHFAADLPLNRCKNRYTNILPYDFSRVRLVSMNEEEGADYINANYIPGYNSPQEYIATQGPLPETRNDFWKMVLQQKSQIIVMLTQCNEKRRVKCDHYWPFTEEPIAYGDITVEMISEEEQDDWACRHFRINYADEMQDVMHFNYTAWPDHGVPTANAAESILQFVHMVRQQATKSKGPMIIHCSAGVGRTGTFIALDRLLQHIRDHEFVDILGLVSEMRSYRMSMVQTEEQYIFIHQCVQLMWMKKKQQFCISDVIYENVSKS

SEQ ID NO: 8 - protein, 653 aa, synthetic construct
RKCFQTAHFYLEDSTSPRVISTPPTPIFPISDDVGAIPIKHFPKHVADLHASSGFTEEFETLKEFYQEVQSCTVDLGITADSSNHPDNKHKNRYINIVAYDHSRVKLAQLAEKDGKLTDYINANYVDGYNRPKAYIAAQGPLKSTAEDFWRMIWEHNVEVIVMITNLVEKGRRKCDQYWPADGSEEYGNFLVTQKSVQVLAYYTVRNFTLRNTKIKKGSQKGRPSGRVVTQYHYTQWPDMGVPEYSLPVLTFVRKAAYAKRHAVGPVVVHCSAGVGRTGTYIVLDSMLQQIQHEGTVNIFGFLKHIRSQRNYLVQTEEQYVFIHDTLVEAILSKETEVLDSHIHAYVNALLIPGPAGKTKLEKQFQLLSQSNIQQSDYSAALKQCNREKNRTSSIIPVERSRVGISSLSGEGTDYINASYIMGYYQSNEFIITQHPLLHTIKDFWRMIWDHNAQLVVMIPDGQNMAEDEFVYWPNKDEPINCESFKVTLMAEEHKCLSNEEKLIIQDFILEATQDDYVLEVRHFQCPKWPNPDSPISKTFELISVIKEEAANRDGPMIVHDEHGGVTAGTFCALTTLMHQLEKENSVDVYQVAKMINLMRPGVFADIEQYQFLYKVILSLVSTRQEENPSTSLDSNGAALPDGNIAESLESLV

SEQ ID NO: 9 - protein, 128 aa, synthetic construct
MAKRKQGNRLGVCGRFLSSRVSGMNPSSVVHHVSDSGPAAELPLDVPHIRLDSPPSFDNTTYTSLPLDSPSGKPSLPAPSSLPPLPPKVLVCSKPVTYATVIFPGGNKGGGTSCGPAQNPPNNQTPSS

SEQ ID NO: 10 - protein, 77 aa, synthetic construct
QLRRRGKTNHYQTTVEKKSLTIYAQVQKPGPLQKKLDSFPAQDPCTTIYVAATEPVPESVQETNSITVYASVTLPES

SEQ ID NO: 11 - protein, 99 aa, synthetic construct
RLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVI

SEQ ID NO: 12 - DNA, 390 bp, synthetic construct
  source 1..390: mol_type = other DNA
aagtgctacttcctgcggaaggccaaggccaagcagatgcccgtggaaatgagcagaccagccgtgcctctgctgaacagcaacaacgagaagatgagcgaccccaacatggaagccaacagccactacggccacaacgacgacgtgcggaatcacgccatgaagcccatcaacgacaacaaagagcccctgaacagcgacgtgcagtacaccgaggtgcaagtgtctagcgccgagagccacaaggacctgggcaagaaagacaccgagacagtgtacagcgaagtgcgcaaggccgtgcctgatgctgtggaaagcagatacagcagaaccgagggcagcctggatggcacaaccgtgtattctgaggtggtgcagtatacagaagtc

SEQ ID NO: 13 - DNA, 285 bp, synthetic construct
  source 1..285: mol_type = other DNA
atggccgaggccatcacctatgccgacctgagatttgtgaaggcccctctgaagaagtccatcagcagcagactcggccaggatccaggcgctgatgatgatggcgagatcacctacgagaacgtgcaggttccagccgtgctgggagtgccatcttctctggctagtagcgtgctgggcgataaggccgccgtgaagtctgaacagcctaccgcctcttggagagccgtgacatctcctgccgtgggcagaatcctgccttgcagaaccacctgtctgcggtac

SEQ ID NO: 14 - DNA, 315 bp, synthetic construct
  source 1..315: mol_type = other DNA
ctgtctagaaaggccggcagaaagcctgccagcgatcctgccagatctcccagcgacagcgatagccaagagcctacctaccacaatgtgcccgcctgggaagaactgcagcccgtgtacaccaacgccaatcctagaggcgagaacgtggtgtacagcgaagtgcggatcatccaagagaagaaaaagcacgccgtggcctccgatcctcggcaccttagaaacaagggcagccccatcatctactccgaagtgaaggtggccagcacacctgtgtccggcagtctgtttctggcctcttctgccccacaccgg

SEQ ID NO: 15 - DNA, 258 bp, synthetic construct
  source 1..258: mol_type = other DNA
cacaagatcagcggcgagagcagcgccaccaatgaacctagaggcgccagcagacccaatcctcaagagttcacctacagcagccccacacctgacatggaagaactgcagcccgtgtacgtgaacgtgggctccgtggatgtggacgtggtgtacagccaagtgtggtccatgcagcagcctgagtccagcgccaacatcagaaccctgctggaaaacaaggactcccaagtgatctacagctccgtgaagaagtcc

SEQ ID NO: 16 - DNA, 339 bp, synthetic construct
  source 1..339: mol_type = other DNA
tggcggatgatgaagtaccagcagaaagccgccggaatgagccccgaacaggttctgcaacctctggaaggcgatctgtgctacgccgatctgacactgcagctggccggaacatctcctcagaaggccaccacaaagctgagcagcgcccaagtggatcaggtggaagtggaatacgtgacaatggccagcctgcctaaagaggacatcagctacgccagcctgacactgggagccgaggatcaagagcccacctactgcaatatgggccacctgagcagccatctgcctggcagaggacctgaggaacctaccgagtacagcaccatcagcagaccc

SEQ ID NO: 17 - DNA, 546 bp, synthetic construct
  source 1..546: mol_type = other DNA
tacagagcccacttcggcaccgacgagacaatcctggacggcaaagaatacgacatctacgtgtcctacgccagaaacgccgaggaagaggaattcgtcctgctgaccctgagaggcgtgctggaaaacgagttcggctacaagctgtgcatcttcgaccgggattctctgcctggcggcatcgtgacagatgagacactgagcttcatccagaagtcccgcagactgctggtcgtgctgagccccaattatgtgctgcagggaacacaggccctgctggaactgaaagccggcctggaaaacatggccagccggggcaacatcaacgtgatcctggtgcagtacaaggccgtgaaagagacaaaagtcaaagagctgaagcgggccaagaccgtgctgaccgtgattaagtggaagggcgagaagtccaagtatccccagggcagattctggaagcagctgcaggttgccatgcctgtgaagaagtccccaagaagaagcagcagcgacgagcagggactgagctacagcagcctgaagaacgtg

SEQ ID NO: 18 - DNA, 1119 bp, synthetic construct
  source 1..1119: mol_type = other DNA
ctgcggaagaaacacctccagatggccagagaatgtggcgccggaaccttcgtgaatttcgcctctctggaacgcgacggcaagctgccttataactggcggagatccatcttcgcctttctgaccctgctgcctagctgcctgtggaccgattacctgctggccttctacatcaacccttggagcaagaacggcctgaagaagcggaagctgacaaaccccgtgcagctcgacgacttcgacgcctacatcaaggacatggccaaggactccgactacaagttcagcctgcagttcgaggaactgaagctgatcggcctggacatccctcacttcgccgctgacctgcctctgaaccggtgcaagaaccggtacaccaacatcctgccttacgacttcagcagagtgcggctggtgtccatgaacgaggaagagggcgccgactacatcaatgccaactacatccccggctacaacagccctcaagagtatatcgccacacagggccctctgccagagacaagaaacgacttctggaagatggtcctgcagcagaaaagccagatcatcgtgatgctgacccagtgcaacgagaagcggagagtgaagtgcgaccactactggcccttcaccgaggaacctatcgcctacggcgacatcacagtggaaatgatcagcgaggaagaacaggacgactgggcctgccggcacttcagaatcaactacgccgacgagatgcaggacgtgatgcacttcaactacaccgcctggcctgatcacggcgtgccaacagctaatgccgccgaatccatcctgcagtttgtgcacatggttcgacagcaggccaccaagagcaagggccccatgatcatccactgttctgctggcgtgggcagaaccggcacctttatcgctctggacagactgctccagcacatccgggatcacgagttcgtggatatcctgggactcgtgtccgagatgcggagctacagaatgagcatggtgcagacagaggaacagtacatcttcatccaccagtgcgtccagctgatgtggatgaagaagaagcagcagttctgcatcagcgacgtcatctacgagaacgtgtccaagagc

SEQ ID NO: 19 - DNA, 1959 bp, synthetic construct
  source 1..1959: mol_type = other DNA
cggaagtgcttccagacagcccacttctacctggaagatagcacaagccccagagtgatcagcacccctccaacacctatcttccccatctccgatgacgtgggcgctatccccatcaagcactttccaaagcacgtggccgatctgcacgccagctctggcttcaccgaggaattcgagacactgaaagaattctaccaagaggtgcagagctgcaccgtggacctgggaatcacagccgacagcagcaatcaccccgacaacaagcacaagaaccggtacatcaacatcgtggcctacgatcacagcagagtgaagctggcccagctggccgagaaggatggcaagctgaccgactacatcaacgccaattacgtggacggctacaacaggcccaaggcctatattgccgctcagggccctctgaagtctaccgccgaggacttttggagaatgatctgggagcacaacgtggaagtgatcgtgatgatcaccaacctggtggaaaaaggcagacggaagtgcgatcagtactggcctgccgatggcagcgaggaatacggcaatttcctggtcacccagaaaagcgtgcaggtcctggcctactacacagtgcggaacttcaccctgcggaacaccaagatcaagaagggctcccagaagggcagaccctctggcagagtggtcacacagtaccactacacccagtggcctgatatgggcgtgccagagtatagcctgccagtgctgacctttgtgcggaaggccgcctatgccaagagacatgctgtgggacctgtggtggtgcactgttctgctggcgtgggaagaaccggcacctacatcgtgctggacagcatgctccagcagattcagcacgagggcaccgtgaatatcttcggcttcctgaagcacatcagaagccagcggaactacctggtgcagaccgaggaacagtacgtgttcatccacgacacactggtggaagccatcctgagcaaagaaaccgaggtgctggactcccacatccacgcctatgtgaacgccctgctgattcctggaccagccggcaagaccaagctggaaaagcagttccagctgctgtcccagagcaacatccagcagagcgactacagcgccgctctgaagcagtgcaacagagagaagaacagaaccagcagcatcatccctgtggaacggtccagagtgggcatctctagcttgtctggcgagggcacagattacatcaatgccagctacatcatgggctactaccagtccaacgagttcatcatcacacagcaccctctgctgcacaccatcaaggatttctggcggatgatttgggaccacaatgctcagctggtggtcatgatccccgacggccagaatatggccgaggacgagtttgtgtactggcccaacaaggacgagcccatcaactgcgagagcttcaaagtgaccctgatggccgaagaacacaagtgcctgagcaacgaggaaaagctgatcatccaggacttcatcctggaagccacacaggacgactacgtgctggaagtgcggcactttcagtgccctaagtggcccaatcctgacagccccatcagcaagaccttcgagctgatctccgtgatcaaagaggaagccgccaaccgcgacggccctatgattgtgcacgatgagcatggcggagtgaccgccggaacattttgtgccctgacaaccctgatgcaccagctcgagaaagaaaactccgtggacgtgtaccaggtggccaagatgatcaacctgatgcggcctggcgtgttcgccgatatcgagcagtaccagttcctgtacaaagtgatcctcagcctggtgtctacccggcaagaggaaaaccctagcaccagcctggatagcaatggcgccgcactgcccgatggaaatatcgccgagtctctggaaagcctcgtt

SEQ ID NO: 20 - DNA, 384 bp, synthetic construct
  source 1..384: mol_type = other DNA
atggccaagcggaagcagggcaatagactgggcgtgtgcggcagattcctgtcctctagagtgtccggcatgaaccccagcagcgtggtgcatcacgtgtccgattctggacctgccgctgagctgccactggatgtgcctcacatcagactggacagcccacctagcttcgacaacaccacctacacaagcctgcctctcgatagcccttccggcaagccatctctgcctgctccatcttctctgcctccactgcctcctaaggtgctcgtgtgtagcaagcctgtgacctacgccaccgtgatcttccctggcggaaacaaaggcggcggaacatcttgtggccccgctcagaaccctcctaacaatcagacacctagcagc

SEQ ID NO: 21 - DNA, 231 bp, synthetic construct
  source 1..231: mol_type = other DNA
cagctgcgtcgccgtggcaagacgaaccattaccagacgaccgtggagaaaaaatccctgaccatttacgcgcaggtgcagaagccggggcccctgcagaagaagctggactcgtttcctgctcaggacccgtgcactacaatctacgtggcggctacggagcccgtacccgaatccgtgcaggagactaactccatcaccgtctacgcttccgtgacccttcctgagagc

SEQ ID NO: 22 - DNA, 297 bp, synthetic construct
  source 1..297: mol_type = other DNA
cgcctgttcaagcgccgccaggggcgcatcttccccgagggctcttgtctcaatacgttcaccaagaacccctatgctgccagcaagaagaccatctacacctacatcatggcctcccgcaacacccagccagcagagtcgcgcatttacgacgagatcctgcaatctaaggtgctgccatccaaggaggaacccgtcaacactgtttactccgaggtgcagttcgcggacaaaatggggaaggcttccacccaggactccaagccgcctggcacctcgagctacgagatcgtgatt

SEQ ID NO: 23 - protein, 27 aa, synthetic construct
FWVLVVVGGVLACYSLLVTVAFIIFWV

SEQ ID NO: 24 - protein, 19 aa, synthetic construct
GLIAVVIIGVIIALLIIAA

SEQ ID NO: 25 - protein, 21 aa, synthetic construct
VIGILVAVILLLLLLLLLFLI

SEQ ID NO: 26 - protein, 21 aa, synthetic construct
VAGGLLSIAGLAAGALLLYCW

SEQ ID NO: 27 - protein, 21 aa, synthetic construct
LWGLFGVLGFTGVALLLYALF

SEQ ID NO: 28 - protein, 21 aa, synthetic construct
VLLPLIFTILLLLLVAASLLA

SEQ ID NO: 29 - protein, 21 aa, synthetic construct
VLLVVILIVVYHVYWLEMVLF

SEQ ID NO: 30 - protein, 21 aa, synthetic construct
ISVLAILSTLLIGLLLVTLII

SEQ ID NO: 31 - protein, 26 aa, synthetic construct
AVIPLVIVSALTFICLVVLVGILIYW

SEQ ID NO: 32 - protein, 21 aa, synthetic construct
LIWGAVLLVGLLVAAVVLFAV

SEQ ID NO: 33 - protein, 21 aa, synthetic construct
WAVYAGLLGGVIMILIMVVIL

SEQ ID NO: 34 - protein, 21 aa, synthetic construct
LLSVLAMFFLLVLILSSVFLF

SEQ ID NO: 35 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 36 - DNA, 57 bp, synthetic construct
  source 1..57: mol_type = other DNA
ggcctgatcgccgtggttatcatcggcgtgatcattgccctgctgattatcgccgcc

SEQ ID NO: 37 - DNA, 63 bp, synthetic construct
  source 1..63: mol_type = other DNA
gttatagggatcctggtggctgtcatactcctcttgctcctcttgttgctgctttttttgata

SEQ ID NO: 38 - DNA, 63 bp, synthetic construct
  source 1..63: mol_type = other DNA
gttgctggtggcctgctgtctattgctggacttgctgctggtgctctgctgctgtactgctgg

SEQ ID NO: 39 - DNA, 63 bp, synthetic construct
  source 1..63: mol_type = other DNA
ctgtggggactgtttggcgtgttgggctttacaggcgtggccctgctgctgtacgccctgttt

SEQ ID NO: 40 - DNA, 63 bp, synthetic construct
  source 1..63: mol_type = other DNA
gttctgctgcccctgatcttcaccatcctgttgctgctgctggtggccgcttctctgctggct

SEQ ID NO: 41 - DNA, 63 bp, synthetic construct
  source 1..63: mol_type = other DNA
gtgctgctggtggtcatcctgatcgtggtgtaccacgtgtactggctggaaatggtgctgttc

SEQ ID NO: 42 - DNA, 63 bp, synthetic construct
  source 1..63: mol_type = other DNA
atctccgtgctggccatcctgagcacactgctgattggactgctgctcgtgaccctgatcatc

SEQ ID NO: 43 - DNA, 78 bp, synthetic construct
  source 1..78: mol_type = other DNA
gccgtgattcctctggttatcgtgtctgccctgaccttcatctgcctggtggtgctcgtgggcatcctgatctattgg

SEQ ID NO: 44 - DNA, 63 bp, synthetic construct
  source 1..63: mol_type = other DNA
ctgatctggggagcagtgctgcttgtgggactgctggtggctgctgtggtgctgtttgccgtg

SEQ ID NO: 45 - DNA, 63 bp, synthetic construct
  source 1..63: mol_type = other DNA
tgggccgtgtacgcaggcctgcttggcggtgtgatcatgatcctgattatggtggtcatcctg

SEQ ID NO: 46 - DNA, 63 bp, synthetic construct
  source 1..63: mol_type = other DNA
ctgctgtccgtcctggccatgttttttctgttggtactcattctatcatccgtgttcctgttc

SEQ ID NO: 47 - skipped

SEQ ID NO: 48 - protein, 6 aa, synthetic construct
GGSGGS

SEQ ID NO: 49 - protein, 9 aa, synthetic construct
GGSGGSGGS

SEQ ID NO: 50 - protein, 12 aa, synthetic construct
GGSGGSGGSGGS

SEQ ID NO: 51 - protein, 15 aa, synthetic construct
GGSGGSGGSGGSGGS

SEQ ID NO: 52 - protein, 4 aa, synthetic construct
GGGS

SEQ ID NO: 53 - protein, 8 aa, synthetic construct
GGGSGGGS

SEQ ID NO: 54 - protein, 12 aa, synthetic construct
GGGSGGGSGGGS

SEQ ID NO: 55 - protein, 16 aa, synthetic construct
GGGSGGGSGGGSGGGS

SEQ ID NO: 56 - protein, 20 aa, synthetic construct
GGGSGGGSGGGSGGGSGGGS

SEQ ID NO: 57 - protein, 5 aa, synthetic construct
GGGGS

SEQ ID NO: 58 - protein, 10 aa, synthetic construct
GGGGSGGGGS

SEQ ID NO: 59 - protein, 15 aa, synthetic construct
GGGGSGGGGSGGGGS

SEQ ID NO: 60 - protein, 20 aa, synthetic construct
GGGGSGGGGSGGGGSGGGGS

SEQ ID NO: 61 - protein, 25 aa, synthetic construct
GGGGSGGGGSGGGGSGGGGSGGGGS

SEQ ID NO: 62 - protein, 75 aa, synthetic construct
TTTPAPRPPTPAPTIALQPLSLRPEACRPAAGGAVHTRGLDFACDQTTPGERSSLPAFYPGTSGSCSGCGSLSLP

SEQ ID NO: 63 - protein, 18 aa, synthetic construct
GGGGSGGGGSGGGGSQSV

SEQ ID NO: 64 - protein, 18 aa, synthetic construct
GSTSGSGKPGSGEGSTKG

SEQ ID NO: 65 - protein, 27 aa, synthetic construct
SGGGGSGGGGSGGGGSGGGGSGGGSLQ

SEQ ID NO: 66 - DNA, 45 bp, synthetic construct
  source 1..45: mol_type = other DNA
ggaggcggaggatctggtggcggaggaagtggcggaggcggttct

SEQ ID NO: 67 - protein, 42 aa, synthetic construct
AAAIEVMYPPPYLDNEKSNGTIIHVKGKHLCPSPLFPGPSKP

SEQ ID NO: 68 - protein, 12 aa, synthetic construct
ESKYGPPCPSCP

SEQ ID NO: 69 - protein, 12 aa, synthetic construct
ESKYGPPAPSAP

SEQ ID NO: 70 - protein, 12 aa, synthetic construct
ESKYGPPCPPCP

SEQ ID NO: 71 - protein, 12 aa, synthetic construct
EPKSCDKTHTCP

SEQ ID NO: 72 - protein, 86 aa, synthetic construct
AAAFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 73 - protein, 45 aa, synthetic construct
TTTPAPRPPTPAPTIALQPLSLRPEACRPAAGGAVHTRGLDFACD

SEQ ID NO: 74 - protein, 132 aa, synthetic construct
ACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEADAEC

SEQ ID NO: 75 - protein, 34 aa, synthetic construct
ACPTGLYTHSGECCKACNLGEGVAQPCGANQTVC

SEQ ID NO: 76 - protein, 20 aa, synthetic construct
AVGQDTQEVIVVPHSLPFKV

SEQ ID NO: 77 - protein, 66 aa, synthetic construct
ALSNSIMYFSHFVPVFLPAKPTTTPAPRPPTPAPTIASQPLSLRPEACRPAAGGAVHTRGLDFACD

SEQ ID NO: 78 - DNA, 126 bp, synthetic construct
  source 1..126: mol_type = other DNA
gcagcagctatcgaggtgatgtatcctccgccctacctggataatgaaaagagtaatgggactatcattcatgtaaaagggaagcatctttgtccttctccccttttccccggtccgtctaaacct

SEQ ID NO: 79 - DNA, 36 bp, synthetic construct
  source 1..36: mol_type = other DNA
gaatccaagtacggccccccagcgcctagtgcccca

SEQ ID NO: 80 - DNA, 36 bp, synthetic construct
  source 1..36: mol_type = other DNA
gaatctaaatatggcccgccatgcccgccttgccca

SEQ ID NO: 81 - DNA, 36 bp, synthetic construct
  source 1..36: mol_type = other DNA
gaaccgaagtcttgtgataaaactcatacgtgcccg

SEQ ID NO: 82 - DNA, 135 bp, synthetic construct
  source 1..135: mol_type = other DNA
accacgacgccagcgccgcgaccaccaacaccggcgcccaccatcgcgttgcagcccctgtccctgcgcccagaggcgtgccggccagcggcggggggcgcagtgcacacgagggggctggacttcgcctgtgat

SEQ ID NO: 83 - protein, 697 aa, synthetic construct
MALPVTALLLPLALLLHAARPQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDGLIAVVIIGVIIALLIIAAKCYFLRKAKAKQMPVEMSRPAVPLLNSNNEKMSDPNMEANSHYGHNDDVRNHAMKPINDNKEPLNSDVQYTEVQVSSAESHKDLGKKDTETVYSEVRKAVPDAVESRYSRTEGSLDGTGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 84 - protein, 676 aa, synthetic construct
MALPVTALLLPLALLLHAARPQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDVIGILVAVILLLLLLLLLFLIMAEAITYADLRFVKAPLKKSISSRLGQDPGADDDGEITYENVQVPAVLGVPSSLASSVLGDKAAVKSEQPTASWRAVTSPAVGRILPCRTTCLRYGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 85 - protein, 686 aa, synthetic construct
MALPVTALLLPLALLLHAARPQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDVAGGLLSIAGLAAGALLLYCWLSRKAGRKPASDPARSPSDSDSQEPTYHNVPAWEELQPVYTNANPRGENVVYSEVRIIQEKKKHAVASDPRHLRNKGSPIIYSEVKVASTPVSGSLFLASSAPHRGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 86 - protein, 667 aa, synthetic construct
MALPVTALLLPLALLLHAARPQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDLWGLFGVLGFTGVALLLYALFHKISGESSATNEPRGASRPNPQEFTYSSPTPDMEELQPVYVNVGSVDVDVVYSQVWSMQQPESSANIRTLLENKDSQVIYSSVKKSGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 87 - protein, 694 aa, synthetic construct
MALPVTALLLPLALLLHAARPQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDVLLPLIFTILLLLLVAASLLAWRMMKYQQKAAGMSPEQVLQPLEGDLCYADLTLQLAGTSPQKATTKLSSAQVDQVEVEYVTMASLPKEDISYASLTLGAEDQEPTYCNMGHLSSHLPGRGPEEPTEYSTISRPGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 88 - protein, 763 aa, synthetic construct
MALPVTALLLPLALLLHAARPQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDVLLVVILIVVYHVYWLEMVLFYRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNVGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 89 - protein, 954 aa, synthetic construct
MALPVTALLLPLALLLHAARPQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDISVLAILSTLLIGLLLVTLIILRKKHLQMARECGAGTFVNFASLERDGKLPYNWRRSIFAFLTLLPSCLWTDYLLAFYINPWSKNGLKKRKLTNPVQLDDFDAYIKDMAKDSDYKFSLQFEELKLIGLDIPHFAADLPLNRCKNRYTNILPYDFSRVRLVSMNEEEGADYINANYIPGYNSPQEYIATQGPLPETRNDFWKMVLQQKSQIIVMLTQCNEKRRVKCDHYWPFTEEPIAYGDITVEMISEEEQDDWACRHFRINYADEMQDVMHFNYTAWPDHGVPTANAAESILQFVHMVRQQATKSKGPMIIHCSAGVGRTGTFIALDRLLQHIRDHEFVDILGLVSEMRSYRMSMVQTEEQYIFIHQCVQLMWMKKKQQFCISDVIYENVSKSGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 90 - protein, 1239 aa, synthetic construct
MALPVTALLLPLALLLHAARPQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDAVIPLVIVSALTFICLVVLVGILIYWRKCFQTAHFYLEDSTSPRVISTPPTPIFPISDDVGAIPIKHFPKHVADLHASSGFTEEFETLKEFYQEVQSCTVDLGITADSSNHPDNKHKNRYINIVAYDHSRVKLAQLAEKDGKLTDYINANYVDGYNRPKAYIAAQGPLKSTAEDFWRMIWEHNVEVIVMITNLVEKGRRKCDQYWPADGSEEYGNFLVTQKSVQVLAYYTVRNFTLRNTKIKKGSQKGRPSGRVVTQYHYTQWPDMGVPEYSLPVLTFVRKAAYAKRHAVGPVVVHCSAGVGRTGTYIVLDSMLQQIQHEGTVNIFGFLKHIRSQRNYLVQTEEQYVFIHDTLVEAILSKETEVLDSHIHAYVNALLIPGPAGKTKLEKQFQLLSQSNIQQSDYSAALKQCNREKNRTSSIIPVERSRVGISSLSGEGTDYINASYIMGYYQSNEFIITQHPLLHTIKDFWRMIWDHNAQLVVMIPDGQNMAEDEFVYWPNKDEPINCESFKVTLMAEEHKCLSNEEKLIIQDFILEATQDDYVLEVRHFQCPKWPNPDSPISKTFELISVIKEEAANRDGPMIVHDEHGGVTAGTFCALTTLMHQLEKENSVDVYQVAKMINLMRPGVFADIEQYQFLYKVILSLVSTRQEENPSTSLDSNGAALPDGNIAESLESLVGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 91 - protein, 709 aa, synthetic construct
MALPVTALLLPLALLLHAARPQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDLIWGAVLLVGLLVAAVVLFAVMAKRKQGNRLGVCGRFLSSRVSGMNPSSVVHHVSDSGPAAELPLDVPHIRLDSPPSFDNTTYTSLPLDSPSGKPSLPAPSSLPPLPPKVLVCSKPVTYATVIFPGGNKGGGTSCGPAQNPPNNQTPSSGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 92 - protein, 658 aa, synthetic construct
MALPVTALLLPLALLLHAARPQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDWAVYAGLLGGVIMILIMVVILQLRRRGKTNHYQTTVEKKSLTIYAQVQKPGPLQKKLDSFPAQDPCTTIYVAATEPVPESVQETNSITVYASVTLPESGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 93 - protein, 680 aa, synthetic construct
MALPVTALLLPLALLLHAARPQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDLLSVLAMFFLLVLILSSVFLFRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVIGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 94 - protein, 497 aa, synthetic construct
METDTLLLWVLLLWVPGSTGAGGSDYKDDDDKGGSQVQLQESGPGLVKPSETLSLTCTVSGYSITSNYWGWIRQPPGKGLEWMGYITYSGSTSYNPSLKSRITISRDTSKNQFSLKLSSVTAADTAVYYCAITTFYYWGQGTLVTVSSGGGGSGGGGSGGGGSDIQMTQSPSSLSASVGDRVTITCRASQDIGNYLRWFQQKPGKAPKLLISGATNLAAGVPSRFSGSGSGSDFTLTISSLQPEDFATYYCLQSKESPWTFGQGTKVEIKRTTTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - protein, 220 aa, synthetic construct
FKVYKWKQSRDLYRAPVSSLYRTPGPSLHADAVRGGLMSPHLYHQVYLTTDSRRSDPLLKKPGAASPLASRQNTLRSCDPVFYRQVLGAESAPPGQQAPPNTDWRFSQAQRPGTSGSQNGDDTGTWPNNQFDTEMLQAMILASASEAADGSSTLGGGAGTMGLSARYGPQFTLQHVPDYRQNVYIPGSNATLTNAAGKRDGKAPAGGNGNKKKSGKKEKK

SEQ ID NO: 96 - protein, 239 aa, synthetic construct
YRKREWIKETFYPDIPNPENCKALQFQKSVCEGSSALKTLEMNPCTPNNVEVLETRSAFPKIEDTEIISPVAERPEDRSDAEPENHVVVSYCPPIIEEEIPNPAADEAGGTAQVIYIDVQSMYQPQAKPEEEQENDPVGGAGYKPQMHLPINSTVEDIAAEEDLDKTAGYRPQANVNTWNLVSPDSPRSIDSNSEIVSFGSPCSINSRQFLIPPKDEDSPKSNGGGWSFTNFFQNKPND

SEQ ID NO: 97 - protein, 299 aa, synthetic construct
WKQRRAKEKLLYEHVTEVDNLLSDHAKEKGKLHKAVKKLRSELKLKRAAANSGWRRARLHFVAVTLDPDTAHPKLILSEDQRCVRLGDRRQPVPDNPQRFDFVVSILGSEYFTTGCHYWEVYVGDKTKWILGVCSESVSRKGKVTASPANGHWLLRQSRGNEYEALTSPQTSFRLKEPPRCVGIFLDYEAGVISFYNVTNKSHIFTFTHNFSGPLRPFFEPCLHDGGKNTAPLVICSELHKSEESIVPRPEGKGHANGDVSLKVNSSLLPPKAPELKDIILSLPPDLGPALQELKAPSF

SEQ ID NO: 98 - protein, 311 aa, synthetic construct
KCYNIELMLFYRQHFGADETNDDNKEYDAYLSYTKVDQDTLDCDNPEEEQFALEVLPDVLEKHYGYKLFIPERDLIPSGTYMEDLTRYVEQSRRLIIVLTPDYILRRGWSIFELESRLHNMLVSGEIKVILIECTELKGKVNCQEVESLKRSIKLLSLIKWKGSKSSKLNSKFWKHLVYEMPIKKKEMLPRCHVLDSAEQGLFGELQPIPSIAMTSTSATLVSSQADLPEFHPSDSMQIRHCCRGYKHEIPATTLPVPSLGNHHTYCNLPLTLLNGQLPLNNTLKDTQEFHRNSSLLPLSSKELSFTSDIW

SEQ ID NO: 99 - protein, 164 aa, synthetic construct
RRRLRKQARAHGKSVPEIHEQLVTYDEEGGGEMDTTSYDVSVLNSVRRGGAKPPRPALDARPSLYAQVQKPPRHAPGAHGGPGEMAAMIEVKKDEADHDGDGPPYDTLHIYGYEGSESIAESLSSLGTDSSDSDVDYDFLNDWGPRFKMLAELYGSDPREELLY

SEQ ID NO: 100 - protein, 86 aa, synthetic construct
SMILAVLYRRKNSKRDYTGCSTSESLSPVKQAPRKSPSDTEGLVKSLPSGSHQGPVIYAQLDHSGGHHSDKINKSESVVYADIRKN

SEQ ID NO: 101 - protein, 69 aa, synthetic construct
RYCWLRRQAALQRRLSAMEKGKLHKPGKDASKRGRQTPVLYAMLDHSRSTKAVSEKKAKGLGESRKDKK

SEQ ID NO: 102 - protein, 70 aa, synthetic construct
VVALIYCRKKRISALPGYPECREMGETLPEKPANPTNPDEADKVGAENTITYSLLMHPDALEEPDDQNRI

SEQ ID NO: 103 - protein, 85 aa, synthetic construct
RVKTRRKKAAQPVQNTDDVNPVMVSGSRGHQHQFQTGIVSDHPAEAGPISEDEQELHYAVLHFHKVQPQEPKVTDTEYSEIKIHK

SEQ ID NO: 104 - protein, 78 aa, synthetic construct
WLHRRLPPQPIRPLPRFAPLVKTEPQRPVKEEEPKIPGDLDQEPSLLYADLDHLALSRPRRLSTADPADASTIYAVVV

SEQ ID NO: 105 - protein, 95 aa, synthetic construct
MLCRKTSQQEHVYEAARAHAREANDSGETMRVAIFASGCSSDEPTSQNLGNNYSDEPCIGQEYQIIAQINGNYARLLDTVPLDYEFLATEGKSVC

SEQ ID NO: 106 - protein, 93 aa, synthetic construct
RSCRKKSARPAVGVGDTGMEDANAVRGSASQGPLIESPADDSPPHHAPPALATPSPEEGEIQYASLSFHKARPQYPQEQEAIGYEYSEINIPK

SEQ ID NO: 107 - protein, 111 aa, synthetic construct
RHRHQSKHRTSAHFYRPAGAAGPEPKDQGLQKRASPVADIQEEILNAAVKDTQPKDGVEMDAPAAASEAPQDVTYAQLHSLTLRREATEPPPSQEREPPAEPSIYAPLAIH

SEQ ID NO: 108 - protein, 107 aa, synthetic construct
HCWRRRKSGVGFLGDETRLPPAPGPGESSHSICPAQVELQSLYVDVHPKKGDLVYSEIQTTQLGEEEEANTSRTLLEDKDVSVVYSEVKTQHPDNSAGKISSKDEES

SEQ ID NO: 109 - protein, 114 aa, synthetic construct
RWCSKKKDAAVMNQEPAGHRTVNREDSDEQDPQEVTYAQLDHCIFTQRKITGPSQRSKRPSTDTSVCIELPNAEPRALSPAHEHHSQALMGSSRETTALSQTQLASSNVPAAGI

SEQ ID NO: 110 - protein, 116 aa, synthetic construct
LHCCCSNKKNAAVMDQEPAGDRTVNREDSDDQDPQEVTYAQLDHCVFTQTKITSPSQRPKTPPTDTTMYMELPNAKPRSLSPAHKHHSQALRGSSRETTALSQNRVASSHVPAAGI

SEQ ID NO: 111 - protein, 91 aa, synthetic construct
RSCRKKSARPAADVGDIGMKDANTIRGSASQGNLTESWADDNPRHHGLAAHSSGEEREIQYAPLSFHKGEPQDLSGQEATNNEYSEIKIPK

SEQ ID NO: 112 - protein, 140 aa, synthetic construct
HYARARRKPGGLSATGTSSHSPSECQEPSSSRPSRIDPQEPTHSKPLAPMELEPMYSNVNPGDSNPIYSQIWSIQHTKENSANCPMMHQEHEELTVLYSELKKTHPDDSAGEASSRGRAHEEDDEENYENVPRVLLASDH

SEQ ID NO: 113 - protein, 230 aa, synthetic construct
KCLQGNADGDGGGGQCCRRQDSPSPDFYKQSSPNLQVSSDGTLKYMEVTLRPTDSQSHCYRTCFSPASDGSDFTFLRPLSVQQPTALALEPDAIRSRSNTLRERSQQAPPNTDWRFSQAQRPGTSGSQNGDDTGTWPNNQFDTEMLQAMILASASEAADGSSTLGGGAGTMGLSARYGPQFTLQHVPDYRQNVYIPGSNATLTNAAGKRDGKAPAGGNGNKKKSGKKEKK

SEQ ID NO: 114 - protein, 156 aa, synthetic construct
RRQKKEPLIVFEEEDVRENIITYDDEGGGEEDTEAFDIATLQNPDGINGFIPRKDIKPEYQYMPRPGLRPAPNSVDVDDFINTRIQEADNDPTAPPYDSIQIYGYEGRGSVAGSLSSLESATTDSDLDYDYLQNWGPRFKKLADLYGSKDTFDDDS

SEQ ID NO: 115 - protein, 121 aa, synthetic construct
YFFIRTLQAHHDRSERESPFSGSSRQPDSLSSIENAVKYNPVYESHRAGCEKYEGPYPQHPFYSSASGDVIGGLSREEIRQMYESSELSREEIQERMRVLELYANDPEFAAFVREQQVEEV

SEQ ID NO: 116 - protein, 396 aa, synthetic construct
RRRRREQRLKRLRDAKSLAEMLMSKNTRTSDTLSKQQQTLRMHIDIPRAQLLIEERDTMETIDDRSTVLLTDADFGEAAKQKSLTVTHTVHYQSVSQATGPLVDVSDARPGTNPTTRRNAKAGPTARNRYASQWTLNRPHPTISAHTLTTDWRLPTPRAAGSVDKESDSYSVSPSQDTDRARSSMVSTESASSTYEELARAYEHAKMEEQLRHAKFTITECFISDTSSEQLTAGTNEYTDSLTSSTPSESGICRFTASPPKPQDGGRVMNMAVPKAHRPGDLIHLPPYLRMDFLLNRGGPGTSRDLSLGQACLEPQKSRTLKRPTVLEPIPMEAASSASSTREGQSWQPGAVATLPQREGAELGQAAKMSSSQESLLDSRGHLKGNNPYAKSYTLV

SEQ ID NO: 117 - DNA, 660 bp, synthetic construct
  source 1..660: mol_type = other DNA
ttcaaggtgtacaagtggaagcagagccgggacctgtacagagcccctgtgtccagcctgtatagaacccctggacctagcctgcatgccgatgctgttagaggcggcctgatgagccctcacctgtaccatcaggtgtacctgaccaccgacagcagaagaagcgaccctctgctgaagaagcctggcgctgcttctccactggccagcagacagaataccctgagaagctgcgaccccgtgttctataggcaggttctgggagccgaatctgcccctcctggacaacaggcccctcctaacaccgattggagattcagccaggctcagaggcctggcacaagcggatctcagaatggcgacgataccggcacctggcctaacaaccagttcgacaccgaaatgctgcaggccatgattctggcctctgcctctgaagccgccgatggcagttctacacttggaggcggagccggcacaatgggactgtctgctagatacggccctcagttcaccctgcagcacgtgcccgattaccggcagaacgtgtacatccccggcagcaatgccacactgacaaacgccgctggcaagagagatggcaaagctcctgctggcggcaacggcaacaagaagaagtccggcaagaaagagaagaag

SEQ ID NO: 118 - DNA, 717 bp, synthetic construct
  source 1..717: mol_type = other DNA
taccggaagagagagtggatcaaagagacattctacccggacatccccaatcctgagaactgcaaagccctgcagttccagaaaagcgtgtgcgagggaagcagcgccctgaaaaccctggaaatgaacccctgcacacccaacaacgtggaagtgctggaaaccagaagcgctttccccaagatcgaggacaccgagatcatcagccccgtggccgaaagacccgaggatagatctgatgccgagcctgagaatcacgtggtggtgtcctactgtcctcctatcatcgaggaagagatccctaatcctgccgccgatgaagccggcggaacagcccaagtgatctacatcgacgtgcagagcatgtaccagcctcaggccaagcctgaggaagaacaagagaacgaccctgttggcggagccggctacaagccccaaatgcatctgcccatcaacagcaccgtggaagatatcgccgccgaagaggacctggataagaccgccggatatagaccccaggccaacgtgaacacctggaacctggtgtccccagacagccccagatccatcgacagcaacagcgagatcgtgtccttcggcagcccctgctctatcaacagccggcagttcctgattcctcctaaggacgaggacagccctaagagcaatggcggcggatggtccttcaccaatttcttccagaacaagcccaacgac

SEQ ID NO: 119 - DNA, 897 bp, synthetic construct
  source 1..897: mol_type = other DNA
tggaagcagcggagagccaaagagaagctgctgtacgagcacgtgaccgaggtggacaacctgctgagcgatcacgccaaagaaaagggcaagctgcacaaggccgtgaagaagctgagaagcgagctgaagctgaagcgggctgccgccaattctggatggcgtagagccagactgcacttcgtggccgtgacactggaccccgatacagcccatcctaagctgatcctgagcgaggaccagagatgtgtgcggctgggagatagaaggcagcccgtgcctgacaaccctcagagattcgacttcgtggtgtccatcctgggcagcgagtacttcaccaccggctgccactactgggaagtgtacgtgggcgacaagaccaagtggatcctgggcgtgtgtagcgagagcgtgtccagaaagggcaaagtgacagctagccccgccaatggacactggctgctgagacagagcagaggcaatgagtacgaggccctgacaagcccacagaccagcttccggctgaaagaacctcctagatgcgtgggcatcttcctggattatgaggccggcgtgatcagcttctacaacgtgaccaacaagagccacatcttcacgttcacccacaacttcagcggccctctgcggccattcttcgagccttgtctgcacgacggcggcaagaatactgcccctctggtcatctgtagcgaactgcacaagagcgaggaatccatcgtgcccagacctgaaggcaagggacatgccaatggggacgtgtccctgaaagtgaacagcagcctgctgcctcctaaggctcctgagctgaaggacatcatcctgagcctgcctccagacctgggacctgctctgcaagaactgaaggcccctagcttc

SEQ ID NO: 120 - DNA, 933 bp, synthetic construct
  source 1..933: mol_type = other DNA
aagtgctacaacatcgagctgatgctgttctaccggcagcactttggcgccgacgagacaaacgacgacaacaaagagtacgacgcctacctgagctacaccaaggtggaccaggacaccctggactgcgacaaccctgaggaagaacagttcgccctcgaggtgctgcccgacgtgctggaaaagcactacggctacaagctgttcatccccgagcgggatctgatccctagcggcacctacatggaagatctgaccagatacgtggaacagagcagacggctgatcatcgtgctgacccctgactacatcctgcggagaggctggtccatcttcgagctggaaagccggctgcacaacatgctggtgtccggcgagatcaaagtgatcctgatcgagtgcaccgagctgaagggcaaagtgaactgccaagaggtggaaagcctgaagcggagcatcaagctgctgagcctgatcaagtggaagggcagcaagagcagcaagctgaacagcaagttctggaagcacctggtgtacgagatgcccatcaaaaagaaagaaatgctgccccggtgccatgtgctggattctgctgagcagggcctgtttggagagctgcagcccattccttctatcgccatgaccagcacctccgccacactggtttctagccaggccgacctgcctgagtttcaccccagcgatagcatgcagatccggcactgctgccggggctataagcacgagattccagccaccacactgcccgtgccttctctgggaaatcaccacacctactgcaacctgcctctgacactgctgaacggccagctgccactgaacaacaccctgaaggacacccaagagttccaccggaacagcagcctgctgcctctgtccagcaaagagctgagcttcaccagcgacatctgg

SEQ ID NO: 121 - DNA, 492 bp, synthetic construct
  source 1..492: mol_type = other DNA
cggcggagactgagaaagcaggctagagcccacggcaagagcgtgccagagattcacgaacagctggtcacctacgacgaggaaggcggaggcgagatggataccacaagctacgatgtgtccgtgctgaacagcgtgcgaagaggcggagccaaacctcctagacctgctctggatgccagacctagcctgtatgcccaggtgcagaagcctccaagacacgctcctggtgctcatggtggacctggcgaaatggccgccatgatcgaagtgaagaaggacgaggccgaccacgatggcgacggccctccatatgataccctgcacatctacggctacgagggcagcgagtctatcgccgagtctctgtctagcctgggcaccgatagcagcgatagcgacgtggactacgacttcctgaacgactggggccctagattcaagatgctggccgagctgtacggcagcgaccctagagaggaactgctgtac

SEQ ID NO: 122 - DNA, 258 bp, synthetic construct
  source 1..258: mol_type = other DNA
tctatgatcctggccgtgctgtaccggcggaagaacagcaagagagactacaccggctgcagcaccagcgagtctctgtctccagtgaagcaggcccctagaaagagcccctctgataccgaaggcctggtcaagtctctgcccagcggatctcatcagggcccagtgatctatgcccagctggaccattctggcggccaccacagcgacaagatcaacaagagcgagagcgtggtgtacgccgacatccggaagaat

SEQ ID NO: 123 - DNA, 207 bp, synthetic construct
  source 1..207: mol_type = other DNA
cggtactgctggctgagaaggcaggctgcactgcagagaaggctgagcgccatggaaaagggcaagctgcacaagcctggcaaggacgcctctaagagaggcagacagacccctgtgctgtacgccatgctggaccacagcagaagcacaaaggccgtcagcgagaagaaggccaaaggcctgggcgagagccggaaggataagaag

SEQ ID NO: 124 - DNA, 210 bp, synthetic construct
  source 1..210: mol_type = other DNA
gtggtggccctgatctactgccggaagaagagaatctctgccctgcctggctaccccgagtgtagagagatgggagagacactgcccgagaagcccgccaatcctaccaatcctgacgaggccgacaaagtgggcgccgagaataccatcacctacagcctgctgatgcaccccgacgctctggaagaacccgacgaccagaacagaatc

SEQ ID NO: 125 - DNA, 255 bp, synthetic construct
  source 1..255: mol_type = other DNA
agagtgaaaacccggcggaagaaagccgctcagcccgtgcagaataccgacgacgtgaaccctgtgatggtgtccggctctagaggacaccagcaccagtttcagaccggcatcgtgtctgatcaccctgccgaagccggacctatcagcgaggatgagcaagagctgcactacgccgtgctgcacttccacaaggtgcagccccaagagcctaaagtgaccgacaccgagtacagcgagatcaagatccacaag

SEQ ID NO: 126 - DNA, 234 bp, synthetic construct
  source 1..234: mol_type = other DNA
tggctgcacagaaggctgcctccacagcctatcagacccctgcctagatttgcccctctggtcaagacagagccccagcggcctgtgaaagaggaagaacctaagatccccggcgacctggaccaagagccttctctgctgtacgccgacctggatcatctggccctgagcagacctagacggctgtctacagccgatcctgccgatgccagcacaatctatgctgtggtggtg

SEQ ID NO: 127 - DNA, 285 bp, synthetic construct
  source 1..285: mol_type = other DNA
atgctgtgcagaaagaccagccagcaagagcacgtgtacgaggccgctagagcccatgccagagaggctaacgatagcggcgagacaatgagagtggccatcttcgccagcggctgtagctctgatgagcccacctctcagaacctgggcaacaactacagcgacgagccctgcatcggccaagagtaccagatcattgcccagatcaacggcaactacgcccggctgctggataccgtgcctctggattatgagttcctggccaccgagggcaagagcgtgtgt

SEQ ID NO: 128 - DNA, 279 bp, synthetic construct
  source 1..279: mol_type = other DNA
cggagctgccggaagaagtctgctagacctgctgttggcgtgggcgataccggaatggaagatgccaatgccgtcagaggcagcgcctctcagggacctctgattgagtctcccgccgacgatagccctcctcatcatgctcctccagctctggccacaccttctccagaggaaggcgagatccagtacgccagcctgagcttccacaaggccagacctcagtaccctcaagagcaagaggccatcggctacgagtacagcgagatcaacatccccaag

SEQ ID NO: 129 - DNA, 333 bp, synthetic construct
  source 1..333: mol_type = other DNA
cgacatcggcatcagagcaaacacaggacatcggcccatttctaccgtcctgcaggggctgcggggccagagcccaaggaccagggcctgcagaagagggccagcccagttgctgacatccaggaggaaattctcaatgctgccgtgaaggacacacagcccaaggacggggtggagatggatgctccggctgctgcatctgaagccccccaggatgtgacctacgcccagctgcacagcttgaccctcagacgggaggcaactgagcctcctccatcccaggaaagggaacctccagctgaacccagcatctacgcccccctggccatccac

SEQ ID NO: 130 - DNA, 321 bp, synthetic construct
  source 1..321: mol_type = other DNA
cactgctggcgtcggaggaagtcaggagttggtttcttgggagacgaaaccaggctccctcccgctccaggcccaggagagtcctcccattccatctgccctgcccaggtggagcttcagtcgttgtatgttgatgtacaccccaaaaagggagatttggtatactctgagatccagactactcagctgggagaagaagaggaagctaatacctccaggacacttctagaggataaggatgtctcagttgtctactctgaggtaaagacacaacacccagataactcagctggaaagatcagctctaaggatgaagaaagt

SEQ ID NO: 131 - DNA, 342 bp, synthetic construct
  source 1..342: mol_type = other DNA
cgctggtgctctaaaaagaaggatgccgcggtgatgaaccaggagcccgctggccaccgcaccgtcaaccgcgaggacagtgacgagcaggatccgcaggaggtgacctacgcgcagttggatcactgtattttcactcagcgcaagattaccggcccttcacagaggtccaagcgcccttcgaccgacacctctgtctgtattgagttgcccaacgcggagccaagagcactgagcccggcgcatgagcaccacagccaggccctgatgggctcgtcccgcgaaacgacagctctctcgcagacccagcttgctagctctaatgtaccagcagccgggatc

SEQ ID NO: 132 - DNA, 348 bp, synthetic construct
  source 1..348: mol_type = other DNA
cttcattgctgctgctccaacaaaaagaatgctgctgtaatggaccaagagcctgccggggacagaacagtgaacagggaggactctgatgatcaagaccctcaggaggtgacatatgcacagttggatcactgcgttttcacacagacaaaaatcacttccccttctcagaggcccaagacacctccaacagataccaccatgtacatggaacttccaaatgctaagccaagatcattgtctcctgcccataagcaccacagtcaggccttgaggggatcttctagggagacaacagccctgtctcaaaaccgggttgctagctcccatgtaccagcagctggaatc

SEQ ID NO: 133 - DNA, 273 bp, synthetic construct
  source 1..273: mol_type = other DNA
aggtcctgcaggaagaaatcggcaaggccagcagcggacgtgggagacataggcatgaaggatgcaaacaccatcaggggctcagcctctcagggtaacctgactgagtcctgggcagatgataacccccgacaccatggcctggctgcccactcctcaggggaggaaagagagatccagtatgcacccctcagctttcataagggggagcctcaggacctatcaggacaagaagccaccaacaatgagtactcagagatcaagatccccaag

SEQ ID NO: 134 - DNA, 420 bp, synthetic construct
  source 1..420: mol_type = other DNA
cattatgccagagccagaagaaagcctggcggcctgtctgccaccggcacatcttctcacagccccagcgagtgtcaagagcccagcagcagcagacccagcagaatcgatccccaagagcctacacacagcaagcccctggctcctatggaactggaacccatgtacagcaacgtgaaccccggcgacagcaaccccatctacagccagatttggagcatccagcacaccaaagagaacagcgccaactgtcccatgatgcaccaagagcacgaggaactgaccgtgctgtactccgagctgaagaaaacacaccccgacgactctgccggcgaggcctcttctagaggcagagcccatgaagaggacgacgaagagaactacgagaacgtgcccagagtgctgctggcctccgatcat

SEQ ID NO: 135 - DNA, 690 bp, synthetic construct
  source 1..690: mol_type = other DNA
aagtgcctgcagggaaatgctgatggcgacggtggcggaggccagtgttgtagaaggcaggatagcccctctccagacttctacaagcagagcagccccaacctccaggtgtcctctgatggcaccctgaagtacatggaagtgaccctgaggcctaccgacagccagagccactgctacagaacctgctttagccctgccagcgacggcagcgactttacctttctgaggcctctgagcgtgcagcagcctacagctctggctctggaacctgacgccatcagaagcagaagcaacaccctgagagagcggagccagcaggcccctcctaataccgattggagattcagccaggctcagcggcctggcacaagcggatctcagaatggcgacgataccggcacctggcctaacaaccagttcgacaccgaaatgctgcaggccatgattctggcctctgcctctgaagccgccgatggcagttctacacttggaggcggagccggcacaatgggactgtctgctagatacggccctcagttcaccctgcagcacgtgcccgactacagacagaacgtgtacatccccggcagcaacgccacactgacaaatgccgccggaaagagagatggcaaagcccctgctggcggcaacggcaacaagaagaagtccggcaagaaagagaagaag

SEQ ID NO: 136 - DNA, 468 bp, synthetic construct
  source 1..468: mol_type = other DNA
cggcggcagaagaaagaacccctgatcgtgttcgaagaggaagatgtgcgcgagaacatcatcacctacgacgacgaaggcggaggcgaagaggataccgaggccttcgatatcgccacactgcagaaccccgacggcatcaacggcttcatccccagaaaggacatcaagcccgagtaccagtacatgcccagacctggactcagacccgctcctaattccgtggacgtggacgacttcatcaacacccggatccaagaggccgacaacgaccctacagctcctccatacgacagcatccagatctacggctacgaaggcagaggaagcgtggccggatctctgagcagtctggaaagcgccaccaccgacagcgacctggattacgactacctgcagaactggggccctagattcaagaagctggccgacctgtacggcagcaaggacaccttcgacgatgacagc

SEQ ID NO: 137 - DNA, 363 bp, synthetic construct
  source 1..363: mol_type = other DNA
tactttttcatccggacactgcaggcccatcacgacagatccgagagagagagccctttcagcggcagcagcagacagcctgatagcctgagcagcatcgagaacgccgtgaagtacaaccccgtgtacgagtctcacagagccggctgcgagaagtacgagggcccttatcctcagcaccccttctacagctctgccagcggagatgtgatcggcggcctgtccagagaagagatccggcagatgtacgagagcagcgagctgagccgggaagagattcaagagcggatgagagtgctggaactgtacgccaacgatcccgagttcgccgcctttgtgcgagaacagcaggtcgaggaagtg

SEQ ID NO: 138 - DNA, 1188 bp, synthetic construct
  source 1..1188: mol_type = other DNA
cggcggagaagaagagagcagcggctgaagagactgcgggatgccaaatctctggccgagatgctgatgagcaagaacaccagaaccagcgacaccctgagcaagcagcaacagaccctgcggatgcacatcgacatccccagagcacagctgctgatcgaggaacgggacaccatggaaaccatcgacgacagatccaccgtgctgctgaccgatgccgattttggagaggccgccaagcagaaaagcctgaccgtgacacacaccgtgcactaccagtctgtgtcccaggctacaggacctctggtggacgtgtcagatgccagacctggcacaaaccccaccaccagaagaaacgccaaggccggacctaccgccagaaacagatatgccagccagtggaccctgaacagaccccatcctaccatcagcgcccacacactgaccaccgattggagactgcccacacctagagccgccggatctgtggacaaagagtccgacagctacagcgtgtcccctagccaggataccgacagagccagatccagcatggtgtctacagagagcgccagcagcacctacgaggaactggccagagcctatgagcacgccaagatggaagaacagctgcgccacgccaagttcaccatcaccgagtgcttcatctccgacaccagcagcgaacagctgaccgccggcaccaatgagtacaccgatagcctgacctccagcacaccttccgagagcggcatctgcaggtttaccgcctctccacctaagcctcaggatggcggcagagtgatgaacatggccgtgcctaaggctcacagacccggcgacctgattcatctgccaccttacctgagaatggacttcctgctgaacagaggcggcccaggcacaagcagagatctgtctcttggccaggcctgcctggaacctcagaagtctagaaccctgaagcggcctaccgtgctggaacccattcctatggaagccgccagctctgcctctagcacaagagagggacagtcttggcagcctggcgctgttgctacactgcctcaaagagaaggcgccgaactgggacaagccgccaaaatgagcagcagccaagagagcctgctggactctagaggccacctgaagggcaacaacccctacgccaagagctacaccctggtg

SEQ ID NO: 139 - protein, 21 aa, synthetic construct
LLLSLILVSVGFVVTVFGVII

SEQ ID NO: 140 - protein, 25 aa, synthetic construct
VGLIIAILIPVAVAVIVGVVTSILC

SEQ ID NO: 141 - protein, 21 aa, synthetic construct
VALAVILPVLVLLIMVCLCLI

SEQ ID NO: 142 - protein, 21 aa, synthetic construct
IELAGGLGAIFLLLVLLVVIY

SEQ ID NO: 143 - protein, 21 aa, synthetic construct
AVVAILLCILTITVITLLIFL

SEQ ID NO: 144 - protein, 21 aa, synthetic construct
FPVWVVVGIVTAVVLGLTLLI

SEQ ID NO: 145 - protein, 26 aa, synthetic construct
YGVVLGAVIGGVLGVVLLLLLLFYVV

SEQ ID NO: 146 - protein, 23 aa, synthetic construct
SSSPMGIIVAVVTGIAVAAIVAA

SEQ ID NO: 147 - protein, 21 aa, synthetic construct
LGAVWGASITTLVFLCVCFIF

SEQ ID NO: 148 - protein, 21 aa, synthetic construct
LLIPLLGAGLVLGLGALGLVW

SEQ ID NO: 149 - protein, 21 aa, synthetic construct
VFAIILIISLCCMVVFTMAYI

SEQ ID NO: 150 - protein, 21 aa, synthetic construct
FGGAGATALVFLYFCIIFVVV

SEQ ID NO: 151 - protein, 21 aa, synthetic construct
VVTGVSVAFVLLLFLLLFLLL

SEQ ID NO: 152 - protein, 21 aa, synthetic construct
VAAGATGGLLSALLLAVALLF

SEQ ID NO: 153 - protein, 21 aa, synthetic construct
AVIRYSVAIILFTILPFFLLH

SEQ ID NO: 154 - protein, 21 aa, synthetic construct
LHILIGTSVAIILFIILFFFL

SEQ ID NO: 155 - protein, 23 aa, synthetic construct
GAVGGAGATALVFLSFCVIFIVV

SEQ ID NO: 156 - protein, 21 aa, synthetic construct
AAGITGLVLSILVLAAAAALL

SEQ ID NO: 157 - protein, 21 aa, synthetic construct
LIVALATVSLLSLVTFTFLSA

SEQ ID NO: 158 - protein, 23 aa, synthetic construct
GALIAILACIVILLVIVVLFVTL

SEQ ID NO: 159 - protein, 21 aa, synthetic construct
VIIGITIASVVGLLVIFSAII

SEQ ID NO: 160 - protein, 21 aa, synthetic construct
LVTISCILVGVLLLFVLLLVV

SEQ ID NO: 161 - DNA, 63 bp, synthetic construct
  source 1..63: mol_type = other DNA
ctgctgctgtctctgatcctggtgtccgtgggctttgtggtcaccgtgttcggcgtgatcatc

SEQ ID NO: 162 - DNA, 75 bp, synthetic construct
  source 1..75: mol_type = other DNA
gtgggactgatcattgccattctgatccctgtggccgtggccgtgattgtgggcgtcgtgacatccatcctgtgc

SEQ ID NO: 163 - DNA, 63 bp, synthetic construct
  source 1..63: mol_type = other DNA
gtggctctggctgtgattctgcctgtgctggtgctgctgatcatggtttgcctgtgcctgatc

SEQ ID NO: 164 - DNA, 63 bp, synthetic construct
  source 1..63: mol_type = other DNA
attgaactggctggcggactgggcgccatctttctgctgctggtgctgctcgtggtcatctac

SEQ ID NO: 165 - DNA, 63 bp, synthetic construct
  source 1..63: mol_type = other DNA
gccgtggttgccatcctgctgtgtatcctgaccatcaccgtgattaccctgctgatcttcctg

SEQ ID NO: 166 - DNA, 63 bp, synthetic construct
  source 1..63: mol_type = other DNA
ttccctgtgtgggttgtcgtgggaatcgtgacagctgtggtgctgggactgaccctgctgatc

SEQ ID NO: 167 - DNA, 78 bp, synthetic construct
  source 1..78: mol_type = other DNA
tatggcgttgtgctgggagctgtgattggcggagttctgggagttgtgctgctgctcctgctgctgttttacgtcgtg

SEQ ID NO: 168 - DNA, 69 bp, synthetic construct
  source 1..69: mol_type = other DNA
agcagctctcccatgggcatcattgtggccgtggtcacaggcattgccgtggccgctatagtggctgct

SEQ ID NO: 169 - DNA, 63 bp, synthetic construct
  source 1..63: mol_type = other DNA
ctgggagctgtttggggcgcctctattacaaccctggtgttcctgtgcgtgtgcttcatcttc

SEQ ID NO: 170 - DNA, 63 bp, synthetic construct
  source 1..63: mol_type = other DNA
ctgctgattcctctgcttggagccggactggtgcttggacttggagcactgggacttgtgtgg

SEQ ID NO: 171 - DNA, 63 bp, synthetic construct
  source 1..63: mol_type = other DNA
gtgttcgccatcatcctgatcatcagcctgtgctgcatggtggtgttcaccatggcctacatc

SEQ ID NO: 172 - DNA, 63 bp, synthetic construct
  source 1..63: mol_type = other DNA
tttggcggagctggtgctacagccctggtgttcctgtacttctgcatcatcttcgtggtcgtg

SEQ ID NO: 173 - DNA, 63 bp, synthetic construct
  source 1..63: mol_type = other DNA
gttgtgactggggtctcagtggccttcgtcctgctgctgttcctcctcctcttcctcctcctc

SEQ ID NO: 174 - DNA, 63 bp, synthetic construct
  source 1..63: mol_type = other DNA
gtcgccgcgggagccactggagggctgctcagtgctcttctcctggctgtggccctgctgttt

SEQ ID NO: 175 - DNA, 63 bp, synthetic construct
  source 1..63: mol_type = other DNA
gctgtgatacgttattccgtggccatcatcctgttcaccatcctacccttcttcctgctgcat

SEQ ID NO: 176 - DNA, 63 bp, synthetic construct
  source 1..63: mol_type = other DNA
ctgcacattctgattgggacctcagtggctatcatcctcttcatcatcctcttcttctttctc

SEQ ID NO: 177 - DNA, 69 bp, synthetic construct
  source 1..69: mol_type = other DNA
ggggcggtcgggggagctggagccacagccctggtcttcctctccttctgtgtcatcttcattgtagtg

SEQ ID NO: 178 - DNA, 63 bp, synthetic construct
  source 1..63: mol_type = other DNA
gccgctggaattacaggcctggtgctgagcattctggtgctggctgcagctgctgccctgctg

SEQ ID NO: 179 - DNA, 63 bp, synthetic construct
  source 1..63: mol_type = other DNA
ctgattgtggccctggccacagtgtctctgctgagcctcgtgaccttcaccttcctgagcgcc

SEQ ID NO: 180 - DNA, 69 bp, synthetic construct
  source 1..69: mol_type = other DNA
ggagccctgattgccatcctggcctgtatcgtgatcctgctggtcatcgtggtgctgttcgtgaccctg

SEQ ID NO: 181 - DNA, 63 bp, synthetic construct
  source 1..63: mol_type = other DNA
gtgatcatcggcatcacaatcgcctctgtcgtgggcctgctggtcatcttcagcgccatcatc

SEQ ID NO: 182 - DNA, 63 bp, synthetic construct
  source 1..63: mol_type = other DNA
ctggtcaccatcagctgtatcctcgtgggagtgctgctgctgttcgtcctgctgctggttgtg

SEQ ID NO: 183 - protein, 228 aa, synthetic construct
ESKYGPPCPSCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 184 - protein, 119 aa, synthetic construct
ESKYGPPCPSCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 185 - protein, 601 aa, synthetic construct
MALPVTALLLPLALLLHAARPKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDLLLSLILVSVGFVVTVFGVIIFKVYKWKQSRDLYRAPVSSLYRTPGPSLHADAVRGGLMSPHLYHQVYLTTDSRRSDPLLKKPGAASPLASRQNTLRSCDPVFYRQVLGAESAPPGQQAPPNTDWRFSQAQRPGTSGSQNGDDTGTWPNNQFDTEMLQAMILASASEAADGSSTLGGGAGTMGLSARYGPQFTLQHVPDYRQNVYIPGSNATLTNAAGKRDGKAPAGGNGNKKKSGKKEKK

SEQ ID NO: 186 - protein, 624 aa, synthetic construct
MALPVTALLLPLALLLHAARPKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDVGLIIAILIPVAVAVIVGVVTSILCYRKREWIKETFYPDIPNPENCKALQFQKSVCEGSSALKTLEMNPCTPNNVEVLETRSAFPKIEDTEIISPVAERPEDRSDAEPENHVVVSYCPPIIEEEIPNPAADEAGGTAQVIYIDVQSMYQPQAKPEEEQENDPVGGAGYKPQMHLPINSTVEDIAAEEDLDKTAGYRPQANVNTWNLVSPDSPRSIDSNSEIVSFGSPCSINSRQFLIPPKDEDSPKSNGGGWSFTNFFQNKPND

SEQ ID NO: 187 - protein, 680 aa, synthetic construct
MALPVTALLLPLALLLHAARPKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDVALAVILPVLVLLIMVCLCLIWKQRRAKEKLLYEHVTEVDNLLSDHAKEKGKLHKAVKKLRSELKLKRAAANSGWRRARLHFVAVTLDPDTAHPKLILSEDQRCVRLGDRRQPVPDNPQRFDFVVSILGSEYFTTGCHYWEVYVGDKTKWILGVCSESVSRKGKVTASPANGHWLLRQSRGNEYEALTSPQTSFRLKEPPRCVGIFLDYEAGVISFYNVTNKSHIFTFTHNFSGPLRPFFEPCLHDGGKNTAPLVICSELHKSEESIVPRPEGKGHANGDVSLKVNSSLLPPKAPELKDIILSLPPDLGPALQELKAPSF

SEQ ID NO: 188 - protein, 692 aa, synthetic construct
MALPVTALLLPLALLLHAARPKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDIELAGGLGAIFLLLVLLVVIYKCYNIELMLFYRQHFGADETNDDNKEYDAYLSYTKVDQDTLDCDNPEEEQFALEVLPDVLEKHYGYKLFIPERDLIPSGTYMEDLTRYVEQSRRLIIVLTPDYILRRGWSIFELESRLHNMLVSGEIKVILIECTELKGKVNCQEVESLKRSIKLLSLIKWKGSKSSKLNSKFWKHLVYEMPIKKKEMLPRCHVLDSAEQGLFGELQPIPSIAMTSTSATLVSSQADLPEFHPSDSMQIRHCCRGYKHEIPATTLPVPSLGNHHTYCNLPLTLLNGQLPLNNTLKDTQEFHRNSSLLPLSSKELSFTSDIW

SEQ ID NO: 189 - protein, 545 aa, synthetic construct
MALPVTALLLPLALLLHAARPKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDAVVAILLCILTITVITLLIFLRRRLRKQARAHGKSVPEIHEQLVTYDEEGGGEMDTTSYDVSVLNSVRRGGAKPPRPALDARPSLYAQVQKPPRHAPGAHGGPGEMAAMIEVKKDEADHDGDGPPYDTLHIYGYEGSESIAESLSSLGTDSSDSDVDYDFLNDWGPRFKMLAELYGSDPREELLY

SEQ ID NO: 190 - protein, 467 aa, synthetic construct
MALPVTALLLPLALLLHAARPKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDFPVWVVVGIVTAVVLGLTLLISMILAVLYRRKNSKRDYTGCSTSESLSPVKQAPRKSPSDTEGLVKSLPSGSHQGPVIYAQLDHSGGHHSDKINKSESVVYADIRKN

SEQ ID NO: 191 - protein, 455 aa, synthetic construct
MALPVTALLLPLALLLHAARPKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDYGVVLGAVIGGVLGVVLLLLLLFYVVRYCWLRRQAALQRRLSAMEKGKLHKPGKDASKRGRQTPVLYAMLDHSRSTKAVSEKKAKGLGESRKDKK

SEQ ID NO: 192 - protein, 453 aa, synthetic construct
MALPVTALLLPLALLLHAARPKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDSSSPMGIIVAVVTGIAVAAIVAAVVALIYCRKKRISALPGYPECREMGETLPEKPANPTNPDEADKVGAENTITYSLLMHPDALEEPDDQNRI

SEQ ID NO: 193 - protein, 466 aa, synthetic construct
MALPVTALLLPLALLLHAARPKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDLGAVWGASITTLVFLCVCFIFRVKTRRKKAAQPVQNTDDVNPVMVSGSRGHQHQFQTGIVSDHPAEAGPISEDEQELHYAVLHFHKVQPQEPKVTDTEYSEIKIHK

SEQ ID NO: 194 - protein, 459 aa, synthetic construct
MALPVTALLLPLALLLHAARPKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDLLIPLLGAGLVLGLGALGLVWWLHRRLPPQPIRPLPRFAPLVKTEPQRPVKEEEPKIPGDLDQEPSLLYADLDHLALSRPRRLSTADPADASTIYAVVV

SEQ ID NO: 195 - protein, 476 aa, synthetic construct
MALPVTALLLPLALLLHAARPKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDVFAIILIISLCCMVVFTMAYIMLCRKTSQQEHVYEAARAHAREANDSGETMRVAIFASGCSSDEPTSQNLGNNYSDEPCIGQEYQIIAQINGNYARLLDTVPLDYEFLATEGKSVC

SEQ ID NO: 196 - protein, 474 aa, synthetic construct
MALPVTALLLPLALLLHAARPKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDFGGAGATALVFLYFCIIFVVVRSCRKKSARPAVGVGDTGMEDANAVRGSASQGPLIESPADDSPPHHAPPALATPSPEEGEIQYASLSFHKARPQYPQEQEAIGYEYSEINIPK

SEQ ID NO: 197 - protein, 471 aa, synthetic construct
MALPVTALLLPLALLLHAARPQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDVVTGVSVAFVLLLFLLLFLLLRHRHQSKHRTSAHFYRPAGAAGPEPKDQGLQKRASPVADIQEEILNAAVKDTQPKDGVEMDAPAAASEAPQDVTYAQLHSLTLRREATEPPPSQEREPPAEPSIYAPLAIH

SEQ ID NO: 198 - protein, 467 aa, synthetic construct
MALPVTALLLPLALLLHAARPQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDVAAGATGGLLSALLLAVALLFHCWRRRKSGVGFLGDETRLPPAPGPGESSHSICPAQVELQSLYVDVHPKKGDLVYSEIQTTQLGEEEEANTSRTLLEDKDVSVVYSEVKTQHPDNSAGKISSKDEES

SEQ ID NO: 199 - protein, 474 aa, synthetic construct
MALPVTALLLPLALLLHAARPQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDAVIRYSVAIILFTILPFFLLHRWCSKKKDAAVMNQEPAGHRTVNREDSDEQDPQEVTYAQLDHCIFTQRKITGPSQRSKRPSTDTSVCIELPNAEPRALSPAHEHHSQALMGSSRETTALSQTQLASSNVPAAGI

SEQ ID NO: 200 - protein, 476 aa, synthetic construct
MALPVTALLLPLALLLHAARPQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDLHILIGTSVAIILFIILFFFLLHCCCSNKKNAAVMDQEPAGDRTVNREDSDDQDPQEVTYAQLDHCVFTQTKITSPSQRPKTPPTDTTMYMELPNAKPRSLSPAHKHHSQALRGSSRETTALSQNRVASSHVPAAGI

SEQ ID NO: 201 - protein, 453 aa, synthetic construct
MALPVTALLLPLALLLHAARPQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDGAVGGAGATALVFLSFCVIFIVVRSCRKKSARPAADVGDIGMKDANTIRGSASQGNLTESWADDNPRHHGLAAHSSGEEREIQYAPLSFHKGEPQDLSGQEATNNEYSEIKIPK

SEQ ID NO: 202 - protein, 500 aa, synthetic construct
MALPVTALLLPLALLLHAARPQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDAAGITGLVLSILVLAAAAALLHYARARRKPGGLSATGTSSHSPSECQEPSSSRPSRIDPQEPTHSKPLAPMELEPMYSNVNPGDSNPIYSQIWSIQHTKENSANCPMMHQEHEELTVLYSELKKTHPDDSAGEASSRGRAHEEDDEENYENVPRVLLASDH

SEQ ID NO: 203 - protein, 590 aa, synthetic construct
MALPVTALLLPLALLLHAARPQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDLIVALATVSLLSLVTFTFLSAKCLQGNADGDGGGGQCCRRQDSPSPDFYKQSSPNLQVSSDGTLKYMEVTLRPTDSQSHCYRTCFSPASDGSDFTFLRPLSVQQPTALALEPDAIRSRSNTLRERSQQAPPNTDWRFSQAQRPGTSGSQNGDDTGTWPNNQFDTEMLQAMILASASEAADGSSTLGGGAGTMGLSARYGPQFTLQHVPDYRQNVYIPGSNATLTNAAGKRDGKAPAGGNGNKKKSGKKEKK

SEQ ID NO: 204 - protein, 518 aa, synthetic construct
MALPVTALLLPLALLLHAARPQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDGALIAILACIVILLVIVVLFVTLRRQKKEPLIVFEEEDVRENIITYDDEGGGEEDTEAFDIATLQNPDGINGFIPRKDIKPEYQYMPRPGLRPAPNSVDVDDFINTRIQEADNDPTAPPYDSIQIYGYEGRGSVAGSLSSLESATTDSDLDYDYLQNWGPRFKKLADLYGSKDTFDDDS

SEQ ID NO: 205 - protein, 481 aa, synthetic construct
MALPVTALLLPLALLLHAARPQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDVIIGITIASVVGLLVIFSAIIYFFIRTLQAHHDRSERESPFSGSSRQPDSLSSIENAVKYNPVYESHRAGCEKYEGPYPQHPFYSSASGDVIGGLSREEIRQMYESSELSREEIQERMRVLELYANDPEFAAFVREQQVEEV

SEQ ID NO: 206 - protein, 756 aa, synthetic construct
MALPVTALLLPLALLLHAARPQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGKPIPNPLLGLDSTNGAATTTPAPRPPTPAPTIALQPLSLRPEACRPAAGGAVHTRGLDFACDLVTISCILVGVLLLFVLLLVVRRRRREQRLKRLRDAKSLAEMLMSKNTRTSDTLSKQQQTLRMHIDIPRAQLLIEERDTMETIDDRSTVLLTDADFGEAAKQKSLTVTHTVHYQSVSQATGPLVDVSDARPGTNPTTRRNAKAGPTARNRYASQWTLNRPHPTISAHTLTTDWRLPTPRAAGSVDKESDSYSVSPSQDTDRARSSMVSTESASSTYEELARAYEHAKMEEQLRHAKFTITECFISDTSSEQLTAGTNEYTDSLTSSTPSESGICRFTASPPKPQDGGRVMNMAVPKAHRPGDLIHLPPYLRMDFLLNRGGPGTSRDLSLGQACLEPQKSRTLKRPTVLEPIPMEAASSASSTREGQSWQPGAVATLPQREGAELGQAAKMSSSQESLLDSRGHLKGNNPYAKSYTLV

SEQ ID NO: 207 - DNA, 36 bp, synthetic construct
  source 1..36: mol_type = other DNA
gaaagcaagtacggtccaccttgccctagctgtccg

SEQ ID NO: 208 - DNA, 396 bp, synthetic construct
  source 1..396: mol_type = other DNA
gcctgcccgaccgggctctacactcatagcggggaatgttgtaaggcatgtaacttgggtgagggcgtcgcacagccctgcggagctaaccaaacagtgtgcgaaccctgcctcgatagtgtgacgttctctgatgttgtatcagctacagagccttgcaaaccatgtactgagtgcgttggacttcagtcaatgagcgctccatgtgtggaggcagatgatgcggtctgtcgatgtgcttacggatactaccaagacgagacaacagggcggtgcgaggcctgtagagtttgtgaggcgggctccgggctggtgttttcatgtcaagacaagcaaaatacggtctgtgaagagtgccctgatggcacctactcagacgaagcagatgcagaatgc

SEQ ID NO: 209 - DNA, 102 bp, synthetic construct
  source 1..102: mol_type = other DNA
gcctgccctacaggactctacacgcatagcggtgagtgttgtaaagcatgcaacctcggggaaggtgtagcccagccatgcggggctaaccaaaccgtttgc

SEQ ID NO: 210 - DNA, 60 bp, synthetic construct
  source 1..60: mol_type = other DNA
gctgtgggccaggacacgcaggaggtcatcgtggtgccacactccttgccctttaaggtg

SEQ ID NO: 211 - DNA, 684 bp, synthetic construct
  source 1..684: mol_type = other DNA
gagtctaagtacggccctccttgtcctagctgccctgctcctcctgttgctggcccttccgtgttcctgtttcctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggacagcccagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctccagtgctggacagcgacggctcattcttcctgtacagcagactgaccgtggacaagagcagatggcaagagggcaacgtgttcagctgcagcgtgatgcacgaggccctgcacaaccactacacccagaagtctctgagcctgagcctgggcaaa

SEQ ID NO: 212 - DNA, 357 bp, synthetic construct
  source 1..357: mol_type = other DNA
gagtctaagtacggccctccttgtcctagctgccctggacagcccagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctccagtgctggacagcgacggctcattcttcctgtacagcagactgaccgtggacaagagcagatggcaagagggcaacgtgttcagctgcagcgtgatgcacgaggccctgcacaaccactacacccagaagtctctgagcctgagcctgggcaaa

SEQ ID NO: 213 - protein, 21 aa, synthetic construct
MALPVTALLLPLALLLHAARP

SEQ ID NO: 214 - DNA, 63 bp, synthetic construct
  source 1..63: mol_type = other DNA
atggccttaccagtgaccgccttgctcctgccgctggccttgctgctccacgccgccaggccg

SEQ ID NO: 215 - protein, 21 aa, synthetic construct
KYLLPTAAAGLLLLAAQPAMA

SEQ ID NO: 216 - DNA, 63 bp, synthetic construct
  source 1..63: mol_type = other DNA
aaatacctattgcctacggcagccgctggattgttattactcgcggcccagccggccatggcc

SEQ ID NO: 217 - protein, 255 aa, synthetic construct
QVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLG

SEQ ID NO: 218 - DNA, 765 bp, synthetic construct
  source 1..765: mol_type = other DNA
caggtgcagctggtgcagtctggggcagaggtgaaaaagcccggggagtctctgaagatctcctgtaagggttctggatacagctttaccagctactggatcgcctgggtgcgccagatgcccgggaaaggcctggagtacatggggctcatctatcctggtgactctgacaccaaatacagcccgtccttccaaggccaggtcaccatctcagtcgacaagtccgtcagcactgcctacttgcaatggagcagtctgaagccctcggacagcgccgtgtatttttgtgcgagacatgacgtgggatattgcaccgaccggacttgcgcaaagtggcctgaatacttccagcattggggccagggcaccctggtcaccgtctcctcaggtggaggcggttcaggcggaggtggctctggcggtggcggatcgcagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatctatgatcacaccaatcggcccgcaggggtccctgaccgattctctggctccaagtctggcacctcagcctccctggccatcagtgggttccggtccgaggatgaggctgattattactgtgcctcctgggactacaccctctcgggctgggtgttcggcggagggaccaagctgaccgtcctaggt

SEQ ID NO: 219 - protein, 18 aa, synthetic construct
GKPIPNPLLGLDSTNGAA

SEQ ID NO: 220 - DNA, 54 bp, synthetic construct
  source 1..54: mol_type = other DNA
gggaagcctatcccgaaccctctgttgggtctcgatagtaccaatggggccgca

(19) United States
(12) Patent Application Publication
(10) Pub. No.: US 2025/0339534 A1
(43) Pub. Date: Nov. 6, 2025

(54) INHIBITORY CHIMERIC RECEPTOR ARCHITECTURES

(71) Applicant: Senti Biosciences, Inc., South San Francisco, CA (US)

(72) Inventors: Nicholas Frankel, San Francisco, CA (US); Russell Morrison Gordley, San Francisco, CA (US); Jun Jie Hua, San Mateo, CA (US)

(21) Appl. No.: 19/036,956

(22) Filed: Jan. 24, 2025

Related U.S. Application Data
(63) Continuation of application No. PCT/US2023/069829, filed on Jul. 7, 2023.
(60) Provisional application No. 63/369,480, filed on Jul. 26, 2022.

(51) Int. Cl. A61K 40/42; A61K 40/15; A61K 40/31; A61P 35/00; C07K 14/725; C07K 16/28; C07K 16/32

(52) U.S. Cl. CPC A61K 40/4205; A61K 40/15; A61K 40/31; A61K 40/4203; A61P 35/00; C07K 14/7051; C07K 16/2863; C07K 16/32; A61K 2239/22; C07K 2317/622

(57) ABSTRACT

Provided herein are inhibitory chimeric antigen receptor compositions and cells comprising such compositions. Also provided are methods of using inhibitory chimeric antigen receptors and cells.

### CROSS REFERENCE TO RELATED APPLICATIONS

This application is a continuation of International Application No. PCT/US2023/069829 filed Jul. 7, 2023, which claims the benefit of and priority to U.S. Provisional Application No. 63/369,480 filed Jul. 26, 2022, the entire disclosure of which is hereby incorporated by reference in its entirety for all purposes.

### SEQUENCE LISTING

The instant application contains a Sequence Listing which has been submitted electronically and is hereby incorporated by reference in its entirety. Said XML copy, created on Aug. 9, 2023, is named STB-040WO, and is 239,068 bytes in size.

### BACKGROUND

Chimeric antigen receptors (CARs) enable targeted in vivo activation of immunomodulatory cells, such as T cells. These recombinant membrane receptors have an antigen-binding domain and one or more signaling domains (e.g., T cell activation domains). These special receptors allow the T cells to recognize a specific protein antigen on tumor cells and induce T cell activation and signaling pathways. Recent results of clinical trials with chimeric receptor-expressing T cells have provided compelling support of their utility as agents for cancer immunotherapy. However, despite these promising results, a number of side effects associated the CAR T-cell therapeutics were identified, raising significant safety concerns. One side effect is “on-target but off-tissue” adverse events from TCR and CAR engineered T cells, in which a CAR T cell binds to its ligand outside of the target tumor tissue and induces an immune response. Therefore, the ability to identify appropriate CAR targets is important to effectively targeting and treating the tumor without damaging normal cells that express the same target antigen.

Inhibitory chimeric antigen receptors (also known as iCARs) are protein constructions that inhibit or reduce immunomodulatory cell activity after binding their cognate ligands on a target cell. Current iCAR designs leverage PD-1 intracellular domains for inhibition, but have proven difficult to reproduce. Thus, alternative inhibitory domains for use in iCARs are needed.

### SUMMARY

One embodiment of the disclosure provides a chimeric inhibitory receptor comprising: —an extracellular protein binding domain; —a transmembrane domain, wherein the transmembrane domain is operably linked to the extracellular protein binding domain; and —one or more intracellular signaling domains, wherein the one or more intracellular signaling domains are operably linked to the transmembrane domain, wherein the one or more intracellular signaling domain are each derived from a protein selected from the group consisting of: MPZL1, IRTA1, LIR8, PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZ, FCGR2B, SIGLEC-6, MPIG6B, VSIG4, SIGLEC-12, KIR2DL4, KIR2DL5, SIGLEC-7, FCRH3, PCDHGC5, CDH11, IMPG2, and DSCAM, and wherein at least one of the one or more intracellular signaling domains is capable of preventing, attenuating, or inhibiting activation of a tumor-targeting chimeric receptor expressed on an immunomodulatory cell.

In some embodiments, the transmembrane domain is derived from the same protein as one of the one or more intracellular signaling domains, optionally wherein the transmembrane domain further comprises at least a portion of an extracellular domain of the same protein; or the transmembrane domain is derived from a first protein and the one or more intracellular signaling domains are derived from proteins that are distinct from the first protein.

In some embodiments, one of the one or more intracellular signaling domains is derived from PECAM-1, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to KCYFLRKAKAKQMPVEMSRPAVPLLNSNNEKMSDPNMEANSHYGHNDDVRNHAMKPINDNK EPLNSDVQYTEVQVSSAESHKDLGKKDTETVYSEVRKAVPDAVESRYSRTEGSLDGT (SEQ ID NO: 1), optionally wherein one of the one or more intracellular signaling domains comprises the amino acid sequence of KCYFLRKAKAKQMPVEMSRPAVPLLNSNNEKMSDPNMEANSHYGHNDDVRNHAMKPINDNK EPLNSDVQYTEVQVSSAESHKDLGKKDTETVYSEVRKAVPDAVESRYSRTEGSLDGT (SEQ ID NO: 1); one of the one or more intracellular signaling domains is derived from CD72, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to MAEAITYADLRFVKAPLKKSISSRLGQDPGADDDGEITYENVQVPAVLGVPSSLASSVLGDKAA VKSEQPTASWRAVTSPAVGRILPCRTTCLRY (SEQ ID NO: 2), optionally wherein one of the one or more intracellular signaling domains comprises the amino acid sequence of MAEAITYADLRFVKAPLKKSISSRLGQDPGADDDGEITYENVQVPAVLGVPSSLASSVLGDKAA VKSEQPTASWRAVTSPAVGRILPCRTTCLRY (SEQ ID NO: 2); one of the one or more intracellular signaling domains is derived from IRTA2, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LSRKAGRKPASDPARSPSDSDSQEPTYHNVPAWEELQPVYTNANPRGENVVYSEVRIIQEKKKH AVASDPRHLRNKGSPIIYSEVKVASTPVSGSLFLASSAPHR (SEQ ID NO: 3), optionally wherein one of the one or more intracellular signaling domains comprises the amino acid sequence of LSRKAGRKPASDPARSPSDSDSQEPTYHNVPAWEELQPVYTNANPRGENVVYSEVRIIQEKKKH AVASDPRHLRNKGSPIIYSEVKVASTPVSGSLFLASSAPHR (SEQ ID NO: 3); one of the one or more intracellular signaling domains is derived from IRTA4, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to HKISGESSATNEPRGASRPNPQEFTYSSPTPDMEELQPVYVNVGSVDVDVVYSQVWSMQQPESS ANIRTLLENKDSQVIYSSVKKS (SEQ ID NO: 4), optionally wherein the intracellular signaling domain comprises the amino acid sequence of HKISGESSATNEPRGASRPNPQEFTYSSPTPDMEELQPVYVNVGSVDVDVVYSQVWSMQQPESS ANIRTLLENKDSQVIYSSVKKS (SEQ ID NO: 4) one of the one or more intracellular signaling domains is derived from NKIR, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to WRMMKYQQKAAGMSPEQVLQPLEGDLCYADLTLQLAGTSPQKATTKLSSAQVDQVEVEYVT MASLPKEDISYASLTLGAEDQEPTYCNMGHLSSHLPGRGPEEPTEYSTISRP (SEQ ID NO: 5), optionally wherein one of the one or more intracellular signaling domains comprises the amino acid sequence of WRMMKYQQKAAGMSPEQVLQPLEGDLCYADLTLQLAGTSPQKATTKLSSAQVDQVEVEYVT MASLPKEDISYASLTLGAEDQEPTYCNMGHLSSHLPGRGPEEPTEYSTISRP (SEQ ID NO: 5); one of the one or more intracellular signaling domains is derived from IL1RAP, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to YRAHFGTDETILDGKEYDIYVSYARNAEEEEFVLLTLRGVLENEFGYKLCIFDRDSLPGGIVTDET LSFIQKSRRLLVVLSPNYVLQGTQALLELKAGLENMASRGNINVILVQYKAVKETKVKELKRAK TVLTVIKWKGEKSKYPQGRFWKQLQVAMPVKKSPRRSSSDEQGLSYSSLKNV (SEQ ID NO: 6), optionally wherein one of the one or more intracellular signaling domains comprises the amino acid sequence of YRAHFGTDETILDGKEYDIYVSYARNAEEEEFVLLTLRGVLENEFGYKLCIFDRDSLPGGIVTDET LSFIQKSRRLLVVLSPNYVLOGTQALLELKAGLENMASRGNINVILVQYKAVKETKVKELKRAK TVLTVIKWKGEKSKYPQGRFWKQLQVAMPVKKSPRRSSSDEQGLSYSSLKNV (SEQ ID NO: 6);
one of the one or more intracellular signaling domains is derived from PTPRO, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LRKKHLQMARECGAGTFVNFASLERDGKLPYNWRRSIFAFLTLLPSCLWTDYLLAFYINPWSKN GLKKRKLTNPVQLDDFDAYIKDMAKDSDYKFSLQFEELKLIGLDIPHFAADLPLNRCKNRYTNIL PYDFSRVRLVSMNEEEGADYINANYIPGYNSPQEYIATQGPLPETRNDFWKMVLQQKSQIIVML TQCNEKRRVKCDHYWPFTEEPIAYGDITVEMISEEEQDDWACRHFRINYADEMQDVMHFNYTA WPDHGVPTANAAESILQFVHMVRQQATKSKGPMIIHCSAGVGRTGTFIALDRLLQHIRDHEFVDI LGLVSEMRSYRMSMVQTEEQYIFIHQCVQLMWMKKKQQFCISDVIYENVSKS (SEQ ID NO: 7), optionally wherein one of the one or more intracellular signaling domains comprises the amino acid sequence of LRKKHLQMARECGAGTFVNFASLERDGKLPYNWRRSIFAFLTLLPSCLWTDYLLAFYINPWSKN GLKKRKLTNPVQLDDFDAYIKDMAKDSDYKFSLQFEELKLIGLDIPHFAADLPLNRCKNRYTNIL PYDFSRVRLVSMNEEEGADYINANYIPGYNSPQEYIATQGPLPETRNDFWKMVLQQKSQIIVML TQCNEKRRVKCDHYWPFTEEPIAYGDITVEMISEEEQDDWACRHFRINYADEMQDVMHFNYTA WPDHGVPTANAAESILQFVHMVRQQATKSKGPMIIHCSAGVGRTGTFIALDRLLQHIRDHEFVDI LGLVSEMRSYRMSMVQTEEQYIFIHQCVQLMWMKKKQQFCISDVIYENVSKS (SEQ ID NO: 7);
one of the one or more intracellular signaling domains is derived from PTPRZ1, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RKCFQTAHFYLEDSTSPRVISTPPTPIFPISDDVGAIPIKHFPKHVADLHASSGFTEEFETLKEFYQE VQSCTVDLGITADSSNHPDNKHKNRYINIVAYDHSRVKLAQLAEKDGKLTDYINANYVDGYNR PKAYIAAQGPLKSTAEDFWRMIWEHNVEVIVMITNLVEKGRRKCDQYWPADGSEEYGNFLVTQ KSVQVLAYYTVRNFTLRNTKIKKGSQKGRPSGRVVTQYHYTQWPDMGVPEYSLPVLTFVRKAA YAKRHAVGPVVVHCSAGVGRTGTYIVLDSMLQQIQHEGTVNIFGFLKHIRSQRNYLVQTEEQYV FIHDTLVEAILSKETEVLDSHIHAYVNALLIPGPAGKTKLEKQFQLLSQSNIQQSDYSAALKQCNR EKNRTSSIIPVERSRVGISSLSGEGTDYINASYIMGYYQSNEFIITQHPLLHTIKDFWRMIWDHNAQ LVVMIPDGQNMAEDEFVYWPNKDEPINCESFKVTLMAEEHKCLSNEEKLIIQDFILEATQDDYV LEVRHFQCPKWPNPDSPISKTFELISVIKEEAANRDGPMIVHDEHGGVTAGTFCALTTLMHQLEK ENSVDVYQVAKMINLMRPGVFADIEQYQFLYKVILSLVSTRQEENPSTSLDSNGAALPDGNIAES LESLV (SEQ ID NO: 8), optionally wherein one of the one or more intracellular signaling domains comprises the amino acid sequence of RKCFQTAHFYLEDSTSPRVISTPPTPIFPISDDVGAIPIKHFPKHVADLHASSGFTEEFETLKEFYQE VQSCTVDLGITADSSNHPDNKHKNRYINIVAYDHSRVKLAQLAEKDGKLTDYINANYVDGYNR PKAYIAAQGPLKSTAEDFWRMIWEHNVEVIVMITNLVEKGRRKCDQYWPADGSEEYGNFLVTQ KSVQVLAYYTVRNFTLRNTKIKKGSQKGRPSGRVVTQYHYTQWPDMGVPEYSLPVLTFVRKAA YAKRHAVGPVVVHCSAGVGRTGTYIVLDSMLQQIQHEGTVNIFGFLKHIRSQRNYLVQTEEQYV FIHDTLVEAILSKETEVLDSHIHAYVNALLIPGPAGKTKLEKQFQLLSQSNIQQSDYSAALKQCNR EKNRTSSIIPVERSRVGISSLSGEGTDYINASYIMGYYQSNEFIITQHPLLHTIKDFWRMIWDHNAQ LVVMIPDGQNMAEDEFVYWPNKDEPINCESFKVTLMAEEHKCLSNEEKLIIQDFILEATQDDYV LEVRHFQCPKWPNPDSPISKTFELISVIKEEAANRDGPMIVHDEHGGVTAGTFCALTTLMHQLEK ENSVDVYQVAKMINLMRPGVFADIEQYQFLYKVILSLVSTRQEENPSTSLDSNGAALPDGNIAES LESLV (SEQ ID NO: 8); one of the one or more intracellular signaling domains is derived from TLT1, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to MAKRKQGNRLGVCGRFLSSRVSGMNPSSVVHHVSDSGPAAELPLDVPHIRLDSPPSFDNTTYTS LPLDSPSGKPSLPAPSSLPPLPPKVLVCSKPVTYATVIFPGGNKGGGTSCGPAQNPPNNQTPSS (SEQ ID NO: 9), optionally wherein one of the one or more intracellular signaling domains comprises the amino acid sequence of MAKRKQGNRLGVCGRFLSSRVSGMNPSSVVHHVSDSGPAAELPLDVPHIRLDSPPSFDNTTYTS LPLDSPSGKPSLPAPSSLPPLPPKVLVCSKPVTYATVIFPGGNKGGGTSCGPAQNPPNNQTPSS (SEQ ID NO: 9); one of the one or more intracellular signaling domains is derived from SLAMF1, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to QLRRRGKTNHYQTTVEKKSLTIYAQVQKPGPLQKKLDSFPAQDPCTTIYVAATEPVPESVQETNS ITVYASVTLPES (SEQ ID NO: 10), optionally wherein one of the one or more intracellular signaling domains comprises the amino acid sequence of QLRRRGKTNHYQTTVEKKSLTIYAQVQKPGPLQKKLDSFPAQDPCTTIYVAATEPVPESVQETNS ITVYASVTLPES (SEQ ID NO: 10); one of the one or more intracellular signaling domains is derived from SLAMF5, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RLFKRRQGRIFPEGSCLNTFTKNPYAASKKTIYTYIMASRNTQPAESRIYDEILQSKVLPSKEEPVN TVYSEVQFADKMGKASTQDSKPPGTSSYEIVI (SEQ ID NO: 11), optionally wherein one of the one or more intracellular signaling domains comprises the amino acid sequence of RLFKRRQGRIFPEGSCLNTFTKNPYAASKKTIYTYIMASRNTQPAESRIYDEILQSKVLPSKEEPVN TVYSEVQFADKMGKASTQDSKPPGTSSYEIVI (SEQ ID NO: 11); one of the one or more intracellular signaling domains is derived from PCDHGC3, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to FKVYKWKQSRDLYRAPVSSLYRTPGPSLHADAVRGGLMSPHLYHQVYLTTDSRRSDPLLKKPG AASPLASRQNTLRSCDPVFYRQVLGAESAPPGQQAPPNTDWRFSQAQRPGTSGSQNGDDTGTW PNNQFDTEMLQAMILASASEAADGSSTLGGGAGTMGLSARYGPQFTLQHVPDYRQNVYIPGSN ATLTNAAGKRDGKAPAGGNGNKKKSGKKEKK (SEQ ID NO: 95), optionally wherein one of the one or more intracellular signaling domains comprises the amino acid sequence of FKVYKWKQSRDLYRAPVSSLYRTPGPSLHADAVRGGLMSPHLYHQVYLTTDSRRSDPLLKKPG AASPLASRQNTLRSCDPVFYRQVLGAESAPPGQQAPPNTDWRFSQAQRPGTSGSQNGDDTGTW PNNQFDTEMLQAMILASASEAADGSSTLGGGAGTMGLSARYGPQFTLQHVPDYRQNVYIPGSN ATLTNAAGKRDGKAPAGGNGNKKKSGKKEKK (SEQ ID NO: 95); one of the one or more intracellular signaling domains is derived from LIFR, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to YRKREWIKETFYPDIPNPENCKALQFQKSVCEGSSALKTLEMNPCTPNNVEVLETRSAFPKIEDT EIISPVAERPEDRSDAEPENHVVVSYCPPIIEEEIPNPAADEAGGTAQVIYIDVQSMYQPQAKPEEE QENDPVGGAGYKPQMHLPINSTVEDIAAEEDLDKTAGYRPQANVNTWNLVSPDSPRSIDSNSEI VSFGSPCSINSRQFLIPPKDEDSPKSNGGGWSFTNFFQNKPND (SEQ ID NO: 96), optionally wherein one of the one or more intracellular signaling domains comprises the amino acid sequence of YRKREWIKETFYPDIPNPENCKALQFQKSVCEGSSALKTLEMNPCTPNNVEVLETRSAFPKIEDT EIISPVAERPEDRSDAEPENHVVVSYCPPIIEEEIPNPAADEAGGTAQVIYIDVQSMYQPQAKPEEE QENDPVGGAGYKPQMHLPINSTVEDIAAEEDLDKTAGYRPQANVNTWNLVSPDSPRSIDSNSEI VSFGSPCSINSRQFLIPPKDEDSPKSNGGGWSFTNFFQNKPND (SEQ ID NO: 96); one of the one or more intracellular signaling domains is derived from ERMAP, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to WKQRRAKEKLLYEHVTEVDNLLSDHAKEKGKLHKAVKKLRSELKLKRAAANSGWRRARLHF VAVTLDPDTAHPKLILSEDQRCVRLGDRRQPVPDNPQRFDFVVSILGSEYFTTGCHYWEVYVGD KTKWILGVCSESVSRKGKVTASPANGHWLLRQSRGNEYEALTSPQTSFRLKEPPRCVGIFLDYE AGVISFYNVTNKSHIFTFTHNFSGPLRPFFEPCLHDGGKNTAPLVICSELHKSEESIVPRPEGKGHA NGDVSLKVNSSLLPPKAPELKDIILSLPPDLGPALQELKAPSF (SEQ ID NO: 97), optionally wherein one of the one or more intracellular signaling domains comprises the amino acid sequence of WKQRRAKEKLLYEHVTEVDNLLSDHAKEKGKLHKAVKKLRSELKLKRAAANSGWRRARLHF VAVTLDPDTAHPKLILSEDQRCVRLGDRRQPVPDNPQRFDFVVSILGSEYFTTGCHYWEVYVGD KTKWILGVCSESVSRKGKVTASPANGHWLLRQSRGNEYEALTSPQTSFRLKEPPRCVGIFLDYE AGVISFYNVTNKSHIFTFTHNFSGPLRPFFEPCLHDGGKNTAPLVICSELHKSEESIVPRPEGKGHA NGDVSLKVNSSLLPPKAPELKDIILSLPPDLGPALQELKAPSF (SEQ ID NO: 97); one of the one or more intracellular signaling domains is derived from IL1RAPL2, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to KCYNIELMLFYRQHFGADETNDDNKEYDAYLSYTKVDQDTLDCDNPEEEQFALEVLPDVLEKH YGYKLFIPERDLIPSGTYMEDLTRYVEQSRRLIIVLTPDYILRRGWSIFELESRLHNMLVSGEIKVI LIECTELKGKVNCQEVESLKRSIKLLSLIKWKGSKSSKLNSKFWKHLVYEMPIKKKEMLPRCHVL DSAEQGLFGELQPIPSIAMTSTSATLVSSQADLPEFHPSDSMQIRHCCRGYKHEIPATTLPVPSLGN HHTYCNLPLTLLNGQLPLNNTLKDTQEFHRNSSLLPLSSKELSFTSDIW (SEQ ID NO: 98), optionally wherein one of the one or more intracellular signaling domains comprises the amino acid sequence of KCYNIELMLFYRQHFGADETNDDNKEYDAYLSYTKVDQDTLDCDNPEEEQFALEVLPDVLEKH YGYKLFIPERDLIPSGTYMEDLTRYVEQSRRLIIVLTPDYILRRGWSIFELESRLHNMLVSGEIKVI LIECTELKGKVNCQEVESLKRSIKLLSLIKWKGSKSSKLNSKFWKHLVYEMPIKKKEMLPRCHVL DSAEQGLFGELQPIPSIAMTSTSATLVSSQADLPEFHPSDSMQIRHCCRGYKHEIPATTLPVPSLGN HHTYCNLPLTLLNGQLPLNNTLKDTQEFHRNSSLLPLSSKELSFTSDIW (SEQ ID NO: 98); one of the one or more intracellular signaling domains is derived from CDH5, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RRRLRKQARAHGKSVPEIHEQLVTYDEEGGGEMDTTSYDVSVLNSVRRGGAKPPRPALDARPS LYAQVQKPPRHAPGAHGGPGEMAAMIEVKKDEADHDGDGPPYDTLHIYGYEGSESIAESLSSLG TDSSDSDVDYDFLNDWGPRFKMLAELYGSDPREELLY (SEQ ID NO: 99), optionally wherein one of the one or more intracellular signaling domains comprises the amino acid sequence of RRRLRKQARAHGKSVPEIHEQLVTYDEEGGGEMDTTSYDVSVLNSVRRGGAKPPRPALDARPS LYAQVQKPPRHAPGAHGGPGEMAAMIEVKKDEADHDGDGPPYDTLHIYGYEGSESIAESLSSLG TDSSDSDVDYDFLNDWGPRFKMLAELYGSDPREELLY (SEQ ID NO: 99); one of the one or more intracellular signaling domains is derived from MPZL1, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SMILAVLYRRKNSKRDYTGCSTSESLSPVKQAPRKSPSDTEGLVKSLPSGSHQGPVIYAQLDHSG GHHSDKINKSESVVYADIRKN (SEQ ID NO: 100), optionally wherein one of the one or more intracellular signaling domains comprises the amino acid sequence of SMILAVLYRRKNSKRDYTGCSTSESLSPVKQAPRKSPSDTEGLVKSLPSGSHQGPVIYAQLDHSG GHHSDKINKSESVVYADIRKN (SEQ ID NO: 100); one of the one or more intracellular signaling domains is derived from MPZ, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RYCWLRRQAALQRRLSAMEKGKLHKPGKDASKRGRQTPVLYAMLDHSRSTKAVSEKKAKGL GESRKDKK (SEQ ID NO: 101), optionally wherein one of the one or more intracellular signaling domains comprises the amino acid sequence of RYCWLRRQAALQRRLSAMEKGKLHKPGKDASKRGRQTPVLYAMLDHSRSTKAVSEKKAKGL GESRKDKK (SEQ ID NO: 101); one of the one or more intracellular signaling domains is derived from FCGR2B, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VVALIYCRKKRISALPGYPECREMGETLPEKPANPTNPDEADKVGAENTITYSLLMHPDALEEPD DQNRI (SEQ ID NO: 102), optionally wherein one of the one or more intracellular signaling domains comprises the amino acid sequence of VVALIYCRKKRISALPGYPECREMGETLPEKPANPTNPDEADKVGAENTITYSLLMHPDALEEPD DQNRI (SEQ ID NO: 102); one of the one or more intracellular signaling domains is derived from SIGLEC-6, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RVKTRRKKAAQPVQNTDDVNPVMVSGSRGHQHQFQTGIVSDHPAEAGPISEDEQELHYAVLHF HKVQPQEPKVTDTEYSEIKIHK (SEQ ID NO: 103), optionally wherein one of the one or more intracellular signaling domains comprises the amino acid sequence of RVKTRRKKAAQPVQNTDDVNPVMVSGSRGHQHQFQTGIVSDHPAEAGPISEDEQELHYAVLHF HKVQPQEPKVTDTEYSEIKIHK (SEQ ID NO: 103); one of the one or more intracellular signaling domains is derived from MPIG6B, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to WLHRRLPPQPIRPLPRFAPLVKTEPQRPVKEEEPKIPGDLDQEPSLLYADLDHLALSRPRRLSTAD PADASTIYAVVV (SEQ ID NO: 104), optionally wherein one of the one or more intracellular signaling domains comprises the amino acid sequence of WLHRRLPPQPIRPLPRFAPLVKTEPQRPVKEEEPKIPGDLDQEPSLLYADLDHLALSRPRRLSTAD PADASTIYAVVV (SEQ ID NO: 104); one of the one or more intracellular signaling domains is derived from VSIG4, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to MLCRKTSQQEHVYEAARAHAREANDSGETMRVAIFASGCSSDEPTSQNLGNNYSDEPCIGQEY QIIAQINGNYARLLDTVPLDYEFLATEGKSVC (SEQ ID NO: 105), optionally wherein one of the one or more intracellular signaling domains comprises the amino acid sequence of MLCRKTSQQEHVYEAARAHAREANDSGETMRVAIFASGCSSDEPTSQNLGNNYSDEPCIGQEY QIIAQINGNYARLLDTVPLDYEFLATEGKSVC (SEQ ID NO: 105); one of the one or more intracellular signaling domains is derived from SIGLEC-12, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RSCRKKSARPAVGVGDTGMEDANAVRGSASQGPLIESPADDSPPHHAPPALATPSPEEGEIQYAS LSFHKARPQYPQEQEAIGYEYSEINIPK (SEQ ID NO: 106), optionally wherein one of the one or more intracellular signaling domains comprises the amino acid sequence of RSCRKKSARPAVGVGDTGMEDANAVRGSASQGPLIESPADDSPPHHAPPALATPSPEEGEIQYAS LSFHKARPQYPQEQEAIGYEYSEINIPK (SEQ ID NO: 106); one of the one or more intracellular signaling domains is derived from LIR8, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RHRHQSKHRTSAHFYRPAGAAGPEPKDQGLQKRASPVADIQEEILNAAVKDTQPKDGVEMDAP AAASEAPQDVTYAQLHSLTLRREATEPPPSQEREPPAEPSIYAPLAIH (SEQ ID NO: 107), optionally wherein one of the one or more intracellular signaling domains comprises the amino acid sequence of RHRHQSKHRTSAHFYRPAGAAGPEPKDQGLQKRASPVADIQEEILNAAVKDTQPKDGVEMDAP AAASEAPQDVTYAQLHSLTLRREATEPPPSQEREPPAEPSIYAPLAIH (SEQ ID NO: 107); of the one or more intracellular signaling domains is derived from IRTA1, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to HCWRRRKSGVGFLGDETRLPPAPGPGESSHSICPAQVELQSLYVDVHPKKGDLVYSEIQTTQLGE EEEANTSRTLLEDKDVSVVYSEVKTQHPDNSAGKISSKDEES (SEQ ID NO: 108), optionally wherein one of the one or more intracellular signaling domains comprises the amino acid sequence of HCWRRRKSGVGFLGDETRLPPAPGPGESSHSICPAQVELQSLYVDVHPKKGDLVYSEIQTTQLGE EEEANTSRTLLEDKDVSVVYSEVKTQHPDNSAGKISSKDEES (SEQ ID NO: 108); one of the one or more intracellular signaling domains is derived from KIR2DL4, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RWCSKKKDAAVMNQEPAGHRTVNREDSDEQDPQEVTYAQLDHCIFTQRKITGPSQRSKRPSTD TSVCIELPNAEPRALSPAHEHHSQALMGSSRETTALSQTQLASSNVPAAGI (SEQ ID NO: 109), optionally wherein one of the one or more intracellular signaling domains comprises the amino acid sequence of RWCSKKKDAAVMNQEPAGHRTVNREDSDEQDPQEVTYAQLDHCIFTQRKITGPSQRSKRPSTD TSVCIELPNAEPRALSPAHEHHSQALMGSSRETTALSQTQLASSNVPAAGI (SEQ ID NO: 109);
one of the one or more intracellular signaling domains is derived from KIR2DL5, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LHCCCSNKKNAAVMDQEPAGDRTVNREDSDDQDPQEVTYAQLDHCVFTQTKITSPSQRPKTPP TDTTMYMELPNAKPRSLSPAHKHHSQALRGSSRETTALSQNRVASSHVPAAGI (SEQ ID NO: 110), optionally wherein one of the one or more intracellular signaling domains comprises the amino acid sequence of LHCCCSNKKNAAVMDQEPAGDRTVNREDSDDQDPQEVTYAQLDHCVFTQTKITSPSQRPKTPP TDTTMYMELPNAKPRSLSPAHKHHSQALRGSSRETTALSQNRVASSHVPAAGI (SEQ ID NO: 110); one of the one or more intracellular signaling domains is derived from SIGLEC-7, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RSCRKKSARPAADVGDIGMKDANTIRGSASQGNLTESWADDNPRHHGLAAHSSGEEREIQYAP LSFHKGEPQDLSGQEATNNEYSEIKIPK (SEQ ID NO: 111), optionally wherein one of the one or more intracellular signaling domains comprises the amino acid sequence of RSCRKKSARPAADVGDIGMKDANTIRGSASQGNLTESWADDNPRHHGLAAHSSGEEREIQYAP LSFHKGEPQDLSGQEATNNEYSEIKIPK (SEQ ID NO: 111);

one of the one or more intracellular signaling domains is derived from FCRH3, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to HYARARRKPGGLSATGTSSHSPSECQEPSSSRPSRIDPQEPTHSKPLAPMELEPMYSNVNPGDSNP IYSQIWSIQHTKENSANCPMMHQEHEELTVLYSELKKTHPDDSAGEASSRGRAHEEDDEENYEN VPRVLLASDH (SEQ ID NO: 112), optionally wherein one of the one or more intracellular signaling domains comprises the amino acid sequence of HYARARRKPGGLSATGTSSHSPSECQEPSSSRPSRIDPQEPTHSKPLAPMELEPMYSNVNPGDSNP IYSQIWSIQHTKENSANCPMMHQEHEELTVLYSELKKTHPDDSAGEASSRGRAHEEDDEENYEN VPRVLLASDH (SEQ ID NO: 112); one of the one or more intracellular signaling domains is derived from PCDHGC5, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to KCLQGNADGDGGGGQCCRRQDSPSPDFYKQSSPNLQVSSDGTLKYMEVTLRPTDSQSHCYRTC FSPASDGSDFTFLRPLSVQQPTALALEPDAIRSRSNTLRERSQQAPPNTDWRFSQAQRPGTSGSQN GDDTGTWPNNQFDTEMLQAMILASASEAADGSSTLGGGAGTMGLSARYGPQFTLQHVPDYRQ NVYIPGSNATLTNAAGKRDGKAPAGGNGNKKKSGKKEKK (SEQ ID NO: 113), optionally wherein one of the one or more intracellular signaling domains comprises the amino acid sequence of KCLQGNADGDGGGGQCCRRQDSPSPDFYKQSSPNLQVSSDGTLKYMEVTLRPTDSQSHCYRTC FSPASDGSDFTFLRPLSVQQPTALALEPDAIRSRSNTLRERSQQAPPNTDWRFSQAQRPGTSGSQN GDDTGTWPNNQFDTEMLQAMILASASEAADGSSTLGGGAGTMGLSARYGPQFTLQHVPDYRQ NVYIPGSNATLTNAAGKRDGKAPAGGNGNKKKSGKKEKK (SEQ ID NO: 113); one of the one or more intracellular signaling domains is derived from CDH11, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RRQKKEPLIVFEEEDVRENIITYDDEGGGEEDTEAFDIATLONPDGINGFIPRKDIKPEYQYMPRP GLRPAPNSVDVDDFINTRIQEADNDPTAPPYDSIQIYGYEGRGSVAGSLSSLESATTDSDLDYDYL QNWGPRFKKLADLYGSKDTFDDDS (SEQ ID NO: 114), optionally wherein one of the one or more intracellular signaling domains comprises the amino acid sequence of RRQKKEPLIVFEEEDVRENIITYDDEGGGEEDTEAFDIATLONPDGINGFIPRKDIKPEYQYMPRP GLRPAPNSVDVDDFINTRIQEADNDPTAPPYDSIQIYGYEGRGSVAGSLSSLESATTDSDLDYDYL QNWGPRFKKLADLYGSKDTFDDDS (SEQ ID NO: 114); one of the one or more intracellular signaling domains is derived from IMPG2, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to YFFIRTLQAHHDRSERESPFSGSSRQPDSLSSIENAVKYNPVYESHRAGCEKYEGPYPQHPFYSSA SGDVIGGLSREEIRQMYESSELSREEIQERMRVLELYANDPEFAAFVREQQVEEV (SEQ ID NO: 115), optionally wherein one of the one or more intracellular signaling domains comprises the amino acid sequence of YFFIRTLQAHHDRSERESPFSGSSRQPDSLSSIENAVKYNPVYESHRAGCEKYEGPYPQHPFYSSA SGDVIGGLSREEIRQMYESSELSREEIQERMRVLELYANDPEFAAFVREQQVEEV (SEQ ID NO: 115); one of the one or more intracellular signaling domains is derived from DSCAM, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RRRRREQRLKRLRDAKSLAEMLMSKNTRTSDTLSKQQQTLRMHIDIPRAQLLIEERDTMETIDD RSTVLLTDADEGEAAKQKSLTVTHTVHYQSVSQATGPLVDVSDARPGTNPTTRRNAKAGPTAR NRYASQWTLNRPHPTISAHTLTTDWRLPTPRAAGSVDKESDSYSVSPSQDTDRARSSMVSTESA SSTYEELARAYEHAKMEEQLRHAKFTITECFISDTSSEQLTAGTNEYTDSLTSSTPSESGICRFTAS PPKPQDGGRVMNMAVPKAHRPGDLIHLPPYLRMDFLLNRGGPGTSRDLSLGQACLEPQKSRTL KRPTVLEPIPMEAASSASSTREGQSWQPGAVATLPQREGAELGQAAKMSSSQESLLDSRGHLKG NNPYAKSYTLV (SEQ ID NO: 116), optionally wherein one of the one or more intracellular signaling domains comprises the amino acid sequence of RRRRREQRLKRLRDAKSLAEMLMSKNTRTSDTLSKQQQTLRMHIDIPRAQLLIEERDTMETIDD RSTVLLTDADEGEAAKQKSLTVTHTVHYQSVSQATGPLVDVSDARPGTNPTTRRNAKAGPTAR NRYASQWTLNRPHPTISAHTLTTDWRLPTPRAAGSVDKESDSYSVSPSQDTDRARSSMVSTESA SSTYEELARAYEHAKMEEQLRHAKFTITECFISDTSSEQLTAGTNEYTDSLTSSTPSESGICRFTAS PPKPQDGGRVMNMAVPKAHRPGDLIHLPPYLRMDFLLNRGGPGTSRDLSLGQACLEPQKSRTL KRPTVLEPIPMEAASSASSTREGQSWQPGAVATLPQREGAELGQAAKMSSSQESLLDSRGHLKG NNPYAKSYTLV (SEQ ID NO: 116).

Another embodiment of the disclosure provides a chimeric inhibitory receptor comprising: —an extracellular protein binding domain; —a transmembrane domain, wherein the transmembrane domain is operably linked to the extracellular protein binding domain; and —one or more intracellular signaling domains, wherein the one or more intracellular signaling domains are operably linked to the transmembrane domain, wherein at least one of the one or more intracellular signaling domains comprises: at least one immunoreceptor tyrosine-based switch motif (ITSM); or at least one ITIM and at least one phosphatase, and wherein at least one of the one or more intracellular signaling domains is capable of preventing, attenuating, or inhibiting activation of a tumor-targeting chimeric receptor expressed on an immunomodulatory cell.

In some embodiments, the chimeric inhibitory receptor comprises at least one ITIM and at least one phosphatase, optionally wherein the phosphatase is a protein tyrosine phosphatase (PTP), optionally wherein the one or more intracellular signaling domains are each derived from a protein selected from the group consisting of: PTPRO and PTPRZ1, optionally wherein: one of the one or more intracellular signaling domains is derived from PTPRO, optionally wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LRKKHLQMARECGAGTFVNFASLERDGKLPYNWRRSIFAFLTLLPSCLWTDYLLAFYINPWSKN GLKKRKLTNPVQLDDFDAYIKDMAKDSDYKFSLQFEELKLIGLDIPHFAADLPLNRCKNRYTNIL PYDFSRVRLVSMNEEEGADYINANYIPGYNSPQEYIATQGPLPETRNDFWKMVLQQKSQIIVML TQCNEKRRVKCDHYWPFTEEPIAYGDITVEMISEEEQDDWACRHFRINYADEMQDVMHFNYTA WPDHGVPTANAAESILQFVHMVRQQATKSKGPMIIHCSAGVGRTGTFIALDRLLQHIRDHEFVDI LGLVSEMRSYRMSMVQTEEQYIFIHQCVQLMWMKKKQQFCISDVIYENVSKS (SEQ ID NO: 7), optionally wherein the intracellular signaling domain comprises the amino acid sequence of LRKKHLQMARECGAGTFVNFASLERDGKLPYNWRRSIFAFLTLLPSCLWTDYLLAFYINPWSKN GLKKRKLTNPVQLDDFDAYIKDMAKDSDYKFSLQFEELKLIGLDIPHFAADLPLNRCKNRYTNIL PYDFSRVRLVSMNEEEGADYINANYIPGYNSPQEYIATQGPLPETRNDFWKMVLQQKSQIIVML TQCNEKRRVKCDHYWPFTEEPIAYGDITVEMISEEEQDDWACRHFRINYADEMQDVMHFNYTA WPDHGVPTANAAESILQFVHMVRQQATKSKGPMIIHCSAGVGRTGTFIALDRLLQHIRDHEFVDI LGLVSEMRSYRMSMVQTEEQYIFIHQCVQLMWMKKKQQFCISDVIYENVSKS (SEQ ID NO: 7);
or one of the one or more intracellular signaling domains is derived from PTPRZ1, optionally wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RKCFQTAHFYLEDSTSPRVISTPPTPIFPISDDVGAIPIKHFPKHVADLHASSGFTEEFETLKEFYQE VQSCTVDLGITADSSNHPDNKHKNRYINIVAYDHSRVKLAQLAEKDGKLTDYINANYVDGYNR PKAYIAAQGPLKSTAEDFWRMIWEHNVEVIVMITNLVEKGRRKCDQYWPADGSEEYGNFLVTQ KSVQVLAYYTVRNFTLRNTKIKKGSQKGRPSGRVVTQYHYTQWPDMGVPEYSLPVLTFVRKAA YAKRHAVGPVVVHCSAGVGRTGTYIVLDSMLQQIQHEGTVNIFGFLKHIRSQRNYLVQTEEQYV FIHDTLVEAILSKETEVLDSHIHAYVNALLIPGPAGKTKLEKQFQLLSQSNIQQSDYSAALKQCNR EKNRTSSIIPVERSRVGISSLSGEGTDYINASYIMGYYQSNEFIITQHPLLHTIKDFWRMIWDHNAQ LVVMIPDGQNMAEDEFVYWPNKDEPINCESFKVTLMAEEHKCLSNEEKLIIQDFILEATQDDYV LEVRHFQCPKWPNPDSPISKTFELISVIKEEAANRDGPMIVHDEHGGVTAGTFCALTTLMHQLEK ENSVDVYQVAKMINLMRPGVFADIEQYQFLYKVILSLVSTRQEENPSTSLDSNGAALPDGNIAES LESLV (SEQ ID NO: 8, optionally wherein the intracellular signaling domain comprises the amino acid sequence of RKCFQTAHFYLEDSTSPRVISTPPTPIFPISDDVGAIPIKHFPKHVADLHASSGFTEEFETLKEFYQE VQSCTVDLGITADSSNHPDNKHKNRYINIVAYDHSRVKLAQLAEKDGKLTDYINANYVDGYNR PKAYIAAQGPLKSTAEDFWRMIWEHNVEVIVMITNLVEKGRRKCDQYWPADGSEEYGNFLVTQ KSVQVLAYYTVRNFTLRNTKIKKGSQKGRPSGRVVTQYHYTQWPDMGVPEYSLPVLTFVRKAA YAKRHAVGPVVVHCSAGVGRTGTYIVLDSMLQQIQHEGTVNIFGFLKHIRSQRNYLVQTEEQYV FIHDTLVEAILSKETEVLDSHIHAYVNALLIPGPAGKTKLEKQFQLLSQSNIQQSDYSAALKQCNR EKNRTSSIIPVERSRVGISSLSGEGTDYINASYIMGYYQSNEFIITQHPLLHTIKDFWRMIWDHNAQ LVVMIPDGQNMAEDEFVYWPNKDEPINCESFKVTLMAEEHKCLSNEEKLIIQDFILEATQDDYV LEVRHFQCPKWPNPDSPISKTFELISVIKEEAANRDGPMIVHDEHGGVTAGTFCALTTLMHQLEK ENSVDVYQVAKMINLMRPGVFADIEQYQFLYKVILSLVSTRQEENPSTSLDSNGAALPDGNIAES LESLV (SEQ ID NO: 8).

In some embodiments, the chimeric inhibitory receptor comprises at least one ITSM, optionally wherein the one or more intracellular signaling domains are each derived from a protein selected from the group consisting of: SLAMF1 and SLAMF5, optionally wherein: one of the one or more intracellular signaling domains is derived from SLAMF1, optionally wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to QLRRRGKTNHYQTTVEKKSLTIYAQVQKPGPLQKKLDSFPAQDPCTTIYVAATEPVPESVQETNS ITVYASVTLPES (SEQ ID NO: 10), optionally wherein the intracellular signaling domain comprises the amino acid sequence of QLRRRGKTNHYQTTVEKKSLTIYAQVQKPGPLQKKLDSFPAQDPCTTIYVAATEPVPESVQETNS ITVYASVTLPES (SEQ ID NO: 10); or one of the one or more intracellular signaling domains is derived from SLAMF5, optionally wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RLFKRRQGRIFPEGSCLNTFTKNPYAASKKTIYTYIMASRNTQPAESRIYDEILQSKVLPSKEEPVN TVYSEVQFADKMGKASTQDSKPPGTSSYEIVI (SEQ ID NO: 11), optionally wherein the intracellular signaling domain comprises the amino acid sequence of RLFKRRQGRIFPEGSCLNTFTKNPYAASKKTIYTYIMASRNTQPAESRIYDEILQSKVLPSKEEPVN TVYSEVQFADKMGKASTQDSKPPGTSSYEIVI (SEQ ID NO: 11).

In some embodiments, the transmembrane domain is derived from a protein selected from the group consisting of: CD8, CD28, CD3ζ, CD4, 4-IBB, OX40, ICOS, 2B4, CD25, CD7, LAX, LAT, LIR1, PCAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM, optionally wherein: the chimeric inhibitory receptor comprises a transmembrane domain derived from PECAM-1, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to GLIAVVIIGVIIALLIIAA (SEQ ID NO: 24), optionally wherein the transmembrane domain comprises the amino acid sequence of GLIAVVIIGVIIALLIIAA (SEQ ID NO: 24); the chimeric inhibitory receptor comprises a transmembrane domain derived from LIR1, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VIGILVAVILLLLLLLLLFLI (SEQ ID NO: 25), optionally wherein the transmembrane domain comprises the amino acid sequence of VIGILVAVILLLLLLLLLFLI (SEQ ID NO: 25); the chimeric inhibitory receptor comprises a transmembrane domain derived from IRTA2, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VAGGLLSIAGLAAGALLLYCW (SEQ ID NO: 26), optionally wherein the transmembrane domain comprises the amino acid sequence of VAGGLLSIAGLAAGALLLYCW (SEQ ID NO: 26); the chimeric inhibitory receptor comprises a transmembrane domain derived from IRTA4, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LWGLFGVLGFTGVALLLYALF (SEQ ID NO: 27), optionally wherein the transmembrane domain comprises the amino acid sequence of LWGLFGVLGFTGVALLLYALF (SEQ ID NO: 27); the chimeric inhibitory receptor comprises a transmembrane domain derived from NKIR, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VLLPLIFTILLLLLVAASLLA (SEQ ID NO: 28), optionally wherein the transmembrane domain comprises the amino acid sequence of VLLPLIFTILLLLLVAASLLA (SEQ ID NO: 28); the chimeric inhibitory receptor comprises a transmembrane domain derived from IL1RAP, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VLLVVILIVVYHVYWLEMVLF (SEQ ID NO: 29), optionally wherein the transmembrane domain comprises the amino acid sequence of VLLVVILIVVYHVYWLEMVLF (SEQ ID NO: 29); the chimeric inhibitory receptor comprises a transmembrane domain derived from PTPRO, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to ISVLAILSTLLIGLLLVTLII (SEQ ID NO: 30), optionally wherein the transmembrane domain comprises the amino acid sequence of ISVLAILSTLLIGLLLVTLII (SEQ ID NO: 30); the chimeric inhibitory receptor comprises a transmembrane domain derived from PTPRZ1, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AVIPLVIVSALTFICLVVLVGILIYW (SEQ ID NO: 31), optionally wherein the transmembrane domain comprises the amino acid sequence of AVIPLVIVSALTFICLVVLVGILIYW (SEQ ID NO: 31); the chimeric inhibitory receptor comprises a transmembrane domain derived from TLT1, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LIWGAVLLVGLLVAAVVLFAV (SEQ ID NO: 32), optionally wherein the transmembrane domain comprises the amino acid sequence of LIWGAVLLVGLLVAAVVLFAV (SEQ ID NO: 32); the chimeric inhibitory receptor comprises a transmembrane domain derived from SLAMF1, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to WAVYAGLLGGVIMILIMVVIL (SEQ ID NO: 33), optionally wherein the transmembrane domain comprises the amino acid sequence of WAVYAGLLGGVIMILIMVVIL (SEQ ID NO: 33); the chimeric inhibitory receptor comprises a transmembrane domain derived from SLAMF5, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LLSVLAMFFLLVLILSSVFLF (SEQ ID NO: 34), optionally wherein the transmembrane domain comprises the amino acid sequence of LLSVLAMFFLLVLILSSVFLF (SEQ ID NO: 34); the chimeric inhibitory receptor comprises a transmembrane domain derived from PCDHGC3, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LLLSLILVSVGFVVTVFGVII (SEQ ID NO: 139), optionally wherein the transmembrane domain comprises the amino acid sequence of LLLSLILVSVGFVVTVFGVII (SEQ ID NO: 139); the chimeric inhibitory receptor comprises a transmembrane domain derived from LIFR, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VGLIIAILIPVAVAVIVGVVTSILC (SEQ ID NO: 140), optionally wherein the transmembrane domain comprises the amino acid sequence of VGLIIAILIPVAVAVIVGVVTSILC (SEQ ID NO: 140); the chimeric inhibitory receptor comprises a transmembrane domain derived from ERMAP, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VALAVILPVLVLLIMVCLCLI (SEQ ID NO: 141), optionally wherein the transmembrane domain comprises the amino acid sequence of VALAVILPVLVLLIMVCLCLI (SEQ ID NO: 141); the chimeric inhibitory receptor comprises a transmembrane domain derived from IL1RAPL2, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to IELAGGLGAIFLLLVLLVVIY (SEQ ID NO: 142), optionally wherein the transmembrane domain comprises the amino acid sequence of IELAGGLGAIFLLLVLLVVIY (SEQ ID NO: 142); the chimeric inhibitory receptor comprises a transmembrane domain derived from CDH5, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AVVAILLCILTITVITLLIFL (SEQ ID NO: 143), optionally wherein the transmembrane domain comprises the amino acid sequence of AVVAILLCILTITVITLLIFL (SEQ ID NO: 143); the chimeric inhibitory receptor comprises a transmembrane domain derived from MPZL1, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to FPVWVVVGIVTAVVLGLTLLI (SEQ ID NO: 144), optionally wherein the transmembrane domain comprises the amino acid sequence of FPVWVVVGIVTAVVLGLTLLI (SEQ ID NO: 144); the chimeric inhibitory receptor comprises a transmembrane domain derived from MPZ, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to YGVVLGAVIGGVLGVVLLLLLLFYVV (SEQ ID NO: 145), optionally wherein the transmembrane domain comprises the amino acid sequence of YGVVLGAVIGGVLGVVLLLLLLFYVV (SEQ ID NO: 145); the chimeric inhibitory receptor comprises a transmembrane domain derived from FCGR2B, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SSSPMGIIVAVVTGIAVAAIVAA (SEQ ID NO: 146), optionally wherein the transmembrane domain comprises the amino acid sequence of SSSPMGIIVAVVTGIAVAAIVAA (SEQ ID NO: 146); the chimeric inhibitory receptor comprises a transmembrane domain derived from SIGLEC-6, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LGAVWGASITTLVFLCVCFIF (SEQ ID NO: 147), optionally wherein the transmembrane domain comprises the amino acid sequence of LGA VWGASITTLVFLCVCFIF (SEQ ID NO: 147); the chimeric inhibitory receptor comprises a transmembrane domain derived from MPIG6B, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LLIPLLGAGLVLGLGALGLVW (SEQ ID NO: 148), optionally wherein the transmembrane domain comprises the amino acid sequence of LLIPLLGAGLVLGLGALGLVW (SEQ ID NO: 148); the chimeric inhibitory receptor comprises a transmembrane domain derived from VSIG4, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VFAIILIISLCCMVVFTMAYI (SEQ ID NO: 149), optionally wherein the transmembrane domain comprises the amino acid sequence of VFAIILIISLCCMVVETMAYI (SEQ ID NO: 149); the chimeric inhibitory receptor comprises a transmembrane domain derived from SIGLEC-12, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to FGGAGATALVFLYFCIIFVVV (SEQ ID NO: 150), optionally wherein the transmembrane domain comprises the amino acid sequence of FGGAGATALVFLYFCIIFVVV (SEQ ID NO: 150); the chimeric inhibitory receptor comprises a transmembrane domain derived from LIR8, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VVTGVSVAFVLLLFLLLFLLL (SEQ ID NO: 151), optionally wherein the transmembrane domain comprises the amino acid sequence of VVTGVSVAFVLLLFLLLFLLL (SEQ ID NO: 151); the chimeric inhibitory receptor comprises a transmembrane domain derived from IRTA1, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VAAGATGGLLSALLLAVALLF (SEQ ID NO: 152), optionally wherein the transmembrane domain comprises the amino acid sequence of VAAGATGGLLSALLLAVALLF (SEQ ID NO: 152); the chimeric inhibitory receptor comprises a transmembrane domain derived from KIR2DL4, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AVIRYSVAIILFTILPFFLLH (SEQ ID NO: 153), optionally wherein the transmembrane domain comprises the amino acid sequence of AVIRYSVAIILFTILPFFLLH (SEQ ID NO: 153); the chimeric inhibitory receptor comprises a transmembrane domain derived from KIR2DL5, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LHILIGTSVAIILFIILFFFL (SEQ ID NO: 154), optionally wherein the transmembrane domain comprises the amino acid sequence of LHILIGTSVAIILFIILFFFL (SEQ ID NO: 154); the chimeric inhibitory receptor comprises a transmembrane domain derived from SIGLEC-7, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to GAVGGAGATALVFLSFCVIFIVV (SEQ ID NO: 155), optionally wherein the transmembrane domain comprises the amino acid sequence of GAVGGAGATALVFLSFCVIFIVV (SEQ ID NO: 155); the chimeric inhibitory receptor comprises a transmembrane domain derived from FCRH3, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AAGITGLVLSILVLAAAAALL (SEQ ID NO: 156), optionally wherein the transmembrane domain comprises the amino acid sequence of AAGITGLVLSILVLAAAAALL (SEQ ID NO: 156); the chimeric inhibitory receptor comprises a transmembrane domain derived from PCDHGC5, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LIVALATVSLLSLVTFTFLSA (SEQ ID NO: 157), optionally wherein the transmembrane domain comprises the amino acid sequence of LIVALATVSLLSLVTFTFLSA (SEQ ID NO: 157); the chimeric inhibitory receptor comprises a transmembrane domain derived from CDH11, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to GALIAILACIVILLVIVVLFVTL (SEQ ID NO: 158), optionally wherein the transmembrane domain comprises the amino acid sequence of GALIAILACIVILLVIVVLFVTL (SEQ ID NO: 158); the chimeric inhibitory receptor comprises a transmembrane domain derived from IMPG2, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VIIGITIASVVGLLVIFSAII (SEQ ID NO: 159), optionally wherein the transmembrane domain comprises the amino acid sequence of VIIGITIASVVGLLVIFSAII (SEQ ID NO: 159); or the chimeric inhibitory receptor comprises a transmembrane domain derived from DSCAM, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LVTISCILVGVLLLFVLLLVV (SEQ ID NO: 160), optionally wherein the transmembrane domain comprises the amino acid sequence of LVTISCILVGVLLLFVLLLVV (SEQ ID NO: 160).

In some embodiments, (i) the protein is not expressed on the target tumor, optionally wherein the protein is expressed on a non-tumor cell, optionally wherein the protein is expressed on a non-tumor cell derived from a tissue selected from the group consisting of brain, neuronal tissue, endocrine, endothelial, bone, bone marrow, immune system, muscle, lung, liver, gallbladder, pancreas, gastrointestinal tract, kidney, urinary bladder, male reproductive organs, female reproductive organs, adipose, soft tissue, and skin; and/or (ii) the extracellular protein binding domain comprises a ligand-binding domain, a receptor-binding domain, and/or an antigen-binding domain, optionally wherein the antigen-binding domain comprises an antibody, an antigen-binding fragment of an antibody, a F(ab) fragment, a F(ab′) fragment, a single chain variable fragment (scFv), or a single-domain antibody (sdAb), optionally wherein the antigen-binding domain comprises a single chain variable fragment (scFv), optionally wherein the scFv comprises a heavy chain variable domain (VH) and a light chain variable domain (VL), optionally wherein the VH and VL are separated by a peptide linker, optionally wherein the peptide linker comprises an amino acid sequence selected from the group consisting of: GGS (SEQ ID NO: 47), GGSGGS (SEQ ID NO: 48), GGSGGSGGS (SEQ ID NO: 49), GGSGGSGGSGGS (SEQ ID NO: 50), GGSGGSGGSGGSGGS (SEQ ID NO: 51), GGGS (SEQ ID NO: 52), GGGSGGGS (SEQ ID NO: 53), GGGSGGGSGGGS (SEQ ID NO: 54), GGGSGGGSGGGSGGGS (SEQ ID NO: 55), GGGSGGGSGGGSGGGSGGGS (SEQ ID NO: 56), GGGGS (SEQ ID NO: 57), GGGGSGGGGS (SEQ ID NO: 58), GGGGSGGGGSGGGGS (SEQ ID NO: 59), GGGGSGGGGSGGGGSGGGGS (SEQ ID NO: 60), GGGGSGGGGSGGGGSGGGGSGGGGS (SEQ ID NO: 61), GGGGSGGGGSGGGGSQSV (SEQ ID NO: 63), GSTSGSGKPGSGEGSTKG (SEQ ID NO: 64), SGGGGSGGGGSGGGGSGGGGSGGGSLQ (SEQ ID NO: 65), and TTTPAPRPPTPAPTIALQPLSLRPEACRPAAGGAVHTRGLDFACDQTTPGERSSLPAFYPGTSGSC SGCGSLSLP (SEQ ID NO: 62), optionally wherein the scFv comprises the structure VH-L-VL or VL-L-VH, wherein VH is the heavy chain variable domain, L is the peptide linker, and VL is the light chain variable domain.

In some embodiments, the chimeric inhibitory receptor further comprises a spacer region positioned between the extracellular protein binding domain and the transmembrane domain and operably linked, e.g., physically linked, to each of the extracellular protein binding domain and the transmembrane domain, optionally wherein the spacer region is derived from a protein selected from the group consisting of: CD8α, CD4, CD7, CD28, IgG1, IgG4, FcγRIIIα, LNGFR, and PDGFR, optionally wherein the spacer region comprises an amino acid sequence selected from the group consisting of:

(SEQ ID NO: 73)
TTTPAPRPPTPAPTIALQPLSLRPEACRPAAGGAVHTRGLDFACD,
(SEQ ID NO: 77)
ALSNSIMYFSHFVPVFLPAKPTTTPAPRPPTPAPTIASQPLSLRPEAC
RPAAGGAVHTRGLDFACD,
(SEQ ID NO: 67)
AAAIEVMYPPPYLDNEKSNGTIIHVKGKHLCPSPLFPGPSKP,
(SEQ ID NO: 68)
ESKYGPPCPSCP,
(SEQ ID NO: 69)
ESKYGPPAPSAP,
(SEQ ID NO: 70)
ESKYGPPCPPCP,
(SEQ ID NO: 71)
EPKSCDKTHTCP,
(SEQ ID NO: 72)
AAAFVPVFLPAKPTTTPAPRPPTPAPTIASQPLSLRPEACRPAAGGAV
HTRGLDFACDIYIWAPLAGTCGVLLLSLVITLYCNHRN,
(SEQ ID NO: 74)
ACPTGLYTHSGECCKACNLGEGVAQPCGANQTVCEPCLDSVTFSDVVS
ATEPCKPCTECVGLQSMSAPCVEADDAVCRCAYGYYQDETTGRCEACR
VCEAGSGLVFSCQDKQNTVCEECPDGTYSDEADAEC,
(SEQ ID NO: 75)
ACPTGLYTHSGECCKACNLGEGVAQPCGANQTVC,
(SEQ ID NO: 76)
AVGQDTQEVIVVPHSLPFKV,
(SEQ ID NO: 183)
ESKYGPPCPSCPAPPVAGPSVFLFPPKPKDTLMISRTPEVTCVVVDVS
QEDPEVQFNWYVDGVEVHNAKTKPREEQFQSTYRVVSVLTVLHQDWLN
GKEYKCKVSNKGLPSSIEKTISKAKGQPREPQVYTLPPSQEEMTKNQV
SLTCLVKGFYPSDIAVEWESNGQPENNYKTTPPVLDSDGSFFLYSRLT
VDKSRWQEGNVFSCSVMHEALHNHYTQKSLSLSLGK,
and
(SEQ ID NO: 184)
ESKYGPPCPSCPGQPREPQVYTLPPSQEEMTKNQVSLTCLVKGFYPSD
IAVEWESNGQPENNYKTTPPVLDSDGSFFLYSRLTVDKSRWQEGNVFS
CSVMHEALHNHYTQKSLSLSLGK.

In some embodiments, the chimeric inhibitory receptor further comprises an intracellular spacer region positioned between the transmembrane domain and one of the one or more intracellular signaling domains and operably linked, e.g., physically linked, to each of the transmembrane domain and one of the one or more intracellular signaling domains. In some embodiments, the inhibitory chimeric receptor further comprises an enzymatic inhibitory domain, optionally wherein the enzymatic inhibitory domain is capable of preventing, attenuating, or inhibiting activation of a tumor-targeting chimeric receptor when expressed on an immunomodulatory cell relative to an otherwise identical chimeric inhibitory receptor lacking the enzymatic inhibitory domain, optionally wherein the enzymatic inhibitory domain comprises an enzyme catalytic domain, optionally wherein the enzyme catalytic domain is derived from an enzyme selected from the group consisting of: CSK, SHP-1, PTEN, CD45, CD148, PTP-MEG1, PTP-PEST, c-CBL, CBL-b, PTPN22, LAR, PTPH1, SHIP-1, and RasGAP.

In some embodiments, the tumor-targeting chimeric receptor is a chimeric antigen receptor (CAR) or an engineered T cell receptor (TCR). In some embodiments, the immunomodulatory cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell, optionally wherein the immunomodulatory cell is a Natural Killer (NK) cell.

Another embodiment of the disclosure provides a composition comprising the chimeric inhibitory receptor of the present disclosure and a pharmaceutically acceptable carrier, a pharmaceutically acceptable excipient, or a combination thereof.

Another embodiment of the disclosure provides an engineered nucleic acid encoding the chimeric inhibitory receptor of the present disclosure.

Another embodiment of the disclosure provides an expression vector comprising the engineered nucleic acid of the present disclosure.

Another embodiment of the disclosure provides a composition comprising the engineered nucleic acid or the expression vector of the present disclosure, and a pharmaceutically acceptable carrier

Another embodiment of the disclosure provides a producer cell or isolated immunomodulatory cell comprising the chimeric inhibitory receptor, the engineered nucleic acid, or the expression vector of the present disclosure.

Another embodiment of the disclosure provides a composition comprising the isolated cell of the present disclosure and a pharmaceutically acceptable carrier, a pharmaceutically acceptable excipient, or a combination thereof.

Another embodiment of the disclosure provides a method of preventing, attenuating, or inhibiting a cell-mediated immune response induced by a tumor-targeting chimeric receptor expressed of the surface of an immunomodulatory cell, comprising: engineering the immunomodulatory cell to express the chimeric inhibitory receptor of the present disclosure on the surface of the immunomodulatory cell, wherein upon binding of a cognate antigen to the chimeric inhibitory receptor, the intracellular signaling domain prevents, attenuates, or inhibits activation of the tumor-targeting chimeric receptor.

Another embodiment of the disclosure provides a method of preventing, attenuating, or inhibiting activation of a tumor-targeting chimeric receptor expressed on the surface of an immunomodulatory cell, comprising: contacting the isolated cell or the composition of the present disclosure with a cognate antigen of the chimeric inhibitory receptor under conditions suitable for the chimeric inhibitory receptor to bind the cognate antigen, wherein upon binding of the antigen to the chimeric inhibitory receptor, the intracellular signaling domain prevents, attenuates, or inhibits activation of the tumor-targeting chimeric receptor.

Another embodiment of the disclosure provides a chimeric inhibitory receptor comprising (1) an extracellular protein binding domain; (2) a transmembrane domain, wherein the transmembrane domain is operably linked to the extracellular protein binding domain; and (3) one or more intracellular signaling domains, wherein the one or more intracellular signaling domains are operably linked to the transmembrane domain, wherein the one or more intracellular signaling domain are each derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM, and wherein at least one of the one or more intracellular signaling domains is capable of preventing, attenuating, or inhibiting activation of a tumor-targeting chimeric receptor expressed on an immunomodulatory cell.

In some embodiments, the transmembrane domain is derived from the same protein as one of the one or more intracellular signaling domains. In some embodiments, the transmembrane domain further comprises at least a portion of an extracellular domain of the same protein. In some embodiments, the transmembrane domain is derived from a first protein and the one or more intracellular signaling domains are derived from proteins that are distinct from the first protein. In some embodiments, the one or more intracellular signaling domains are two intracellular signaling domains.

In some embodiments, the chimeric inhibitory receptor comprises a first intracellular signaling domain derived from PECAM-1 and a second intracellular signaling domain derived from a protein selected from the group consisting of: CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from CD72 and a second intracellular signaling domain derived from a protein selected from the group consisting of: PECAM-1, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from IRTA2 and a second intracellular signaling domain derived from a protein selected from the group consisting of: PECAM-1, CD72, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from IRTA4 and a second intracellular signaling domain derived from a protein selected from the group consisting of: PECAM-1, CD72, IRTA2, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from NKIR and a second intracellular signaling domain derived from a protein selected from the group consisting of: PECAM-1, CD72, IRTA2, IRTA4, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from IL1RAP and a second intracellular signaling domain derived from a protein selected from the group consisting of: PECAM-1, CD72, IRTA2, IRTA4, NKIR, PTPRO, PTPRZ1, TLT1, SLAMF1, and SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from PTPRO and a second intracellular signaling domain derived from a protein selected from the group consisting of: PECAM-1, CD72, IRTA2, IRTA4, NKIR, IL1RAP, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from PTPRZ1 and a second intracellular signaling domain derived from a protein selected from the group consisting of: PECAM-1, CD72, IRTA2, IRTA4, NKIR, IL1RAP, PTPRO, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from TLT1 and a second intracellular signaling domain derived from a protein selected from the group consisting of: PECAM-1, CD72, IRTA2, IRTA4, NKIR, IL1RAP, PTPRO, PTPRZ1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from SLAMF1 and a second intracellular signaling domain derived from a protein selected from the group consisting of: PECAM-1, CD72, IRTA2, IRTA4, NKIR, IL1RAP, PTPRO, PTPRZ1, TLT1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from SLAMF5 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from PCDHGC3 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from LIFR and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from ERMAP and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from IL1RAPL2 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from CDH5 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, MPZL1, MPZ, FCGR2B. SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from MPZL1 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from MPZ and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from FCGR2B and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from SIGLEC-6 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from MPIG6B and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from VSIG4 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from SIGLEC-12 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from LIR8 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from IRTA1 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from KIR2DL4 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL5, SIGLEC-7, FCRH3, PCDHGC5, CDH11, IMPG2, and DSCAM.

In some embodiments, the chimeric inhibitory receptor comprises a first intracellular signaling domain derived from KIR2DL5 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, SIGLEC-7, FCRH3, PCDHGC5, CDH11, IMPG2, and DSCAM.

In some embodiments, the chimeric inhibitory receptor comprises a first intracellular signaling domain derived from SIGLEC-7 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, FCRH3, PCDHGC5, CDH11, IMPG2, and DSCAM.

In some embodiments, the chimeric inhibitory receptor comprises a first intracellular signaling domain derived from FCRH3 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, PCDHGC5, CDH11, IMPG2, and DSCAM.

In some embodiments, the chimeric inhibitory receptor comprises a first intracellular signaling domain derived from PCDHGC5 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, CDH11, IMPG2, and DSCAM.

In some embodiments, the chimeric inhibitory receptor comprises a first intracellular signaling domain derived from CDH11 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, IMPG2, and DSCAM.

In some embodiments, the chimeric inhibitory receptor comprises a first intracellular signaling domain derived from IMPG2 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, CDH11, and DSCAM.

In some embodiments, the chimeric inhibitory receptor comprises a first intracellular signaling domain derived from DSCAM and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, CDH11, and IMPG2

In some embodiments, one of the one or more intracellular signaling domains is derived from PECAM-1. In some embodiments, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to KCYFLRKAKAKQMPVEMSRPAVPLLNSNNEKMSDPNMEANSHYGHNDDVRNHAMKPINDNK EPLNSDVQYTEVQVSSAESHKDLGKKDTETVYSEVRKAVPDAVESRYSRTEGSLDGT (SEQ ID NO: 1). In some embodiments, the intracellular signaling domain comprises the amino acid sequence of KCYFLRKAKAKQMPVEMSRPAVPLLNSNNEKMSDPNMEANSHYGHNDDVRNHAMKPINDNK EPLNSDVQYTEVQVSSAESHKDLGKKDTETVYSEVRKAVPDAVESRYSRTEGSLDGT (SEQ ID NO: 1).

In some embodiments, one of the one or more intracellular signaling domains is derived from CD72. In some embodiments, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to MAEAITYADLRFVKAPLKKSISSRLGQDPGADDDGEITYENVQVPAVLGVPSSLASSVLGDKAA VKSEQPTASWRAVTSPAVGRILPCRTTCLRY (SEQ ID NO: 2). In some embodiments, the intracellular signaling domain comprises the amino acid sequence of MAEAITYADLRFVKAPLKKSISSRLGQDPGADDDGEITYENVQVPAVLGVPSSLASSVLGDKAA VKSEQPTASWRAVTSPAVGRILPCRTTCLRY (SEQ ID NO: 2).

In some embodiments, one of the one or more intracellular signaling domains is derived from IRTA2. In some embodiments, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LSRKAGRKPASDPARSPSDSDSQEPTYHNVPAWEELQPVYTNANPRGENVVYSEVRIIQEKKKH AVASDPRHLRNKGSPIIYSEVKVASTPVSGSLFLASSAPHR (SEQ ID NO: 3). In some embodiments, the intracellular signaling domain comprises the amino acid sequence of LSRKAGRKPASDPARSPSDSDSQEPTYHNVPAWEELQPVYTNANPRGENVVYSEVRIIQEKKKH AVASDPRHLRNKGSPIIYSEVKVASTPVSGSLFLASSAPHR (SEQ ID NO: 3).

In some embodiments, one of the one or more intracellular signaling domains is derived from IRTA4. In some embodiments, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to HKISGESSATNEPRGASRPNPQEFTYSSPTPDMEELQPVYVNVGSVDVDVVYSQVWSMQQPESS ANIRTLLENKDSQVIYSSVKKS (SEQ ID NO: 4). In some embodiments, the intracellular signaling domain comprises the amino acid sequence of HKISGESSATNEPRGASRPNPQEFTYSSPTPDMEELQPVYVNVGSVDVDVVYSQVWSMQQPESS ANIRTLLENKDSQVIYSSVKKS (SEQ ID NO: 4).

In some embodiments, one of the one or more intracellular signaling domains is derived from NKIR. In some embodiments, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to WRMMKYQQKAAGMSPEQVLQPLEGDLCYADLTLQLAGTSPQKATTKLSSAQVDQVEVEYVT MASLPKEDISYASLTLGAEDQEPTYCNMGHLSSHLPGRGPEEPTEYSTISRP (SEQ ID NO: 5). In some embodiments, the intracellular signaling domain comprises the amino acid sequence of WRMMKYQQKAAGMSPEQVLQPLEGDLCYADLTLQLAGTSPQKATTKLSSAQVDQVEVEYVT MASLPKEDISYASLTLGAEDQEPTYCNMGHLSSHLPGRGPEEPTEYSTISRP (SEQ ID NO: 5).

In some embodiments, one of the one or more intracellular signaling domains is derived from IL1RAP. In some embodiments, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to YRAHFGTDETILDGKEYDIYVSYARNAEEEEFVLLTLRGVLENEFGYKLCIFDRDSLPGGIVTDET LSFIQKSRRLLVVLSPNYVLQGTQALLELKAGLENMASRGNINVILVQYKAVKETKVKELKRAK TVLTVIKWKGEKSKYPQGRFWKQLQVAMPVKKSPRRSSSDEQGLSYSSLKNV (SEQ ID NO: 6).

In some embodiments, the intracellular signaling domain comprises the amino acid sequence of YRAHFGTDETILDGKEYDIYVSYARNAEEEEFVLLTLRGVLENEFGYKLCIFDRDSLPGGIVTDET LSFIQKSRRLLVVLSPNYVLQGTQALLELKAGLENMASRGNINVILVQYKAVKETKVKELKRAK TVLTVIKWKGEKSKYPQGRFWKQLQVAMPVKKSPRRSSSDEQGLSYSSLKNV (SEQ ID NO: 6).

In some embodiments, one of the one or more intracellular signaling domains is derived from PTPRO. In some embodiments, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LRKKHLQMARECGAGTFVNFASLERDGKLPYNWRRSIFAFLTLLPSCLWTDYLLAFYINPWSKN GLKKRKLTNPVQLDDFDAYIKDMAKDSDYKFSLQFEELKLIGLDIPHFAADLPLNRCKNRYTNIL PYDFSRVRLVSMNEEEGADYINANYIPGYNSPQEYIATQGPLPETRNDFWKMVLQQKSQIIVML TQCNEKRRVKCDHYWPFTEEPIAYGDITVEMISEEEQDDWACRHFRINYADEMQDVMHFNYTA WPDHGVPTANAAESILQFVHMVRQQATKSKGPMIIHCSAGVGRTGTFIALDRLLQHIRDHEFVDI LGLVSEMRSYRMSMVQTEEQYIFIHQCVQLMWMKKKQQFCISDVIYENVSKS (SEQ ID NO: 7). In some embodiments, the intracellular signaling domain comprises the amino acid sequence of LRKKHLQMARECGAGTFVNFASLERDGKLPYNWRRSIFAFLTLLPSCLWTDYLLAFYINPWSKN GLKKRKLTNPVQLDDFDAYIKDMAKDSDYKFSLQFEELKLIGLDIPHFAADLPLNRCKNRYTNIL PYDFSRVRLVSMNEEEGADYINANYIPGYNSPQEYIATQGPLPETRNDFWKMVLQQKSQIIVML TQCNEKRRVKCDHYWPFTEEPIAYGDITVEMISEEEQDDWACRHFRINYADEMQDVMHFNYTA WPDHGVPTANAAESILQFVHMVRQQATKSKGPMIIHCSAGVGRTGTFIALDRLLQHIRDHEFVDI LGLVSEMRSYRMSMVQTEEQYIFIHQCVQLMWMKKKQQFCISDVIYENVSKS (SEQ ID NO: 7).

In some embodiments, one of the one or more intracellular signaling domains is derived from PTPRZ1. In some embodiments, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RKCFQTAHFYLEDSTSPRVISTPPTPIFPISDDVGAIPIKHFPKHVADLHASSGFTEEFETLKEFYQE VQSCTVDLGITADSSNHPDNKHKNRYINIVAYDHSRVKLAQLAEKDGKLTDYINANYVDGYNR PKAYIAAQGPLKSTAEDFWRMIWEHNVEVIVMITNLVEKGRRKCDQYWPADGSEEYGNFLVTQ KSVQVLAYYTVRNFTLRNTKIKKGSQKGRPSGRVVTQYHYTQWPDMGVPEYSLPVLTFVRKAA YAKRHAVGPVVVHCSAGVGRTGTYIVLDSMLQQIQHEGTVNIFGFLKHIRSQRNYLVQTEEQYV FIHDTLVEAILSKETEVLDSHIHAYVNALLIPGPAGKTKLEKQFQLLSQSNIQQSDYSAALKQCNR EKNRTSSIIPVERSRVGISSLSGEGTDYINASYIMGYYQSNEFIITQHPLLHTIKDFWRMIWDHNAQ LVVMIPDGQNMAEDEFVYWPNKDEPINCESFKVTLMAEEHKCLSNEEKLIIQDFILEATQDDYV LEVRHFQCPKWPNPDSPISKTFELISVIKEEAANRDGPMIVHDEHGGVTAGTFCALTTLMHQLEK ENSVDVYQVAKMINLMRPGVFADIEQYQFLYKVILSLVSTRQEENPSTSLDSNGAALPDGNIAES LESLV (SEQ ID NO: 8). In some embodiments, the intracellular signaling domain comprises the amino acid sequence of RKCFQTAHFYLEDSTSPRVISTPPTPIFPISDDVGAIPIKHFPKHVADLHASSGFTEEFETLKEFYQE VQSCTVDLGITADSSNHPDNKHKNRYINIVAYDHSRVKLAQLAEKDGKLTDYINANYVDGYNR PKAYIAAQGPLKSTAEDFWRMIWEHNVEVIVMITNLVEKGRRKCDQYWPADGSEEYGNFLVTQ KSVQVLAYYTVRNFTLRNTKIKKGSQKGRPSGRVVTQYHYTQWPDMGVPEYSLPVLTFVRKAA YAKRHAVGPVVVHCSAGVGRTGTYIVLDSMLQQIQHEGTVNIFGFLKHIRSQRNYLVQTEEQYV FIHDTLVEAILSKETEVLDSHIHAYVNALLIPGPAGKTKLEKQFQLLSQSNIQQSDYSAALKQCNR EKNRTSSIIPVERSRVGISSLSGEGTDYINASYIMGYYQSNEFIITQHPLLHTIKDFWRMIWDHNAQ LVVMIPDGQNMAEDEFVYWPNKDEPINCESFKVTLMAEEHKCLSNEEKLIIQDFILEATQDDYV LEVRHFQCPKWPNPDSPISKTFELISVIKEEAANRDGPMIVHDEHGGVTAGTFCALTTLMHQLEK ENSVDVYQVAKMINLMRPGVFADIEQYQFLYKVILSLVSTRQEENPSTSLDSNGAALPDGNIAES LESLV (SEQ ID NO: 8).

In some embodiments, one of the one or more intracellular signaling domains is derived from TLT1. In some embodiments, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to MAKRKQGNRLGVCGRFLSSRVSGMNPSSVVHHVSDSGPAAELPLDVPHIRLDSPPSFDNTTYTS LPLDSPSGKPSLPAPSSLPPLPPKVLVCSKPVTYATVIFPGGNKGGGTSCGPAQNPPNNQTPSS (SEQ ID NO: 9). In some embodiments, the intracellular signaling domain comprises the amino acid sequence of MAKRKQGNRLGVCGRFLSSRVSGMNPSSVVHHVSDSGPAAELPLDVPHIRLDSPPSFDNTTYTS LPLDSPSGKPSLPAPSSLPPLPPKVLVCSKPVTYATVIFPGGNKGGGTSCGPAQNPPNNQTPSS (SEQ ID NO: 9).

In some embodiments, one of the one or more intracellular signaling domains is derived from SLAMF1. In some embodiments, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to QLRRRGKTNHYQTTVEKKSLTIYAQVQKPGPLQKKLDSFPAQDPCTTIYVAATEPVPESVQETNS ITVYASVTLPES (SEQ ID NO: 10). In some embodiments, the intracellular signaling domain comprises the amino acid sequence of QLRRRGKTNHYQTTVEKKSLTIYAQVQKPGPLQKKLDSFPAQDPCTTIYVAATEPVPESVQETNS ITVYASVTLPES (SEQ ID NO: 10).

In some embodiments, one of the one or more intracellular signaling domains is derived from SLAMF5. In some embodiments, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RLFKRRQGRIFPEGSCLNTFTKNPYAASKKTIYTYIMASRNTQPAESRIYDEILQSKVLPSKEEPVN TVYSEVQFADKMGKASTQDSKPPGTSSYEIVI (SEQ ID NO: 11). In some embodiments, the intracellular signaling domain comprises the amino acid sequence of RLFKRRQGRIFPEGSCLNTFTKNPYAASKKTIYTYIMASRNTQPAESRIYDEILQSKVLPSKEEPVN TVYSEVQFADKMGKASTQDSKPPGTSSYEIVI (SEQ ID NO: 11).

In some embodiments, one of the one or more intracellular signaling domains is derived from PCDHGC3. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to FKVYKWKQSRDLYRAPVSSLYRTPGPSLHADAVRGGLMSPHLYHQVYLTTDSRRSDPLLKKPG AASPLASRQNTLRSCDPVFYRQVLGAESAPPGQQAPPNTDWRFSQAQRPGTSGSQNGDDTGTW PNNQFDTEMLQAMILASASEAADGSSTLGGGAGTMGLSARYGPQFTLQHVPDYRQNVYIPGSN ATLTNAAGKRDGKAPAGGNGNKKKSGKKEKK (SEQ ID NO: 95). In some embodiments, one of the one or more intracellular signaling domain comprises the amino acid sequence of FKVYKWKQSRDLYRAPVSSLYRTPGPSLHADAVRGGLMSPHLYHQVYLTTDSRRSDPLLKKPG AASPLASRQNTLRSCDPVFYRQVLGAESAPPGQQAPPNTDWRFSQAQRPGTSGSQNGDDTGTW PNNQFDTEMLQAMILASASEAADGSSTLGGGAGTMGLSARYGPQFTLQHVPDYRQNVYIPGSN ATLTNAAGKRDGKAPAGGNGNKKKSGKKEKK (SEQ ID NO: 95)

In some embodiments, wherein one of the one or more intracellular signaling domains is derived from LIFR. In some embodiments, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to YRKREWIKETFYPDIPNPENCKALQFQKSVCEGSSALKTLEMNPCTPNNVEVLETRSAFPKIEDT EIISPVAERPEDRSDAEPENHVVVSYCPPIIEEEIPNPAADEAGGTAQVIYIDVQSMYQPQAKPEEE QENDPVGGAGYKPQMHLPINSTVEDIAAEEDLDKTAGYRPQANVNTWNLVSPDSPRSIDSNSEI VSFGSPCSINSRQFLIPPKDEDSPKSNGGGWSFTNFFQNKPND (SEQ ID NO: 96). In some embodiments, one of the one or more intracellular signaling domain comprises the amino acid sequence of YRKREWIKETFYPDIPNPENCKALQFQKSVCEGSSALKTLEMNPCTPNNVEVLETRSAFPKIEDT EIISPVAERPEDRSDAEPENHVVVSYCPPIIEEEIPNPAADEAGGTAQVIYIDVQSMYQPQAKPEEE QENDPVGGAGYKPQMHLPINSTVEDIAAEEDLDKTAGYRPQANVNTWNLVSPDSPRSIDSNSEI VSFGSPCSINSRQFLIPPKDEDSPKSNGGGWSFTNFFQNKPND (SEQ ID NO: 96).

In some embodiments, one of the one or more intracellular signaling domains is derived from ERMAP. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to WKQRRAKEKLLYEHVTEVDNLLSDHAKEKGKLHKAVKKLRSELKLKRAAANSGWRRARLHF VAVTLDPDTAHPKLILSEDQRCVRLGDRRQPVPDNPQRFDFVVSILGSEYFTTGCHYWEVYVGD KTKWILGVCSESVSRKGKVTASPANGHWLLRQSRGNEYEALTSPQTSFRLKEPPRCVGIFLDYE AGVISFYNVTNKSHIFTFTHNFSGPLRPFFEPCLHDGGKNTAPLVICSELHKSEESIVPRPEGKGHA NGDVSLKVNSSLLPPKAPELKDIILSLPPDLGPALQELKAPSF (SEQ ID NO: 97). In some embodiments, one of the one or more intracellular signaling domain comprises the amino acid sequence of WKQRRAKEKLLYEHVTEVDNLLSDHAKEKGKLHKAVKKLRSELKLKRAAANSGWRRARLHF VAVTLDPDTAHPKLILSEDQRCVRLGDRRQPVPDNPQRFDFVVSILGSEYFTTGCHYWEVYVGD KTKWILGVCSESVSRKGKVTASPANGHWLLRQSRGNEYEALTSPQTSFRLKEPPRCVGIFLDYE AGVISFYNVTNKSHIFTFTHNFSGPLRPFFEPCLHDGGKNTAPLVICSELHKSEESIVPRPEGKGHA NGDVSLKVNSSLLPPKAPELKDIILSLPPDLGPALQELKAPSF (SEQ ID NO: 97).

In some embodiments, one of the one or more intracellular signaling domains is derived from IL1RAPL2. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to KCYNIELMLFYRQHFGADETNDDNKEYDAYLSYTKVDQDTLDCDNPEEEQFALEVLPDVLEKH YGYKLFIPERDLIPSGTYMEDLTRYVEQSRRLIIVLTPDYILRRGWSIFELESRLHNMLVSGEIKVI LIECTELKGKVNCQEVESLKRSIKLLSLIKWKGSKSSKLNSKFWKHLVYEMPIKKKEMLPRCHVL DSAEQGLFGELQPIPSIAMTSTSATLVSSQADLPEFHPSDSMQIRHCCRGYKHEIPATTLPVPSLGN HHTYCNLPLTLLNGQLPLNNTLKDTQEFHRNSSLLPLSSKELSFTSDIW (SEQ ID NO: 98). In some embodiments, one of the one or more intracellular signaling domain comprises the amino acid sequence of KCYNIELMLFYRQHFGADETNDDNKEYDAYLSYTKVDQDTLDCDNPEEEQFALEVLPDVLEKH YGYKLFIPERDLIPSGTYMEDLTRYVEQSRRLIIVLTPDYILRRGWSIFELESRLHNMLVSGEIKVI LIECTELKGKVNCQEVESLKRSIKLLSLIKWKGSKSSKLNSKFWKHLVYEMPIKKKEMLPRCHVL DSAEQGLFGELQPIPSIAMTSTSATLVSSQADLPEFHPSDSMQIRHCCRGYKHEIPATTLPVPSLGN HHTYCNLPLTLLNGQLPLNNTLKDTQEFHRNSSLLPLSSKELSFTSDIW (SEQ ID NO: 98).

In some embodiments, one of the one or more intracellular signaling domains is derived from CDH5. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RRRLRKQARAHGKSVPEIHEQLVTYDEEGGGEMDTTSYDVSVLNSVRRGGAKPPRPALDARPS LYAQVQKPPRHAPGAHGGPGEMAAMIEVKKDEADHDGDGPPYDTLHIYGYEGSESIAESLSSLG TDSSDSDVDYDFLNDWGPRFKMLAELYGSDPREELLY (SEQ ID NO: 99). In some embodiments, one of the one or more intracellular signaling domain comprises the amino acid sequence of RRRLRKQARAHGKSVPEIHEQLVTYDEEGGGEMDTTSYDVSVLNSVRRGGAKPPRPALDARPS LYAQVQKPPRHAPGAHGGPGEMAAMIEVKKDEADHDGDGPPYDTLHIYGYEGSESIAESLSSLG TDSSDSDVDYDFLNDWGPRFKMLAELYGSDPREELLY (SEQ ID NO: 99).

In some embodiments, one of the one or more intracellular signaling domains is derived from MPZL1. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SMILAVLYRRKNSKRDYTGCSTSESLSPVKQAPRKSPSDTEGLVKSLPSGSHQGPVIYAQLDHSG GHHSDKINKSESVVYADIRKN (SEQ ID NO: 100). In some embodiments, one of the one or more intracellular signaling domain comprises the amino acid sequence of SMILAVLYRRKNSKRDYTGCSTSESLSPVKQAPRKSPSDTEGLVKSLPSGSHQGPVIYAQLDHSG GHHSDKINKSESVVYADIRKN (SEQ ID NO: 100).

In some embodiments, one of the one or more intracellular signaling domains is derived from MPZ. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RYCWLRRQAALQRRLSAMEKGKLHKPGKDASKRGRQTPVLYAMLDHSRSTKAVSEKKAKGL GESRKDKK (SEQ ID NO: 101). In some embodiments, one of the one or more intracellular signaling domain comprises the amino acid sequence of RYCWLRRQAALQRRLSAMEKGKLHKPGKDASKRGRQTPVLYAMLDHSRSTKAVSEKKAKGL GESRKDKK (SEQ ID NO: 101).

In some embodiments, one of the one or more intracellular signaling domains is derived from FCGR2B. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VVALIYCRKKRISALPGYPECREMGETLPEKPANPTNPDEADKVGAENTITYSLLMHPDALEEPD DQNRI (SEQ ID NO: 102). In some embodiments, one of the one or more intracellular signaling domain comprises the amino acid sequence of VVALIYCRKKRISALPGYPECREMGETLPEKPANPTNPDEADKVGAENTITYSLLMHPDALEEPD DQNRI (SEQ ID NO: 102).

In some embodiments, one of the one or more intracellular signaling domains is derived from SIGLEC-6. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RVKTRRKKAAQPVQNTDDVNPVMVSGSRGHQHQFQTGIVSDHPAEAGPISEDEQELHYAVLHF HKVQPQEPKVTDTEYSEIKIHK (SEQ ID NO: 103). In some embodiments, one of the one or more intracellular signaling domain comprises the amino acid sequence of RVKTRRKKAAQPVQNTDDVNPVMVSGSRGHQHQFQTGIVSDHPAEAGPISEDEQELHYAVLHF HKVQPQEPKVTDTEYSEIKIHK (SEQ ID NO: 103).

In some embodiments, one of the one or more intracellular signaling domains is derived from MPIG6B. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to WLHRRLPPQPIRPLPRFAPLVKTEPQRPVKEEEPKIPGDLDQEPSLLYADLDHLALSRPRRLSTAD PADASTIYAVVV (SEQ ID NO: 104). In some embodiments, one of the one or more intracellular signaling domain comprises the amino acid sequence of WLHRRLPPQPIRPLPRFAPLVKTEPQRPVKEEEPKIPGDLDQEPSLLYADLDHLALSRPRRLSTAD PADASTIYAVVV (SEQ ID NO: 104).

In some embodiments, one of the one or more intracellular signaling domains is derived from VSIG4. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to MLCRKTSQQEHVYEAARAHAREANDSGETMRVAIFASGCSSDEPTSQNLGNNYSDEPCIGQEY QIIAQINGNYARLLDTVPLDYEFLATEGKSVC (SEQ ID NO: 105). In some embodiments, one of the one or more intracellular signaling domain comprises the amino acid sequence of MLCRKTSQQEHVYEAARAHAREANDSGETMRVAIFASGCSSDEPTSQNLGNNYSDEPCIGQEY QIIAQINGNYARLLDTVPLDYEFLATEGKSVC (SEQ ID NO: 105).

In some embodiments, one of the one or more intracellular signaling domains is derived from SIGLEC-12. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RSCRKKSARPAVGVGDTGMEDANAVRGSASQGPLIESPADDSPPHHAPPALATPSPEEGEIQYAS LSFHKARPQYPQEQEAIGYEYSEINIPK (SEQ ID NO: 106). In some embodiments, one of the one or more intracellular signaling domain comprises the amino acid sequence of RSCRKKSARPAVGVGDTGMEDANAVRGSASQGPLIESPADDSPPHHAPPALATPSPEEGEIQYAS LSFHKARPQYPQEQEAIGYEYSEINIPK (SEQ ID NO: 106).

In some embodiments, one of the one or more intracellular signaling domains is derived from LIR8. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RHRHQSKHRTSAHFYRPAGAAGPEPKDQGLQKRASPVADIQEEILNAAVKDTQPKDGVEMDAP AAASEAPQDVTYAQLHSLTLRREATEPPPSQEREPPAEPSIYAPLAIH (SEQ ID NO: 107). In some embodiments, one of the one or more intracellular signaling domain comprises the amino acid sequence of RHRHQSKHRTSAHFYRPAGAAGPEPKDQGLQKRASPVADIQEEILNAAVKDTQPKDGVEMDAP AAASEAPQDVTYAQLHSLTLRREATEPPPSQEREPPAEPSIYAPLAIH (SEQ ID NO: 107).

In some embodiments, one of the one or more intracellular signaling domains is derived from IRTA1. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to HCWRRRKSGVGFLGDETRLPPAPGPGESSHSICPAQVELQSLYVDVHPKKGDLVYSEIQTTQLGE EEEANTSRTLLEDKDVSVVYSEVKTQHPDNSAGKISSKDEES (SEQ ID NO: 108). In some embodiments, one of the one or more intracellular signaling domain comprises the amino acid sequence of HCWRRRKSGVGFLGDETRLPPAPGPGESSHSICPAQVELQSLYVDVHPKKGDLVYSEIQTTQLGE EEEANTSRTLLEDKDVSVVYSEVKTQHPDNSAGKISSKDEES (SEQ ID NO: 108).

In some embodiments, one of the one or more intracellular signaling domains is derived from KIR2DL4. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RWCSKKKDAAVMNQEPAGHRTVNREDSDEQDPQEVTYAQLDHCIFTQRKITGPSQRSKRPSTD TSVCIELPNAEPRALSPAHEHHSQALMGSSRETTALSQTQLASSNVPAAGI (SEQ ID NO: 109). In some embodiments, one of the one or more intracellular signaling domain comprises the amino acid sequence of RWCSKKKDAAVMNQEPAGHRTVNREDSDEQDPQEVTYAQLDHCIFTQRKITGPSQRSKRPSTD TSVCIELPNAEPRALSPAHEHHSQALMGSSRETTALSQTQLASSNVPAAGI. (SEQ ID NO: 109).

In some embodiments, one of the one or more intracellular signaling domains is derived from KIR2DL5. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LHCCCSNKKNAAVMDQEPAGDRTVNREDSDDQDPQEVTYAQLDHCVFTQTKITSPSQRPKTPP TDTTMYMELPNAKPRSLSPAHKHHSQALRGSSRETTALSQNRVASSHVPAAGI (SEQ ID NO: 110). In some embodiments, one of the one or more intracellular signaling domain comprises the amino acid sequence of LHCCCSNKKNAAVMDQEPAGDRTVNREDSDDQDPQEVTYAQLDHCVFTQTKITSPSQRPKTPP TDTTMYMELPNAKPRSLSPAHKHHSQALRGSSRETTALSQNRVASSHVPAAGI (SEQ ID NO: 110).

In some embodiments, one of the one or more intracellular signaling domains is derived from SIGLEC-7. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RSCRKKSARPAADVGDIGMKDANTIRGSASQGNLTESWADDNPRHHGLAAHSSGEEREIQYAP LSFHKGEPQDLSGQEATNNEYSEIKIPK (SEQ ID NO: 111). In some embodiments, one of the one or more intracellular signaling domain comprises the amino acid sequence of RSCRKKSARPAADVGDIGMKDANTIRGSASQGNLTESWADDNPRHHGLAAHSSGEEREIQYAP LSFHKGEPQDLSGQEATNNEYSEIKIPK (SEQ ID NO: 111).

In some embodiments, one of the one or more intracellular signaling domains is derived from FCRH3. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to HYARARRKPGGLSATGTSSHSPSECQEPSSSRPSRIDPQEPTHSKPLAPMELEPMYSNVNPGDSNP IYSQIWSIQHTKENSANCPMMHQEHEELTVLYSELKKTHPDDSAGEASSRGRAHEEDDEENYEN VPRVLLASDH (SEQ ID NO: 112). In some embodiments, one of the one or more intracellular signaling domain comprises the amino acid sequence of HYARARRKPGGLSATGTSSHSPSECQEPSSSRPSRIDPQEPTHSKPLAPMELEPMYSNVNPGDSNP IYSQIWSIQHTKENSANCPMMHQEHEELTVLYSELKKTHPDDSAGEASSRGRAHEEDDEENYEN VPRVLLASDH (SEQ ID NO: 112).

In some embodiments, one of the one or more intracellular signaling domains is derived from PCDHGC5. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to KCLQGNADGDGGGGQCCRRQDSPSPDFYKQSSPNLQVSSDGTLKYMEVTLRPTDSQSHCYRTC FSPASDGSDFTFLRPLSVQQPTALALEPDAIRSRSNTLRERSQQAPPNTDWRFSQAQRPGTSGSQN GDDTGTWPNNQFDTEMLQAMILASASEAADGSSTLGGGAGTMGLSARYGPQFTLQHVPDYRQ NVYIPGSNATLTNAAGKRDGKAPAGGNGNKKKSGKKEKK (SEQ ID NO: 113). In some embodiments, one of the one or more intracellular signaling domain comprises the amino acid sequence of KCLQGNADGDGGGGQCCRRQDSPSPDFYKQSSPNLQVSSDGTLKYMEVTLRPTDSQSHCYRTC FSPASDGSDFTFLRPLSVQQPTALALEPDAIRSRSNTLRERSQQAPPNTDWRFSQAQRPGTSGSQN GDDTGTWPNNQFDTEMLQAMILASASEAADGSSTLGGGAGTMGLSARYGPQFTLQHVPDYRQ NVYIPGSNATLTNAAGKRDGKAPAGGNGNKKKSGKKEKK (SEQ ID NO: 113).

In some embodiments, one of the one or more intracellular signaling domains is derived from CDH11. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RRQKKEPLIVFEEEDVRENIITYDDEGGGEEDTEAFDIATLQNPDGINGFIPRKDIKPEYQYMPRP GLRPAPNSVDVDDFINTRIQEADNDPTAPPYDSIQIYGYEGRGSVAGSLSSLESATTDSDLDYDYL QNWGPRFKKLADLYGSKDTFDDDS (SEQ ID NO: 114). In some embodiments, one of the one or more intracellular signaling domain comprises the amino acid sequence of RRQKKEPLIVFEEEDVRENIITYDDEGGGEEDTEAFDIATLONPDGINGFIPRKDIKPEYQYMPRP GLRPAPNSVDVDDFINTRIQEADNDPTAPPYDSIQIYGYEGRGSVAGSLSSLESATTDSDLDYDYL QNWGPRFKKLADLYGSKDTFDDDS (SEQ ID NO: 114).

In some embodiments, one of the one or more intracellular signaling domains is derived from IMPG2. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to YFFIRTLQAHHDRSERESPFSGSSRQPDSLSSIENAVKYNPVYESHRAGCEKYEGPYPQHPFYSSA SGDVIGGLSREEIRQMYESSELSREEIQERMRVLELYANDPEFAAFVREQQVEEV (SEQ ID NO: 115). In some embodiments, one of the one or more intracellular signaling domain comprises the amino acid sequence of YFFIRTLQAHHDRSERESPFSGSSRQPDSLSSIENAVKYNPVYESHRAGCEKYEGPYPQHPFYSSA SGDVIGGLSREEIRQMYESSELSREEIQERMRVLELYANDPEFAAFVREQQVEEV (SEQ ID NO: 115).

In some embodiments, one of the one or more intracellular signaling domains is derived from DSCAM. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RRRRREQRLKRLRDAKSLAEMLMSKNTRTSDTLSKQQQTLRMHIDIPRAQLLIEERDTMETIDD RSTVLLTDADEGEAAKQKSLTVTHTVHYQSVSQATGPLVDVSDARPGTNPTTRRNAKAGPTAR NRYASQWTLNRPHPTISAHTLTTDWRLPTPRAAGSVDKESDSYSVSPSQDTDRARSSMVSTESA SSTYEELARAYEHAKMEEQLRHAKFTITECFISDTSSEQLTAGTNEYTDSLTSSTPSESGICRFTAS PPKPQDGGRVMNMAVPKAHRPGDLIHLPPYLRMDFLLNRGGPGTSRDLSLGQACLEPQKSRTL KRPTVLEPIPMEAASSASSTREGQSWQPGAVATLPQREGAELGQAAKMSSSQESLLDSRGHLKG NNPYAKSYTLV (SEQ ID NO: 116). In some embodiments, one of the one or more intracellular signaling domain comprises the amino acid sequence of RRRRREQRLKRLRDAKSLAEMLMSKNTRTSDTLSKQQQTLRMHIDIPRAQLLIEERDTMETIDD RSTVLLTDADEGEAAKQKSLTVTHTVHYQSVSQATGPLVDVSDARPGTNPTTRRNAKAGPTAR NRYASQWTLNRPHPTISAHTLTTDWRLPTPRAAGSVDKESDSYSVSPSQDTDRARSSMVSTESA SSTYEELARAYEHAKMEEQLRHAKFTITECFISDTSSEQLTAGTNEYTDSLTSSTPSESGICRFTAS PPKPQDGGRVMNMAVPKAHRPGDLIHLPPYLRMDFLLNRGGPGTSRDLSLGQACLEPQKSRTL KRPTVLEPIPMEAASSASSTREGQSWQPGAVATLPQREGAELGQAAKMSSSQESLLDSRGHLKG NNPYAKSYTLV (SEQ ID NO: 116).

In another embodiment, the present disclosure provides a chimeric inhibitory receptor comprising (1) an extracellular protein binding domain; (2) a transmembrane domain, wherein the transmembrane domain is operably linked to the extracellular protein binding domain; and (3) one or more intracellular signaling domains, wherein the one or more intracellular signaling domains are operably linked to the transmembrane domain, wherein at least one of the one or more intracellular signaling domains comprises: at least one immunoreceptor tyrosine-based switch motif (ITSM); or at least one ITIM and at least one phosphatase, and wherein at least one of the one or more intracellular signaling domains is capable of preventing, attenuating, or inhibiting activation of a tumor-targeting chimeric receptor expressed on an immunomodulatory cell.

In some embodiments, one or more intracellular signaling domains comprises at least one ITIM and at least one phosphatase. In some embodiments, the phosphatase is a protein tyrosine phosphatase (PTP). In some embodiments, the one or more intracellular signaling domains are each derived from a protein selected from the group consisting of: PTPRO and PTPRZ1.

In some embodiments, one of the one or more intracellular signaling domains is derived from PTPRO. In some embodiments, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LRKKHLQMARECGAGTFVNFASLERDGKLPYNWRRSIFAFLTLLPSCLWTDYLLAFYINPWSKN GLKKRKLTNPVQLDDFDAYIKDMAKDSDYKFSLQFEELKLIGLDIPHFAADLPLNRCKNRYTNIL PYDFSRVRLVSMNEEEGADYINANYIPGYNSPQEYIATQGPLPETRNDFWKMVLQQKSQIIVML TQCNEKRRVKCDHYWPFTEEPIAYGDITVEMISEEEQDDWACRHFRINYADEMQDVMHFNYTA WPDHGVPTANAAESILQFVHMVRQQATKSKGPMIIHCSAGVGRTGTFIALDRLLQHIRDHEFVDI LGLVSEMRSYRMSMVQTEEQYIFIHQCVQLMWMKKKQQFCISDVIYENVSKS (SEQ ID NO: 7). In some embodiments, the intracellular signaling domain comprises the amino acid sequence of LRKKHLQMARECGAGTFVNFASLERDGKLPYNWRRSIFAFLTLLPSCLWTDYLLAFYINPWSKN GLKKRKLTNPVQLDDFDAYIKDMAKDSDYKFSLQFEELKLIGLDIPHFAADLPLNRCKNRYTNIL PYDFSRVRLVSMNEEEGADYINANYIPGYNSPQEYIATQGPLPETRNDFWKMVLQQKSQIIVML TQCNEKRRVKCDHYWPFTEEPIAYGDITVEMISEEEQDDWACRHFRINYADEMQDVMHFNYTA WPDHGVPTANAAESILQFVHMVRQQATKSKGPMIIHCSAGVGRTGTFIALDRLLQHIRDHEFVDI LGLVSEMRSYRMSMVQTEEQYIFIHQCVQLMWMKKKQQFCISDVIYENVSKS (SEQ ID NO: 7).

In some embodiments, one of the one or more intracellular signaling domains is derived from PTPRZ1. In some embodiments, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RKCFQTAHFYLEDSTSPRVISTPPTPIFPISDDVGAIPIKHFPKHVADLHASSGFTEEFETLKEFYQE VQSCTVDLGITADSSNHPDNKHKNRYINIVAYDHSRVKLAQLAEKDGKLTDYINANYVDGYNR PKAYIAAQGPLKSTAEDFWRMIWEHNVEVIVMITNLVEKGRRKCDQYWPADGSEEYGNFLVTQ KSVQVLAYYTVRNFTLRNTKIKKGSQKGRPSGRVVTQYHYTQWPDMGVPEYSLPVLTFVRKAA YAKRHAVGPVVVHCSAGVGRTGTYIVLDSMLQQIQHEGTVNIFGFLKHIRSQRNYLVQTEEQYV FIHDTLVEAILSKETEVLDSHIHAYVNALLIPGPAGKTKLEKQFQLLSQSNIQQSDYSAALKQCNR EKNRTSSIIPVERSRVGISSLSGEGTDYINASYIMGYYQSNEFIITQHPLLHTIKDFWRMIWDHNAQ LVVMIPDGQNMAEDEFVYWPNKDEPINCESFKVTLMAEEHKCLSNEEKLIIQDFILEATQDDYV LEVRHFQCPKWPNPDSPISKTFELISVIKEEAANRDGPMIVHDEHGGVTAGTFCALTTLMHQLEK ENSVDVYQVAKMINLMRPGVFADIEQYQFLYKVILSLVSTRQEENPSTSLDSNGAALPDGNIAES LESLV (SEQ ID NO: 8). In some embodiments, the intracellular signaling domain comprises the amino acid sequence of RKCFQTAHFYLEDSTSPRVISTPPTPIFPISDDVGAIPIKHFPKHVADLHASSGFTEEFETLKEFYQE VQSCTVDLGITADSSNHPDNKHKNRYINIVAYDHSRVKLAQLAEKDGKLTDYINANYVDGYNR PKAYIAAQGPLKSTAEDFWRMIWEHNVEVIVMITNLVEKGRRKCDQYWPADGSEEYGNFLVTQ KSVQVLAYYTVRNFTLRNTKIKKGSQKGRPSGRVVTQYHYTQWPDMGVPEYSLPVLTFVRKAA YAKRHAVGPVVVHCSAGVGRTGTYIVLDSMLQQIQHEGTVNIFGFLKHIRSQRNYLVQTEEQYV FIHDTLVEAILSKETEVLDSHIHAYVNALLIPGPAGKTKLEKQFQLLSQSNIQQSDYSAALKQCNR EKNRTSSIIPVERSRVGISSLSGEGTDYINASYIMGYYQSNEFIITQHPLLHTIKDFWRMIWDHNAQ LVVMIPDGQNMAEDEFVYWPNKDEPINCESFKVTLMAEEHKCLSNEEKLIIQDFILEATQDDYV LEVRHFQCPKWPNPDSPISKTFELISVIKEEAANRDGPMIVHDEHGGVTAGTFCALTTLMHQLEK ENSVDVYQVAKMINLMRPGVFADIEQYQFLYKVILSLVSTRQEENPSTSLDSNGAALPDGNIAES LESLV (SEQ ID NO: 8).

In some embodiments, the chimeric inhibitory receptor comprises at least one ITSM. In some embodiments, one or more intracellular signaling domains are each derived from a protein selected from the group consisting of: SLAMF1 and SLAMF5.

In some embodiments, one of the one or more intracellular signaling domains is derived from SLAMF1. In some embodiments, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to QLRRRGKTNHYQTTVEKKSLTIYAQVQKPGPLQKKLDSFPAQDPCTTIYVAATEPVPESVQETNS ITVYASVTLPES (SEQ ID NO: 10). In some embodiments, the intracellular signaling domain comprises the amino acid sequence of QLRRRGKTNHYQTTVEKKSLTIYAQVQKPGPLQKKLDSFPAQDPCTTIYVAATEPVPESVQETNS ITVYASVTLPES (SEQ ID NO: 10).

In some embodiments, one of the one or more intracellular signaling domains is derived from SLAMF5. In some embodiments, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RLFKRRQGRIFPEGSCLNTFTKNPYAASKKTIYTYIMASRNTQPAESRIYDEILQSKVLPSKEEPVN TVYSEVQFADKMGKASTQDSKPPGTSSYEIVI (SEQ ID NO: 11). In some embodiments, the intracellular signaling domain comprises the amino acid sequence of RLFKRRQGRIFPEGSCLNTFTKNPYAASKKTIYTYIMASRNTQPAESRIYDEILQSKVLPSKEEPVN TVYSEVQFADKMGKASTQDSKPPGTSSYEIVI (SEQ ID NO: 11).

In some embodiments, the transmembrane domain is derived from a protein selected from the group consisting of: CD8, CD28, CD3ζ, CD4, 4-IBB, OX40, ICOS, 2B4, CD25, CD7, LAX, LAT, LIR1, PCAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a transmembrane domain derived from PECAM-1. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to GLIAVVIIGVIIALLIIAA (SEQ ID NO: 24). In some embodiments, the transmembrane domain comprises the amino acid sequence of GLIAVVIIGVIIALLIIAA (SEQ ID NO: 24).

In some embodiments, the chimeric inhibitory receptor comprises a transmembrane domain derived from LIR1. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VIGILVAVILLLLLLLLLFLI (SEQ ID NO: 25). In some embodiments, the transmembrane domain comprises the amino acid sequence of VIGILVAVILLLLLLLLLFLI (SEQ ID NO: 25).

In some embodiments, the chimeric inhibitory receptor comprises a transmembrane domain derived from IRTA2. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VAGGLLSIAGLAAGALLLYCW (SEQ ID NO: 26). In some embodiments, the transmembrane domain comprises the amino acid sequence of VAGGLLSIAGLAAGALLLYCW (SEQ ID NO: 26).

In some embodiments, the chimeric inhibitory receptor comprises a transmembrane domain derived from IRTA4. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LWGLFGVLGFTGVALLLYALF (SEQ ID NO: 27). In some embodiments, the transmembrane domain comprises the amino acid sequence of LWGLFGVLGFTGVALLLYALF (SEQ ID NO: 27).

In some embodiments, the chimeric inhibitory receptor comprises a transmembrane domain derived from NKIR. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VLLPLIFTILLLLLVAASLLA (SEQ ID NO: 28). In some embodiments, the transmembrane domain comprises the amino acid sequence of VLLPLIFTILLLLLVAASLLA (SEQ ID NO: 28).

In some embodiments, the chimeric inhibitory receptor comprises a transmembrane domain derived from IL1RAP. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VLLVVILIVVYHVYWLEMVLF (SEQ ID NO: 29). In some embodiments, the transmembrane domain comprises the amino acid sequence of VLLVVILIVVYHVYWLEMVLF (SEQ ID NO: 29).

In some embodiments, the chimeric inhibitory receptor comprises a transmembrane domain derived from PTPRO. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to ISVLAILSTLLIGLLLVTLII (SEQ ID NO: 30). In some embodiments, the transmembrane domain comprises the amino acid sequence of ISVLAILSTLLIGLLLVTLII (SEQ ID NO: 30).

In some embodiments, the chimeric inhibitory receptor comprises a transmembrane domain derived from PTPRZ1. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AVIPLVIVSALTFICLVVLVGILIYW (SEQ ID NO: 31). In some embodiments, the transmembrane domain comprises the amino acid sequence of AVIPLVIVSALTFICLVVLVGILIYW (SEQ ID NO: 31).

In some embodiments, the chimeric inhibitory receptor comprises a transmembrane domain derived from TLT1. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LIWGAVLLVGLLVAAVVLFAV (SEQ ID NO: 32). In some embodiments, the transmembrane domain comprises the amino acid sequence of LIWGAVLLVGLLVAAVVLFAV (SEQ ID NO: 32).

In some embodiments, the chimeric inhibitory receptor comprises a transmembrane domain derived from SLAMF1. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to WAVYAGLLGGVIMILIMVVIL (SEQ ID NO: 33). In some embodiments, the transmembrane domain comprises the amino acid sequence of WAVYAGLLGGVIMILIMVVIL (SEQ ID NO: 33).

In some embodiments, the chimeric inhibitory receptor comprises a transmembrane domain derived from SLAMF5. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LLSVLAMFFLLVLILSSVFLF (SEQ ID NO: 34). In some embodiments, the transmembrane domain comprises the amino acid sequence of LLSVLAMFFLLVLILSSVFLF (SEQ ID NO: 34).

In some embodiments, the chimeric inhibitory receptor comprises a transmembrane domain is derived from PCDHGC3. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LLLSLILVSVGFVVTVFGVII (SEQ ID NO: 139). In some embodiments, the transmembrane domain comprises the amino acid sequence of LLLSLILVSVGFVVTVFGVII (SEQ ID NO: 139).

In some embodiments, the chimeric inhibitory receptor comprises a transmembrane domain is derived from LIFR. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VGLIIAILIPVAVAVIVGVVTSILC (SEQ ID NO: 140). In some embodiments, the transmembrane domain comprises the amino acid sequence of VGLIIAILIPVAVAVIVGVVTSILC (SEQ ID NO: 140).

In some embodiments, the chimeric inhibitory receptor comprises a transmembrane domain is derived from ERMAP. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VALAVILPVLVLLIMVCLCLI (SEQ ID NO: 141). In some embodiments, the transmembrane domain comprises the amino acid sequence of VALAVILPVLVLLIMVCLCLI (SEQ ID NO: 141).

In some embodiments, the chimeric inhibitory receptor comprises a transmembrane domain is derived from IL1RAPL2. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to IELAGGLGAIFLLLVLLVVIY (SEQ ID NO: 142). In some embodiments, the transmembrane domain comprises the amino acid sequence of IELAGGLGAIFLLLVLLVVIY (SEQ ID NO: 142).

In some embodiments, wherein the chimeric inhibitory receptor comprises a transmembrane domain is derived from CDH5. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AVVAILLCILTITVITLLIFL (SEQ ID NO: 143). In some embodiments, the transmembrane domain comprises the amino acid sequence of AVVAILLCILTITVITLLIFL (SEQ ID NO: 143).

In some embodiments, wherein the chimeric inhibitory receptor comprises a transmembrane domain is derived from MPZL1. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to FPVWVVVGIVTAVVLGLTLLI (SEQ ID NO: 144). In some embodiments, the transmembrane domain comprises the amino acid sequence of FPVWVVVGIVTAVVLGLTLLI (SEQ ID NO: 144).

In some embodiments, the chimeric inhibitory receptor comprises a transmembrane domain is derived from MPZ. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to YGVVLGAVIGGVLGVVLLLLLLFYVV (SEQ ID NO: 145). In some embodiments, the transmembrane domain comprises the amino acid sequence of YGVVLGAVIGGVLGVVLLLLLLFYVV (SEQ ID NO: 145).

In some embodiments, the chimeric inhibitory receptor comprises a transmembrane domain is derived from FCGR2B. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SSSPMGIIVAVVTGIAVAAIVAA (SEQ ID NO: 146). In some embodiments, the transmembrane domain comprises the amino acid sequence of SSSPMGIIVAVVTGIAVAAIVAA (SEQ ID NO: 146).

In some embodiments, the chimeric inhibitory receptor comprises a transmembrane domain is derived from SIGLEC-6. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LGAVWGASITTLVFLCVCFIF (SEQ ID NO: 147). In some embodiments, the transmembrane domain comprises the amino acid sequence of LGAVWGASITTLVFLCVCFIF (SEQ ID NO: 147).

In some embodiments, the chimeric inhibitory receptor comprises a transmembrane domain is derived from MPIG6B. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LLIPLLGAGLVLGLGALGLVW (SEQ ID NO: 148). In some embodiments, the transmembrane domain comprises the amino acid sequence of LLIPLLGAGLVLGLGALGLVW (SEQ ID NO: 148).

In some embodiments, wherein the chimeric inhibitory receptor comprises a transmembrane domain is derived from VSIG4. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VFAIILIISLCCMVVETMAYI (SEQ ID NO: 149). In some embodiments, the transmembrane domain comprises the amino acid sequence of VFAIILIISLCCMVVFTMAYI (SEQ ID NO: 149).

In some embodiments, wherein the chimeric inhibitory receptor comprises a transmembrane domain is derived from SIGLEC-12. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to FGGAGATALVFLYFCIIFVVV (SEQ ID NO: 150). In some embodiments, the transmembrane domain comprises the amino acid sequence of FGGAGATALVFLYFCIIFVVV (SEQ ID NO: 150).

In some embodiments, wherein the chimeric inhibitory receptor comprises a transmembrane domain is derived from LIR8. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VVTGVSVAFVLLLFLLLFLLL (SEQ ID NO: 151). In some embodiments, the transmembrane domain comprises the amino acid sequence of VVTGVSVAFVLLLFLLLFLLL (SEQ ID NO: 151).

In some embodiments, wherein the chimeric inhibitory receptor comprises a transmembrane domain is derived from IRTA1. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VAAGATGGLLSALLLAVALLF (SEQ ID NO: 152). In some embodiments, the transmembrane domain comprises the amino acid sequence of VAAGATGGLLSALLLAVALLF (SEQ ID NO: 152).

In some embodiments, wherein the chimeric inhibitory receptor comprises a transmembrane domain is derived from KIR2DL4. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AVIRYSVAIILFTILPFFLLH (SEQ ID NO: 153). In some embodiments, the transmembrane domain comprises the amino acid sequence of AVIRYSVAIILFTILPFFLLH (SEQ ID NO: 153).

In some embodiments, wherein the chimeric inhibitory receptor comprises a transmembrane domain is derived from KIR2DL5. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LHILIGTSVAIILFIILFFFL (SEQ ID NO: 154). In some embodiments, the transmembrane domain comprises the amino acid sequence of LHILIGTSVAIILFIILFFFL (SEQ ID NO: 154).

In some embodiments, wherein the chimeric inhibitory receptor comprises a transmembrane domain is derived from SIGLEC-7. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to GAVGGAGATALVFLSFCVIFIVV (SEQ ID NO: 155). In some embodiments, the transmembrane domain comprises the amino acid sequence of GAVGGAGATALVFLSFCVIFIVV (SEQ ID NO: 155).

In some embodiments, wherein the chimeric inhibitory receptor comprises a transmembrane domain is derived from FCRH3. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AAGITGLVLSILVLAAAAALL (SEQ ID NO: 156). In some embodiments, the transmembrane domain comprises the amino acid sequence of AAGITGLVLSILVLAAAAALL (SEQ ID NO: 156).

In some embodiments, the chimeric inhibitory receptor comprises a transmembrane domain is derived from PCDHGC5. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LIVALATVSLLSLVTFTFLSA (SEQ ID NO: 157). In some embodiments, the transmembrane domain comprises the amino acid sequence of LIVALATVSLLSLVTFTFLSA (SEQ ID NO: 157).

In some embodiments, wherein the chimeric inhibitory receptor comprises a transmembrane domain is derived from CDH11. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to GALIAILACIVILLVIVVLFVTL (SEQ ID NO: 158). In some embodiments, the transmembrane domain comprises the amino acid sequence of GALIAILACIVILLVIVVLFVTL (SEQ ID NO: 158).

In some embodiments, wherein the chimeric inhibitory receptor comprises a transmembrane domain is derived from IMPG2. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VIIGITIASVVGLLVIFSAII (SEQ ID NO: 159). In some embodiments, the transmembrane domain comprises the amino acid sequence of VIIGITIASVVGLLVIFSAII (SEQ ID NO: 159).

In some embodiments, wherein the chimeric inhibitory receptor comprises a transmembrane domain is derived from DSCAM. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LVTISCILVGVLLLFVLLLVV (SEQ ID NO: 160). In some embodiments, the transmembrane domain comprises the amino acid sequence of LVTISCILVGVLLLFVLLLVV (SEQ ID NO: 160).

In some embodiments, wherein the protein is not expressed on the target tumor.

In some embodiments, wherein the protein is expressed on a non-tumor cell.

In some embodiments, wherein the protein is expressed on a non-tumor cell derived from a tissue selected from the group consisting of brain, neuronal tissue, endocrine, endothelial, bone, bone marrow, immune system, muscle, lung, liver, gallbladder, pancreas, gastrointestinal tract, kidney, urinary bladder, male reproductive organs, female reproductive organs, adipose, soft tissue, and skin.

In some embodiments, wherein the extracellular protein binding domain comprises a ligand-binding domain. In some embodiments, wherein the extracellular protein binding domain comprises a receptor-binding domain. In some embodiments, wherein the extracellular protein binding domain comprises an antigen-binding domain. In some embodiments, wherein the antigen-binding domain comprises an antibody, an antigen-binding fragment of an antibody, a F(ab) fragment, a F(ab′) fragment, a single chain variable fragment (scFv), or a single-domain antibody (sdAb).

In some embodiments, wherein the antigen-binding domain comprises a single chain variable fragment (scFv). In some embodiments, each scFv comprises a heavy chain variable domain (VH) and a light chain variable domain (VL). In some embodiments, the VH and VL are separated by a peptide linker. In some embodiments, the peptide linker comprises an amino acid sequence selected from the group consisting of:

(SEQ ID NO: 47)
GGS,
(SEQ ID NO: 48)
GGSGGS,
(SEQ ID NO: 49)
GGSGGSGGS,
(SEQ ID NO: 50)
GGSGGSGGSGGS,
(SEQ ID NO: 51)
GGSGGSGGSGGSGGS,
(SEQ ID NO: 52)
GGGS,
(SEQ ID NO: 53)
GGGSGGGS,
(SEQ ID NO: 54)
GGGSGGGSGGGS,
(SEQ ID NO: 55)
GGGSGGGSGGGSGGGS,
(SEQ ID NO: 56)
GGGSGGGSGGGSGGGSGGGS,
(SEQ ID NO: 57)
GGGGS,
(SEQ ID NO: 58)
GGGGSGGGGS,
(SEQ ID NO: 59)
GGGGSGGGGSGGGGS,
(SEQ ID NO: 60)
GGGGSGGGGSGGGGSGGGGS,
(SEQ ID NO: 61)
GGGGSGGGGSGGGGSGGGGSGGGGS,
(SEQ ID NO: 63)
GGGGSGGGGSGGGGSQSV,
(SEQ ID NO: 64)
GSTSGSGKPGSGEGSTKG,
(SEQ ID NO: 65)
SGGGGSGGGGSGGGGSGGGGSGGGSLQ,
and
(SEQ ID NO: 62)
TTTPAPRPPTPAPTIALQPLSLRPEACRPAAGGAVHTRGLDFACDQTT
PGERSSLPAFYPGTSGSCSGCGSLSLP.

In some embodiments, the scFv comprises the structure VH-L-VL or VL-L-VH, wherein VH is the heavy chain variable domain, L is the peptide linker, and VL is the light chain variable domain.

In some embodiments, the transmembrane domain is physically linked to the extracellular protein binding domain. In some embodiments, one of the one or more intracellular signaling domains is physically linked to the transmembrane domain. In some embodiments, the transmembrane domain is physically linked to the extracellular protein binding domain and one of the one or more intracellular signaling domains is physically linked to the transmembrane domain.

In some embodiments, the extracellular protein binding domain has a high binding affinity. In some embodiments, extracellular protein binding domain has a low binding affinity.

In some embodiments, the chimeric inhibitory receptor is capable of suppressing cytokine production from an activated immunomodulatory cell.

In some embodiments, the chimeric inhibitory receptor is capable of suppressing a cell-mediated immune response to a target cell, wherein the immune response is induced by activation of the immunomodulatory cell.

In some embodiments, the target cell is a tumor cell.

In some embodiments, the one or more intracellular signaling domains comprise one or more modifications. In some embodiments, the one or more modifications modulate sensitivity of the chimeric inhibitory receptor relative to the otherwise identical, unmodified receptor. In some embodiments, the one or more modifications increase sensitivity of the chimeric inhibitory receptor relative to the otherwise identical, unmodified receptor. In some embodiments, the one or more modifications reduce sensitivity of the chimeric inhibitory receptor relative to the otherwise identical, unmodified receptor. In some embodiments, the one or more modifications modulate potency of the chimeric inhibitory receptor relative to the otherwise identical, unmodified receptor. In some embodiments, the one or more modifications increase potency of the chimeric inhibitory receptor relative to the otherwise identical, unmodified receptor. In some embodiments, the one or more modifications reduce potency of the chimeric inhibitory receptor relative to the otherwise identical, unmodified receptor. In some embodiments, the one or more modifications modulate basal prevention, attenuation, or inhibition of activation of the tumor-targeting chimeric receptor when expressed on an immunomodulatory cell relative to the otherwise identical, unmodified receptor. In some embodiments, the one or more modifications reduce basal prevention, attenuation, or inhibition relative to the otherwise identical, unmodified receptor. In some embodiments, the one or more modifications increase basal prevention, attenuation, or inhibition relative to the otherwise identical, unmodified receptor.

In some embodiments, the chimeric inhibitory receptor further comprises a spacer region positioned between the extracellular protein binding domain and the transmembrane domain and operably linked to each of the extracellular protein binding domain and the transmembrane domain. In some embodiments, the chimeric inhibitory receptor further comprises a spacer region positioned between the extracellular protein binding domain and the transmembrane domain and physically linked to each of the extracellular protein binding and the transmembrane domain. In some embodiments, the spacer region is derived from a protein selected from the group consisting of: CD8α, CD4, CD7, CD28, IgG1, IgG4, FcγRIIIα, LNGFR, and PDGFR.

In some embodiments, the spacer region comprises an amino acid sequence selected from the group consisting of:

(SEQ ID NO: 73)
TTTPAPRPPTPAPTIALQPLSLRPEACRPAAGGAVHTRGLDFACD,
(SEQ ID NO: 77)
ALSNSIMYFSHFVPVFLPAKPTTTPAPRPPTPAPTIASQPLSLRPEAC
RPAAGGAVHTRGLDFACD,
(SEQ ID NO: 67)
AAAIEVMYPPPYLDNEKSNGTIIHVKGKHLCPSPLFPGPSKP,
(SEQ ID NO: 68)
ESKYGPPCPSCP,
(SEQ ID NO: 69)
ESKYGPPAPSAP,
(SEQ ID NO: 70)
ESKYGPPCPPCP,
(SEQ ID NO: 71)
EPKSCDKTHTCP,
(SEQ ID NO: 72)
AAAFVPVFLPAKPTTTPAPRPPTPAPTIASQPLSLRPEACRPAAGGAV
HTRGLDFACDIYIWAPLAGTCGVLLLSLVITLYCNHRN,
(SEQ ID NO: 74)
ACPTGLYTHSGECCKACNLGEGVAQPCGANQTVCEPCLDSVTFSDVVS
ATEPCKPCTECVGLQSMSAPCVEADDAVCRCAYGYYQDETTGRCEACR
VCEAGSGLVFSCQDKQNTVCEECPDGTYSDEADAEC,
(SEQ ID NO: 75)
ACPTGLYTHSGECCKACNLGEGVAQPCGANQTVC,
(SEQ ID NO: 76)
AVGQDTQEVIVVPHSLPFKV,
(SEQ ID NO: 183)
ESKYGPPCPSCPAPPVAGPSVFLFPPKPKDTLMISRTPEVTCVVVDVS
QEDPEVQFNWYVDGVEVHNAKTKPREEQFQSTYRVVSVLTVLHQDWLN
GKEYKCKVSNKGLPSSIEKTISKAKGQPREPQVYTLPPSQEEMTKNQV
SLTCLVKGFYPSDIAVEWESNGQPENNYKTTPPVLDSDGSFFLYSRLT
VDKSRWQEGNVFSCSVMHEALHNHYTQKSLSLSLGK,
and
(SEQ ID NO: 184)
ESKYGPPCPSCPGQPREPQVYTLPPSQEEMTKNQVSLTCLVKGFYPSD
IAVEWESNGQPENNYKTTPPVLDSDGSFFLYSRLTVDKSRWQEGNVFS
CSVMHEALHNHYTQKSLSLSLGK.

In some embodiments, the spacer region modulates sensitivity of the chimeric inhibitory receptor relative to an otherwise identical chimeric inhibitory receptor lacking the spacer region. In some embodiments, the spacer region increases sensitivity of the chimeric inhibitory receptor relative to an otherwise identical chimeric inhibitory receptor lacking the spacer region. In some embodiments, the spacer region reduces sensitivity of the chimeric inhibitory receptor relative to an otherwise identical chimeric inhibitory receptor lacking the spacer region. In some embodiments, the spacer region modulates potency of the chimeric inhibitory receptor relative to an otherwise identical chimeric inhibitory receptor lacking the spacer region. In some embodiments, the spacer region increases potency of the chimeric inhibitory receptor relative to an otherwise identical chimeric inhibitory receptor lacking the spacer region. In some embodiments, the spacer region reduces potency of the chimeric inhibitory receptor relative to an otherwise identical chimeric inhibitory receptor lacking the spacer region. In some embodiments, the spacer region modulates basal prevention, attenuation, or inhibition of activation of the tumor-targeting chimeric receptor when expressed on an immunomodulatory cell relative to an otherwise identical chimeric inhibitory receptor lacking the spacer region. In some embodiments, the spacer region reduces basal prevention, attenuation, or inhibition relative to an otherwise identical chimeric inhibitory receptor lacking the spacer region. In some embodiments, the spacer region increases basal prevention, attenuation, or inhibition relative to an otherwise identical chimeric inhibitory receptor lacking the spacer region. In some embodiments, the chimeric inhibitory receptor further comprises an intracellular spacer region positioned between the transmembrane domain and one of the one or more intracellular signaling domains and operably linked to each of the transmembrane domain and one of the one or more intracellular signaling domains. In some embodiments, the chimeric inhibitory receptor further comprises an intracellular spacer region positioned between the transmembrane domain and one of the one or more intracellular signaling domains and physically linked to each of the transmembrane domain and one of the one or more intracellular signaling domains. In some embodiments, the intracellular spacer region modulates sensitivity of the chimeric inhibitory receptor relative to an otherwise identical chimeric inhibitory receptor lacking the intracellular spacer region. In some embodiments, the intracellular spacer region increases sensitivity of the chimeric inhibitory receptor relative to an otherwise identical chimeric inhibitory receptor lacking the intracellular spacer region.

In some embodiments, the intracellular spacer region reduces sensitivity of the chimeric inhibitory receptor relative to an otherwise identical chimeric inhibitory receptor lacking the intracellular spacer region. In some embodiments, the intracellular spacer region modulates potency of the chimeric inhibitory receptor relative to an otherwise identical chimeric inhibitory receptor lacking the intracellular spacer region. In some embodiments, the intracellular spacer region increases potency of the chimeric inhibitory receptor relative to an otherwise identical chimeric inhibitory receptor lacking the intracellular spacer region. In some embodiments, the intracellular spacer region reduces potency of the chimeric inhibitory receptor relative to an otherwise identical chimeric inhibitory receptor lacking the intracellular spacer region. In some embodiments, the intracellular spacer region modulates basal prevention, attenuation, or inhibition of activation of the tumor-targeting chimeric receptor when expressed on an immunomodulatory cell relative to an otherwise identical chimeric inhibitory receptor lacking the intracellular spacer region. In some embodiments, the intracellular spacer region reduces basal prevention, attenuation, or inhibition relative to an otherwise identical chimeric inhibitory receptor lacking the intracellular spacer region. In some embodiments, the intracellular spacer region increases basal prevention, attenuation, or inhibition relative to an otherwise identical chimeric inhibitory receptor lacking the intracellular spacer region.

In some embodiments, the inhibitory chimeric receptor further comprises an enzymatic inhibitory domain. In some embodiments, the enzymatic inhibitory domain is capable of preventing, attenuating, or inhibiting activation of a tumor-targeting chimeric receptor when expressed on an immunomodulatory cell relative to an otherwise identical chimeric inhibitory receptor lacking the enzymatic inhibitory domain.

In some embodiments, the enzymatic inhibitory domain comprises an enzyme catalytic domain. In some embodiments, the enzyme catalytic domain is derived from an enzyme selected from the group consisting of: CSK, SHP-1, PTEN, CD45, CD148, PTP-MEG1, PTP-PEST, c-CBL, CBL-b, PTPN22, LAR, PTPH1, SHIP-1, and RasGAP.

In some embodiments, the enzymatic inhibitory domain comprises one or more modifications that modulate basal prevention, attenuation, or inhibition.

In some embodiments, the one or more modifications reduce basal prevention, attenuation, or inhibition relative to an otherwise identical enzymatic inhibitory domain lacking the one or more modifications. In some embodiments, the one or more modifications increase basal prevention, attenuation, or inhibition relative to an otherwise identical enzymatic inhibitory domain lacking the one or more modifications.

In some embodiments, the tumor-targeting chimeric receptor is a chimeric antigen receptor (CAR) or an engineered T cell receptor (TCR). In some embodiments, the immunomodulatory cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell. In some embodiments, the immunomodulatory cell is a Natural Killer (NK) cell.

Another embodiment of the disclosure provides a composition comprising the chimeric inhibitory receptor of the present disclosure and a pharmaceutically acceptable carrier, a pharmaceutically acceptable excipient, or a combination thereof.

Another embodiment of the disclosure provides an engineered nucleic acid encoding the chimeric inhibitory receptor of the present disclosure.

Another embodiment of the disclosure provides an expression vector comprising the engineered nucleic acid of the present disclosure.

Another embodiment of the disclosure provides a composition comprising the engineered nucleic acid of the present disclosure or the expression vector of the present disclosure, and a pharmaceutically acceptable carrier.

Another embodiment of the disclosure provides a producer cell comprising the chimeric inhibitory receptor of the present disclosure, the engineered nucleic acid of the present disclosure, or the expression vector of the present disclosure.

Another embodiment of the disclosure provides an isolated immunomodulatory cell comprising the chimeric inhibitory receptor of the present disclosure, the engineered nucleic acid of the present disclosure, or the expression vector of the present disclosure. In some embodiments, the cell further comprises a tumor-targeting chimeric receptor expressed on the surface of the cell. In some embodiments, upon binding of the protein to the chimeric inhibitory receptor, the chimeric inhibitory receptor prevents, attenuates, or inhibits activation of the tumor-targeting chimeric receptor relative to an otherwise identical cell lacking a chimeric inhibitory receptor.

Another embodiment of the disclosure provides an isolated immunomodulatory cell comprising a chimeric inhibitory receptor, wherein the chimeric inhibitory receptor comprises an extracellular protein binding domain, a transmembrane domain, wherein the transmembrane domain is operably linked to the extracellular protein binding domain, and one or more intracellular signaling domains, wherein the one or more intracellular signaling domains are operably linked to the transmembrane domain, and wherein the one or more intracellular signaling domain are each derived from a protein selected from the group consisting of: PECAM-1, CD72, IRTA2, IRTA4, NKIR, IL1RAP, PTPRO, PTPRZ1, TLT1, SLAMF1, sSLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM; and wherein upon binding of the protein to the chimeric inhibitory receptor, the chimeric inhibitory receptor prevents, attenuates, or inhibits activation of a tumor-targeting chimeric receptor expressed on the surface of the cell. In some embodiments, the cell further comprises a tumor-targeting chimeric receptor expressed on the surface of the cell.

Another embodiment of the disclosure provides an isolated cell comprising a chimeric inhibitory receptor, wherein and the chimeric inhibitory receptor comprises: an extracellular protein binding domain, a transmembrane domain, wherein the transmembrane domain is operably linked to the extracellular protein binding domain, and one or more intracellular signaling domains, wherein the one or more intracellular signaling domains are operably linked to the transmembrane domain, and wherein the one or more intracellular signaling domain are each derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM; and a tumor-targeting chimeric receptor expressed on the surface of the cell, wherein upon binding of the protein to the chimeric inhibitory receptor, the chimeric inhibitory receptor prevents, attenuates, or inhibits activation of the tumor-targeting chimeric receptor. In some embodiments, the chimeric inhibitory receptor is recombinantly expressed. In some embodiments, the chimeric inhibitory receptor is expressed from a vector or a selected locus from the genome of the cell.

In some embodiments, the tumor-targeting chimeric receptor is a chimeric antigen receptor (CAR) or an engineered T cell receptor. In some embodiments, prior to binding of the protein to the chimeric inhibitory receptor, the tumor-targeting chimeric receptor is capable of activating the cell. In some embodiments, upon binding of the protein to the chimeric inhibitory receptor, the chimeric inhibitory receptor suppresses cytokine production from the activated cell. In some embodiments, upon binding of the protein to the chimeric inhibitory receptor, the chimeric inhibitory receptor suppresses a cell-mediated immune response to a target cell, wherein the immune response is induced by activation of the immunomodulatory cell.

In some embodiments, the transmembrane domain is physically linked to the extracellular protein binding domain. In some embodiments, the intracellular signaling domain is physically linked to the transmembrane domain. In some embodiments, the transmembrane domain is physically linked to the extracellular protein binding domain and one of the one or more intracellular signaling domains is physically linked to the transmembrane domain. In some embodiments, the target cell is a tumor cell.

In some embodiments, the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell. In some embodiments, the cell is a Natural Killer (NK) cell. In some embodiments, the cell is autologous. In some embodiments, the cell is allogeneic.

Another embodiment of the disclosure provides a composition comprising the isolated cell of the present disclosure and a pharmaceutically acceptable carrier, a pharmaceutically acceptable excipient, or a combination thereof.

Another embodiment of the disclosure provides a method of preventing, attenuating, or inhibiting a cell-mediated immune response induced by a tumor-targeting chimeric receptor expressed of the surface of an immunomodulatory cell, comprising: engineering the immunomodulatory cell to express the chimeric inhibitory receptor of any one of the present disclosure on the surface of the immunomodulatory cell, wherein upon binding of a cognate antigen to the chimeric inhibitory receptor, the intracellular signaling domain prevents, attenuates, or inhibits activation of the tumor-targeting chimeric receptor.

Another embodiment of the disclosure provides a method of preventing, attenuating, or inhibiting activation of a tumor-targeting chimeric receptor expressed on the surface of an immunomodulatory cell, comprising: contacting the isolated cell of the present disclosure or the composition of the present disclosure with a cognate antigen of the chimeric inhibitory receptor under conditions suitable for the chimeric inhibitory receptor to bind the cognate antigen, wherein upon binding of the antigen to the chimeric inhibitory receptor, the intracellular signaling domain prevents, attenuates, or inhibits activation of the tumor-targeting chimeric receptor.

In some embodiments, the tumor-targeting chimeric receptor is a chimeric antigen receptor (CAR) or an engineered T cell receptor. In some embodiments, the CAR binds one or more antigens expressed on the surface of a tumor cell.

### BRIEF DESCRIPTION OF THE DRAWINGS

These and other features, aspects, and advantages of the present invention will become better understood with regard to the following description, and accompanying drawings, where:

FIG. 1 shows an exemplary diagram of a T cell co-expressing an anti-CD19-SLAP iCAR and an anti-CD20-CD28/CD3ζ aCAR contacting a target cell expressing CD19 and CD20.

FIG. 2A shows killing of SEM cells using a NK cells expressing a combination of aCARs and iCARs with different iCAR intracellular domains. FIG. 2B shows secretion of TNF-α (TNFa) from NK cells expressing the same aCAR and iCARs.

FIG. 3 shows the expression levels of the different aCAR-iCAR combinations as visualized by flow cytometry. Flow cytometry to assess expression levels were conducted 6 days after transduction with the aCAR-iCAR constructs.

FIG. 4A shows aCAR specific killing of SEM cells using donor-derived NK cells transduced with aCAR and different iCAR constructs having variable intracellular domains. FIG. 4B shows expression of interferon-γ (IFNg) of the same cells of FIG. 4A. FIG. 4C shows expression of TNF-α (TNFa) of the same cells of FIG. 4A.

FIG. 5A shows characterization of different aCAR-iCAR combinations in NK cells as determined by flow cytometry. The top panel shows the mean fluorescence intensity of aCAR/iCARs in transduced NK cells. FIG. 5B shows the characterization of NK-mediated cell killing of SEM target cells expressing the target antigen and/or the safety antigen.

FIG. 6 shows expression of VSIG2-targeting iCARs with various inhibitor ICDs in engineered NK cells, and the protective effect of the VSIG2 targeting iCARs towards VSIG2 expressing cells.

### DETAILED DESCRIPTION

### Definitions

Terms used in the claims and specification are defined as set forth below unless otherwise specified.

The term “inhibitory chimeric receptor” or “inhibitory chimeric antigen receptor” or “chimeric inhibitory receptor” as used herein refers to a polypeptide or a set of polypeptides, which when expressed in an immune effector cell, provides the cell with specificity for a target cell, and with inhibitory intracellular signal generation. Inhibitory chimeric receptors typically include an extracellular protein binding domain (e.g., antibody fragment as an antigen-binding domain), a spacer domain, a transmembrane domain, and one or more intracellular signaling/co-signaling domains. An inhibitory chimeric receptor may also be called an “iCAR.”

The term “tumor targeting chimeric receptor” refers to activating chimeric receptors, tumor-targeting chimeric antigen receptors (CARs), or engineered T cell receptors. A tumor targeting chimeric receptor may also be called an “aCAR.”

The term “chimeric antigen receptor” or alternatively a “CAR” as used herein refers to a polypeptide or a set of polypeptides, which when expressed in an immune effector cell, provides the cell with specificity for a target cell, and with intracellular signal generation. CARs typically include an extracellular protein binding domain (e.g., antibody fragment as an antigen-binding domain), a spacer domain, a transmembrane domain, and one or more intracellular signaling/co-signaling domains. In some embodiments, a CAR comprises at least an extracellular antigen binding domain, a transmembrane domain and a cytoplasmic signaling domain (also referred to herein as “an intracellular signaling domain”) comprising a functional signaling domain derived from a inhibitory molecule or a stimulatory molecule and/or costimulatory molecule. In some aspects, the set of polypeptides that comprise the inhibitory chimeric receptor or tumor targeting chimeric receptor are contiguous with each other. In some embodiments, the inhibitory chimeric receptor or tumor targeting chimeric receptor further comprises a spacer domain between the extracellular antigen binding domain and the transmembrane domain. In some embodiments, the set of polypeptides include recruitment domains, such as dimerization or multimerization domains, that can couple the polypeptides to one another. In some embodiments, an inhibitory chimeric receptor comprises a chimeric fusion protein comprising an extracellular antigen binding domain, a transmembrane domain and an intracellular signaling domain comprising a functional signaling domain derived from an inhibitory molecule or a stimulatory molecule. In one aspect, an inhibitory chimeric receptor comprises a chimeric fusion protein comprising an extracellular antigen binding domain, a transmembrane domain and an intracellular signaling domain comprising a functional inhibitory domain derived from an inhibitory molecule. In one aspect, a tumor targeting chimeric receptor comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule.

The term, “intracellular signaling domain” as used herein, refers to a functional domain of the inhibitory chimeric receptor or the tumor targeting chimeric receptor located inside the cell. In some embodiments, the intracellular signaling domain is an inhibitory signaling domain. Following binding of the molecular binding domain to an protein, for example, an inhibitory signaling domain represses receptor signaling while an activation signaling domain transmits a signal (e.g., proliferative/survival signal) to the cell.

The term. “transmembrane domain” as used herein, refers to a domain that spans a cellular membrane. In some embodiments, a transmembrane domain comprises a hydrophobic alpha helix.

The term. “extracellular protein binding domain” or “extracellular antigen binding domain” as used herein, refers to a molecular binding domain which is typically an ectodomain of a cell receptor or the antigen binding domains of an antibody and is located outside the cell, exposed to the extracellular space. An extracellular antigen binding domain can include any molecule (e.g., protein or peptide) capable of binding to another protein or peptide. In some embodiments, an extracellular protein or antigen binding domain comprises an antibody, an antigen-binding fragment thereof, F(ab), F(ab′), a single chain variable fragment (scFv), or a single-domain antibody (sdAb). In some embodiments, an extracellular protein or antigen binding domain binds to a cell-surface ligand (e.g., an antigen, such as a cancer antigen or a protein expressed on the surface of a cell).

The term “tumor” refers to tumor cells and the associated tumor microenvironment (TME). In some embodiments, tumor refers to a tumor cell or tumor mass. In some embodiments, tumor refers to the tumor microenvironment.

The term “not expressed” refers to expression that is at least 2-fold lower than the level of expression in non-tumor cells that would result in activation of the tumor-targeting chimeric antigen receptor. In some embodiments, the expression is at least 2-fold, at least 3-fold, at least 4-fold, at least 5-fold, at least 6-fold, at least 7-fold, at least 8-fold, at least 9-fold, or at least 10-fold or more lower than the level of expression in non-tumor cells that would result in activation of the tumor-targeting chimeric antigen receptor.

The term “ameliorating” refers to any therapeutically beneficial result in the treatment of a disease state, e.g., a cancer disease state, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines.

The term percent “identity.” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

It must be noted that, as used in the specification and the appended claims, the singular forms “a,” “an” and “the” include plural referents unless the context clearly dictates otherwise.

### Chimeric Inhibitory Receptors

In one aspect, provided herein are chimeric inhibitory receptors comprising (i) an extracellular protein binding domain; (ii) a transmembrane domain, wherein the transmembrane domain is operably linked to the extracellular protein binding domain; and (iii) one or more intracellular signaling domains, wherein the one or more intracellular signaling domains are operably linked to the transmembrane domain, and wherein at least one of the one or more intracellular signaling domains is capable of preventing, attenuating, or inhibiting activation of a tumor-targeting chimeric receptor expressed on an immunomodulatory cell. In some embodiments, a chimeric inhibitory receptor of the present disclosure comprises two or more, three or more, four or more, or five or more intracellular signaling domains. In some embodiments, a chimeric inhibitory receptor of the present disclosure comprises one intracellular signaling domain. In some embodiments, a chimeric inhibitory receptor of the present disclosure comprises two intracellular signaling domains. In some embodiments, a chimeric inhibitory receptor of the present disclosure comprises three intracellular signaling domains. In some embodiments, a chimeric inhibitory receptor of the present disclosure comprises four intracellular signaling domains. In some embodiments, a chimeric inhibitory receptor of the present disclosure comprises five intracellular signaling domains.

Generally, an inhibitory or tumor targeting chimeric receptor is designed for a T cell, or NK cell, and is a chimera of an intracellular signaling domain and an antigen-recognizing domain (e.g., a single chain fragment (scFv) of an antibody) (Enblad et al., Human Gene Therapy. 2015; 26 (8): 498-505). A T cell that expresses a chimeric antigen receptor (CAR) is known in the art as a CAR T cell. An activating or tumor targeting CAR generally induces T cell signaling pathways upon binding to its cognate ligand via an intracellular signaling domain that results in activation of the T cell and an immune response. Activation CAR, activating CAR, and tumor-targeting CAR are interchangeable terms.

An inhibitory chimeric receptor, generally, is an artificial immune cell receptor engineered to recognize and bind to proteins expressed by cells. Inhibitory chimeric receptors generally recognize proteins that are not expressed on tumor cells, while activating or tumor targeting chimeric receptors (e.g., aCARs) generally recognize proteins that are expressed on tumor cells. In some embodiments, chimeric inhibitory receptors of the present disclosure specifically bind to one or more proteins that are expressed on normal cells (e.g., cells generally considered healthy) but not on tumor cells. Chimeric receptors in general typically include an antibody fragment as an extracellular protein binding domain, a spacer or hinge domains, a hydrophobic alpha helix transmembrane domain, and one or more intracellular signaling/co-signaling domains.

An inhibitory chimeric receptor generally follows the structure of activating CARs (aCARs) but uses an inhibitory domain for the intracellular signaling domain, instead of an activation signaling domain derived from a T-cell receptor (TCR). The intracellular signaling/co-signaling domain are inhibitory domains that reduce or inhibit signaling by other receptor proteins in the same cell. An inhibitory chimeric receptor cell can contain an antigen-specific inhibitory receptor, for example, to block nonspecific immunoactivation, which may result from extra-tumor target expression. In some embodiments, an inhibitory chimeric receptor blocks T cell responses in T cells activated by either their endogenous T cell receptor or an activating or tumor-targeting CAR. For example, an immunomodulatory cell can express both an inhibitory chimeric receptor that recognizes a non-tumor protein target and a tumor-targeting chimeric receptor that recognizes a tumor protein. When such an immunomodulatory cell contacts a tumor cell, only the tumor-targeting receptor recognizes and binds its cognate ligand and is activated, resulting in induction of cell signaling pathways and immune cell activation. In contrast, when the immunomodulatory cell contacts a non-tumor target, the inhibitory chimeric receptor binds to its cognate ligand and represses or inhibits any signaling induced by the activation of the tumor-targeting chimeric receptor. Thus, the immunomodulatory cell can be constructed so that immune signaling only occurs when the cell contacts tumor cells.

Inhibitory chimeric receptors of the present disclosure can inhibit the function of an aCAR through mechanisms that dampen the downstream signaling of the aCAR. The inhibitory chimeric receptor can comprise an inhibitory tyrosine-based inhibitory motif (ITIM), an inhibitory tyrosine-based switch motif (ITSM), or a phosphatase, such as a protein tyrosine phosphatase (PTP). ITIMs typically comprise the amino acid motif of S/I/V/LxYxxI/V/L, where x is any amino acid, Y is a tyrosine residue that can be phosphorylated, S is the amino acid Serine, I is the amino acid Isoleucine, and V is the amino acid Valine. ITIMs recruit SH2 domain-containing phosphatases, which inhibit cellular activation by targeting Immunoreceptor tyrosine-based activation motif (ITAM)-containing receptors (aCARs), resulting in inhibition of the aCAR within a cell. ITSMs can provide an activating or inhibitory signal. PTPs can remove phosphates from phosphorylated tyrosine residues, thereby modulating signaling in a cell.

In some embodiments, the protein bound by the inhibitory chimeric receptor is not expressed on the target tumor. In some embodiments, the expression is at least 2-fold, at least 3-fold, at least 4-fold, at least 5-fold, at least 6-fold, at least 7-fold, at least 8-fold, at least 9-fold, or at least 10-fold or more lower than the level of expression in non-tumor cells that would result in activation of the tumor-targeting chimeric antigen receptor.

In some embodiments, the protein bound by the inhibitory chimeric receptor is expressed on a non-tumor cell.

In some embodiments, the protein bound by the inhibitory chimeric receptor is expressed on a non-tumor cell derived from a tissue selected from the group consisting of brain, neuronal tissue, endocrine, endothelial, bone, bone marrow, immune system, muscle, lung, liver, gallbladder, pancreas, gastrointestinal tract, kidney, urinary bladder, male reproductive organs, female reproductive organs, adipose, soft tissue, and skin.

### Intracellular Signaling Domains

The inhibitory chimeric receptors of the present disclosure comprise intracellular signaling domains that are capable of preventing, attenuating, or inhibiting activation of a tumor-targeting chimeric receptor expressed on an immunomodulatory cell. In some embodiments, the chimeric inhibitory receptor comprises one or more intracellular signaling domains. In some embodiments, the one or more intracellular signaling domains comprise at least one ITIM, at least one ITSM, at least one phosphatase, or any combination thereof. In some embodiments, the one or more intracellular signaling domains comprise at least one ITIM. In some embodiments, the at least one intracellular signaling domains comprise at least one ITSM. In some embodiments, the at least one intracellular signaling domains comprise at least one phosphatase. In some embodiments, the at least one intracellular domain comprises at least one ITIM and at least one phosphatase. In some embodiments, the at least one intracellular domain comprises at least one ITIM and at least one ITSM. In some embodiments, the at least one intracellular domain comprises at least one ITSM and at least one phosphatase. In some embodiments, the phosphatase is a protein tyrosine phosphatase.

In some embodiments, the intracellular signaling domain comprises one or more modifications. In some embodiments, the one or more modifications modulate sensitivity of the chimeric inhibitory receptor relative to the otherwise identical, unmodified receptor. In some embodiments, the one or more modifications increase sensitivity of the chimeric inhibitory receptor relative to the otherwise identical, unmodified receptor. In some embodiments, the one or more modifications reduce sensitivity of the chimeric inhibitory receptor relative to the otherwise identical, unmodified receptor. In some embodiments, the one or more modifications modulate potency of the chimeric inhibitory receptor relative to the otherwise identical, unmodified receptor. In some embodiments, the one or more modifications increase potency of the chimeric inhibitory receptor relative to the otherwise identical, unmodified receptor. In some embodiments, the one or more modifications reduce potency of the chimeric inhibitory receptor relative to the otherwise identical, unmodified receptor.

In some embodiments, the one or more modifications modulate basal prevention, attenuation, or inhibition of activation of the tumor-targeting chimeric receptor expressed on an immunomodulatory cell relative to the otherwise identical, unmodified receptor. In some embodiments, the one or more modifications reduce basal prevention, attenuation, or inhibition relative to the otherwise identical, unmodified receptor. In some embodiments, the one or more modifications increase basal prevention, attenuation, or inhibition relative to the otherwise identical, unmodified receptor.

### Inhibitory Domains

In some embodiments, the inhibitory intracellular signaling domain is derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM. In some embodiments, the inhibitory intracellular signaling domain is a PECAM-1 domain. In some embodiments, the inhibitory intracellular signaling domain is a CD72 domain. In some embodiments, the inhibitory intracellular signaling domain is a IRTA2 domain. In some embodiments, the inhibitory intracellular signaling domain is a IRTA4 domain. In some embodiments, the inhibitory intracellular signaling domain is a NKIR domain. In some embodiments, the inhibitory intracellular signaling domain is a IL1RAP domain. In some embodiments, the inhibitory intracellular signaling domain is a PTPRO domain. In some embodiments, the inhibitory intracellular signaling domain is a PTPRZ1 domain. In some embodiments, the inhibitory intracellular signaling domain is a TLT1 domain. In some embodiments, the inhibitory intracellular signaling domain is a SLAMF1. In some embodiments, the inhibitory intracellular signaling domain is a SLAMF5. In some embodiments, the inhibitory intracellular signaling domain is a PCDHGC3. In some embodiments, the inhibitory intracellular signaling domain is a LIFR. In some embodiments, the inhibitory intracellular signaling domain is a ERMAP. In some embodiments, the inhibitory intracellular signaling domain is a IL1RAPL2. In some embodiments, the inhibitory intracellular signaling domain is a CDH5. In some embodiments, the inhibitory intracellular signaling domain is a MPZL1. In some embodiments, the inhibitory intracellular signaling domain is a MPZ. In some embodiments, the inhibitory intracellular signaling domain is a FCGR2B. In some embodiments, the inhibitory intracellular signaling domain is a SIGLEC-6. In some embodiments, the inhibitory intracellular signaling domain is a MPIG6B. In some embodiments, the inhibitory intracellular signaling domain is a VSIG4. In some embodiments, the inhibitory intracellular signaling domain is a SIGLEC-12. In some embodiments, the inhibitory intracellular signaling domain is a LIR8. In some embodiments, the inhibitory intracellular signaling domain is a IRTA1. In some embodiments, the inhibitory intracellular signaling domain is a KIR2DL4. In some embodiments, the inhibitory intracellular signaling domain is a KIR2DL5. In some embodiments, the inhibitory intracellular signaling domain is a SIGLEC-7. In some embodiments, the inhibitory intracellular signaling domain is a FCRH3. In some embodiments, the inhibitory intracellular signaling domain is a PCDHGC5. In some embodiments, the inhibitory intracellular signaling domain is a CDH11. In some embodiments, the inhibitory intracellular signaling domain is a IMPG2. In some embodiments, the inhibitory intracellular signaling domain is a DSCAM.

Platelet endothelial cell adhesion molecule (PECAM-1), also known as CD31, is a cell-cell adhesion molecule involved in the activation of neutrophils, monocytes, and leukocytes. Cluster of differentiation 72 (CD72) is a regulatory protein expressed on B-lymphocytes and a negative regulator of B-cell responsiveness. Immunoglobulin superfamily receptor translocation associated 2 (IRTA2) and immunoglobulin superfamily receptor translocation associated 4 (IRTA4) encode a cell surface receptor homologous to the family of Fc receptors. Natural killer inhibitory receptor (NKIR) is an inhibitory receptor present on natural killer cells.

Interleukin-1 receptor accessory protein (IL1RAP or ILIR3) is a component f the interleukin 1 receptor complex, which initiates signaling upon binding to IL-1. Receptor-type tyrosine-protein phosphatase O (PTPRO or GLEPP1) is a receptor-type protein tyrosine phosphatase and is involved in signaling in immune cells. Receptor-type tyrosine-protein phosphatase zeta (PTPRZ1 or phosphacan) encodes a single pass type I membrane protein containing two cytoplasmic tyrosine phosphatase domains, an alpha anhydrase domain and a fibronectin III domain. TREM-like transcript-1 (TLT-1) is a membrane receptor which belongs to the immunoreceptor tyrosine-based inhibition motif (ITIM)-containing receptor family. SLAMF5 belongs to the Signaling lymphocyte activation molecule family (SLAMF) of receptors. It is a cell surface receptor modulator of autophagy. Protocadherin gamma-C3 (PCDHGC3) belongs to the larger cadherin superfamily of proteins which mediate cell-cell adhesion and contain immunoglobulin like organization. Leukemia inhibitory factor receptor (LIFR) is a receptor protein, whose signaling can control several cellular processes, including growth and division (proliferation), maturation (differentiation), and survival. Erythroid membrane-associated protein (ERMAP) is a receptor protein that regulates T cell and macrophage response. Interleukin 1 receptor accessory protein like 2 (IL1RAPL2) is an accessory protein that is part of the interleukin 1 receptor family. CDH5 encodes VE-cadherin (also known as Cadherin 5), which is a protein involved in calcium dependent cell-cell adhesion. Myelin protein zero-like protein 1 (MPZL1) is a transmembrane glycoprotein that promotes carcinoma cell migration and is involved in extracellular matrix-induced signal transduction. Myelin protein zero (MPZ) is a single membrane glycoprotein expressed in the on cells of the myelin sheath. Fc fragment of IgG receptor IIb (FCGR2B) is an inhibitory receptor for binding to IgG. SIGLEC-6 is an immunoreceptor containing ITIM and ITSM motifs. Megakaryocyte and platelet inhibitory receptor G6b (MPIG6B) is an inhibitory receptor that is involved in hematopoietic lineage differentiation, megakaryocyte function and platelet production. V-set and immunoglobulin domain containing 4 (VSIG4) is structurally related to the B7 family of immune regulatory proteins and is a negative regulator of T cell response. SIGLEC-12 is a cell surface protein that contains ITIMs that recruit phosphatases that can inhibit immune cell signaling. LIR8 is a member of the leukocyte immunoglobulin-like receptor (LIR) family and is involved in the modulation of cellular response. IRTA1, or FCRL4, encodes a member of the immunoglobulin receptor superfamily and is one of several Fc receptor-like glycoproteins. Killer cell immunoglobulin-like receptor 2DL4 (KIR2DL4) is a transmembrane glycoprotein that acts as an inhibitory receptor, expressed on NK cells and CD8+ T cells. KIR2DL5 is an inhibitory receptor that is thought to play a role in innate immunity signaling. SIGLEC-7 is an inhibitory receptor that is expressed on NK cells. Fc Receptor Homolog 3 (FCRH3) belongs to the immunoglobulin receptor superfamily and plays a role in the regulation of the immune system and contains both ITIM and ITAM domains. Protocadherin gamma-C5 (PCDHGC5) is a receptor molecule that plays a role in calcium dependent cell adhesion. Cadherin-11 (CDH11) is a membrane protein that is involved in calcium dependent cell-cell adhesion. Interphotoreceptor matrix proteoglycan 2 (IMPG2) is a receptor that participates in the maturation and maintenance of photoreceptors. Down syndrome cell adhesion molecule (DSCAM) is a receptor that is involved in the signaling in neuronal processes.

Exemplary inhibitory intracellular signaling domain protein sequences are shown in Table 1. Exemplary inhibitory intracellular signaling domain nucleotide sequences are shown in Table 2.

TABLE 1
Amino Acid Sequences of Exemplary Inhibitory Intracellular Domains

| SEQ ID NO | Description | Amino Acid Sequence |
|---|---|---|
| 1 | PECAM-1 ICD | KCYFLRKAKAKQMPVEMSRPAVPLLNSNNEKMSDPN |
| | | MEANSHYGHNDDVRNHAMKPINDNKEPLNSDVQYTE |
| | | VQVSSAESHKDLGKKDTETVYSEVRKAVPDAVESRYS |
| | | RTEGSLDGT |
| 2 | CD72 ICD | MAEAITYADLRFVKAPLKKSISSRLGQDPGADDDGEIT |
| | | YENVQVPAVLGVPSSLASSVLGDKAAVKSEQPTASWR |
| | | AVTSPAVGRILPCRTTCLRY |
| 3 | IRTA2 ICD | LSRKAGRKPASDPARSPSDSDSQEPTYHNVPAWEELQP |
| | | VYTNANPRGENVVYSEVRIIQEKKKHAVASDPRHLRN |
| | | KGSPIIYSEVKVASTPVSGSLFLASSAPHR |
| 4 | IRTA4 ICD | HKISGESSATNEPRGASRPNPQEFTYSSPTPDMEELQPV |
| | | YVNVGSVDVDVVYSQVWSMQQPESSANIRTLLENKD |
| | | SQVIYSSVKKS |
| 5 | NKIR ICD | WRMMKYQQKAAGMSPEQVLQPLEGDLCYADLTLQL |
| | | AGTSPQKATTKLSSAQVDQVEVEYVTMASLPKEDISY |
| | | ASLTLGAEDQEPTYCNMGHLSSHLPGRGPEEPTEYSTIS |
| | | RP |
| 6 | IL1RAP ICD | YRAHFGTDETILDGKEYDIYVSYARNAEEEEFVLLTLR |
| | | GVLENEFGYKLCIFDRDSLPGGIVTDETLSFIQKSRRLL |
| | | VVLSPNYVLQGTQALLELKAGLENMASRGNINVILVQ |
| | | YKAVKETKVKELKRAKTVLTVIKWKGEKSKYPQGRF |
| | | WKQLQVAMPVKKSPRRSSSDEQGLSYSSLKNV |
| 7 | PTPRO ICD | LRKKHLQMARECGAGTFVNFASLERDGKLPYNWRRSI |
| | | FAFLTLLPSCLWTDYLLAFYINPWSKNGLKKRKLTNPV |
| | | QLDDFDAYIKDMAKDSDYKFSLQFEELKLIGLDIPHFA |
| | | ADLPLNRCKNRYTNILPYDFSRVRLVSMNEEEGADYIN |
| | | ANYIPGYNSPQEYIATQGPLPETRNDFWKMVLQQKSQI |
| | | IVMLTQCNEKRRVKCDHYWPFTEEPIAYGDITVEMISE |
| | | EEQDDWACRHFRINYADEMQDVMHFNYTAWPDHGV |
| | | PTANAAESILQFVHMVRQQATKSKGPMIIHCSAGVGRT |
| | | GTFIALDRLLQHIRDHEFVDILGLVSEMRSYRMSMVQT |
| | | EEQYIFIHQCVQLMWMKKKQQFCISDVIYENVSKS |
| 8 | PTPRZ1 ICD | RKCFQTAHFYLEDSTSPRVISTPPTPIFPISDDVGAIPIKH |
| | | FPKHVADLHASSGFTEEFETLKEFYQEVQSCTVDLGIT |
| | | ADSSNHPDNKHKNRYINIVAYDHSRVKLAQLAEKDGK |
| | | LTDYINANYVDGYNRPKAYIAAQGPLKSTAEDFWRMI |
| | | WEHNVEVIVMITNLVEKGRRKCDQYWPADGSEEYGN |
| | | FLVTQKSVQVLAYYTVRNFTLRNTKIKKGSQKGRPSG |
| | | RVVTQYHYTQWPDMGVPEYSLPVLTFVRKAAYAKRH |
| | | AVGPVVVHCSAGVGRTGTYIVLDSMLQQIQHEGTVNI |
| | | FGFLKHIRSQRNYLVQTEEQYVFIHDTLVEAILSKETEV |
| | | LDSHIHAYVNALLIPGPAGKTKLEKQFQLLSQSNIQQS |
| | | DYSAALKQCNREKNRTSSIIPVERSRVGISSLSGEGTDY |
| | | INASYIMGYYQSNEFIITQHPLLHTIKDFWRMIWDHNA |
| | | QLVVMIPDGQNMAEDEFVYWPNKDEPINCESFKVTLM |
| | | AEEHKCLSNEEKLIIQDFILEATQDDYVLEVRHFQCPK |
| | | WPNPDSPISKTFELISVIKEEAANRDGPMIVHDEHGGVT |
| | | AGTFCALTTLMHQLEKENSVDVYQVAKMINLMRPGV |
| | | FADIEQYQFLYKVILSLVSTRQEENPSTSLDSNGAALPD |
| | | GNIAESLESLV |
| 9 | TLT1 ICD | MAKRKQGNRLGVCGRFLSSRVSGMNPSSVVHHVSDS |
| | | GPAAELPLDVPHIRLDSPPSFDNTTYTSLPLDSPSGKPSL |
| | | PAPSSLPPLPPKVLVCSKPVTYATVIFPGGNKGGGTSCG |
| | | PAQNPPNNQTPSS |
| 10 | SLAMF1 ICD | QLRRRGKTNHYQTTVEKKSLTIYAQVQKPGPLQKKLD |
| | | SFPAQDPCTTIYVAATEPVPESVQETNSITVYASVTLPE |
| | | S |
| 11 | SLAMF5 ICD | RLFKRRQGRIFPEGSCLNTFTKNPYAASKKTIYTYIMAS |
| | | RNTQPAESRIYDEILQSKVLPSKEEPVNTVYSEVQFAD |
| | | KMGKASTQDSKPPGTSSYEIVI |
| 95 | PCDHGC3 ICD | FKVYKWKQSRDLYRAPVSSLYRTPGPSLHADAVRGGL |
| | | MSPHLYHQVYLTTDSRRSDPLLKKPGAASPLASRQNT |
| | | LRSCDPVFYRQVLGAESAPPGQQAPPNTDWRFSQAQR |
| | | PGTSGSQNGDDTGTWPNNQFDTEMLQAMILASASEAA |
| | | DGSSTLGGGAGTMGLSARYGPQFTLQHVPDYRQNVYI |
| | | PGSNATLTNAAGKRDGKAPAGGNGNKKKSGKKEKK |
| 96 | LIFR ICD | YRKREWIKETFYPDIPNPENCKALQFQKSVCEGSSALK |
| | | TLEMNPCTPNNVEVLETRSAFPKIEDTEIISPVAERPEDR |
| | | SDAEPENHVVVSYCPPIIEEEIPNPAADEAGGTAQVIYI |
| | | DVQSMYQPQAKPEEEQENDPVGGAGYKPQMHLPINST |
| | | VEDIAAEEDLDKTAGYRPQANVNTWNLVSPDSPRSIDS |
| | | NSEIVSFGSPCSINSRQFLIPPKDEDSPKSNGGGWSFTNF |
| | | FQNKPND |
| 97 | ERMAP ICD | WKQRRAKEKLLYEHVTEVDNLLSDHAKEKGKLHKAV |
| | | KKLRSELKLKRAAANSGWRRARLHFVAVTLDPDTAH |
| | | PKLILSEDQRCVRLGDRRQPVPDNPQRFDFVVSILGSEY |
| | | FTTGCHYWEVYVGDKTKWILGVCSESVSRKGKVTASP |
| | | ANGHWLLRQSRGNEYEALTSPQTSFRLKEPPRCVGIFL |
| | | DYEAGVISFYNVTNKSHIFTFTHNFSGPLRPFFEPCLHD |
| | | GGKNTAPLVICSELHKSEESIVPRPEGKGHANGDVSLK |
| | | VNSSLLPPKAPELKDIILSLPPDLGPALQELKAPSF |
| 98 | IL1RAPL2 ICD | KCYNIELMLFYRQHFGADETNDDNKEYDAYLSYTKV |
| | | DQDTLDCDNPEEEQFALEVLPDVLEKHYGYKLFIPERD |
| | | LIPSGTYMEDLTRYVEQSRRLIIVLTPDYILRRGWSIFEL |
| | | ESRLHNMLVSGEIKVILIECTELKGKVNCQEVESLKRSI |
| | | KLLSLIKWKGSKSSKLNSKFWKHLVYEMPIKKKEMLP |
| | | RCHVLDSAEQGLFGELQPIPSIAMTSTSATLVSSQADLP |
| | | EFHPSDSMQIRHCCRGYKHEIPATTLPVPSLGNHHTYC |
| | | NLPLTLLNGQLPLNNTLKDTQEFHRNSSLLPLSSKELSF |
| | | TSDIW |
| 99 | CDH5 ICD | RRRLRKQARAHGKSVPEIHEQLVTYDEEGGGEMDTTS |
| | | YDVSVLNSVRRGGAKPPRPALDARPSLYAQVQKPPRH |
| | | APGAHGGPGEMAAMIEVKKDEADHDGDGPPYDTLHI |
| | | YGYEGSESIAESLSSLGTDSSDSDVDYDFLNDWGPRFK |
| | | MLAELYGSDPREELLY |
| 100 | MPZL1 ICD | SMILAVLYRRKNSKRDYTGCSTSESLSPVKQAPRKSPS |
| | | DTEGLVKSLPSGSHQGPVIYAQLDHSGGHHSDKINKSE |
| | | SVVYADIRKN |
| 101 | MPZ ICD | RYCWLRRQAALQRRLSAMEKGKLHKPGKDASKRGRQ |
| | | TPVLYAMLDHSRSTKAVSEKKAKGLGESRKDKK |
| 102 | FCGR2B ICD | VVALIYCRKKRISALPGYPECREMGETLPEKPANPTNP |
| | | DEADKVGAENTITYSLLMHPDALEEPDDQNRI |
| 103 | SIGLEC-6 ICD | RVKTRRKKAAQPVQNTDDVNPVMVSGSRGHQHQFQT |
| | | GIVSDHPAEAGPISEDEQELHYAVLHFHKVQPQEPKVT |
| | | DTEYSEIKIHK |
| 104 | MPIG6B ICD | WLHRRLPPQPIRPLPRFAPLVKTEPQRPVKEEEPKIPGD |
| | | LDQEPSLLYADLDHLALSRPRRLSTADPADASTIYAVV |
| | | V |
| 105 | VSIG4 ICD | MLCRKTSQQEHVYEAARAHAREANDSGETMRVAIFA |
| | | SGCSSDEPTSQNLGNNYSDEPCIGQEYQIIAQINGNYAR |
| | | LLDTVPLDYEFLATEGKSVC |
| 106 | SIGLEC-12 ICD | RSCRKKSARPAVGVGDTGMEDANAVRGSASQGPLIES |
| | | PADDSPPHHAPPALATPSPEEGEIQYASLSFHKARPQYP |
| | | QEQEAIGYEYSEINIPK |
| 107 | LIR8 ICD | RHRHQSKHRTSAHFYRPAGAAGPEPKDQGLQKRASPV |
| | | ADIQEEILNAAVKDTQPKDGVEMDAPAAASEAPQDVT |
| | | YAQLHSLTLRREATEPPPSQEREPPAEPSIYAPLAIH |
| 108 | IRTA1 ICD | HCWRRRKSGVGFLGDETRLPPAPGPGESSHSICPAQVE |
| | | LQSLYVDVHPKKGDLVYSEIQTTQLGEEEEANTSRTLL |
| | | EDKDVSVVYSEVKTQHPDNSAGKISSKDEES |
| 109 | KIR2DL4 ICD | RWCSKKKDAAVMNQEPAGHRTVNREDSDEQDPQEVT |
| | | YAQLDHCIFTQRKITGPSQRSKRPSTDTSVCIELPNAEP |
| | | RALSPAHEHHSQALMGSSRETTALSQTQLASSNVPAA |
| | | GI |
| 110 | KIR2DL5 ICD | LHCCCSNKKNAAVMDQEPAGDRTVNREDSDDQDPQE |
| | | VTYAQLDHCVFTQTKITSPSQRPKTPPTDTTMYMELPN |
| | | AKPRSLSPAHKHHSQALRGSSRETTALSQNRVASSHVP |
| | | AAGI |
| 111 | SIGLEC-7 ICD | RSCRKKSARPAADVGDIGMKDANTIRGSASQGNLTES |
| | | WADDNPRHHGLAAHSSGEEREIQYAPLSFHKGEPQDL |
| | | SGQEATNNEYSEIKIPK |
| 112 | FCRH3 ICD | HYARARRKPGGLSATGTSSHSPSECQEPSSSRPSRIDPQ |
| | | EPTHSKPLAPMELEPMYSNVNPGDSNPIYSQIWSIQHT |
| | | KENSANCPMMHQEHEELTVLYSELKKTHPDDSAGEAS |
| | | SRGRAHEEDDEENYENVPRVLLASDH |
| 113 | PCDHGC5 ICD | KCLQGNADGDGGGGQCCRRQDSPSPDFYKQSSPNLQV |
| | | SSDGTLKYMEVTLRPTDSQSHCYRTCFSPASDGSDFTF |
| | | LRPLSVQQPTALALEPDAIRSRSNTLRERSQQAPPNTD |
| | | WRFSQAQRPGTSGSQNGDDTGTWPNNQFDTEMLQAM |
| | | ILASASEAADGSSTLGGGAGTMGLSARYGPQFTLQHV |
| | | PDYRQNVYIPGSNATLTNAAGKRDGKAPAGGNGNKK |
| | | KSGKKEKK |
| 114 | CDH11 ICD | RRQKKEPLIVFEEEDVRENIITYDDEGGGEEDTEAFDIA |
| | | TLQNPDGINGFIPRKDIKPEYQYMPRPGLRPAPNSVDV |
| | | DDFINTRIQEADNDPTAPPYDSIQIYGYEGRGSVAGSLS |
| | | SLESATTDSDLDYDYLQNWGPRFKKLADLYGSKDTFD |
| | | DDS |
| 115 | IMPG2 ICD | YFFIRTLQAHHDRSERESPFSGSSRQPDSLSSIENAVKY |
| | | NPVYESHRAGCEKYEGPYPQHPFYSSASGDVIGGLSRE |
| | | EIRQMYESSELSREEIQERMRVLELYANDPEFAAFVRE |
| | | QQVEEV |
| 116 | DSCAM ICD | RRRRREQRLKRLRDAKSLAEMLMSKNTRTSDTLSKQQ |
| | | QTLRMHIDIPRAQLLIEERDTMETIDDRSTVLLTDADFG |
| | | EAAKQKSLTVTHTVHYQSVSQATGPLVDVSDARPGTN |
| | | PTTRRNAKAGPTARNRYASQWTLNRPHPTISAHTLTTD |
| | | WRLPTPRAAGSVDKESDSYSVSPSQDTDRARSSMVST |
| | | ESASSTYEELARAYEHAKMEEQLRHAKFTITECFISDTS |
| | | SEQLTAGTNEYTDSLTSSTPSESGICRFTASPPKPQDGG |
| | | RVMNMAVPKAHRPGDLIHLPPYLRMDFLLNRGGPGTS |
| | | RDLSLGQACLEPQKSRTLKRPTVLEPIPMEAASSASSTR |
| | | EGQSWQPGAVATLPQREGAELGQAAKMSSSQESLLDS |
| | | RGHLKGNNPYAKSYTLV |

TABLE 2
Nucleotide Sequences of Exemplary Inhibitory Intracellular Domains

| SEQ ID NO | Description | Nucleotide Sequence |
|---|---|---|
| 12 | PECAM-1 | AAGTGCTACTTCCTGCGGAAGGCCAAGGCCAAGCAGA |
| | ICD | TGCCCGTGGAAATGAGCAGACCAGCCGTGCCTCTGCTG |
| | | AACAGCAACAACGAGAAGATGAGCGACCCCAACATGG |
| | | AAGCCAACAGCCACTACGGCCACAACGACGACGTGCG |
| | | GAATCACGCCATGAAGCCCATCAACGACAACAAAGAG |
| | | CCCCTGAACAGCGACGTGCAGTACACCGAGGTGCAAG |
| | | TGTCTAGCGCCGAGAGCCACAAGGACCTGGGCAAGAA |
| | | AGACACCGAGACAGTGTACAGCGAAGTGCGCAAGGCC |
| | | GTGCCTGATGCTGTGGAAAGCAGATACAGCAGAACCG |
| | | AGGGCAGCCTGGATGGCACAACCGTGTATTCTGAGGTG |
| | | GTGCAGTATACAGAAGTC |
| 13 | CD72 ICD | ATGGCCGAGGCCATCACCTATGCCGACCTGAGATTTGT |
| | | GAAGGCCCCTCTGAAGAAGTCCATCAGCAGCAGACTC |
| | | GGCCAGGATCCAGGCGCTGATGATGATGGCGAGATCA |
| | | CCTACGAGAACGTGCAGGTTCCAGCCGTGCTGGGAGTG |
| | | CCATCTTCTCTGGCTAGTAGCGTGCTGGGCGATAAGGC |
| | | CGCCGTGAAGTCTGAACAGCCTACCGCCTCTTGGAGAG |
| | | CCGTGACATCTCCTGCCGTGGGCAGAATCCTGCCTTGC |
| | | AGAACCACCTGTCTGCGGTAC |
| 14 | IRTA2 ICD | CTGTCTAGAAAGGCCGGCAGAAAGCCTGCCAGCGATC |
| | | CTGCCAGATCTCCCAGCGACAGCGATAGCCAAGAGCCT |
| | | ACCTACCACAATGTGCCCGCCTGGGAAGAACTGCAGCC |
| | | CGTGTACACCAACGCCAATCCTAGAGGCGAGAACGTG |
| | | GTGTACAGCGAAGTGCGGATCATCCAAGAGAAGAAAA |
| | | AGCACGCCGTGGCCTCCGATCCTCGGCACCTTAGAAAC |
| | | AAGGGCAGCCCCATCATCTACTCCGAAGTGAAGGTGG |
| | | CCAGCACACCTGTGTCCGGCAGTCTGTTTCTGGCCTCTT |
| | | CTGCCCCACACCGG |
| 15 | IRTA4 ICD | CACAAGATCAGCGGCGAGAGCAGCGCCACCAATGAAC |
| | | CTAGAGGCGCCAGCAGACCCAATCCTCAAGAGTTCACC |
| | | TACAGCAGCCCCACACCTGACATGGAAGAACTGCAGC |
| | | CCGTGTACGTGAACGTGGGCTCCGTGGATGTGGACGTG |
| | | GTGTACAGCCAAGTGTGGTCCATGCAGCAGCCTGAGTC |
| | | CAGCGCCAACATCAGAACCCTGCTGGAAAACAAGGAC |
| | | TCCCAAGTGATCTACAGCTCCGTGAAGAAGTCC |
| 16 | NKIR ICD | TGGCGGATGATGAAGTACCAGCAGAAAGCCGCCGGAA |
| | | TGAGCCCCGAACAGGTTCTGCAACCTCTGGAAGGCGAT |
| | | CTGTGCTACGCCGATCTGACACTGCAGCTGGCCGGAAC |
| | | ATCTCCTCAGAAGGCCACCACAAAGCTGAGCAGCGCC |
| | | CAAGTGGATCAGGTGGAAGTGGAATACGTGACAATGG |
| | | CCAGCCTGCCTAAAGAGGACATCAGCTACGCCAGCCTG |
| | | ACACTGGGAGCCGAGGATCAAGAGCCCACCTACTGCA |
| | | ATATGGGCCACCTGAGCAGCCATCTGCCTGGCAGAGG |
| | | ACCTGAGGAACCTACCGAGTACAGCACCATCAGCAGA |
| | | CCC |
| 17 | IL1RAP ICD | TACAGAGCCCACTTCGGCACCGACGAGACAATCCTGG |
| | | ACGGCAAAGAATACGACATCTACGTGTCCTACGCCAG |
| | | AAACGCCGAGGAAGAGGAATTCGTCCTGCTGACCCTG |
| | | AGAGGCGTGCTGGAAAACGAGTTCGGCTACAAGCTGT |
| | | GCATCTTCGACCGGGATTCTCTGCCTGGCGGCATCGTG |
| | | ACAGATGAGACACTGAGCTTCATCCAGAAGTCCCGCA |
| | | GACTGCTGGTCGTGCTGAGCCCCAATTATGTGCTGCAG |
| | | GGAACACAGGCCCTGCTGGAACTGAAAGCCGGCCTGG |
| | | AAAACATGGCCAGCCGGGGCAACATCAACGTGATCCT |
| | | GGTGCAGTACAAGGCCGTGAAAGAGACAAAAGTCAAA |
| | | GAGCTGAAGCGGGCCAAGACCGTGCTGACCGTGATTA |
| | | AGTGGAAGGGCGAGAAGTCCAAGTATCCCCAGGGCAG |
| | | ATTCTGGAAGCAGCTGCAGGTTGCCATGCCTGTGAAGA |
| | | AGTCCCCAAGAAGAAGCAGCAGCGACGAGCAGGGACT |
| | | GAGCTACAGCAGCCTGAAGAACGTG |
| 18 | PTPRO ICD | CTGCGGAAGAAACACCTCCAGATGGCCAGAGAATGTG |
| | | GCGCCGGAACCTTCGTGAATTTCGCCTCTCTGGAACGC |
| | | GACGGCAAGCTGCCTTATAACTGGCGGAGATCCATCTT |
| | | CGCCTTTCTGACCCTGCTGCCTAGCTGCCTGTGGACCG |
| | | ATTACCTGCTGGCCTTCTACATCAACCCTTGGAGCAAG |
| | | AACGGCCTGAAGAAGCGGAAGCTGACAAACCCCGTGC |
| | | AGCTCGACGACTTCGACGCCTACATCAAGGACATGGCC |
| | | AAGGACTCCGACTACAAGTTCAGCCTGCAGTTCGAGGA |
| | | ACTGAAGCTGATCGGCCTGGACATCCCTCACTTCGCCG |
| | | CTGACCTGCCTCTGAACCGGTGCAAGAACCGGTACACC |
| | | AACATCCTGCCTTACGACTTCAGCAGAGTGCGGCTGGT |
| | | GTCCATGAACGAGGAAGAGGGCGCCGACTACATCAAT |
| | | GCCAACTACATCCCCGGCTACAACAGCCCTCAAGAGTA |
| | | TATCGCCACACAGGGCCCTCTGCCAGAGACAAGAAAC |
| | | GACTTCTGGAAGATGGTCCTGCAGCAGAAAAGCCAGA |
| | | TCATCGTGATGCTGACCCAGTGCAACGAGAAGCGGAG |
| | | AGTGAAGTGCGACCACTACTGGCCCTTCACCGAGGAAC |
| | | CTATCGCCTACGGCGACATCACAGTGGAAATGATCAGC |
| | | GAGGAAGAACAGGACGACTGGGCCTGCCGGCACTTCA |
| | | GAATCAACTACGCCGACGAGATGCAGGACGTGATGCA |
| | | CTTCAACTACACCGCCTGGCCTGATCACGGCGTGCCAA |
| | | CAGCTAATGCCGCCGAATCCATCCTGCAGTTTGTGCAC |
| | | ATGGTTCGACAGCAGGCCACCAAGAGCAAGGGCCCCA |
| | | TGATCATCCACTGTTCTGCTGGCGTGGGCAGAACCGGC |
| | | ACCTTTATCGCTCTGGACAGACTGCTCCAGCACATCCG |
| | | GGATCACGAGTTCGTGGATATCCTGGGACTCGTGTCCG |
| | | AGATGCGGAGCTACAGAATGAGCATGGTGCAGACAGA |
| | | GGAACAGTACATCTTCATCCACCAGTGCGTCCAGCTGA |
| | | TGTGGATGAAGAAGAAGCAGCAGTTCTGCATCAGCGA |
| | | CGTCATCTACGAGAACGTGTCCAAGAGC |
| 19 | PTPRZ1 ICD | CGGAAGTGCTTCCAGACAGCCCACTTCTACCTGGAAGA |
| | | TAGCACAAGCCCCAGAGTGATCAGCACCCCTCCAACAC |
| | | CTATCTTCCCCATCTCCGATGACGTGGGCGCTATCCCC |
| | | ATCAAGCACTTTCCAAAGCACGTGGCCGATCTGCACGC |
| | | CAGCTCTGGCTTCACCGAGGAATTCGAGACACTGAAAG |
| | | AATTCTACCAAGAGGTGCAGAGCTGCACCGTGGACCTG |
| | | GGAATCACAGCCGACAGCAGCAATCACCCCGACAACA |
| | | AGCACAAGAACCGGTACATCAACATCGTGGCCTACGA |
| | | TCACAGCAGAGTGAAGCTGGCCCAGCTGGCCGAGAAG |
| | | GATGGCAAGCTGACCGACTACATCAACGCCAATTACGT |
| | | GGACGGCTACAACAGGCCCAAGGCCTATATTGCCGCTC |
| | | AGGGCCCTCTGAAGTCTACCGCCGAGGACTTTTGGAGA |
| | | ATGATCTGGGAGCACAACGTGGAAGTGATCGTGATGA |
| | | TCACCAACCTGGTGGAAAAAGGCAGACGGAAGTGCGA |
| | | TCAGTACTGGCCTGCCGATGGCAGCGAGGAATACGGC |
| | | AATTTCCTGGTCACCCAGAAAAGCGTGCAGGTCCTGGC |
| | | CTACTACACAGTGCGGAACTTCACCCTGCGGAACACCA |
| | | AGATCAAGAAGGGCTCCCAGAAGGGCAGACCCTCTGG |
| | | CAGAGTGGTCACACAGTACCACTACACCCAGTGGCCTG |
| | | ATATGGGCGTGCCAGAGTATAGCCTGCCAGTGCTGACC |
| | | TTTGTGCGGAAGGCCGCCTATGCCAAGAGACATGCTGT |
| | | GGGACCTGTGGTGGTGCACTGTTCTGCTGGCGTGGGAA |
| | | GAACCGGCACCTACATCGTGCTGGACAGCATGCTCCAG |
| | | CAGATTCAGCACGAGGGCACCGTGAATATCTTCGGCTT |
| | | CCTGAAGCACATCAGAAGCCAGCGGAACTACCTGGTG |
| | | CAGACCGAGGAACAGTACGTGTTCATCCACGACACACT |
| | | GGTGGAAGCCATCCTGAGCAAAGAAACCGAGGTGCTG |
| | | GACTCCCACATCCACGCCTATGTGAACGCCCTGCTGAT |
| | | TCCTGGACCAGCCGGCAAGACCAAGCTGGAAAAGCAG |
| | | TTCCAGCTGCTGTCCCAGAGCAACATCCAGCAGAGCGA |
| | | CTACAGCGCCGCTCTGAAGCAGTGCAACAGAGAGAAG |
| | | AACAGAACCAGCAGCATCATCCCTGTGGAACGGTCCA |
| | | GAGTGGGCATCTCTAGCTTGTCTGGCGAGGGCACAGAT |
| | | TACATCAATGCCAGCTACATCATGGGCTACTACCAGTC |
| | | CAACGAGTTCATCATCACACAGCACCCTCTGCTGCACA |
| | | CCATCAAGGATTTCTGGCGGATGATTTGGGACCACAAT |
| | | GCTCAGCTGGTGGTCATGATCCCCGACGGCCAGAATAT |
| | | GGCCGAGGACGAGTTTGTGTACTGGCCCAACAAGGAC |
| | | GAGCCCATCAACTGCGAGAGCTTCAAAGTGACCCTGAT |
| | | GGCCGAAGAACACAAGTGCCTGAGCAACGAGGAAAAG |
| | | CTGATCATCCAGGACTTCATCCTGGAAGCCACACAGGA |
| | | CGACTACGTGCTGGAAGTGCGGCACTTTCAGTGCCCTA |
| | | AGTGGCCCAATCCTGACAGCCCCATCAGCAAGACCTTC |
| | | GAGCTGATCTCCGTGATCAAAGAGGAAGCCGCCAACC |
| | | GCGACGGCCCTATGATTGTGCACGATGAGCATGGCGG |
| | | AGTGACCGCCGGAACATTTTGTGCCCTGACAACCCTGA |
| | | TGCACCAGCTCGAGAAAGAAAACTCCGTGGACGTGTA |
| | | CCAGGTGGCCAAGATGATCAACCTGATGCGGCCTGGC |
| | | GTGTTCGCCGATATCGAGCAGTACCAGTTCCTGTACAA |
| | | AGTGATCCTCAGCCTGGTGTCTACCCGGCAAGAGGAAA |
| | | ACCCTAGCACCAGCCTGGATAGCAATGGCGCCGCACTG |
| | | CCCGATGGAAATATCGCCGAGTCTCTGGAAAGCCTCGT |
| | | T |
| 20 | TLT1 ICD | ATGGCCAAGCGGAAGCAGGGCAATAGACTGGGCGTGT |
| | | GCGGCAGATTCCTGTCCTCTAGAGTGTCCGGCATGAAC |
| | | CCCAGCAGCGTGGTGCATCACGTGTCCGATTCTGGACC |
| | | TGCCGCTGAGCTGCCACTGGATGTGCCTCACATCAGAC |
| | | TGGACAGCCCACCTAGCTTCGACAACACCACCTACACA |
| | | AGCCTGCCTCTCGATAGCCCTTCCGGCAAGCCATCTCT |
| | | GCCTGCTCCATCTTCTCTGCCTCCACTGCCTCCTAAGGT |
| | | GCTCGTGTGTAGCAAGCCTGTGACCTACGCCACCGTGA |
| | | TCTTCCCTGGCGGAAACAAAGGCGGCGGAACATCTTGT |
| | | GGCCCCGCTCAGAACCCTCCTAACAATCAGACACCTAG |
| | | CAGC |
| 21 | SLAMF1 ICD | CAGCTGCGTCGCCGTGGCAAGACGAACCATTACCAGA |
| | | CGACCGTGGAGAAAAAATCCCTGACCATTTACGCGCA |
| | | GGTGCAGAAGCCGGGGCCCCTGCAGAAGAAGCTGGAC |
| | | TCGTTTCCTGCTCAGGACCCGTGCACTACAATCTACGT |
| | | GGCGGCTACGGAGCCCGTACCCGAATCCGTGCAGGAG |
| | | ACTAACTCCATCACCGTCTACGCTTCCGTGACCCTTCCT |
| | | GAGAGC |
| 22 | SLAMF5 ICD | CGCCTGTTCAAGCGCCGCCAGGGGCGCATCTTCCCCGA |
| | | GGGCTCTTGTCTCAATACGTTCACCAAGAACCCCTATG |
| | | CTGCCAGCAAGAAGACCATCTACACCTACATCATGGCC |
| | | TCCCGCAACACCCAGCCAGCAGAGTCGCGCATTTACGA |
| | | CGAGATCCTGCAATCTAAGGTGCTGCCATCCAAGGAGG |
| | | AACCCGTCAACACTGTTTACTCCGAGGTGCAGTTCGCG |
| | | GACAAAATGGGGAAGGCTTCCACCCAGGACTCCAAGC |
| | | CGCCTGGCACCTCGAGCTACGAGATCGTGATT |
| 117 | PCDHGC3 | TTCAAGGTGTACAAGTGGAAGCAGAGCCGGGACCTGT |
| | ICD | ACAGAGCCCCTGTGTCCAGCCTGTATAGAACCCCTGGA |
| | | CCTAGCCTGCATGCCGATGCTGTTAGAGGCGGCCTGAT |
| | | GAGCCCTCACCTGTACCATCAGGTGTACCTGACCACCG |
| | | ACAGCAGAAGAAGCGACCCTCTGCTGAAGAAGCCTGG |
| | | CGCTGCTTCTCCACTGGCCAGCAGACAGAATACCCTGA |
| | | GAAGCTGCGACCCCGTGTTCTATAGGCAGGTTCTGGGA |
| | | GCCGAATCTGCCCCTCCTGGACAACAGGCCCCTCCTAA |
| | | CACCGATTGGAGATTCAGCCAGGCTCAGAGGCCTGGC |
| | | ACAAGCGGATCTCAGAATGGCGACGATACCGGCACCT |
| | | GGCCTAACAACCAGTTCGACACCGAAATGCTGCAGGC |
| | | CATGATTCTGGCCTCTGCCTCTGAAGCCGCCGATGGCA |
| | | GTTCTACACTTGGAGGCGGAGCCGGCACAATGGGACT |
| | | GTCTGCTAGATACGGCCCTCAGTTCACCCTGCAGCACG |
| | | TGCCCGATTACCGGCAGAACGTGTACATCCCCGGCAGC |
| | | AATGCCACACTGACAAACGCCGCTGGCAAGAGAGATG |
| | | GCAAAGCTCCTGCTGGCGGCAACGGCAACAAGAAGAA |
| | | GTCCGGCAAGAAAGAGAAGAAG |
| 118 | LIFR ICD | TACCGGAAGAGAGAGTGGATCAAAGAGACATTCTACC |
| | | CGGACATCCCCAATCCTGAGAACTGCAAAGCCCTGCAG |
| | | TTCCAGAAAAGCGTGTGCGAGGGAAGCAGCGCCCTGA |
| | | AAACCCTGGAAATGAACCCCTGCACACCCAACAACGT |
| | | GGAAGTGCTGGAAACCAGAAGCGCTTTCCCCAAGATC |
| | | GAGGACACCGAGATCATCAGCCCCGTGGCCGAAAGAC |
| | | CCGAGGATAGATCTGATGCCGAGCCTGAGAATCACGT |
| | | GGTGGTGTCCTACTGTCCTCCTATCATCGAGGAAGAGA |
| | | TCCCTAATCCTGCCGCCGATGAAGCCGGCGGAACAGCC |
| | | CAAGTGATCTACATCGACGTGCAGAGCATGTACCAGCC |
| | | TCAGGCCAAGCCTGAGGAAGAACAAGAGAACGACCCT |
| | | GTTGGCGGAGCCGGCTACAAGCCCCAAATGCATCTGCC |
| | | CATCAACAGCACCGTGGAAGATATCGCCGCCGAAGAG |
| | | GACCTGGATAAGACCGCCGGATATAGACCCCAGGCCA |
| | | ACGTGAACACCTGGAACCTGGTGTCCCCAGACAGCCCC |
| | | AGATCCATCGACAGCAACAGCGAGATCGTGTCCTTCGG |
| | | CAGCCCCTGCTCTATCAACAGCCGGCAGTTCCTGATTC |
| | | CTCCTAAGGACGAGGACAGCCCTAAGAGCAATGGCGG |
| | | CGGATGGTCCTTCACCAATTTCTTCCAGAACAAGCCCA |
| | | ACGAC |
| 119 | ERMAP ICD | TGGAAGCAGCGGAGAGCCAAAGAGAAGCTGCTGTACG |
| | | AGCACGTGACCGAGGTGGACAACCTGCTGAGCGATCA |
| | | CGCCAAAGAAAAGGGCAAGCTGCACAAGGCCGTGAAG |
| | | AAGCTGAGAAGCGAGCTGAAGCTGAAGCGGGCTGCCG |
| | | CCAATTCTGGATGGCGTAGAGCCAGACTGCACTTCGTG |
| | | GCCGTGACACTGGACCCCGATACAGCCCATCCTAAGCT |
| | | GATCCTGAGCGAGGACCAGAGATGTGTGCGGCTGGGA |
| | | GATAGAAGGCAGCCCGTGCCTGACAACCCTCAGAGAT |
| | | TCGACTTCGTGGTGTCCATCCTGGGCAGCGAGTACTTC |
| | | ACCACCGGCTGCCACTACTGGGAAGTGTACGTGGGCG |
| | | ACAAGACCAAGTGGATCCTGGGCGTGTGTAGCGAGAG |
| | | CGTGTCCAGAAAGGGCAAAGTGACAGCTAGCCCCGCC |
| | | AATGGACACTGGCTGCTGAGACAGAGCAGAGGCAATG |
| | | AGTACGAGGCCCTGACAAGCCCACAGACCAGCTTCCG |
| | | GCTGAAAGAACCTCCTAGATGCGTGGGCATCTTCCTGG |
| | | ATTATGAGGCCGGCGTGATCAGCTTCTACAACGTGACC |
| | | AACAAGAGCCACATCTTCACGTTCACCCACAACTTCAG |
| | | CGGCCCTCTGCGGCCATTCTTCGAGCCTTGTCTGCACG |
| | | ACGGCGGCAAGAATACTGCCCCTCTGGTCATCTGTAGC |
| | | GAACTGCACAAGAGCGAGGAATCCATCGTGCCCAGAC |
| | | CTGAAGGCAAGGGACATGCCAATGGGGACGTGTCCCT |
| | | GAAAGTGAACAGCAGCCTGCTGCCTCCTAAGGCTCCTG |
| | | AGCTGAAGGACATCATCCTGAGCCTGCCTCCAGACCTG |
| | | GGACCTGCTCTGCAAGAACTGAAGGCCCCTAGCTTC |
| 120 | IL1RAPL2 | AAGTGCTACAACATCGAGCTGATGCTGTTCTACCGGCA |
| | ICD | GCACTTTGGCGCCGACGAGACAAACGACGACAACAAA |
| | | GAGTACGACGCCTACCTGAGCTACACCAAGGTGGACC |
| | | AGGACACCCTGGACTGCGACAACCCTGAGGAAGAACA |
| | | GTTCGCCCTCGAGGTGCTGCCCGACGTGCTGGAAAAGC |
| | | ACTACGGCTACAAGCTGTTCATCCCCGAGCGGGATCTG |
| | | ATCCCTAGCGGCACCTACATGGAAGATCTGACCAGATA |
| | | CGTGGAACAGAGCAGACGGCTGATCATCGTGCTGACC |
| | | CCTGACTACATCCTGCGGAGAGGCTGGTCCATCTTCGA |
| | | GCTGGAAAGCCGGCTGCACAACATGCTGGTGTCCGGC |
| | | GAGATCAAAGTGATCCTGATCGAGTGCACCGAGCTGA |
| | | AGGGCAAAGTGAACTGCCAAGAGGTGGAAAGCCTGAA |
| | | GCGGAGCATCAAGCTGCTGAGCCTGATCAAGTGGAAG |
| | | GGCAGCAAGAGCAGCAAGCTGAACAGCAAGTTCTGGA |
| | | AGCACCTGGTGTACGAGATGCCCATCAAAAAGAAAGA |
| | | AATGCTGCCCCGGTGCCATGTGCTGGATTCTGCTGAGC |
| | | AGGGCCTGTTTGGAGAGCTGCAGCCCATTCCTTCTATC |
| | | GCCATGACCAGCACCTCCGCCACACTGGTTTCTAGCCA |
| | | GGCCGACCTGCCTGAGTTTCACCCCAGCGATAGCATGC |
| | | AGATCCGGCACTGCTGCCGGGGCTATAAGCACGAGATT |
| | | CCAGCCACCACACTGCCCGTGCCTTCTCTGGGAAATCA |
| | | CCACACCTACTGCAACCTGCCTCTGACACTGCTGAACG |
| | | GCCAGCTGCCACTGAACAACACCCTGAAGGACACCCA |
| | | AGAGTTCCACCGGAACAGCAGCCTGCTGCCTCTGTCCA |
| | | GCAAAGAGCTGAGCTTCACCAGCGACATCTGG |
| 121 | CDH5 ICD | CGGCGGAGACTGAGAAAGCAGGCTAGAGCCCACGGCA |
| | | AGAGCGTGCCAGAGATTCACGAACAGCTGGTCACCTA |
| | | CGACGAGGAAGGCGGAGGCGAGATGGATACCACAAGC |
| | | TACGATGTGTCCGTGCTGAACAGCGTGCGAAGAGGCG |
| | | GAGCCAAACCTCCTAGACCTGCTCTGGATGCCAGACCT |
| | | AGCCTGTATGCCCAGGTGCAGAAGCCTCCAAGACACG |
| | | CTCCTGGTGCTCATGGTGGACCTGGCGAAATGGCCGCC |
| | | ATGATCGAAGTGAAGAAGGACGAGGCCGACCACGATG |
| | | GCGACGGCCCTCCATATGATACCCTGCACATCTACGGC |
| | | TACGAGGGCAGCGAGTCTATCGCCGAGTCTCTGTCTAG |
| | | CCTGGGCACCGATAGCAGCGATAGCGACGTGGACTAC |
| | | GACTTCCTGAACGACTGGGGCCCTAGATTCAAGATGCT |
| | | GGCCGAGCTGTACGGCAGCGACCCTAGAGAGGAACTG |
| | | CTGTAC |
| 122 | MPZL1 ICD | TCTATGATCCTGGCCGTGCTGTACCGGCGGAAGAACAG |
| | | CAAGAGAGACTACACCGGCTGCAGCACCAGCGAGTCT |
| | | CTGTCTCCAGTGAAGCAGGCCCCTAGAAAGAGCCCCTC |
| | | TGATACCGAAGGCCTGGTCAAGTCTCTGCCCAGCGGAT |
| | | CTCATCAGGGCCCAGTGATCTATGCCCAGCTGGACCAT |
| | | TCTGGCGGCCACCACAGCGACAAGATCAACAAGAGCG |
| | | AGAGCGTGGTGTACGCCGACATCCGGAAGAAT |
| 123 | MPZ ICD | CGGTACTGCTGGCTGAGAAGGCAGGCTGCACTGCAGA |
| | | GAAGGCTGAGCGCCATGGAAAAGGGCAAGCTGCACAA |
| | | GCCTGGCAAGGACGCCTCTAAGAGAGGCAGACAGACC |
| | | CCTGTGCTGTACGCCATGCTGGACCACAGCAGAAGCAC |
| | | AAAGGCCGTCAGCGAGAAGAAGGCCAAAGGCCTGGGC |
| | | GAGAGCCGGAAGGATAAGAAG |
| 124 | FCGR2B ICD | GTGGTGGCCCTGATCTACTGCCGGAAGAAGAGAATCTC |
| | | TGCCCTGCCTGGCTACCCCGAGTGTAGAGAGATGGGAG |
| | | AGACACTGCCCGAGAAGCCCGCCAATCCTACCAATCCT |
| | | GACGAGGCCGACAAAGTGGGCGCCGAGAATACCATCA |
| | | CCTACAGCCTGCTGATGCACCCCGACGCTCTGGAAGAA |
| | | CCCGACGACCAGAACAGAATC |
| 125 | SIGLEC-6 | AGAGTGAAAACCCGGCGGAAGAAAGCCGCTCAGCCCG |
| | ICD | TGCAGAATACCGACGACGTGAACCCTGTGATGGTGTCC |
| | | GGCTCTAGAGGACACCAGCACCAGTTTCAGACCGGCAT |
| | | CGTGTCTGATCACCCTGCCGAAGCCGGACCTATCAGCG |
| | | AGGATGAGCAAGAGCTGCACTACGCCGTGCTGCACTTC |
| | | CACAAGGTGCAGCCCCAAGAGCCTAAAGTGACCGACA |
| | | CCGAGTACAGCGAGATCAAGATCCACAAG |
| 126 | MPIG6B ICD | TGGCTGCACAGAAGGCTGCCTCCACAGCCTATCAGACC |
| | | CCTGCCTAGATTTGCCCCTCTGGTCAAGACAGAGCCCC |
| | | AGCGGCCTGTGAAAGAGGAAGAACCTAAGATCCCCGG |
| | | CGACCTGGACCAAGAGCCTTCTCTGCTGTACGCCGACC |
| | | TGGATCATCTGGCCCTGAGCAGACCTAGACGGCTGTCT |
| | | ACAGCCGATCCTGCCGATGCCAGCACAATCTATGCTGT |
| | | GGTGGTG |
| 127 | VSIG4 ICD | ATGCTGTGCAGAAAGACCAGCCAGCAAGAGCACGTGT |
| | | ACGAGGCCGCTAGAGCCCATGCCAGAGAGGCTAACGA |
| | | TAGCGGCGAGACAATGAGAGTGGCCATCTTCGCCAGC |
| | | GGCTGTAGCTCTGATGAGCCCACCTCTCAGAACCTGGG |
| | | CAACAACTACAGCGACGAGCCCTGCATCGGCCAAGAG |
| | | TACCAGATCATTGCCCAGATCAACGGCAACTACGCCCG |
| | | GCTGCTGGATACCGTGCCTCTGGATTATGAGTTCCTGG |
| | | CCACCGAGGGCAAGAGCGTGTGT |
| 128 | SIGLEC-12 | CGGAGCTGCCGGAAGAAGTCTGCTAGACCTGCTGTTGG |
| | ICD | CGTGGGCGATACCGGAATGGAAGATGCCAATGCCGTC |
| | | AGAGGCAGCGCCTCTCAGGGACCTCTGATTGAGTCTCC |
| | | CGCCGACGATAGCCCTCCTCATCATGCTCCTCCAGCTC |
| | | TGGCCACACCTTCTCCAGAGGAAGGCGAGATCCAGTAC |
| | | GCCAGCCTGAGCTTCCACAAGGCCAGACCTCAGTACCC |
| | | TCAAGAGCAAGAGGCCATCGGCTACGAGTACAGCGAG |
| | | ATCAACATCCCCAAG |
| 129 | LIR8 ICD | CGACATCGGCATCAGAGCAAACACAGGACATCGGCCC |
| | | ATTTCTACCGTCCTGCAGGGGCTGCGGGGCCAGAGCCC |
| | | AAGGACCAGGGCCTGCAGAAGAGGGCCAGCCCAGTTG |
| | | CTGACATCCAGGAGGAAATTCTCAATGCTGCCGTGAAG |
| | | GACACACAGCCCAAGGACGGGGTGGAGATGGATGCTC |
| | | CGGCTGCTGCATCTGAAGCCCCCCAGGATGTGACCTAC |
| | | GCCCAGCTGCACAGCTTGACCCTCAGACGGGAGGCAA |
| | | CTGAGCCTCCTCCATCCCAGGAAAGGGAACCTCCAGCT |
| | | GAACCCAGCATCTACGCCCCCCTGGCCATCCAC |
| 130 | IRTA1 ICD | CACTGCTGGCGTCGGAGGAAGTCAGGAGTTGGTTTCTT |
| | | GGGAGACGAAACCAGGCTCCCTCCCGCTCCAGGCCCA |
| | | GGAGAGTCCTCCCATTCCATCTGCCCTGCCCAGGTGGA |
| | | GCTTCAGTCGTTGTATGTTGATGTACACCCCAAAAAGG |
| | | GAGATTTGGTATACTCTGAGATCCAGACTACTCAGCTG |
| | | GGAGAAGAAGAGGAAGCTAATACCTCCAGGACACTTC |
| | | TAGAGGATAAGGATGTCTCAGTTGTCTACTCTGAGGTA |
| | | AAGACACAACACCCAGATAACTCAGCTGGAAAGATCA |
| | | GCTCTAAGGATGAAGAAAGT |
| 131 | KIR2DL4 | CGCTGGTGCTCTAAAAAGAAGGATGCCGCGGTGATGA |
| | ICD | ACCAGGAGCCCGCTGGCCACCGCACCGTCAACCGCGA |
| | | GGACAGTGACGAGCAGGATCCGCAGGAGGTGACCTAC |
| | | GCGCAGTTGGATCACTGTATTTTCACTCAGCGCAAGAT |
| | | TACCGGCCCTTCACAGAGGTCCAAGCGCCCTTCGACCG |
| | | ACACCTCTGTCTGTATTGAGTTGCCCAACGCGGAGCCA |
| | | AGAGCACTGAGCCCGGCGCATGAGCACCACAGCCAGG |
| | | CCCTGATGGGCTCGTCCCGCGAAACGACAGCTCTCTCG |
| | | CAGACCCAGCTTGCTAGCTCTAATGTACCAGCAGCCGG |
| | | GATC |
| 132 | KIR2DL5 | CTTCATTGCTGCTGCTCCAACAAAAAGAATGCTGCTGT |
| | ICD | AATGGACCAAGAGCCTGCCGGGGACAGAACAGTGAAC |
| | | AGGGAGGACTCTGATGATCAAGACCCTCAGGAGGTGA |
| | | CATATGCACAGTTGGATCACTGCGTTTTCACACAGACA |
| | | AAAATCACTTCCCCTTCTCAGAGGCCCAAGACACCTCC |
| | | AACAGATACCACCATGTACATGGAACTTCCAAATGCTA |
| | | AGCCAAGATCATTGTCTCCTGCCCATAAGCACCACAGT |
| | | CAGGCCTTGAGGGGATCTTCTAGGGAGACAACAGCCCT |
| | | GTCTCAAAACCGGGTTGCTAGCTCCCATGTACCAGCAG |
| | | CTGGAATC |
| 133 | SIGLEC-7 | AGGTCCTGCAGGAAGAAATCGGCAAGGCCAGCAGCGG |
| | ICD | ACGTGGGAGACATAGGCATGAAGGATGCAAACACCAT |
| | | CAGGGGCTCAGCCTCTCAGGGTAACCTGACTGAGTCCT |
| | | GGGCAGATGATAACCCCCGACACCATGGCCTGGCTGCC |
| | | CACTCCTCAGGGGAGGAAAGAGAGATCCAGTATGCAC |
| | | CCCTCAGCTTTCATAAGGGGGAGCCTCAGGACCTATCA |
| | | GGACAAGAAGCCACCAACAATGAGTACTCAGAGATCA |
| | | AGATCCCCAAG |
| 134 | FCRH3 ICD | CATTATGCCAGAGCCAGAAGAAAGCCTGGCGGCCTGT |
| | | CTGCCACCGGCACATCTTCTCACAGCCCCAGCGAGTGT |
| | | CAAGAGCCCAGCAGCAGCAGACCCAGCAGAATCGATC |
| | | CCCAAGAGCCTACACACAGCAAGCCCCTGGCTCCTATG |
| | | GAACTGGAACCCATGTACAGCAACGTGAACCCCGGCG |
| | | ACAGCAACCCCATCTACAGCCAGATTTGGAGCATCCAG |
| | | CACACCAAAGAGAACAGCGCCAACTGTCCCATGATGC |
| | | ACCAAGAGCACGAGGAACTGACCGTGCTGTACTCCGA |
| | | GCTGAAGAAAACACACCCCGACGACTCTGCCGGCGAG |
| | | GCCTCTTCTAGAGGCAGAGCCCATGAAGAGGACGACG |
| | | AAGAGAACTACGAGAACGTGCCCAGAGTGCTGCTGGC |
| | | CTCCGATCAT |
| 135 | PCDHGC5 | AAGTGCCTGCAGGGAAATGCTGATGGCGACGGTGGCG |
| | ICD | GAGGCCAGTGTTGTAGAAGGCAGGATAGCCCCTCTCCA |
| | | GACTTCTACAAGCAGAGCAGCCCCAACCTCCAGGTGTC |
| | | CTCTGATGGCACCCTGAAGTACATGGAAGTGACCCTGA |
| | | GGCCTACCGACAGCCAGAGCCACTGCTACAGAACCTG |
| | | CTTTAGCCCTGCCAGCGACGGCAGCGACTTTACCTTTC |
| | | TGAGGCCTCTGAGCGTGCAGCAGCCTACAGCTCTGGCT |
| | | CTGGAACCTGACGCCATCAGAAGCAGAAGCAACACCC |
| | | TGAGAGAGCGGAGCCAGCAGGCCCCTCCTAATACCGA |
| | | TTGGAGATTCAGCCAGGCTCAGCGGCCTGGCACAAGC |
| | | GGATCTCAGAATGGCGACGATACCGGCACCTGGCCTA |
| | | ACAACCAGTTCGACACCGAAATGCTGCAGGCCATGATT |
| | | CTGGCCTCTGCCTCTGAAGCCGCCGATGGCAGTTCTAC |
| | | ACTTGGAGGCGGAGCCGGCACAATGGGACTGTCTGCT |
| | | AGATACGGCCCTCAGTTCACCCTGCAGCACGTGCCCGA |
| | | CTACAGACAGAACGTGTACATCCCCGGCAGCAACGCC |
| | | ACACTGACAAATGCCGCCGGAAAGAGAGATGGCAAAG |
| | | CCCCTGCTGGCGGCAACGGCAACAAGAAGAAGTCCGG |
| | | CAAGAAAGAGAAGAAG |
| 136 | CDH11 ICD | CGGCGGCAGAAGAAAGAACCCCTGATCGTGTTCGAAG |
| | | AGGAAGATGTGCGCGAGAACATCATCACCTACGACGA |
| | | CGAAGGCGGAGGCGAAGAGGATACCGAGGCCTTCGAT |
| | | ATCGCCACACTGCAGAACCCCGACGGCATCAACGGCTT |
| | | CATCCCCAGAAAGGACATCAAGCCCGAGTACCAGTAC |
| | | ATGCCCAGACCTGGACTCAGACCCGCTCCTAATTCCGT |
| | | GGACGTGGACGACTTCATCAACACCCGGATCCAAGAG |
| | | GCCGACAACGACCCTACAGCTCCTCCATACGACAGCAT |
| | | CCAGATCTACGGCTACGAAGGCAGAGGAAGCGTGGCC |
| | | GGATCTCTGAGCAGTCTGGAAAGCGCCACCACCGACA |
| | | GCGACCTGGATTACGACTACCTGCAGAACTGGGGCCCT |
| | | AGATTCAAGAAGCTGGCCGACCTGTACGGCAGCAAGG |
| | | ACACCTTCGACGATGACAGC |
| 137 | IMPG2 ICD | TACTTTTTCATCCGGACACTGCAGGCCCATCACGACAG |
| | | ATCCGAGAGAGAGAGCCCTTTCAGCGGCAGCAGCAGA |
| | | CAGCCTGATAGCCTGAGCAGCATCGAGAACGCCGTGA |
| | | AGTACAACCCCGTGTACGAGTCTCACAGAGCCGGCTGC |
| | | GAGAAGTACGAGGGCCCTTATCCTCAGCACCCCTTCTA |
| | | CAGCTCTGCCAGCGGAGATGTGATCGGCGGCCTGTCCA |
| | | GAGAAGAGATCCGGCAGATGTACGAGAGCAGCGAGCT |
| | | GAGCCGGGAAGAGATTCAAGAGCGGATGAGAGTGCTG |
| | | GAACTGTACGCCAACGATCCCGAGTTCGCCGCCTTTGT |
| | | GCGAGAACAGCAGGTCGAGGAAGTG |
| 138 | DSCAM ICD | CGGCGGAGAAGAAGAGAGCAGCGGCTGAAGAGACTGC |
| | | GGGATGCCAAATCTCTGGCCGAGATGCTGATGAGCAA |
| | | GAACACCAGAACCAGCGACACCCTGAGCAAGCAGCAA |
| | | CAGACCCTGCGGATGCACATCGACATCCCCAGAGCAC |
| | | AGCTGCTGATCGAGGAACGGGACACCATGGAAACCAT |
| | | CGACGACAGATCCACCGTGCTGCTGACCGATGCCGATT |
| | | TTGGAGAGGCCGCCAAGCAGAAAAGCCTGACCGTGAC |
| | | ACACACCGTGCACTACCAGTCTGTGTCCCAGGCTACAG |
| | | GACCTCTGGTGGACGTGTCAGATGCCAGACCTGGCACA |
| | | AACCCCACCACCAGAAGAAACGCCAAGGCCGGACCTA |
| | | CCGCCAGAAACAGATATGCCAGCCAGTGGACCCTGAA |
| | | CAGACCCCATCCTACCATCAGCGCCCACACACTGACCA |
| | | CCGATTGGAGACTGCCCACACCTAGAGCCGCCGGATCT |
| | | GTGGACAAAGAGTCCGACAGCTACAGCGTGTCCCCTA |
| | | GCCAGGATACCGACAGAGCCAGATCCAGCATGGTGTC |
| | | TACAGAGAGCGCCAGCAGCACCTACGAGGAACTGGCC |
| | | AGAGCCTATGAGCACGCCAAGATGGAAGAACAGCTGC |
| | | GCCACGCCAAGTTCACCATCACCGAGTGCTTCATCTCC |
| | | GACACCAGCAGCGAACAGCTGACCGCCGGCACCAATG |
| | | AGTACACCGATAGCCTGACCTCCAGCACACCTTCCGAG |
| | | AGCGGCATCTGCAGGTTTACCGCCTCTCCACCTAAGCC |
| | | TCAGGATGGCGGCAGAGTGATGAACATGGCCGTGCCT |
| | | AAGGCTCACAGACCCGGCGACCTGATTCATCTGCCACC |
| | | TTACCTGAGAATGGACTTCCTGCTGAACAGAGGCGGCC |
| | | CAGGCACAAGCAGAGATCTGTCTCTTGGCCAGGCCTGC |
| | | CTGGAACCTCAGAAGTCTAGAACCCTGAAGCGGCCTAC |
| | | CGTGCTGGAACCCATTCCTATGGAAGCCGCCAGCTCTG |
| | | CCTCTAGCACAAGAGAGGGACAGTCTTGGCAGCCTGG |
| | | CGCTGTTGCTACACTGCCTCAAAGAGAAGGCGCCGAAC |
| | | TGGGACAAGCCGCCAAAATGAGCAGCAGCCAAGAGAG |
| | | CCTGCTGGACTCTAGAGGCCACCTGAAGGGCAACAAC |
| | | CCCTACGCCAAGAGCTACACCCTGGTG |

In some embodiments, one of the one or more intracellular signaling domains is derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the transmembrane domain is derived from the same protein as one of the one or more intracellular signaling domains. In some embodiments, the transmembrane domain is derived from a first protein and one of the one or more the intracellular signaling domains is derived from a second protein that is distinct from the first protein.

In some embodiments, one of the one or more intracellular signaling domains is derived from PECAM-1.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to KCYFLRKAKAKQMPVEMSRPAVPLLNSNNEKMSDPNMEANSHYGHNDDVRNHAMKPINDNK EPLNSDVQYTEVQVSSAESHKDLGKKDTETVYSEVRKAVPDAVESRYSRTEGSLDGT (SEQ ID NO: 1)

In some embodiments, one of the one or more intracellular signaling domain comprises the amino acid sequence of KCYFLRKAKAKQMPVEMSRPAVPLLNSNNEKMSDPNMEANSHYGHNDDVRNHAMKPINDNK EPLNSDVQYTEVQVSSAESHKDLGKKDTETVYSEVRKAVPDAVESRYSRTEGSLDGT (SEQ ID NO: 1).

In some embodiments, one of the one or more intracellular signaling domain is derived from CD72.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to MAEAITYADLRFVKAPLKKSISSRLGQDPGADDDGEITYENVQVPAVLGVPSSLASSVLGDKAA VKSEQPTASWRAVTSPAVGRILPCRTTCLRY (SEQ ID NO: 2).

In some embodiments, one of the one or more intracellular signaling domain comprises the amino acid sequence of MAEAITYADLRFVKAPLKKSISSRLGQDPGADDDGEITYENVQVPAVLGVPSSLASSVLGDKAA VKSEQPTASWRAVTSPAVGRILPCRTTCLRY (SEQ ID NO: 2).

In some embodiments, one of the one or more intracellular signaling domain is derived from IRTA2.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LSRKAGRKPASDPARSPSDSDSQEPTYHNVPAWEELQPVYTNANPRGENVVYSEVRIIQEKKKH AVASDPRHLRNKGSPIIYSEVKVASTPVSGSLFLASSAPHR (SEQ ID NO: 3).

In some embodiments, one of the one or more intracellular signaling domain comprises the amino acid sequence of LSRKAGRKPASDPARSPSDSDSQEPTYHNVPAWEELQPVYTNANPRGENVVYSEVRIIQEKKKH AVASDPRHLRNKGSPIIYSEVKVASTPVSGSLFLASSAPHR (SEQ ID NO: 3).

In some embodiments, one of the one or more intracellular signaling domain is derived from IRTA4.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to HKISGESSATNEPRGASRPNPQEFTYSSPTPDMEELQPVYVNVGSVDVDVVYSQVWSMQQPESS ANIRTLLENKDSQVIYSSVKKS (SEQ ID NO: 4).

In some embodiments, one of the one or more intracellular signaling domain comprises the amino acid sequence of HKISGESSATNEPRGASRPNPQEFTYSSPTPDMEELQPVYVNVGSVDVDVVYSQVWSMQQPESS ANIRTLLENKDSQVIYSSVKKS (SEQ ID NO: 4).

In some embodiments, one of the one or more intracellular signaling domain is derived from NKIR.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to WRMMKYQQKAAGMSPEQVLQPLEGDLCYADLTLQLAGTSPQKATTKLSSAQVDQVEVEYVT MASLPKEDISYASLTLGAEDQEPTYCNMGHLSSHLPGRGPEEPTEYSTISRP (SEQ ID NO: 5).

In some embodiments, one of the one or more intracellular signaling domain comprises the amino acid sequence of WRMMKYQQKAAGMSPEQVLQPLEGDLCYADLTLQLAGTSPQKATTKLSSAQVDQVEVEYVT MASLPKEDISYASLTLGAEDQEPTYCNMGHLSSHLPGRGPEEPTEYSTISRP (SEQ ID NO: 5).

In some embodiments, one of the one or more intracellular signaling domain is derived from IL1RAP.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to YRAHFGTDETILDGKEYDIYVSYARNAEEEEFVLLTLRGVLENEFGYKLCIFDRDSLPGGIVTDET LSFIQKSRRLLVVLSPNYVLQGTQALLELKAGLENMASRGNINVILVQYKAVKETKVKELKRAK TVLTVIKWKGEKSKYPQGRFWKQLQVAMPVKKSPRRSSSDEQGLSYSSLKNV (SEQ ID NO: 6).

In some embodiments, one of the one or more intracellular signaling domain comprises the amino acid sequence of YRAHFGTDETILDGKEYDIYVSYARNAEEEEFVLLTLRGVLENEFGYKLCIFDRDSLPGGIVTDET LSFIQKSRRLLVVLSPNYVLOGTQALLELKAGLENMASRGNINVILVQYKAVKETKVKELKRAK TVLTVIKWKGEKSKYPQGRFWKQLQVAMPVKKSPRRSSSDEQGLSYSSLKNV (SEQ ID NO: 6).

In some embodiments, one of the one or more intracellular signaling domain is derived from PTPRO.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LRKKHLQMARECGAGTFVNFASLERDGKLPYNWRRSIFAFLTLLPSCLWTDYLLAFYINPWSKN GLKKRKLTNPVQLDDFDAYIKDMAKDSDYKFSLQFEELKLIGLDIPHFAADLPLNRCKNRYTNIL PYDFSRVRLVSMNEEEGADYINANYIPGYNSPQEYIATQGPLPETRNDFWKMVLQQKSQIIVML TQCNEKRRVKCDHYWPFTEEPIAYGDITVEMISEEEQDDWACRHFRINYADEMQDVMHFNYTA WPDHGVPTANAAESILQFVHMVRQQATKSKGPMIIHCSAGVGRTGTFIALDRLLQHIRDHEFVDI LGLVSEMRSYRMSMVQTEEQYIFIHQCVQLMWMKKKQQFCISDVIYENVSKS (SEQ ID NO: 7).

In some embodiments, one of the one or more intracellular signaling domain comprises the amino acid sequence of LRKKHLQMARECGAGTFVNFASLERDGKLPYNWRRSIFAFLTLLPSCLWTDYLLAFYINPWSKN GLKKRKLTNPVQLDDFDAYIKDMAKDSDYKFSLQFEELKLIGLDIPHFAADLPLNRCKNRYTNIL PYDFSRVRLVSMNEEEGADYINANYIPGYNSPQEYIATQGPLPETRNDFWKMVLQQKSQIIVML TQCNEKRRVKCDHYWPFTEEPIAYGDITVEMISEEEQDDWACRHFRINYADEMQDVMHFNYTA WPDHGVPTANAAESILQFVHMVRQQATKSKGPMIIHCSAGVGRTGTFIALDRLLQHIRDHEFVDI LGLVSEMRSYRMSMVQTEEQYIFIHQCVQLMWMKKKQQFCISDVIYENVSKS (SEQ ID NO: 7).

In some embodiments, one of the one or more intracellular signaling domain is derived from PTPRZ1.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RKCFQTAHFYLEDSTSPRVISTPPTPIFPISDDVGAIPIKHFPKHVADLHASSGFTEEFETLKEFYQE VQSCTVDLGITADSSNHPDNKHKNRYINIVAYDHSRVKLAQLAEKDGKLTDYINANYVDGYNR PKAYIAAQGPLKSTAEDFWRMIWEHNVEVIVMITNLVEKGRRKCDQYWPADGSEEYGNFLVTQ KSVQVLAYYTVRNFTLRNTKIKKGSQKGRPSGRVVTQYHYTQWPDMGVPEYSLPVLTFVRKAA YAKRHAVGPVVVHCSAGVGRTGTYIVLDSMLQQIQHEGTVNIFGFLKHIRSQRNYLVQTEEQYV FIHDTLVEAILSKETEVLDSHIHAYVNALLIPGPAGKTKLEKQFQLLSQSNIQQSDYSAALKQCNR EKNRTSSIIPVERSRVGISSLSGEGTDYINASYIMGYYQSNEFIITQHPLLHTIKDFWRMIWDHNAQ LVVMIPDGQNMAEDEFVYWPNKDEPINCESFKVTLMAEEHKCLSNEEKLIIQDFILEATQDDYV LEVRHFQCPKWPNPDSPISKTFELISVIKEEAANRDGPMIVHDEHGGVTAGTFCALTTLMHQLEK ENSVDVYQVAKMINLMRPGVFADIEQYQFLYKVILSLVSTRQEENPSTSLDSNGAALPDGNIAES LESLV (SEQ ID NO: 8).

In some embodiments, one of the one or more intracellular signaling domain comprises the amino acid sequence of RKCFQTAHFYLEDSTSPRVISTPPTPIFPISDDVGAIPIKHFPKHVADLHASSGFTEEFETLKEFYQE VQSCTVDLGITADSSNHPDNKHKNRYINIVAYDHSRVKLAQLAEKDGKLTDYINANYVDGYNR PKAYIAAQGPLKSTAEDFWRMIWEHNVEVIVMITNLVEKGRRKCDQYWPADGSEEYGNFLVTQ KSVQVLAYYTVRNFTLRNTKIKKGSQKGRPSGRVVTQYHYTQWPDMGVPEYSLPVLTFVRKAA YAKRHAVGPVVVHCSAGVGRTGTYIVLDSMLQQIQHEGTVNIFGFLKHIRSQRNYLVQTEEQYV FIHDTLVEAILSKETEVLDSHIHAYVNALLIPGPAGKTKLEKQFQLLSQSNIQQSDYSAALKQCNR EKNRTSSIIPVERSRVGISSLSGEGTDYINASYIMGYYQSNEFIITQHPLLHTIKDFWRMIWDHNAQ LVVMIPDGQNMAEDEFVYWPNKDEPINCESFKVTLMAEEHKCLSNEEKLIIQDFILEATQDDYV LEVRHFQCPKWPNPDSPISKTFELISVIKEEAANRDGPMIVHDEHGGVTAGTFCALTTLMHQLEK ENSVDVYQVAKMINLMRPGVFADIEQYQFLYKVILSLVSTRQEENPSTSLDSNGAALPDGNIAES LESLV (SEQ ID NO: 8).

In some embodiments, one of the one or more intracellular signaling domain is derived from TLT1.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to MAKRKQGNRLGVCGRFLSSRVSGMNPSSVVHHVSDSGPAAELPLDVPHIRLDSPPSFDNTTYTS LPLDSPSGKPSLPAPSSLPPLPPKVLVCSKPVTYATVIFPGGNKGGGTSCGPAQNPPNNQTPSS (SEQ ID NO: 9).

In some embodiments, one of the one or more intracellular signaling domain comprises the amino acid sequence of MAKRKQGNRLGVCGRFLSSRVSGMNPSSVVHHVSDSGPAAELPLDVPHIRLDSPPSFDNTTYTS LPLDSPSGKPSLPAPSSLPPLPPKVLVCSKPVTYATVIFPGGNKGGGTSCGPAQNPPNNQTPSS (SEQ ID NO: 9).

In some embodiments, one of the one or more intracellular signaling domain is derived from SLAMF1.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to QLRRRGKTNHYQTTVEKKSLTIYAQVQKPGPLQKKLDSFPAQDPCTTIYVAATEPVPESVQETNS ITVYASVTLPES (SEQ ID NO: 10).

In some embodiments, one of the one or more intracellular signaling domain comprises the amino acid sequence of QLRRRGKTNHYQTTVEKKSLTIYAQVQKPGPLQKKLDSFPAQDPCTTIYVAATEPVPESVQETNS ITVYASVTLPES (SEQ ID NO: 10).

In some embodiments, one of the one or more intracellular signaling domain is derived from SLAMF5.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RLFKRRQGRIFPEGSCLNTFTKNPYAASKKTIYTYIMASRNTQPAESRIYDEILQSKVLPSKEEPVN TVYSEVQFADKMGKASTQDSKPPGTSSYEIVI (SEQ ID NO: 11).

In some embodiments, one of the one or more intracellular signaling domain comprises the amino acid sequence of RLFKRRQGRIFPEGSCLNTFTKNPYAASKKTIYTYIMASRNTQPAESRIYDEILQSKVLPSKEEPVN TVYSEVQFADKMGKASTQDSKPPGTSSYEIVI (SEQ ID NO: 11).

In some embodiments, one of the one or more intracellular signaling domains is derived from PCDHGC3.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to FKVYKWKQSRDLYRAPVSSLYRTPGPSLHADAVRGGLMSPHLYHQVYLTTDSRRSDPLLKKPG AASPLASRQNTLRSCDPVFYRQVLGAESAPPGQQAPPNTDWRFSQAQRPGTSGSQNGDDTGTW PNNQFDTEMLQAMILASASEAADGSSTLGGGAGTMGLSARYGPQFTLQHVPDYRQNVYIPGSN ATLTNAAGKRDGKAPAGGNGNKKKSGKKEKK (SEQ ID NO: 95).

In some embodiments, one of the one or more intracellular signaling domain comprises the amino acid sequence of FKVYKWKQSRDLYRAPVSSLYRTPGPSLHADAVRGGLMSPHLYHQVYLTTDSRRSDPLLKKPG AASPLASRQNTLRSCDPVFYRQVLGAESAPPGQQAPPNTDWRFSQAQRPGTSGSQNGDDTGTW PNNQFDTEMLQAMILASASEAADGSSTLGGGAGTMGLSARYGPQFTLQHVPDYRQNVYIPGSN ATLTNAAGKRDGKAPAGGNGNKKKSGKKEKK (SEQ ID NO: 95).

In some embodiments, one of the one or more intracellular signaling domains is derived from LIFR.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to YRKREWIKETFYPDIPNPENCKALQFQKSVCEGSSALKTLEMNPCTPNNVEVLETRSAFPKIEDT EIISPVAERPEDRSDAEPENHVVVSYCPPIIEEEIPNPAADEAGGTAQVIYIDVQSMYQPQAKPEEE QENDPVGGAGYKPQMHLPINSTVEDIAAEEDLDKTAGYRPQANVNTWNLVSPDSPRSIDSNSEI VSFGSPCSINSRQFLIPPKDEDSPKSNGGGWSFTNFFQNKPND. (SEQ ID NO: 96).

In some embodiments, one of the one or more intracellular signaling domain comprises the amino acid sequence of YRKREWIKETFYPDIPNPENCKALQFQKSVCEGSSALKTLEMNPCTPNNVEVLETRSAFPKIEDT EIISPVAERPEDRSDAEPENHVVVSYCPPIIEEEIPNPAADEAGGTAQVIYIDVQSMYQPQAKPEEE QENDPVGGAGYKPQMHLPINSTVEDIAAEEDLDKTAGYRPQANVNTWNLVSPDSPRSIDSNSEI VSFGSPCSINSRQFLIPPKDEDSPKSNGGGWSFTNFFQNKPND. (SEQ ID NO: 96).

In some embodiments, one of the one or more intracellular signaling domains is derived from ERMAP.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to WKQRRAKEKLLYEHVTEVDNLLSDHAKEKGKLHKAVKKLRSELKLKRAAANSGWRRARLHF VAVTLDPDTAHPKLILSEDQRCVRLGDRRQPVPDNPQRFDFVVSILGSEYFTTGCHYWEVYVGD KTKWILGVCSESVSRKGKVTASPANGHWLLRQSRGNEYEALTSPQTSFRLKEPPRCVGIFLDYE AGVISFYNVTNKSHIFTFTHNFSGPLRPFFEPCLHDGGKNTAPLVICSELHKSEESIVPRPEGKGHA NGDVSLKVNSSLLPPKAPELKDIILSLPPDLGPALQELKAPSF. (SEQ ID NO: 97).

In some embodiments, one of the one or more intracellular signaling domain comprises the amino acid sequence of WKQRRAKEKLLYEHVTEVDNLLSDHAKEKGKLHKAVKKLRSELKLKRAAANSGWRRARLHF VAVTLDPDTAHPKLILSEDQRCVRLGDRRQPVPDNPQRFDFVVSILGSEYFTTGCHYWEVYVGD KTKWILGVCSESVSRKGKVTASPANGHWLLRQSRGNEYEALTSPQTSFRLKEPPRCVGIFLDYE AGVISFYNVTNKSHIFTFTHNFSGPLRPFFEPCLHDGGKNTAPLVICSELHKSEESIVPRPEGKGHA NGDVSLKVNSSLLPPKAPELKDIILSLPPDLGPALQELKAPSF. (SEQ ID NO: 97).

In some embodiments, one of the one or more intracellular signaling domains is derived from IL1RAPL2.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to KCYNIELMLFYRQHFGADETNDDNKEYDAYLSYTKVDQDTLDCDNPEEEQFALEVLPDVLEKH YGYKLFIPERDLIPSGTYMEDLTRYVEQSRRLIIVLTPDYILRRGWSIFELESRLHNMLVSGEIKVI LIECTELKGKVNCQEVESLKRSIKLLSLIKWKGSKSSKLNSKFWKHLVYEMPIKKKEMLPRCHVL DSAEQGLFGELQPIPSIAMTSTSATLVSSQADLPEFHPSDSMQIRHCCRGYKHEIPATTLPVPSLGN HHTYCNLPLTLLNGQLPLNNTLKDTQEFHRNSSLLPLSSKELSFTSDIW. (SEQ ID NO: 98).

In some embodiments, one of the one or more intracellular signaling domain comprises the amino acid sequence of KCYNIELMLFYRQHFGADETNDDNKEYDAYLSYTKVDQDTLDCDNPEEEQFALEVLPDVLEKH YGYKLFIPERDLIPSGTYMEDLTRYVEQSRRLIIVLTPDYILRRGWSIFELESRLHNMLVSGEIKVI LIECTELKGKVNCQEVESLKRSIKLLSLIKWKGSKSSKLNSKFWKHLVYEMPIKKKEMLPRCHVL DSAEQGLFGELQPIPSIAMTSTSATLVSSQADLPEFHPSDSMQIRHCCRGYKHEIPATTLPVPSLGN HHTYCNLPLTLLNGQLPLNNTLKDTQEFHRNSSLLPLSSKELSFTSDIW. (SEQ ID NO: 98).

In some embodiments, one of the one or more intracellular signaling domains is derived from CDH5.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RRRLRKQARAHGKSVPEIHEQLVTYDEEGGGEMDTTSYDVSVLNSVRRGGAKPPRPALDARPS LYAQVQKPPRHAPGAHGGPGEMAAMIEVKKDEADHDGDGPPYDTLHIYGYEGSESIAESLSSLG TDSSDSDVDYDFLNDWGPRFKMLAELYGSDPREELLY. (SEQ ID NO: 99).

In some embodiments, one of the one or more intracellular signaling domain comprises the amino acid sequence of RRRLRKQARAHGKSVPEIHEQLVTYDEEGGGEMDTTSYDVSVLNSVRRGGAKPPRPALDARPS LYAQVQKPPRHAPGAHGGPGEMAAMIEVKKDEADHDGDGPPYDTLHIYGYEGSESIAESLSSLG TDSSDSDVDYDFLNDWGPRFKMLAELYGSDPREELLY. (SEQ ID NO: 99).

In some embodiments, one of the one or more intracellular signaling domains is derived from MPZL1.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SMILAVLYRRKNSKRDYTGCSTSESLSPVKQAPRKSPSDTEGLVKSLPSGSHQGPVIYAQLDHSG GHHSDKINKSESVVYADIRKN. (SEQ ID NO: 100).

In some embodiments, one of the one or more intracellular signaling domain comprises the amino acid sequence of SMILAVLYRRKNSKRDYTGCSTSESLSPVKQAPRKSPSDTEGLVKSLPSGSHQGPVIYAQLDHSG GHHSDKINKSESVVYADIRKN. (SEQ ID NO: 100).

In some embodiments, one of the one or more intracellular signaling domains is derived from MPZ.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RYCWLRRQAALQRRLSAMEKGKLHKPGKDASKRGRQTPVLYAMLDHSRSTKAVSEKKAKGL GESRKDKK. (SEQ ID NO: 101).

In some embodiments, one of the one or more intracellular signaling domain comprises the amino acid sequence of RYCWLRRQAALQRRLSAMEKGKLHKPGKDASKRGRQTPVLYAMLDHSRSTKAVSEKKAKGL GESRKDKK. (SEQ ID NO: 101).

In some embodiments, one of the one or more intracellular signaling domains is derived from FCGR2B.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VVALIYCRKKRISALPGYPECREMGETLPEKPANPTNPDEADKVGAENTITYSLLMHPDALEEPD DQNRI. (SEQ ID NO: 102).

In some embodiments, one of the one or more intracellular signaling domain comprises the amino acid sequence of VVALIYCRKKRISALPGYPECREMGETLPEKPANPTNPDEADKVGAENTITYSLLMHPDALEEPD DQNRI. (SEQ ID NO: 102).

In some embodiments, one of the one or more intracellular signaling domains is derived from SIGLEC-6.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RVKTRRKKAAQPVQNTDDVNPVMVSGSRGHQHQFQTGIVSDHPAEAGPISEDEQELHYAVLHF HKVQPQEPKVTDTEYSEIKIHK. (SEQ ID NO: 103).

In some embodiments, one of the one or more intracellular signaling domain comprises the amino acid sequence of RVKTRRKKAAQPVQNTDDVNPVMVSGSRGHQHQFQTGIVSDHPAEAGPISEDEQELHYAVLHF HKVQPQEPKVTDTEYSEIKIHK. (SEQ ID NO: 103).

In some embodiments, one of the one or more intracellular signaling domains is derived from MPIG6B.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to WLHRRLPPQPIRPLPRFAPLVKTEPQRPVKEEEPKIPGDLDQEPSLLYADLDHLALSRPRRLSTAD PADASTIYAVVV. (SEQ ID NO: 104).

In some embodiments, one of the one or more intracellular signaling domain comprises the amino acid sequence of WLHRRLPPQPIRPLPRFAPLVKTEPQRPVKEEEPKIPGDLDQEPSLLYADLDHLALSRPRRLSTAD PADASTIYAVVV. (SEQ ID NO: 104).

In some embodiments, one of the one or more intracellular signaling domains is derived from VSIG4.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to MLCRKTSQQEHVYEAARAHAREANDSGETMRVAIFASGCSSDEPTSQNLGNNYSDEPCIGQEY QIIAQINGNYARLLDTVPLDYEFLATEGKSVC. (SEQ ID NO: 105).

In some embodiments, one of the one or more intracellular signaling domain comprises the amino acid sequence of MLCRKTSQQEHVYEAARAHAREANDSGETMRVAIFASGCSSDEPTSQNLGNNYSDEPCIGQEY QIIAQINGNYARLLDTVPLDYEFLATEGKSVC. (SEQ ID NO: 105).

In some embodiments, one of the one or more intracellular signaling domains is derived from SIGLEC-12.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RSCRKKSARPAVGVGDTGMEDANAVRGSASQGPLIESPADDSPPHHAPPALATPSPEEGEIQYAS LSFHKARPQYPQEQEAIGYEYSEINIPK (SEQ ID NO: 106).

In some embodiments, one of the one or more intracellular signaling domain comprises the amino acid sequence of RSCRKKSARPAVGVGDTGMEDANAVRGSASQGPLIESPADDSPPHHAPPALATPSPEEGEIQYAS LSFHKARPQYPQEQEAIGYEYSEINIPK (SEQ ID NO: 106).

In some embodiments, one of the one or more intracellular signaling domains is derived from LIR8.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RHRHQSKHRTSAHFYRPAGAAGPEPKDQGLQKRASPVADIQEEILNAAVKDTQPKDGVEMDAP AAASEAPQDVTYAQLHSLTLRREATEPPPSQEREPPAEPSIYAPLAIH. (SEQ ID NO: 107).

In some embodiments, one of the one or more intracellular signaling domain comprises the amino acid sequence of RHRHQSKHRTSAHFYRPAGAAGPEPKDQGLQKRASPVADIQEEILNAAVKDTQPKDGVEMDAP AAASEAPQDVTYAQLHSLTLRREATEPPPSQEREPPAEPSIYAPLAIH. (SEQ ID NO: 107).

In some embodiments, one of the one or more intracellular signaling domains is derived from IRTA1.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to HCWRRRKSGVGFLGDETRLPPAPGPGESSHSICPAQVELQSLYVDVHPKKGDLVYSEIQTTQLGE EEEANTSRTLLEDKDVSVVYSEVKTQHPDNSAGKISSKDEES. (SEQ ID NO: 108).

In some embodiments, one of the one or more intracellular signaling domain comprises the amino acid sequence of HCWRRRKSGVGFLGDETRLPPAPGPGESSHSICPAQVELQSLYVDVHPKKGDLVYSEIQTTQLGE EEEANTSRTLLEDKDVSVVYSEVKTQHPDNSAGKISSKDEES. (SEQ ID NO: 108).

In some embodiments, one of the one or more intracellular signaling domains is derived from KIR2DL4.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RWCSKKKDAAVMNQEPAGHRTVNREDSDEQDPQEVTYAQLDHCIFTQRKITGPSQRSKRPSTD TSVCIELPNAEPRALSPAHEHHSQALMGSSRETTALSQTQLASSNVPAAGI. (SEQ ID NO: 109).

In some embodiments, one of the one or more intracellular signaling domain comprises the amino acid sequence of RWCSKKKDAAVMNQEPAGHRTVNREDSDEQDPQEVTYAQLDHCIFTQRKITGPSQRSKRPSTD TSVCIELPNAEPRALSPAHEHHSQALMGSSRETTALSQTQLASSNVPAAGI. (SEQ ID NO: 109).

In some embodiments, one of the one or more intracellular signaling domains is derived from KIR2DL5.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LHCCCSNKKNAAVMDQEPAGDRTVNREDSDDQDPQEVTYAQLDHCVFTQTKITSPSQRPKTPP TDTTMYMELPNAKPRSLSPAHKHHSQALRGSSRETTALSQNRVASSHVPAAGI. (SEQ ID NO: 110).

In some embodiments, one of the one or more intracellular signaling domain comprises the amino acid sequence of LHCCCSNKKNAAVMDQEPAGDRTVNREDSDDQDPQEVTYAQLDHCVFTQTKITSPSQRPKTPP TDTTMYMELPNAKPRSLSPAHKHHSQALRGSSRETTALSQNRVASSHVPAAGI. (SEQ ID NO: 110).

In some embodiments, one of the one or more intracellular signaling domains is derived from SIGLEC-7.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RSCRKKSARPAADVGDIGMKDANTIRGSASQGNLTESWADDNPRHHGLAAHSSGEEREIQYAP LSFHKGEPQDLSGQEATNNEYSEIKIPK. (SEQ ID NO: 111).

In some embodiments, one of the one or more intracellular signaling domain comprises the amino acid sequence of RSCRKKSARPAADVGDIGMKDANTIRGSASQGNLTESWADDNPRHHGLAAHSSGEEREIQYAP LSFHKGEPQDLSGQEATNNEYSEIKIPK. (SEQ ID NO: 111).

In some embodiments, one of the one or more intracellular signaling domains is derived from FCRH3.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to HYARARRKPGGLSATGTSSHSPSECQEPSSSRPSRIDPQEPTHSKPLAPMELEPMYSNVNPGDSNP IYSQIWSIQHTKENSANCPMMHQEHEELTVLYSELKKTHPDDSAGEASSRGRAHEEDDEENYEN VPRVLLASDH. (SEQ ID NO: 112).

In some embodiments, one of the one or more intracellular signaling domain comprises the amino acid sequence of HYARARRKPGGLSATGTSSHSPSECQEPSSSRPSRIDPQEPTHSKPLAPMELEPMYSNVNPGDSNP IYSQIWSIQHTKENSANCPMMHQEHEELTVLYSELKKTHPDDSAGEASSRGRAHEEDDEENYEN VPRVLLASDH. (SEQ ID NO: 112).

In some embodiments, one of the one or more intracellular signaling domains is derived from PCDHGC5.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to KCLQGNADGDGGGGQCCRRQDSPSPDFYKQSSPNLQVSSDGTLKYMEVTLRPTDSQSHCYRTC FSPASDGSDFTFLRPLSVQQPTALALEPDAIRSRSNTLRERSQQAPPNTDWRFSQAQRPGTSGSQN GDDTGTWPNNQFDTEMLQAMILASASEAADGSSTLGGGAGTMGLSARYGPQFTLQHVPDYRQ NVYIPGSNATLTNAAGKRDGKAPAGGNGNKKKSGKKEKK. (SEQ ID NO: 113).

In some embodiments, one of the one or more intracellular signaling domain comprises the amino acid sequence of KCLQGNADGDGGGGQCCRRQDSPSPDFYKQSSPNLQVSSDGTLKYMEVTLRPTDSQSHCYRTC FSPASDGSDFTFLRPLSVQQPTALALEPDAIRSRSNTLRERSQQAPPNTDWRFSQAQRPGTSGSQN GDDTGTWPNNQFDTEMLQAMILASASEAADGSSTLGGGAGTMGLSARYGPQFTLQHVPDYRQ NVYIPGSNATLTNAAGKRDGKAPAGGNGNKKKSGKKEKK. (SEQ ID NO: 113).

In some embodiments, one of the one or more intracellular signaling domains is derived from CDH11.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RRQKKEPLIVFEEEDVRENIITYDDEGGGEEDTEAFDIATLONPDGINGFIPRKDIKPEYQYMPRP GLRPAPNSVDVDDFINTRIQEADNDPTAPPYDSIQIYGYEGRGSVAGSLSSLESATTDSDLDYDYL QNWGPRFKKLADLYGSKDTFDDDS. (SEQ ID NO: 114).

In some embodiments, one of the one or more intracellular signaling domain comprises the amino acid sequence of RRQKKEPLIVFEEEDVRENIITYDDEGGGEEDTEAFDIATLONPDGINGFIPRKDIKPEYQYMPRP GLRPAPNSVDVDDFINTRIQEADNDPTAPPYDSIQIYGYEGRGSVAGSLSSLESATTDSDLDYDYL QNWGPRFKKLADLYGSKDTFDDDS. (SEQ ID NO: 114).

In some embodiments, one of the one or more intracellular signaling domains is derived from IMPG2.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to YFFIRTLQAHHDRSERESPFSGSSRQPDSLSSIENAVKYNPVYESHRAGCEKYEGPYPQHPFYSSA SGDVIGGLSREEIRQMYESSELSREEIQERMRVLELYANDPEFAAFVREQQVEEV. (SEQ ID NO: 115).

In some embodiments, one of the one or more intracellular signaling domain comprises the amino acid sequence of YFFIRTLQAHHDRSERESPFSGSSRQPDSLSSIENAVKYNPVYESHRAGCEKYEGPYPQHPFYSSA SGDVIGGLSREEIRQMYESSELSREEIQERMRVLELYANDPEFAAFVREQQVEEV. (SEQ ID NO: 115).

In some embodiments, one of the one or more intracellular signaling domains is derived from DSCAM.

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RRRRREQRLKRLRDAKSLAEMLMSKNTRTSDTLSKQQQTLRMHIDIPRAQLLIEERDTMETIDD RSTVLLTDADEGEAAKQKSLTVTHTVHYQSVSQATGPLVDVSDARPGTNPTTRRNAKAGPTAR NRYASQWTLNRPHPTISAHTLTTDWRLPTPRAAGSVDKESDSYSVSPSQDTDRARSSMVSTESA SSTYEELARAYEHAKMEEQLRHAKFTITECFISDTSSEQLTAGTNEYTDSLTSSTPSESGICRFTAS PPKPQDGGRVMNMAVPKAHRPGDLIHLPPYLRMDFLLNRGGPGTSRDLSLGQACLEPQKSRTL KRPTVLEPIPMEAASSASSTREGQSWQPGAVATLPQREGAELGQAAKMSSSQESLLDSRGHLKG NNPYAKSYTLV. (SEQ ID NO: 116).

In some embodiments, one of the one or more intracellular signaling domain comprises the amino acid sequence of RRRRREQRLKRLRDAKSLAEMLMSKNTRTSDTLSKQQQTLRMHIDIPRAQLLIEERDTMETIDD RSTVLLTDADEGEAAKQKSLTVTHTVHYQSVSQATGPLVDVSDARPGTNPTTRRNAKAGPTAR NRYASQWTLNRPHPTISAHTLTTDWRLPTPRAAGSVDKESDSYSVSPSQDTDRARSSMVSTESA SSTYEELARAYEHAKMEEQLRHAKFTITECFISDTSSEQLTAGTNEYTDSLTSSTPSESGICRFTAS PPKPQDGGRVMNMAVPKAHRPGDLIHLPPYLRMDFLLNRGGPGTSRDLSLGQACLEPQKSRTL KRPTVLEPIPMEAASSASSTREGQSWQPGAVATLPQREGAELGQAAKMSSSQESLLDSRGHLKG NNPYAKSYTLV. (SEQ ID NO: 116).

In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 1. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 2. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 3. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 4. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 7. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 8. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 9. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 10. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 11. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 95. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 96. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 97. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 98. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 99. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 100. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 101. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 102. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 103. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 104. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 105. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 106. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 107. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 108. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 109. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 110. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 112. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 113. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 114. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 115. In some embodiments, one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 116.

In some embodiments, the transmembrane domain and one of the one or more intracellular signaling domain are derived from the same protein. In some embodiments, the transmembrane domain is derived from a first protein and one of the one or more intracellular signaling domain is derived from a second protein that is distinct from the first protein.

### Enzymatic Inhibitory Domains

In some embodiments, the inhibitory chimeric receptor comprises an enzymatic inhibitory domain. In some embodiments, the enzymatic inhibitory domain is also capable of preventing, attenuating, or inhibiting activation of a chimeric receptor when expressed on an immunomodulatory cell relative to an otherwise identical chimeric inhibitory receptor lacking the enzymatic inhibitory domain.

In some embodiments, the enzymatic inhibitory domain comprises an enzyme catalytic domain. In some embodiments, the enzyme catalytic domain is derived from an enzyme selected from the group consisting of: CSK, SHP-1, PTEN, CD45, CD148, PTP-MEG1, PTP-PEST, c-CBL, CBL-b, PTPN22, LAR, PTPH1, SHIP-1, and RasGAP.

In some embodiments, the enzymatic inhibitory domain comprises one or more modifications that modulate basal prevention, attenuation, or inhibition relative to an otherwise identical enzymatic inhibitory domain lacking the one or more modifications. In some embodiments, the one or more modifications reduce basal prevention, attenuation, or inhibition relative to an otherwise identical enzymatic inhibitory domain lacking the one or more modifications. In some embodiments, the one or more modifications increase basal prevention, attenuation, or inhibition relative to an otherwise identical enzymatic inhibitory domain lacking the one or more modifications.

### Activation and Co-Stimulatory Domains

In some embodiments, a cell disclosed herein can further comprise at least one tumor-targeting chimeric receptor or T cell receptor comprising an activating intracellular domain or a co-stimulatory intracellular domain. In some embodiments, the cell comprises at least one inhibitory chimeric receptor and at least one tumor-targeting chimeric receptor. The cell can comprise at least 1, at least 2, at least 3, at least 4, at least 5, at least 6, at least 7, at least 8, at least 9, or at least 10 or more tumor-targeting CARs and at least 1, at least 2, at least 3, at least 4, at least 5, at least 6, at least 7, at least 8, at least 9, or at least 10 or more inhibitory chimeric receptors.

In some embodiments, the activating signaling domain is a CD3-zeta protein, which includes three immunoreceptor tyrosine-based activation motifs (ITAMs). Other examples of activating signaling domains include CD28, 4-1BB, and OX40. In some embodiments, a cell receptor comprises more than one activating signaling domain, each referred to as a co-stimulatory domain.

In some embodiments, the tumor-targeting chimeric receptor is a chimeric antigen receptor (CAR) or an engineered T cell receptor. In some embodiments, the CAR binds one or more proteins expressed on the surface of a tumor cell.

In some embodiments, prior to binding of the protein to the chimeric inhibitory receptor, the tumor-targeting chimeric receptor is capable of activating the cell.

### Transmembrane Domains

The inhibitory chimeric receptors can contain transmembrane domains that link the protein binding domain to the intracellular domain. Different transmembrane domains result in different receptor stability. Suitable transmembrane domains include, but are not limited to, CD8, CD28, CD3zeta, CD4, 4-IBB, OX40, ICOS, 2B4, CD25, CD7, LAX, LAT, LAIR1, PECAM-1, LIR, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM

In some embodiments, the transmembrane domain is derived from a protein selected from the group consisting of: CD8, CD28, CD3zeta, CD4, 4-IBB, OX40, ICOS, 2B4, CD25, CD7, LAX, LAT, LAIR1, PECAM-1, LIR1, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM. In some embodiments, a transmembrane domain of a cell receptor is an PECAM-1 transmembrane domain. In some embodiments, a transmembrane domain of a cell receptor is an LIR1 transmembrane domain. In some embodiments, a transmembrane domain of a cell receptor is an IRTA2 transmembrane domain. In some embodiments, a transmembrane domain of a cell receptor is an IRTA4 transmembrane domain. In some embodiments, a transmembrane domain of a cell receptor is an NKIR transmembrane domain. In some embodiments, a transmembrane domain of a cell receptor is an IL1RAP transmembrane domain. In some embodiments, a transmembrane domain of a cell receptor is an PTPRO transmembrane domain. In some embodiments, a transmembrane domain of a cell receptor is an PTPRZ1 transmembrane domain. In some embodiments, a transmembrane domain of a cell receptor is an TLT1 transmembrane domain. In some embodiments, a transmembrane domain of a cell receptor is an SLAMF1 transmembrane domain. In some embodiments, a transmembrane domain of a cell receptor is an SLAMF5 transmembrane domain. In some embodiments, a transmembrane domain of a cell receptor is an PCDHGC3 transmembrane domain. In some embodiments, a transmembrane domain of a cell receptor is an LIFR transmembrane domain. In some embodiments, a transmembrane domain of a cell receptor is an ERMAP transmembrane domain. In some embodiments, a transmembrane domain of a cell receptor is an IL1RAPL2 transmembrane domain. In some embodiments, a transmembrane domain of a cell receptor is an CDH5 transmembrane domain. In some embodiments, a transmembrane domain of a cell receptor is an MPZL1 transmembrane domain. In some embodiments, a transmembrane domain of a cell receptor is an MPZ transmembrane domain. In some embodiments, a transmembrane domain of a cell receptor is an FCGR2B transmembrane domain. In some embodiments, a transmembrane domain of a cell receptor is an SIGLEC-6 transmembrane domain. In some embodiments, a transmembrane domain of a cell receptor is an MPIG6B transmembrane domain. In some embodiments, a transmembrane domain of a cell receptor is an VSIG4 transmembrane domain. In some embodiments, a transmembrane domain of a cell receptor is an SIGLEC-12 transmembrane domain. In some embodiments, a transmembrane domain of a cell receptor is an LIR8 transmembrane domain. In some embodiments, a transmembrane domain of a cell receptor is an IRTA1 transmembrane domain. In some embodiments, a transmembrane domain of a cell receptor is an KIR2DL4 transmembrane domain. In some embodiments, a transmembrane domain of a cell receptor is an KIR2DL5 transmembrane domain. In some embodiments, a transmembrane domain of a cell receptor is an SIGLEC-7 transmembrane domain. In some embodiments, a transmembrane domain of a cell receptor is an FCRH3 transmembrane domain. In some embodiments, a transmembrane domain of a cell receptor is an PCDHGC5 transmembrane domain. In some embodiments, a transmembrane domain of a cell receptor is an CDH11 transmembrane domain. In some embodiments, a transmembrane domain of a cell receptor is an IMPG2 transmembrane domain. In some embodiments, a transmembrane domain of a cell receptor is an DSCAM transmembrane domain.

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from CD28.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to FWVLVVVGGVLACYSLLVTVAFIIFWV (SEQ ID NO: 23). In some embodiments, the transmembrane domain comprises the amino acid sequence of FWVLVVVGGVLACYSLLVTVAFIIFWV (SEQ ID NO:23).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from PECAM-1.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to GLIAVVIIGVIIALLIIAA (SEQ ID NO:24). In some embodiments, the transmembrane domain comprises the amino acid sequence of GLIAVVIIGVIIALLIIAA (SEQ ID NO:24).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from LIR1.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VIGILVAVILLLLLLLLLFLI (SEQ ID NO: 25). In some embodiments, the transmembrane domain comprises the amino acid sequence of VIGILVAVILLLLLLLLLFLI (SEQ ID NO: 25).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from IRTA2.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VAGGLLSIAGLAAGALLLYCW (SEQ ID NO: 26). In some embodiments, the transmembrane domain comprises the amino acid sequence of VAGGLLSIAGLAAGALLLYCW (SEQ ID NO: 26).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from IRTA4.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LWGLFGVLGFTGVALLLYALF (SEQ ID NO: 27). In some embodiments, the transmembrane domain comprises the amino acid sequence of LWGLFGVLGFTGVALLLYALF (SEQ ID NO: 27).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from NKIR.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VLLPLIFTILLLLLVAASLLA (SEQ ID NO: 28). In some embodiments, the transmembrane domain comprises the amino acid sequence of VLLPLIFTILLLLLVAASLLA (SEQ ID NO: 28).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from IL1RAP.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VLLVVILIVVYHVYWLEMVLF (SEQ ID NO: 29). In some embodiments, the transmembrane domain comprises the amino acid sequence of VLLVVILIVVYHVYWLEMVLF (SEQ ID NO: 29).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from PTPRO.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to ISVLAILSTLLIGLLLVTLII (SEQ ID NO: 30). In some embodiments, the transmembrane domain comprises the amino acid sequence of ISVLAILSTLLIGLLLVTLII (SEQ ID NO: 30).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from PTPRZ1.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AVIPLVIVSALTFICLVVLVGILIYW (SEQ ID NO: 31). In some embodiments, the transmembrane domain comprises the amino acid sequence of AVIPLVIVSALTFICLVVLVGILIYW (SEQ ID NO: 31).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from TLT1.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LIWGAVLLVGLLVAAVVLFAV (SEQ ID NO: 32). In some embodiments, the transmembrane domain comprises the amino acid sequence of LIWGAVLLVGLLVAAVVLFAV (SEQ ID NO: 32).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from SLAMF1.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to WAVYAGLLGGVIMILIMVVIL (SEQ ID NO: 33). In some embodiments, the transmembrane domain comprises the amino acid sequence of WAVYAGLLGGVIMILIMVVIL (SEQ ID NO: 33).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from SLAMF5.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LLSVLAMFFLLVLILSSVFLF (SEQ ID NO: 34). In some embodiments, the transmembrane domain comprises the amino acid sequence of LLSVLAMFFLLVLILSSVFLF (SEQ ID NO: 34).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from PCDHGC3.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LLLSLILVSVGFVVTVFGVII (SEQ ID NO: 139). In some embodiments, the transmembrane domain comprises the amino acid sequence of LLLSLILVSVGFVVTVFGVII (SEQ ID NO: 139).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from LIFR.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VGLIIAILIPVAVAVIVGVVTSILC (SEQ ID NO: 140). In some embodiments, the transmembrane domain comprises the amino acid sequence of VGLIIAILIPVAVAVIVGVVTSILC (SEQ ID NO: 140).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from ERMAP.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VALAVILPVLVLLIMVCLCLI (SEQ ID NO: 141). In some embodiments, the transmembrane domain comprises the amino acid sequence of VALAVILPVLVLLIMVCLCLI (SEQ ID NO: 141).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from IL1RAPL2.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to IELAGGLGAIFLLLVLLVVIY (SEQ ID NO: 142). In some embodiments, the transmembrane domain comprises the amino acid sequence of IELAGGLGAIFLLLVLLVVIY (SEQ ID NO: 142).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from CDH5.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AVVAILLCILTITVITLLIFL (SEQ ID NO: 143). In some embodiments, the transmembrane domain comprises the amino acid sequence of AVVAILLCILTITVITLLIFL (SEQ ID NO: 143).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from MPZL1.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to FPVWVVVGIVTAVVLGLTLLI (SEQ ID NO: 144). In some embodiments, the transmembrane domain comprises the amino acid sequence of FPVWVVVGIVTAVVLGLTLLI (SEQ ID NO: 144).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from MPZ.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to YGVVLGAVIGGVLGVVLLLLLLFYVV (SEQ ID NO: 145). In some embodiments, the transmembrane domain comprises the amino acid sequence of YGVVLGAVIGGVLGVVLLLLLLFYVV (SEQ ID NO: 145).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from FCGR2B.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SSSPMGIIVAVVTGIAVAAIVAA (SEQ ID NO: 146). In some embodiments, the transmembrane domain comprises the amino acid sequence of SSSPMGIIVAVVTGIAVAAIVAA (SEQ ID NO: 146).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from SIGLEC-6.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LGAVWGASITTLVFLCVCFIF (SEQ ID NO: 147). In some embodiments, the transmembrane domain comprises the amino acid sequence of LGAVWGASITTLVFLCVCFIF (SEQ ID NO: 147).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from MPIG6B.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LLIPLLGAGLVLGLGALGLVW (SEQ ID NO: 148). In some embodiments, the transmembrane domain comprises the amino acid sequence of LLIPLLGAGLVLGLGALGLVW (SEQ ID NO: 148).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from VSIG4.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VFAIILIISLCCMVVFTMAYI (SEQ ID NO: 149). In some embodiments, the transmembrane domain comprises the amino acid sequence of VFAIILIISLCCMVVFTMAYI (SEQ ID NO: 149).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from SIGLEC-12.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to FGGAGATALVFLYFCIIFVVV (SEQ ID NO: 150). In some embodiments, the transmembrane domain comprises the amino acid sequence of FGGAGATALVFLYFCIIFVVV (SEQ ID NO: 150).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from LIR8.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VVTGVSVAFVLLLFLLLFLLL (SEQ ID NO: 151). In some embodiments, the transmembrane domain comprises the amino acid sequence of VVTGVSVAFVLLLFLLLFLLL (SEQ ID NO: 151).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from IRTA1.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VAAGATGGLLSALLLAVALLF (SEQ ID NO: 152). In some embodiments, the transmembrane domain comprises the amino acid sequence of VAAGATGGLLSALLLAVALLF (SEQ ID NO: 152).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from KIR2DL4.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AVIRYSVAIILFTILPFFLLH (SEQ ID NO: 153). In some embodiments, the transmembrane domain comprises the amino acid sequence of AVIRYSVAIILFTILPFFLLH (SEQ ID NO: 153).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from KIR2DL5.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LHILIGTSVAIILFIILFFFL (SEQ ID NO: 154). In some embodiments, the transmembrane domain comprises the amino acid sequence of LHILIGTSVAIILFIILFFFL (SEQ ID NO: 154).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from SIGLEC-7.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to GAVGGAGATALVFLSFCVIFIVV (SEQ ID NO: 155). In some embodiments, the transmembrane domain comprises the amino acid sequence of GAVGGAGATALVFLSFCVIFIVV (SEQ ID NO: 155).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from FCRH3.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AAGITGLVLSILVLAAAAALL (SEQ ID NO: 156). In some embodiments, the transmembrane domain comprises the amino acid sequence of AAGITGLVLSILVLAAAAALL (SEQ ID NO: 156).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from PCDHGC5.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LIVALATVSLLSLVTFTFLSA (SEQ ID NO: 157). In some embodiments, the transmembrane domain comprises the amino acid sequence of LIVALATVSLLSLVTFTFLSA (SEQ ID NO: 157).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from CDH11.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to GALIAILACIVILLVIVVLFVTL (SEQ ID NO: 158). In some embodiments, the transmembrane domain comprises the amino acid sequence of GALIAILACIVILLVIVVLFVTL (SEQ ID NO: 158).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from IMPG2.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VIIGITIASVVGLLVIFSAII (SEQ ID NO: 159). In some embodiments, the transmembrane domain comprises the amino acid sequence of VIIGITIASVVGLLVIFSAII (SEQ ID NO: 159).

In some embodiments, the transmembrane domain and the intracellular signaling domain are derived from the same protein. In some embodiments, the transmembrane domain is derived from a first protein and the intracellular signaling domain is derived from a second protein that is distinct from the first protein, wherein the chimeric inhibitory receptor comprises a transmembrane domain is derived from DSCAM.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LVTISCILVGVLLLFVLLLVV (SEQ ID NO: 160). In some embodiments, the transmembrane domain comprises the amino acid sequence of LVTISCILVGVLLLFVLLLVV (SEQ ID NO: 160).

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 24. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 25. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 26. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 27. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 28. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 29. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 30. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 31. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 32. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 33. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 34. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 139. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 140. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 141. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 142. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 143. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 144. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 145. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 146. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 147. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 148. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 149. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 150. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 151. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 152. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 153. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 154. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 155. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 156. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 157. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 158. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 159. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 160.

Exemplary transmembrane domain protein sequences are shown in Table 3. Exemplary transmembrane domain nucleotide sequences are shown in Table 4.

TABLE 3

| | SEQ ID | |
|---|---|---|
| Amino Acid Sequence | NO: | Description |
| FWVLVVVGGVLACYSLLVTVA | 23 | CD28 TM |
| FIIFWV | | |
| GLIAVVIIGVIIALLIIAA | 24 | PECAM-1 TM |
| VIGILVAVILLLLLLLLLFLI | 25 | LIR1 TM |
| VAGGLLSIAGLAAGALLLYCW | 26 | IRTA2 TM |
| LWGLFGVLGFTGVALLLYALF | 27 | IRTA4 TM |
| VLLPLIFTILLLLLVAASLLA | 28 | NKIR TM |
| VLLVVILIVVYHVYWLEMVLF | 29 | IL1RAP TM |
| ISVLAILSTLLIGLLLVTLII | 30 | PTPRO TM |
| AVIPLVIVSALTFICLVVLVG | 31 | PTPRZ1 TM |
| ILIYW | | |
| LIWGAVLLVGLLVAAVVLFAV | 32 | TLT1 TM |
| WAVYAGLLGGVIMILIMVVIL | 33 | SLAMF1 TM |
| LLSVLAMFFLLVLILSSVFLF | 34 | SLAMF5 TM |
| LLLSLILVSVGFVVTVFGVII | 139 | PCDHGC3 |
| VGLIIAILIPVAVAVIVGVVT | 140 | LIFR |
| SILC | | |
| VALAVILPVLVLLIMVCLCLI | 141 | ERMAP |
| IELAGGLGAIFLLLVLLVVIY | 142 | IL1RAPL2 |
| AVVAILLCILTITVITLLIFL | 143 | CDH5 |
| FPVWVVVGIVTAVVLGLTLLI | 144 | MPZL1 |
| YGVVLGAVIGGVLGVVLLLLL | 145 | MPZ |
| LFYVV | | |
| SSSPMGIIVAVVTGIAVAAIV | 146 | FCGR2B |
| AA | | |
| LGAVWGASITTLVFLCVCFIF | 147 | SIGLEC-6 |
| LLIPLLGAGLVLGLGALGLVW | 148 | MPIG6B |
| VFAIILIISLCCMVVFTMAYI | 149 | VSIG4 |
| FGGAGATALVFLYFCIIFVVV | 150 | SIGLEC-12 |
| VVTGVSVAFVLLLFLLLFLLL | 151 | LIR8 |
| VAAGATGGLLSALLLAVALLF | 152 | IRTA1 |
| AVIRYSVAIILFTILPFFLLH | 153 | KIR2DL4 |
| LHILIGTSVAIILFIILFFFL | 154 | KIR2DL5 |
| GAVGGAGATALVFLSFCVIFI | 155 | SIGLEC-7 |
| VV | | |
| AAGITGLVLSILVLAAAAALL | 156 | FCRH3 |
| LIVALATVSLLSLVTFTFLSA | 157 | PCDHGC5 |
| GALIAILACIVILLVIVVLFV | 158 | CDH11 |
| TL | | |
| VIIGITIASVVGLLVIFSAII | 159 | IMPG2 |
| LVTISCILVGVLLLFVLLLVV | 160 | DSCAM |

TABLE 4

| | SEQ ID | Descrip- |
|---|---|---|
| Nucleotide Sequence | NO: | tion |
| TTTTGGGTGCTGGTGGTGGTTGGTGGAGTCCT | 35 | CD28 TM |
| GGCTTGCTATAGCTTGCTAGTAACAGTGGCCT | | |
| TTATTATTTTCTGGGTG | | |
| GGCCTGATCGCCGTGGTTATCATCGGCGTGAT | 36 | PECAM-1 |
| CATTGCCCTGCTGATTATCGCCGCC | | TM |
| GTTATAGGGATCCTGGTGGCTGTCATACTCCT | 37 | LIR1 TM |
| CTTGCTCCTCTTGTTGCTGCTTTTTTTGATA | | |
| GTTGCTGGTGGCCTGCTGTCTATTGCTGGACT | 38 | IRTA2 TM |
| TGCTGCTGGTGCTCTGCTGCTGTACTGCTGG | | |
| CTGTGGGGACTGTTTGGCGTGTTGGGCTTTAC | 39 | IRTA4 TM |
| AGGCGTGGCCCTGCTGCTGTACGCCCTGTTT | | |
| GTTCTGCTGCCCCTGATCTTCACCATCCTGTT | 40 | NKIR TM |
| GCTGCTGCTGGTGGCCGCTTCTCTGCTGGCT | | |
| GTGCTGCTGGTGGTCATCCTGATCGTGGTGTA | 41 | IL1RAP TM |
| CCACGTGTACTGGCTGGAAATGGTGCTGTTC | | |
| ATCTCCGTGCTGGCCATCCTGAGCACACTGCT | 42 | PTPRO TM |
| GATTGGACTGCTGCTCGTGACCCTGATCATC | | |
| GCCGTGATTCCTCTGGTTATCGTGTCTGCCCT | 43 | PTPRZ1 TM |
| GACCTTCATCTGCCTGGTGGTGCTCGTGGGCA | | |
| TCCTGATCTATTGG | | |
| CTGATCTGGGGAGCAGTGCTGCTTGTGGGACT | 44 | TLT1 TM |
| GCTGGTGGCTGCTGTGGTGCTGTTTGCCGTG | | |
| TGGGCCGTGTACGCAGGCCTGCTTGGCGGTGT | 45 | SLAMF1 TM |
| GATCATGATCCTGATTATGGTGGTCATCCTG | | |
| CTGCTGTCCGTCCTGGCCATGTTTTTTCTGTT | 46 | SLAMF5 TM |
| GGTACTCATTCTATCATCCGTGTTCCTGTTC | | |
| CTGCTGCTGTCTCTGATCCTGGTGTCCGTGGG | 161 | PCDHGC3 |
| CTTTGTGGTCACCGTGTTCGGCGTGATCATC | | |
| GTGGGACTGATCATTGCCATTCTGATCCCTGT | 162 | LIFR |
| GGCCGTGGCCGTGATTGTGGGCGTCGTGACAT | | |
| CCATCCTGTGC | | |
| GTGGCTCTGGCTGTGATTCTGCCTGTGCTGGT | 163 | ERMAP |
| GCTGCTGATCATGGTTTGCCTGTGCCTGATC | | |
| ATTGAACTGGCTGGCGGACTGGGCGCCATCTT | 164 | IL1RAPL2 |
| TCTGCTGCTGGTGCTGCTCGTGGTCATCTAC | | |
| GCCGTGGTTGCCATCCTGCTGTGTATCCTGAC | 165 | CDH5 |
| CATCACCGTGATTACCCTGCTGATCTTCCTG | | |
| TTCCCTGTGTGGGTTGTCGTGGGAATCGTGAC | 166 | MPZL1 |
| AGCTGTGGTGCTGGGACTGACCCTGCTGATC | | |
| TATGGCGTTGTGCTGGGAGCTGTGATTGGCGG | 167 | MPZ |
| AGTTCTGGGAGTTGTGCTGCTGCTCCTGCTGC | | |
| TGTTTTACGTCGTG | | |
| AGCAGCTCTCCCATGGGCATCATTGTGGCCGT | 168 | FCGR2B |
| GGTCACAGGCATTGCCGTGGCCGCTATAGTGG | | |
| CTGCT | | |
| CTGGGAGCTGTTTGGGGCGCCTCTATTACAAC | 169 | SIGLEC-6 |
| CCTGGTGTTCCTGTGCGTGTGCTTCATCTTC | | |
| CTGCTGATTCCTCTGCTTGGAGCCGGACTGGT | 170 | MPIG6B |
| GCTTGGACTTGGAGCACTGGGACTTGTGTGG | | |
| GTGTTCGCCATCATCCTGATCATCAGCCTGTG | 171 | VSIG4 |
| CTGCATGGTGGTGTTCACCATGGCCTACATC | | |
| TTTGGCGGAGCTGGTGCTACAGCCCTGGTGTT | 172 | SIGLEC-12 |
| CCTGTACTTCTGCATCATCTTCGTGGTCGTG | | |
| GTTGTGACTGGGGTCTCAGTGGCCTTCGTCCT | 173 | LIR8 |
| GCTGCTGTTCCTCCTCCTCTTCCTCCTCCTC | | |
| GTCGCCGCGGGAGCCACTGGAGGGCTGCTCAG | 174 | IRTA1 |
| TGCTCTTCTCCTGGCTGTGGCCCTGCTGTTT | | |
| GCTGTGATACGTTATTCCGTGGCCATCATCCT | 175 | KIR2DL4 |
| GTTCACCATCCTACCCTTCTTCCTGCTGCAT | | |
| CTGCACATTCTGATTGGGACCTCAGTGGCTAT | 176 | KIR2DL5 |
| CATCCTCTTCATCATCCTCTTCTTCTTTCTC | | |
| GGGGCGGTCGGGGGAGCTGGAGCCACAGCCCT | 177 | SIGLEC-7 |
| GGTCTTCCTCTCCTTCTGTGTCATCTTCATTG | | |
| TAGTG | | |
| GCCGCTGGAATTACAGGCCTGGTGCTGAGCAT | 178 | FCRH3 |
| TCTGGTGCTGGCTGCAGCTGCTGCCCTGCTG | | |
| CTGATTGTGGCCCTGGCCACAGTGTCTCTGCT | 179 | PCDHGC5 |
| GAGCCTCGTGACCTTCACCTTCCTGAGCGCC | | |
| GGAGCCCTGATTGCCATCCTGGCCTGTATCGT | 180 | CDH11 |
| GATCCTGCTGGTCATCGTGGTGCTGTTCGTGA | | |
| CCCTG | | |
| GTGATCATCGGCATCACAATCGCCTCTGTCGT | 181 | IMPG2 |
| GGGCCTGCTGGTCATCTTCAGCGCCATCATC | | |
| CTGGTCACCATCAGCTGTATCCTCGTGGGAGT | 182 | DSCAM |
| GCTGCTGCTGTTCGTCCTGCTGCTGGTTGTG | | |

In some embodiments, the transmembrane domain is physically linked to the extracellular protein binding domain. In some embodiments, the intracellular signaling domain is physically linked to the transmembrane domain. In some embodiments, the transmembrane domain is physically linked to the extracellular protein binding domain and the intracellular signaling domain is physically linked to the transmembrane domain.

In some embodiments, the one or more intracellular signaling domains are two intracellular signaling domains.

In some embodiments, the chimeric inhibitory receptor comprises a first intracellular signaling domain and a second intracellular signaling domain. In some embodiments, the chimeric inhibitory receptor comprises a first intracellular signaling domain derived from PECAM-1 and a second intracellular signaling domain derived from a protein selected from the group consisting of: CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from CD72 and a second intracellular signaling domain derived from a protein selected from the group consisting of: PECAM-1, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from IRTA2 and a second intracellular signaling domain derived from a protein selected from the group consisting of: PECAM-1, CD72, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from IRTA4 and a second intracellular signaling domain derived from a protein selected from the group consisting of: PECAM-1, CD72, IRTA2, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from NKIR and a second intracellular signaling domain derived from a protein selected from the group consisting of: PECAM-1, CD72, IRTA2, IRTA4, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from IL1RAP and a second intracellular signaling domain derived from a protein selected from the group consisting of: PECAM-1, CD72, IRTA2, IRTA4, NKIR, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from PTPRO and a second intracellular signaling domain derived from a protein selected from the group consisting of: PECAM-1, CD72, IRTA2, IRTA4, NKIR, IL1RAP, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from PTPRZ1 and a second intracellular signaling domain derived from a protein selected from the group consisting of: PECAM-1, CD72, IRTA2, IRTA4, NKIR, IL1RAP, PTPRO, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from TLT1 and a second intracellular signaling domain derived from a protein selected from the group consisting of: PECAM-1, CD72, IRTA2, IRTA4, NKIR, IL1RAP, PTPRO, PTPRZ1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from SLAMF1 and a second intracellular signaling domain derived from a protein selected from the group consisting of: PECAM-1, CD72, IRTA2, IRTA4, NKIR, IL1RAP, PTPRO, PTPRZ1, TLT1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from SLAMF5 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from PCDHGC3 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from LIFR and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3. ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from ERMAP and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from IL1RAPL2 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from CDH5 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, MPZL1, MPZ, FCGR2B. SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from MPZL1 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from MPZ and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from FCGR2B and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from SIGLEC-6 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from MPIG6B and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, VSIG4, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from VSIG4 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, SIGLEC-12, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from SIGLEC-12 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, LIR8, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from LIR8 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, IRTA1, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from IRTA1 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, KIR2DL4, KIR2DL5, SIGLEC-7, FCRH3, PCDHGC5, CDH11, IMPG2, and DSCAM.

In some embodiments, the chimeric inhibitory receptor comprises a first intracellular signaling domain derived from KIR2DL4 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL5, SIGLEC-7, FCRH3, PCDHGC5, CDH11, IMPG2, and DSCAM.

In some embodiments, the chimeric inhibitory receptor comprises a first intracellular signaling domain derived from KIR2DL5 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, SIGLEC-7, FCRH3, PCDHGC5, CDH11, IMPG2, and DSCAM.

In some embodiments, the chimeric inhibitory receptor comprises a first intracellular signaling domain derived from SIGLEC-7 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, FCRH3, PCDHGC5, CDH11, IMPG2, and DSCAM.

In some embodiments, the chimeric inhibitory receptor comprises a first intracellular signaling domain derived from FCRH3 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, PCDHGC5, CDH11, IMPG2, and DSCAM.

In some embodiments, the chimeric inhibitory receptor comprises a first intracellular signaling domain derived from CDH11 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, IMPG2, and DSCAM.

In some embodiments, the chimeric inhibitory receptor comprises a first intracellular signaling domain derived from IMPG2 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, CDH11, and DSCAM.

In some embodiments, the chimeric inhibitory receptor comprises a first intracellular signaling domain derived from DSCAM and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, CDH11, and IMPG2.

### Extracellular Protein Binding Domains

The inhibitory chimeric receptors described herein further comprise extracellular protein binding domains.

In some embodiments, immune cells expressing an inhibitory chimeric receptor are genetically modified to recognize multiple targets or antigens, which permits the recognition of unique target or protein expression patterns on tumor cells.

In some embodiments, the protein is not expressed on the target tumor. In some embodiments, the expression in non-tumor cells is at least 2-fold, at least 3-fold, at least 4-fold, at least 5-fold, at least 6-fold, at least 7-fold, at least 8-fold, at least 9-fold, or at least 10-fold or more lower than the level of expression that would result in activation of the tumor-targeting chimeric antigen receptor.

In some embodiments, the protein is expressed on a non-tumor cell.

In some embodiments, the protein is expressed on a non-tumor cell derived from a tissue selected from the group consisting of brain, neuronal tissue, endocrine, endothelial, bone, bone marrow, immune system, muscle, lung, liver, gallbladder, pancreas, gastrointestinal tract, kidney, urinary bladder, male reproductive organs, female reproductive organs, adipose, soft tissue, and skin.

In some embodiments, the extracellular protein binding domain comprises a ligand-binding domain. In some embodiments, the ligand-binding domain can be a domain from a receptor, wherein the receptor is selected from the group consisting of a T cell receptor (TCR), a B cell receptor (BCR), a cytokine receptor, an RTK receptor, a serine/threonine kinase receptor, a hormone receptor, an immunoglobulin superfamily receptor, and a TNFR-superfamily receptor. In some embodiments, the extracellular protein binding domain comprises a receptor-binding domain. In some embodiments, the extracellular protein binding domain comprises an antigen-binding domain.

In some embodiments, an extracellular protein binding domain of a inhibitory chimeric receptor of the disclosure comprises an antigen binding domain, such as a single chain Fv (scFv) specific for a tumor antigen. In some embodiments, an extracellular protein binding domain comprises an antibody, an antigen-binding fragment thereof, F(ab), F(ab′), a single chain variable fragment (scFv), or a single-domain antibody (sdAb).

The term “single-chain” refers to a molecule comprising amino acid monomers linearly linked by peptide bonds. In a particular such embodiment, the C-terminus of the Fab light chain is connected to the N-terminus of the Fab heavy chain in the single-chain Fab molecule. As described in more detail herein, an scFv has a variable domain of light chain (VL) connected from its C-terminus to the N-terminal end of a variable domain of heavy chain (VH) by a polypeptide chain. Alternately the scFv comprises of polypeptide chain where in the C-terminal end of the VH is connected to the N-terminal end of VL by a polypeptide chain.

The “Fab fragment” (also referred to as fragment antigen-binding) contains the constant domain (CL) of the light chain and the first constant domain (CH1) of the heavy chain along with the variable domains VL and VH on the light and heavy chains respectively. The variable domains comprise the complementarity determining loops (CDR, also referred to as hypervariable region) that are involved in antigen-binding. Fab′ fragments differ from Fab fragments by the addition of a few residues at the carboxy terminus of the heavy chain CH1 domain including one or more cysteines from the antibody hinge region.

“F(ab′)2” fragments contain two Fab fragments joined, near the hinge region, by disulfide bonds. F(ab′)2 fragments may be generated, for example, by recombinant methods or by pepsin digestion of an intact antibody. The F(ab′) fragments can be dissociated, for example, by treatment with β-mercaptoethanol.

“Fv” fragments comprise a non-covalently-linked dimer of one heavy chain variable domain and one light chain variable domain.

“Single-chain Fv” or “sFv” or “scFv” includes the VH and VL domains of an antibody, wherein these domains are present in a single polypeptide chain. In one embodiment, the Fv polypeptide further comprises a polypeptide linker between the VH and VL domains which enables the scFv to form the desired structure for antigen-binding.

The term “single domain antibody” or “sdAb” refers to a molecule in which one variable domain of an antibody specifically binds to an antigen without the presence of the other variable domain. Single domain antibodies, and fragments thereof, are described in Arabi Ghahroudi et al., FEBS Letters. 1998, 414:521-526 and Muyldermans et al., Trends in Biochem. Sci., 2001, 26:230-245, each of which is incorporated by reference in its entirety. Single domain antibodies are also known as sdAbs or nanobodies. Sdabs are fairly stable and easy to express as fusion partner with the Fc chain of an antibody (Harmsen M M, De Haard H J (2007). “Properties, production, and applications of camelid single-domain antibody fragments”. Appl. Microbiol Biotechnol. 77 (1): 13-22).

An “antibody fragment” comprises a portion of an intact antibody, such as the antigen-binding or variable region of an intact antibody. Antibody fragments include, for example, Fv fragments, Fab fragments, F(ab′)2 fragments, Fab′ fragments, scFv (sFv) fragments, and scFv-Fc fragments.

In some embodiments, the antigen-binding domain comprises an antibody, an antigen-binding fragment of an antibody, a F(ab) fragment, a F(ab′) fragment, a single chain variable fragment (scFv), or a single-domain antibody (sdAb). In some embodiments, the antigen-binding domain comprises a single chain variable fragment (scFv). In some embodiments, each scFv comprises a heavy chain variable domain (VH) and a light chain variable domain (VL). In some embodiments, the VH and VL are separated by a peptide linker.

In some embodiments, the extracellular protein binding domain comprises a ligand-binding domain. The ligand-binding domain can be a domain from a receptor, wherein the receptor is selected from the group consisting of TCR. BCR, a cytokine receptor. RTK receptors, serine/threonine kinase receptors, hormone receptors, immunoglobulin superfamily receptors, and TNFR-superfamily of receptors. In some embodiments, an extracellular protein binding domain binds to a target protein comprising Her2, CD20, or CD19.

The choice of binding domain depends upon the type and number of ligands that define the surface of a target cell. For example, the extracellular protein binding domain may be chosen to recognize a ligand that acts as a cell surface marker on target cells associated with non-disease states, such as “self” or normal tissue, or the extracellular protein binding domain may be chosen to recognize a ligand that acts as a cell surface marker on targets associated with a particular disease state, such as cancer or an autoimmune disease. In general, an inhibitory chimeric receptor binding domain may be selected from a non-disease state cell surface marker, while a tumor-targeting chimeric receptor binding domain may be selected from a disease state cell surface marker. Thus, examples of cell surface markers that may act as ligands for the extracellular protein binding domain in the inhibitory chimeric receptor of the present disclosure include those associated with normal tissue and examples of cell surface markers that may act as ligands for the protein binding domain in a tumor-targeting chimeric receptor include those associated with cancer cells and/or other forms of diseased cells. In some embodiments, an inhibitory chimeric receptor is engineered to target a non-tumor protein of interest by way of engineering a desired protein binding domain that specifically binds to a protein on a non-tumor cell encoded by an engineered nucleic acid.

An extracellular protein binding domain (e.g., an scFv) that specifically binds to a target or an epitope is a term understood in the art, and methods to determine such specific binding are also known in the art. A molecule is said to exhibit specific binding if it reacts or associates more frequently, more rapidly, with greater duration and/or with greater affinity with a particular target protein than it does with alternative targets. An extracellular protein binding domain (e.g., an scFv) that specifically binds to a first target protein may or may not specifically bind to a second target protein. As such, specific binding does not necessarily require (although it can include) exclusive binding. In some embodiments, an extracellular protein binding domain is an antigen-binding domain.

In some embodiments, the extracellular protein binding domain has a high binding affinity.

In some embodiments, the extracellular protein binding domain has a low binding affinity.

### Linkers

In some embodiments, the inhibitory chimeric receptor comprises a peptide linker. A linker is generally used to link two peptides of a protein binding domain, such as the peptides of an scFv or sdAb. Any appropriate linker known in the art may be used, including glycerin-serine based linkers. In some embodiments, the heavy chain variable domain (VH) and light chain variable domain (VL) of an scFv are separated by a peptide linker. In some embodiments, the scFv comprises the structure VH-L-VL or VL-L-VH, wherein VH is the heavy chain variable domain, L is the peptide linker, and VL is the light chain variable domain. In some embodiments, the peptide linker comprises an amino acid sequence selected from the group consisting of GGS (SEQ ID NO: 47), GGSGGS (SEQ ID NO: 48), GGSGGSGGS (SEQ ID NO: 49), GGSGGSGGSGGS (SEQ ID NO: 50), GGSGGSGGSGGSGGS (SEQ ID NO: 51), GGGS (SEQ ID NO: 52) GGGSGGGS (SEQ ID NO: 53), GGGSGGGSGGGS (SEQ ID NO: 54), GGGSGGGSGGGSGGGS (SEQ ID NO: 55), GGGSGGGSGGGSGGGSGGGS (SEQ ID NO: 56), GGGGS (SEQ ID NO: 57), GGGGSGGGGS (SEQ ID NO: 58), GGGGSGGGGSGGGGS (SEQ ID NO: 59), GGGGSGGGGSGGGGSGGGGS (SEQ ID NO: 60), GGGGSGGGGSGGGGSGGGGSGGGGS (SEQ ID NO: 61), GGGGSGGGGSGGGGSQSV (SEQ ID NO: 63), GSTSGSGKPGSGEGSTKG (SEQ ID NO: 64), SGGGGSGGGGSGGGGSGGGGSGGGSLQ (SEQ ID NO: 65), and TTTPAPRPPTPAPTIALQPLSLRPEACRPAAGGAVHTRGLDFACDQTTPGERSSLPAFYPGTSGSC SGCGSLSLP (SEQ ID NO: 62).

Exemplary linker protein sequences are shown in Table 5. An exemplary linker nucleotide sequence is shown in Table 6.

TABLE 5

| SEQ ID | | |
|---|---|---|
| NO: | Amino Acid Sequence | Description |
| 47 | GGS | (G₂S)₁ scFv |
| | | linker |
| 48 | GGSGGS | (G₂S)₂ scFv |
| | | linker |
| 49 | GGSGGSGGS | (G₂S)₃ scFv |
| | | linker |
| 50 | GGSGGSGGSGGS | (G₂S)₄ SCFv |
| | | linker |
| 51 | GGSGGSGGSGGSGGS | (G₂S)₅ scFv |
| | | linker |
| 52 | GGGS | (G₃S)₁ scFv |
| | | linker |
| 53 | GGGSGGGS | (G₃S)₂ scFv |
| | | linker |
| 54 | GGGSGGGSGGGS | (G₃S)₃ scFv |
| | | linker |
| 55 | GGGSGGGSGGGSGGGS | (G₃S)₄ SCFv |
| | | linker |
| 56 | GGGSGGGSGGGSGGGSGGGS | (G₃S)₅ scFv |
| | | linker |
| 57 | GGGGS | (G₄S)₁ scFv |
| | | linker |
| 58 | GGGGSGGGGS | (G₄S)₂ scFv |
| | | linker |
| 59 | GGGGSGGGGSGGGGS | (G₄S)₃ scFv |
| | | linker |
| 60 | GGGGSGGGGSGGGGSGGGGS | (G₄S)₄ ScFv |
| | | linker |
| 61 | GGGGSGGGGSGGGGSGGGGSGGGGS | (G₄S)₅ scFv |
| | | linker |
| 62 | TTTPAPRPPTPAPTIALQPLSLRPE | linker |
| | ACRPAAGGAVHTRGLDFACDQTTPG | |
| | ERSSLPAFYPGTSGSCSGCGSLSLP | |
| 63 | GGGGSGGGGSGGGGSQSV | linker |
| 64 | GSTSGSGKPGSGEGSTKG | linker |
| 65 | SGGGGSGGGGSGGGGSGGGGSGGGS | linker |
| | LQ | |

TABLE 6

| | SEQ ID | |
|---|---|---|
| Nucleic Acid Sequence | NO: | Description |
| GGAGGCGGAGGATCTGGTGGC | 66 | (G₄S)₃ scFv |
| GGAGGAAGTGGCGGAGGCGGT | | linker |
| TCT | | |

### Spacers or Hinge Domains

Chimeric receptors can also contain spacer or hinge domains in the polypeptide. In some embodiments, a spacer domain or a hinge domain is located between an extracellular domain (e.g., comprising the protein binding domain) and a transmembrane domain of an inhibitory chimeric receptor or tumor-targeting chimeric receptor, or between a intracellular signaling domain and a transmembrane domain of the inhibitory chimeric receptor or tumor-targeting chimeric receptor. A spacer or hinge domain is any oligopeptide or polypeptide that functions to link the transmembrane domain to the extracellular domain and/or the intracellular signaling domain in the polypeptide chain. Spacer or hinge domains provide flexibility to the inhibitory chimeric receptor or tumor-targeting chimeric receptor, or domains thereof, or prevent steric hindrance of the inhibitory chimeric receptor or tumor-targeting chimeric receptor, or domains thereof. In some embodiments, a spacer domain or hinge domain may comprise up to 300 amino acids (e.g., 10 to 100 amino acids, or 5 to 20 amino acids). In some embodiments, one or more spacer domain(s) may be included in other regions of an inhibitory chimeric receptor or tumor-targeting chimeric receptor.

Exemplary spacer or hinge domain protein sequences are shown in Table 7. Exemplary spacer or hinge domain nucleotide sequences are shown in Table 8.

TABLE 7

| SEQ ID | | |
|---|---|---|
| NO: | Amino Acid Sequence | Description |
| 67 | AAAIEVMYPPPYLDNEKSNGTIIHVKGKHLCPSPLFP | CD28 hinge |
| | GPSKP | |
| 68 | ESKYGPPCPSCP | IgG4 minimal hinge |
| 69 | ESKYGPPAPSAP | IgG4 minimal hinge, |
| | | no disulfides |
| 70 | ESKYGPPCPPCP | IgG4 S228P minimal |
| | | hinge, enhanced |
| | | disulfide formation |
| 71 | EPKSCDKTHTCP | IgG1 minimal hinge |
| 72 | AAAFVPVFLPAKPTTTPAPRPPTPAPTIASQPLSLRP | Extended CD8a hinge |
| | EACRPAAGGAVHTRGLDFACDIYIWAPLAGTCGVLLL | |
| | SLVITLYCNHRN | |
| 73 | TTTPAPRPPTPAPTIALQPLSLRPEACRPAAGGAVHT | CD8a hinge |
| | RGLDFACD | |
| 74 | ACPTGLYTHSGECCKACNLGEGVAQPCGANQTVCEPC | LNGFR hinge |
| | LDSVTFSDVVSATEPCKPCTECVGLQSMSAPCVEADD | |
| | AVCRCAYGYYQDETTGRCEACRVCEAGSGLVFSCQDK | |
| | QNTVCEECPDGTYSDEADAEC | |
| 75 | ACPTGLYTHSGECCKACNLGEGVAQPCGANQTVC | Truncated LNGFR |
| | | hinge (TNFR-Cys1) |
| 76 | AVGQDTQEVIVVPHSLPFKV | PDGFR-beta |
| | | extracellular linker |
| 77 | ALSNSIMYFSHFVPVFLPAKPTTTPAPRPPTPAPTIA | CD8 hinge |
| | SQPLSLRPEACRPAAGGAVHTRGLDFACD | |
| 183 | ESKYGPPCPSCPAPPVAGPSVFLFPPKPKDTLMISRT | IgG4CH2mutCH3 |
| | PEVTCVVVDVSQEDPEVQFNWYVDGVEVHNAKTKPRE | |
| | EQFQSTYRVVSVLTVLHQDWLNGKEYKCKVSNKGLPS | |
| | SIEKTISKAKGQPREPQVYTLPPSQEEMTKNQVSLTC | |
| | LVKGFYPSDIAVEWESNGQPENNYKTTPPVLDSDGSF | |
| | FLYSRLTVDKSRWQEGNVFSCSVMHEALHNHYTQKSL | |
| | SLSLGK | |
| 184 | ESKYGPPCPSCPGQPREPQVYTLPPSQEEMTKNQVSL | IgG4CH3 |
| | TCLVKGFYPSDIAVEWESNGQPENNYKTTPPVLDSDG | |
| | SFFLYSRLTVDKSRWQEGNVFSCSVMHEALHNHYTQK | |
| | SLSLSLGK | |

TABLE 8

| SEQ ID | | |
|---|---|---|
| NO: | Nucleic Acid Sequence | Description |
| 78 | GCAGCAGCTATCGAGGTGATGTATCCTCCGCCCTA | CD28 hinge |
| | CCTGGATAATGAAAAGAGTAATGGGACTATCATTC | |
| | ATGTAAAAGGGAAGCATCTTTGTCCTTCTCCCCTT | |
| | TTCCCCGGTCCGTCTAAACCT | |
| 207 | GAA AGC AAG TAC GGT CCA CCT TGC CCT | IgG4 minimal hinge |
| | AGC TGT CCG | |
| 79 | GAA TCC AAG TAC GGC CCC CCA GCG CCT | IgG4 minimal hinge, no |
| | AGT GCC CCA | disulfides |
| 80 | GAA TCT AAA TAT GGC CCG CCA TGC CCG | IgG4 S228P minimal hinge, |
| | CCT TGC CCA | enhanced disulfide formation |
| 81 | GAA CCG AAG TCT TGT GAT AAA ACT CAT | IgG1 minimal hinge |
| | ACG TGC CCG | |
| 82 | ACCACGACGCCAGCGCCGCGACCACCAACACCGGC | CD8 hinge |
| | GCCCACCATCGCGTTGCAGCCCCTGTCCCTGCGCC | |
| | CAGAGGCGTGCCGGCCAGCGGCGGGGGGCGCAGTG | |
| | CACACGAGGGGGCTGGACTTCGCCTGTGAT | |
| 208 | GCC TGC CCG ACC GGG CTC TAC ACT CAT | LNGFR hinge |
| | AGC GGG GAA TGT TGT AAG GCA TGT AAC | |
| | TTG GGT GAG GGC GTC GCA CAG CCC TGC | |
| | GGA GCT AAC CAA ACA GTG TGC GAA CCC | |
| | TGC CTC GAT AGT GTG ACG TTC TCT GAT | |
| | GTT GTA TCA GCT ACA GAG CCT TGC AAA | |
| | CCA TGT ACT GAG TGC GTT GGA CTT CAG | |
| | TCA ATG AGC GCT CCA TGT GTG GAG GCA | |
| | GAT GAT GCG GTC TGT CGA TGT GCT TAC | |
| | GGA TAC TAC CAA GAC GAG ACA ACA GGG | |
| | CGG TGC GAG GCC TGT AGA GTT TGT GAG | |
| | GCG GGC TCC GGG CTG GTG TTT TCA TGT | |
| | CAA GAC AAG CAA AAT ACG GTC TGT GAA | |
| | GAG TGC CCT GAT GGC ACC TAC TCA GAC | |
| | GAA GCA GAT GCA GAA TGC | |
| 209 | GCC TGC CCT ACA GGA CTC TAC ACG CAT | Truncated LNGFR hinge |
| | AGC GGT GAG TGT TGT AAA GCA TGC AAC | (TNFR-Cys1) |
| | CTC GGG GAA GGT GTA GCC CAG CCA TGC | |
| | GGG GCT AAC CAA ACC GTT TGC | |
| 210 | GCTGTGGGCCAGGACACGCAGGAGGTCATCGTGGT | PDGFR-beta extracellular |
| | GCCACACTCCTTGCCCTTTAAGGTG | linker |
| 211 | GAGTCTAAGTACGGCCCTCCTTGTCCTAGCTGCCC | IgG4CH2mutCH3 |
| | TGCTCCTCCTGTTGCTGGCCCTTCCGTGTTCCTGT | |
| | TTCCTCCAAAGCCTAAGGACACCCTGATGATCAGC | |
| | AGAACCCCTGAAGTGACCTGCGTGGTGGTGGACGT | |
| | GTCCCAAGAGGATCCTGAGGTGCAGTTCAATTGGT | |
| | ACGTGGACGGCGTGGAAGTGCACAACGCCAAGACC | |
| | AAGCCTAGAGAGGAACAGTTCCAGAGCACCTACAG | |
| | AGTGGTGTCCGTGCTGACAGTGCTGCACCAGGATT | |
| | GGCTGAACGGCAAAGAGTACAAGTGCAAGGTGTCC | |
| | AACAAGGGCCTGCCTAGCAGCATCGAGAAAACCAT | |
| | CAGCAAGGCCAAGGGACAGCCCAGAGAACCCCAGG | |
| | TGTACACACTGCCTCCAAGCCAAGAGGAAATGACC | |
| | AAGAACCAGGTGTCCCTGACCTGCCTGGTCAAGGG | |
| | CTTCTACCCTTCCGATATCGCCGTGGAATGGGAGA | |
| | GCAATGGCCAGCCTGAGAACAACTACAAGACCACA | |
| | CCTCCAGTGCTGGACAGCGACGGCTCATTCTTCCT | |
| | GTACAGCAGACTGACCGTGGACAAGAGCAGATGGC | |
| | AAGAGGGCAACGTGTTCAGCTGCAGCGTGATGCAC | |
| | GAGGCCCTGCACAACCACTACACCCAGAAGTCTCT | |
| | GAGCCTGAGCCTGGGCAAA | |
| 212 | GAGTCTAAGTACGGCCCTCCTTGTCCTAGCTGCCC | IgG4CH3 |
| | TGGACAGCCCAGAGAACCCCAGGTGTACACACTGC | |
| | CTCCAAGCCAAGAGGAAATGACCAAGAACCAGGTG | |
| | TCCCTGACCTGCCTGGTCAAGGGCTTCTACCCTTC | |
| | CGATATCGCCGTGGAATGGGAGAGCAATGGCCAGC | |
| | CTGAGAACAACTACAAGACCACACCTCCAGTGCTG | |
| | GACAGCGACGGCTCATTCTTCCTGTACAGCAGACT | |
| | GACCGTGGACAAGAGCAGATGGCAAGAGGGCAACG | |
| | TGTTCAGCTGCAGCGTGATGCACGAGGCCCTGCAC | |
| | AACCACTACACCCAGAAGTCTCTGAGCCTGAGCCT | |
| | GGGCAAA | |

In some embodiments, the chimeric inhibitory receptor further comprises a spacer region between the protein binding domain and the transmembrane domain.

Exemplary spacers and/or linkers are described in US 2022/0089718 and US 2021/0206863 which are hereby incorporated by reference in its entirety.

In some embodiments, the spacer region is derived from a protein selected from the group consisting of: CD8α, CD4, CD7, CD28, IgG1, IgG4, FcγRIIIα, LNGFR, and PDGFR. In some embodiments, the spacer region comprises an amino acid sequence selected from the group consisting of:

(SEQ ID NO: 67)
AAAIEVMYPPPYLDNEKSNGTIIHVKGKHLCPSPLFPGPSKP,
(SEQ ID NO: 68)
ESKYGPPCPSCP,
(SEQ ID NO: 69)
ESKYGPPAPSAP,
(SEQ ID NO: 70)
ESKYGPPCPPCP,
(SEQ ID NO: 71)
EPKSCDKTHTCP,
(SEQ ID NO: 72)
AAAFVPVFLPAKPTTTPAPRPPTPAPTIASQPLSLRPEACRPAAGGAVHT
RGLDFACDIYIWAPLAGTCGVLLLSLVITLYCNHRN,
(SEQ ID NO: 73)
TTTPAPRPPTPAPTIALQPLSLRPEACRPAAGGAVHTRGLDFACD,
(SEQ ID NO: 74)
ACPTGLYTHSGECCKACNLGEGVAQPCGANQTVCEPCLDSVTFSDVVSAT
EPCKPCTECVGLQSMSAPCVEADDAVCRCAYGYYQDETTGRCEACRVCEA
GSGLVFSCQDKQNTVCEECPDGTYSDEADAEC,
(SEQ ID NO: 75)
ACPTGLYTHSGECCKACNLGEGVAQPCGANQTVC,
(SEQ ID NO: 77)
ALSNSIMYFSHFVPVFLPAKPTTTPAPRPPTPAPTIASQPLSLRPEACRP
AAGGAVHTRGLDFACD
(SEQ ID NO: 76)
AVGQDTQEVIVVPHSLPFKV,
(SEQ ID NO: 183)
ESKYGPPCPSCPAPPVAGPSVFLFPPKPKDTLMISRTPEVTCVVVDVSQE
DPEVQFNWYVDGVEVHNAKTKPREEQFQSTYRVVSVLTVLHQDWLNGKEY
KCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQE
GNVFSCSVMHEALHNHYTQKSLSLSLGK,
and
(SEQ ID NO: 184)
ESKYGPPCPSCPGQPREPQVYTLPPSQEEMTKNQVSLTCLVKGFYPSDIA
VEWESNGQPENNYKTTPPVLDSDGSFFLYSRLTVDKSRWQEGNVFSCSVM
HEALHNHYTQKSLSLSLGK.

In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 67. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 68. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 69. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 70. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 71. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 72. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 73. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 74. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 75. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 76. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 77. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 183. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 184.

In some embodiments, the spacer region modulates sensitivity of the chimeric inhibitory receptor. In some embodiments, the spacer region increases sensitivity of the chimeric inhibitory receptor relative to an otherwise identical chimeric inhibitory receptor lacking the spacer region. In some embodiments, the spacer region reduces sensitivity of the chimeric inhibitory receptor relative to an otherwise identical chimeric inhibitory receptor lacking the spacer region. In some embodiments, the spacer region modulates potency of the chimeric inhibitory receptor relative to an otherwise identical chimeric inhibitory receptor lacking the spacer region. In some embodiments, the spacer region increases potency of the chimeric inhibitory receptor relative to an otherwise identical chimeric inhibitory receptor lacking the spacer region. In some embodiments, the spacer region reduces potency of the chimeric inhibitory receptor relative to an otherwise identical chimeric inhibitory receptor lacking the spacer region. In some embodiments, the spacer region modulates basal prevention, attenuation, or inhibition of activation of the tumor-targeting chimeric receptor expressed on the immunomodulatory cell relative to an otherwise identical chimeric inhibitory receptor lacking the spacer region. In some embodiments, the spacer region reduces basal prevention, attenuation, or inhibition relative to an otherwise identical chimeric inhibitory receptor lacking the spacer region. In some embodiments, the spacer region increases basal prevention, attenuation, or inhibition relative to an otherwise identical chimeric inhibitory receptor lacking the spacer region.

In some embodiments, wherein the chimeric inhibitory receptor further comprises an intracellular spacer region positioned between the transmembrane domain and the intracellular signaling domain and operably linked to each of the transmembrane domain and the intracellular signaling domain. In some embodiments, the chimeric inhibitory receptor further comprises an intracellular spacer region positioned between the transmembrane domain and the intracellular signaling domain and physically linked to each of the transmembrane domain and the intracellular signaling domain.

In some embodiments, the intracellular spacer region modulates sensitivity of the chimeric inhibitory receptor relative to an otherwise identical chimeric inhibitory receptor lacking the intracellular spacer region. In some embodiments, the intracellular spacer region increases sensitivity of the chimeric inhibitory receptor relative to an otherwise identical chimeric inhibitory receptor lacking the intracellular spacer region. In some embodiments, the intracellular spacer region reduces sensitivity of the chimeric inhibitory receptor relative to an otherwise identical chimeric inhibitory receptor lacking the intracellular spacer region. In some embodiments, the intracellular spacer region modulates potency of the chimeric inhibitory receptor relative to an otherwise identical chimeric inhibitory receptor lacking the intracellular spacer region.

In some embodiments, the intracellular spacer region increases potency of the chimeric inhibitory receptor relative to an otherwise identical chimeric inhibitory receptor lacking the intracellular spacer region. In some embodiments, the intracellular spacer region reduces potency of the chimeric inhibitory receptor relative to an otherwise identical chimeric inhibitory receptor lacking the intracellular spacer region. In some embodiments, the intracellular spacer region modulates basal prevention, attenuation, or inhibition of activation of the tumor-targeting chimeric receptor expressed on the immunomodulatory cell when expressed on an immunomodulatory cell relative to an otherwise identical chimeric inhibitory receptor lacking the intracellular spacer region. In some embodiments, the intracellular spacer region reduces basal prevention, attenuation, or inhibition relative to an otherwise identical chimeric inhibitory receptor lacking the intracellular spacer region. In some embodiments, the intracellular spacer region increases basal prevention, attenuation, or inhibition relative to an otherwise identical chimeric inhibitory receptor lacking the intracellular spacer region.

### Extracellular Protein Binding Domain

As used herein, the term “extracellular protein binding domain” refers to an extracellular domain of a chimeric inhibitory protein of the present disclosure that binds to a specific protein. Examples of protein binding domains are known to those having skill in the art and include, but are not limited to, single-chain variable fragments (scFv), natural receptor/ligand domains, and orthogonal dimerization domains such as leucine zippers that engage with a soluble targeting molecule.

In some embodiments, the extracellular protein binding domain comprises an antigen-binding domain. In some embodiments, the extracellular protein binding domain comprises two antigen-binding domains.

Antigen-binding domains of the present disclosure can include any domain that binds to the antigen including, without limitation, a monoclonal antibody, a polyclonal antibody, a recombinant antibody, a bispecific antibody, a conjugated antibody, a human antibody, a humanized antibody, and a functional fragment thereof, including but not limited to a single-domain antibody (sdAb) such as a heavy chain variable domain (VH), a light chain variable domain (VL) and a variable domain (VHH) of camelid derived nanobody, and to an alternative scaffold known in the art to function as an antigen-binding domain, such as a recombinant fibronectin domain, a T cell receptor (TCR), a recombinant TCR with enhanced affinity, or a fragment thereof, e.g., single chain TCR, and the like.

In some embodiments, the extracellular protein binding domain comprises an antibody, or antigen-binding fragment thereof. In some embodiments, the extracellular protein binding domain comprises a F(ab) fragment, a F(ab′) fragment, a single chain variable fragment (scFv), or a single-domain antibody (sdAb).

The term “single-chain” refers to a molecule comprising amino acid monomers linearly linked by peptide bonds. In certain embodiments, the amino acid monomers are linearly linked by peptide linkers, including, but not limited to, comprises any of the amino acid sequences shown in Table 5.

“Single-chain Fv” or “sFv” or “scFv” includes the VH and VL domains of an antibody, wherein these domains are present in a single polypeptide chain. In one embodiment, the Fv polypeptide further comprises a polypeptide linker between the VH and VL domains which enables the scFv to form the desired structure for antigen-binding. As described in more detail herein, an scFv has a variable domain of light chain (VL) connected from its C-terminus to the N-terminal end of a variable domain of heavy chain (VH) by a polypeptide chain. Alternatively, the scFv comprises of polypeptide chain where in the C-terminal end of the VH is connected to the N-terminal end of VL by a polypeptide chain. In certain embodiments, the VH and VL are separated by a peptide linker. In certain embodiments, the scFv peptide linker comprises any of the amino acid sequences shown in Table 2. In certain embodiments, the scFv comprises the structure VH-L-VL or VL-L-VH, wherein VH is the heavy chain variable domain. L is the peptide linker, and VL is the light chain variable domain. In some embodiments, each of the one or more scFvs comprises the structure VH-L-VL or VL-L-VH, wherein VH is the heavy chain variable domain. L is the peptide linker, and VL is the light chain variable domain. When there are two or more scFv linked together, each scFv can be linked to the next scFv with a peptide linked. In some embodiments, each of the one or more scFvs is separated by a peptide linker.

The “Fab fragment” (also referred to as fragment antigen-binding) contains the constant domain (CL) of the light chain and the first constant domain (CH1) of the heavy chain along with the variable domains VL and VH on the light and heavy chains respectively. The variable domains comprise the complementarity determining loops (CDR, also referred to as hypervariable region) that are involved in antigen-binding. Fab′ fragments differ from Fab fragments by the addition of a few residues at the carboxy terminus of the heavy chain CH1 domain including one or more cysteines from the antibody hinge region. In a particular such embodiment, the C-terminus of the Fab light chain is connected to the N-terminus of the Fab heavy chain in a single-chain Fab molecule.

“F(ab′)2” fragments contain two Fab fragments joined, near the hinge region, by disulfide bonds. F(ab′)2 fragments may be generated, for example, by recombinant methods or by pepsin digestion of an intact antibody. The F(ab′) fragments can be dissociated, for example, by treatment with β-mercaptoethanol.

“Fv” fragments comprise a non-covalently-linked dimer of one heavy chain variable domain and one light chain variable domain.

The term “single domain antibody” or “sdAb” refers to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety. Single domain antibodies are also known as sdAbs or nanobodies. Sdabs are fairly stable and easy to express as fusion partner with the Fc chain of an antibody (Harmsen M M, De Haard H J (2007). “Properties, production, and applications of camelid single-domain antibody fragments”. Appl. Microbiol Biotechnol. 77 (1): 13-22).

An “antibody fragment” comprises a portion of an intact antibody, such as the antigen-binding or variable region of an intact antibody. Antibody fragments include, for example, Fv fragments, Fab fragments, F(ab′)2 fragments, Fab′ fragments, scFv (sFv) fragments, and scFv-Fc fragments.

### Polynucleotides Encoding Inhibitory Chimeric Receptors

In another aspect, presented herein are a polynucleotide or set of polynucleotides encoding an inhibitory chimeric receptor, and a vector comprising such a polynucleotide. When the inhibitory chimeric receptor is a multichain receptor, a set of polynucleotides is used. In this case, the set of polynucleotides can be cloned into a single vector or a plurality of vectors. In some embodiments, the polynucleotide comprises a sequence encoding an inhibitory chimeric receptor, wherein the sequence encoding an extracellular protein binding domain is contiguous with and in the same reading frame as a sequence encoding an intracellular signaling domain and a transmembrane domain.

The polynucleotide can be codon optimized for expression in a mammalian cell. In some embodiments, the entire sequence of the polynucleotide has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

The polynucleotide encoding an inhibitory chimeric receptor can be obtained using recombinant methods known in the art, such as, for example by screening libraries from cells expressing the polynucleotide, by deriving it from a vector known to include the same, or by isolating directly from cells and tissues containing the same, using standard techniques. Alternatively, the polynucleotide can be produced synthetically, rather than cloned.

The polynucleotide can be cloned into a vector. In some embodiments, an expression vector known in the art is used. Accordingly, the present disclosure includes retroviral and lentiviral vector constructs expressing an inhibitory chimeric receptor that can be directly transduced into a cell.

The present disclosure also includes an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequence (“UTR”) (e.g., a 3′ and/or 5′ UTR described herein), a 5′ cap (e.g., a 5′ cap described herein) and/or Internal Ribosome Entry Site (IRES) (e.g., an IRES described herein), the nucleic acid to be expressed, and a polyA tail. RNA so produced can efficiently transfect different kinds of cells. In some embodiments, an RNA inhibitory chimeric receptor vector is transduced into a cell, e.g., a T cell or a NK cell, by electroporation.

### Cells

In one aspect, the present disclosure provides inhibitory chimeric receptor-modified cells. The cells can be stem cells, producer cells, progenitor cells, and/or immune cells modified to express an inhibitory chimeric receptor described herein. In some embodiments, a cell line derived from an immune cell is used. Non-limiting examples of cells, as provided herein, include mesenchymal stem cells (MSCs), natural killer (NK) cells, NKT cells, innate lymphoid cells, mast cells, eosinophils, basophils, macrophages, neutrophils, mesenchymal stem cells, dendritic cells, T cells (e.g., CD8+ T cells, CD4+ T cells, gamma-delta T cells, and T regulatory cells (CD4+, FOXP3+, CD25+)) and B cells. In some embodiments, the cell a stem cell, such as pluripotent stem cell, embryonic stem cell, adult stem cell, bone-marrow stem cell, umbilical cord stem cells, or other stem cell.

The cells can be modified to express an inhibitory chimeric receptor provided herein. Accordingly, the present disclosure provides a cell (e.g., a population of cells) engineered to express an inhibitory chimeric receptor, wherein the inhibitory chimeric receptor comprises a protein binding domain, a transmembrane domain, and an inhibitory intracellular signaling domain.

In some embodiments, the immunomodulatory cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell. In some embodiments, the immunomodulatory cell is a CD8+ T cell. In some embodiments, the immunomodulatory cell is a CD4+ T cell. In some embodiments, the immunomodulatory cell is a Natural Killer T (NKT) cell. In some embodiments, the immunomodulatory cell is a Natural Killer (NK) cell.

In some embodiments, the cell is autologous. In some embodiments, the cell is allogeneic.

In some embodiments, an immunomodulatory cell comprises a chimeric inhibitory receptor, wherein the chimeric inhibitory receptor comprises: an extracellular protein binding domain; a transmembrane domain, wherein the transmembrane domain is operably linked to the extracellular protein binding domain; and an intracellular signaling domain, wherein the intracellular signaling domain is operably linked to the transmembrane domain, and wherein upon binding of the protein to the chimeric inhibitory receptor, the chimeric inhibitory receptor prevents, attenuates, or inhibits activation of a tumor-targeting chimeric receptor expressed on the surface of the cell.

In some embodiments, the cell further comprises a tumor-targeting chimeric receptor expressed on the surface of the cell. In some embodiments, the chimeric inhibitory receptor is recombinantly expressed.

In some embodiments, prior to binding of the protein to the chimeric inhibitory receptor, the tumor-targeting chimeric receptor is capable of activating the cell. In some embodiments, upon binding of the protein to the chimeric inhibitory receptor, the chimeric inhibitory receptor suppresses cytokine production from the activated cell. In some embodiments, upon binding of the protein to the chimeric inhibitory receptor, the chimeric inhibitory receptor suppresses a cell-mediated immune response to a target cell, wherein the immune response is induced by activation of the immunomodulatory cell. In some embodiments, the target cell is a tumor cell. In some embodiments, the target cell is a non-tumor cell.

### Cells Expressing Multiple Chimeric Receptors

The cells can be modified to express an inhibitory chimeric receptor provided herein. The cells can also be modified to express an inhibitory chimeric receptor (e.g., an iCAR) and a tumor-targeting CAR (e.g., an aCAR). If a cell is modified to express at least one inhibitory chimeric receptor and at least one tumor-targeting CAR, the cells can express multiple inhibitory and/or tumor-targeting chimeric receptor proteins and/or polynucleotides. In some embodiments, the cell expresses at least 1, at least 2, at least 3, at least 4, at least 5, at least 6, at least 7, at least 8, at least 9, or at least 10 or more inhibitory chimeric receptor polynucleotide and/or polypeptide. In some embodiments, the cell contains at least 1, at least 2, at least 3, at least 4, at least 5, at least 6, at least 7, at least 8, at least 9, or at least 10 or more tumor-targeting chimeric receptor polynucleotide and/or polypeptide.

### Methods of Preparing Inhibitory Chimeric Receptor-Modified Cells

In one aspect, the present disclosure provides a method of preparing a modified immune cells comprising an inhibitory chimeric receptor for experimental or therapeutic use.

Ex vivo procedures for making therapeutic inhibitory chimeric receptor-modified cells are well known in the art. For example, cells are isolated from a mammal (e.g., a human) and genetically modified (i.e., transduced or transfected in vitro) with a vector expressing a inhibitory chimeric receptor disclosed herein. The inhibitory chimeric receptor-modified cell can be administered to a mammalian recipient to provide a therapeutic benefit. The mammalian recipient may be a human and the inhibitory chimeric receptor-modified cell can be autologous with respect to the recipient. Alternatively, the cells can be allogeneic, syngeneic or xenogeneic with respect to the recipient. The procedure for ex vivo expansion of hematopoietic stem and progenitor cells is described in U.S. Pat. No. 5,199,942, incorporated herein by reference, can be applied to the cells of the present disclosure. Other suitable methods are known in the art; therefore the present disclosure is not limited to any particular method of ex vivo expansion of the cells. Briefly, ex vivo culture and expansion of immune effector cells (e.g., T cells, NK cells) comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In some embodiments, the methods comprise culturing the population of cells (e.g. in cell culture media) to a desired cell density (e.g., a cell density sufficient for a particular cell-based therapy). In some embodiments, the population of cells are cultured in the absence of an agent that represses activity of the repressible protease or in the presence of an agent that represses activity of the repressible protease.

In some embodiments, the population of cells is cultured for a period of time that results in the production of an expanded cell population that comprises at least 2-fold the number of cells of the starting population. In some embodiments, the population of cells is cultured for a period of time that results in the production of an expanded cell population that comprises at least 4-fold the number of cells of the starting population. In some embodiments, the population of cells is cultured for a period of time that results in the production of an expanded cell population that comprises at least 16-fold the number of cells of the starting population.

### Methods of Use

Methods for treatment of immune-related disorders, such as cancers, are also encompassed. Said methods include administering an inhibitory chimeric receptor or immunoresponsive inhibitory chimeric receptor-modified cell as described herein. In some embodiments, compositions comprising chimeric receptors or genetically modified immunoresponsive cells that express such chimeric receptors can be provided systemically or directly to a subject for the treatment of a proliferative disorder, such as a cancer.

In one aspect, the present disclosure provides a method of preparing a modified immune cells comprising at least one inhibitory chimeric receptor (e.g., inhibitory chimeric receptor (iCAR)-modified cells) for experimental or therapeutic use. In some embodiments, the modified immune cells further comprise at least one tumor-targeting chimeric receptor (e.g., iCAR and aCAR-modified cells).

In some aspects, methods of use encompass methods of preventing, attenuating, or inhibiting a cell-mediated immune response induced by a chimeric receptor expressed of the surface of an immunomodulatory cell, comprising: engineering the immunomodulatory cell to express the chimeric inhibitory receptor described herein on the surface of the immunomodulatory cell, wherein upon binding of a cognate protein to the chimeric inhibitory receptor, the intracellular signaling domain prevents, attenuates, or inhibits activation of the chimeric receptor. In other aspects, methods of use encompass methods of preventing, attenuating, or inhibiting activation of a chimeric receptor expressed on the surface of an immunomodulatory cell, comprising: contacting an isolated cell or a composition as described herein with a cognate protein of the chimeric inhibitory receptor under conditions suitable for the chimeric inhibitory receptor to bind the cognate protein, wherein upon binding of the protein to the chimeric inhibitory receptor, the intracellular signaling domain prevents, attenuates, or inhibits activation of the chimeric receptor.

In general, the inhibitory chimeric receptor is used to prevent, attenuate, inhibit, or suppress an immune response initiated by a tumor targeting chimeric receptor (e.g., an activating CAR). For example, an immunomodulator cell expresses an inhibitory chimeric antigen that recognizes an antigen target 1 (e.g., a non-tumor antigen) and a tumor-targeting chimeric receptor that recognizes an antigen target 2 (e.g., a tumor target). When the exemplary immunomodulatory cell contacts a target cell, the inhibitory and tumor targeting chimeric receptors may or may not bind to their cognate antigen. In exemplary instances where the target cell is a non-tumor cell that expresses both antigen target 1 and antigen target 2, both the inhibitory chimeric receptor and the tumor-targeting receptor can be activated. In such cases, the activation of the inhibitory chimeric receptor results in the prevention, attenuation, or inhibition of the tumor targeting chimeric receptor signaling and the immunomodulatory cell is not activated. Similarly, in exemplary instances where the target cell is a non-tumor cell that expresses only antigen target 1, only the inhibitory chimeric receptor can be activated. In contrast, in exemplary instances where the target cell is a tumor cell that expresses only antigen target 2, the inhibitory chimeric receptor cannot be activated while the tumor-targeting chimeric receptor can be activated, resulting in signal transduction that results in activation of the immunomodulatory cell.

Attenuation of an immune response initiated by a tumor targeting chimeric receptor can be a decrease or reduction in the activation of the tumor targeting chimeric receptor, a decrease or reduction in the signal transduction of a tumor targeting chimeric receptor, or a decrease or reduction in the activation of the immunomodulatory cell. The inhibitory chimeric receptor can attenuate activation of the tumor targeting chimeric receptor, signal transduction by the tumor targeting chimeric receptor, or activation of the immunomodulatory cell by the tumor targeting chimeric receptor 1-fold, 2-fold, 3-fold, 4-fold, 5-fold. 6-fold, 7-fold, 8-fold, 9-fold, 10-fold, 20-fold, 30-fold, 40-fold, 50-fold, 60-fold, 70-fold, 80-fold, 90-fold, 100-fold or more as compared to the activation of the tumor targeting chimeric receptor, signal transduction, or activation of the immunomodulatory cell as compared to an immunomodulatory cell lacking an inhibitory chimeric receptor. In some embodiments, attenuation refers to a decrease or reduction of the activity of a tumor targeting chimeric receptor after it has been activated.

Prevention of an immune response initiated by a tumor targeting chimeric receptor can be an inhibition or reduction in the activation of the tumor targeting chimeric receptor, an inhibition or reduction in the signal transduction of a tumor targeting chimeric receptor, or an inhibition or reduction in the activation of the immunomodulatory cell. The inhibitory chimeric receptor can prevent activation of the tumor targeting chimeric receptor, signal transduction by the tumor targeting chimeric receptor, or activation of the immunomodulatory cell by the tumor targeting chimeric receptor by about 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold or more as compared to the activation of the tumor targeting chimeric receptor, signal transduction, or activation of the immunomodulatory cell as compared to an immunomodulatory cell lacking an inhibitory chimeric receptor. In some embodiments, prevention refers to a blockage of the activity of a tumor targeting chimeric receptor before it has been activated.

Inhibition of an immune response initiated by a tumor targeting chimeric receptor can be an inhibition or reduction in the activation of the tumor targeting chimeric receptor, an inhibition or reduction in the signal transduction of a tumor targeting chimeric receptor, or an inhibition or reduction in the activation of the immunomodulatory cell. The inhibitory chimeric receptor can inhibit activation of the tumor targeting chimeric receptor, signal transduction by the tumor targeting chimeric receptor, or activation of the immunomodulatory cell by the tumor targeting chimeric receptor by about 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold or more as compared to the activation of the tumor targeting chimeric receptor, signal transduction, or activation of the immunomodulatory cell as compared to an immunomodulatory cell lacking an inhibitory chimeric receptor. In some embodiments, inhibition refers to a decrease or reduction of the activity of a tumor targeting chimeric receptor before or after it has been activated.

Suppression of an immune response initiated by a tumor targeting chimeric receptor can be an inhibition or reduction in the activation of the tumor targeting chimeric receptor, an inhibition or reduction in the signal transduction of a tumor targeting chimeric receptor, or an inhibition or reduction in the activation of the immunomodulatory cell. The inhibitory chimeric receptor can suppress activation of the tumor targeting chimeric receptor, signal transduction by the tumor targeting chimeric receptor, or activation of the immunomodulatory cell by the tumor targeting chimeric receptor by about 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold or more as compared to the activation of the tumor targeting chimeric receptor, signal transduction, or activation of the immunomodulatory cell as compared to an immunomodulatory cell lacking an inhibitory chimeric receptor. In some embodiments, suppression refers to a decrease or reduction of the activity of a tumor targeting chimeric receptor before or after it has been activated.

The immune response can be cytokine or chemokine production and secretion from an activated immunomodulatory cell. The immune response can be a cell-mediated immune response to a target cell.

In some embodiments, the chimeric inhibitory receptor is capable of suppressing cytokine production from an activated immunomodulatory cell. In some embodiments, the chimeric inhibitory receptor is capable of suppressing a cell-mediated immune response to a target cell, wherein the immune response is induced by activation of the immunomodulatory cell.

In one aspect, the present disclosure provides a type of cell therapy where immune cells are genetically modified to express an inhibitory chimeric receptor provided herein and the modified immune cells are administered to a subject in need thereof.

Thus, in some embodiments, the methods comprise delivering cells of the expanded population of cells to a subject in need of a cell-based therapy to treat a condition or disorder. In some embodiments, the subject is a human subject. In some embodiments, the condition or disorder is an autoimmune condition. In some embodiments, the condition or disorder is an immune related condition. In some embodiments, the condition or disorder is a cancer (e.g., a primary cancer or a metastatic cancer). In some embodiments, the cancer is a solid cancer. In some embodiments, the cancer is a liquid cancer, such as a myeloid disorder.

### Pharmaceutical Compositions

The inhibitory chimeric receptor or immunoresponsive cell can be formulated in pharmaceutical compositions. Pharmaceutical compositions of the present disclosure can comprise an inhibitory chimeric receptor (e.g., an iCAR) or immunoresponsive cell (e.g., a plurality of inhibitory chimeric receptor-expressing cells), as described herein, in combination with one or more pharmaceutically or physiologically acceptable carriers, diluents or excipients. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes. In certain embodiments, the composition is directly injected into an organ of interest (e.g., an organ affected by a disorder). Alternatively, the composition may be provided indirectly to the organ of interest, for example, by administration into the circulatory system (e.g., the tumor vasculature). Expansion and differentiation agents can be provided prior to, during, or after administration of the composition to increase production of T cells, NK cells, or CTL cells in vitro or in vivo.

In certain embodiments, the compositions are pharmaceutical compositions comprising genetically modified cells, such as immunoresponsive cells or their progenitors and a pharmaceutically acceptable carrier. Administration can be autologous or heterologous. For example, immunoresponsive cells, or progenitors can be obtained from one subject, and administered to the same subject or a different, compatible subject. In some embodiments, immunoresponsive cells of the present disclosure or their progeny may be derived from peripheral blood cells (e.g., in vivo, ex vivo, or in vitro derived) and may be administered via localized injection, including catheter administration, systemic injection, localized injection, intravenous injection, or parenteral administration. When administering a therapeutic composition of the present disclosure (e.g., a pharmaceutical composition containing a genetically modified cell of the present disclosure), it will generally be formulated in a unit dosage injectable form (solution, suspension, emulsion).

Certain aspects of the present disclosure relate to formulations of compositions comprising chimeric receptors of the present disclosure or genetically modified cells (e.g., immunoresponsive cells of the present disclosure) expressing such chimeric receptors. In some embodiments, compositions of the present disclosure comprising genetically modified cells may be provided as sterile liquid preparations, including without limitation isotonic aqueous solutions, suspensions, emulsions, dispersions, and viscous compositions, which may be buffered to a selected pH. Liquid preparations are typically easier to prepare than gels, other viscous compositions, and solid compositions. Additionally, liquid compositions may be more convenient to administer, especially by injection. In some embodiments, viscous compositions can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (e.g., glycerol, propylene glycol, liquid polyethylene glycol, etc.) and suitable mixtures thereof.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection. Ringer's Injection. Lactated Ringer's Injection. Preservatives, stabilizers, buffers, antioxidants and/or other additives can be included, as required. In some embodiments, compositions of the present disclosure can be isotonic, i.e., having the same osmotic pressure as blood and lacrimal fluid. In some embodiments, the desired isotonicity may be achieved using, for example, sodium chloride, dextrose, boric acid, sodium tartrate, propylene glycol, or other inorganic or organic solutes.

In some embodiments, compositions of the present disclosure may further include various additives that may enhance the stability and sterility of the compositions. Examples of such additives include, without limitation, antimicrobial preservatives, antioxidants, chelating agents, and buffers. In some embodiments, microbial contamination may be prevented by the inclusions of any of various antibacterial and antifungal agents, including without limitation parabens, chlorobutanol, phenol, sorbic acid, and the like. Prolonged absorption of an injectable pharmaceutical formulation of the present disclosure can be brought about by the use of suitable agents that delay absorption, such as aluminum monostearate and gelatin. In some embodiments, sterile injectable solutions can be prepared by incorporating genetically modified cells of the present disclosure in a sufficient amount of the appropriate solvent with various amounts of any other ingredients, as desired. Such compositions may be in admixture with a suitable carrier, diluent, or excipient such as sterile water, physiological saline, glucose, dextrose, or the like. In some embodiments, the compositions can also be lyophilized. The compositions can contain auxiliary substances such as wetting, dispersing agents. pH buffering agents, and antimicrobials depending upon the route of administration and the preparation desired.

In some embodiments, the components of the formulations of the present disclosure are selected to be chemically inert and to not affect the viability or efficacy of the genetically modified cells of the present disclosure.

One consideration concerning the therapeutic use of the genetically modified cells of the present disclosure is the quantity of cells needed to achieve optimal efficacy. In some embodiments, the quantity of cells to be administered will vary for the subject being treated. In certain embodiments, the quantity of genetically modified cells that are administered to a subject in need thereof may range from 1×10⁴ cells to 1×10¹⁰ cells. In some embodiments, the precise quantity of cells that would be considered an effective dose may be based on factors individual to each subject, including their size, age, sex, weight, and condition of the particular subject. Dosages can be readily ascertained by those skilled in the art based on the present disclosure and the knowledge in the art.

Whether it is a polypeptide, antibody, nucleic acid, small molecule or other pharmaceutically useful compound according to the present invention that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (cd), 1980.

A composition can be administered alone or in combination with other treatments, either simultaneously or sequentially dependent upon the condition to be treated.

### Kits

Certain aspects of the present disclosure relate to kits for the treatment and/or prevention of a cancer or other diseases (e.g., immune-related or autoimmune disorders). In certain embodiments, the kit includes a therapeutic or prophylactic composition comprising an effective amount of one or more chimeric receptors of the present disclosure, isolated nucleic acids of the present disclosure, vectors of the present disclosure, and/or cells of the present disclosure (e.g., immunoresponsive cells). In some embodiments, the kit comprises a sterile container. In some embodiments, such containers can be boxes, ampules, bottles, vials, tubes, bags, pouches, blister-packs, or other suitable container forms known in the art. The container may be made of plastic, glass, laminated paper, metal foil, or other materials suitable for holding medicaments.

In some embodiments, therapeutic or prophylactic composition is provided together with instructions for administering the therapeutic or prophylactic composition to a subject having or at risk of developing a cancer or immune-related disorder. In some embodiments, the instructions may include information about the use of the composition for the treatment and/or prevention of the disorder. In some embodiments, the instructions include, without limitation, a description of the therapeutic or prophylactic composition, a dosage schedule, an administration schedule for treatment or prevention of the disorder or a symptom thereof, precautions, warnings, indications, counter-indications, over-dosage information, adverse reactions, animal pharmacology, clinical studies, and/or references. In some embodiments, the instructions can be printed directly on the container (when present), or as a label applied to the container, or as a separate sheet, pamphlet, card, or folder supplied in or with the container.

### ADDITIONAL EMBODIMENTS

Embodiment 1: A chimeric inhibitory receptor comprising:
an extracellular protein binding domain;
a transmembrane domain, wherein the transmembrane domain is operably linked to the extracellular protein binding domain; and
one or more intracellular signaling domains, wherein the one or more intracellular signaling domains are operably linked to the transmembrane domain,
   wherein the one or more intracellular signaling domain are each derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM, and
   wherein at least one of the one or more intracellular signaling domains is capable of preventing, attenuating, or inhibiting activation of a tumor-targeting chimeric receptor expressed on an immunomodulatory cell.

Embodiment 2: The chimeric inhibitory receptor of embodiment 1, wherein the transmembrane domain is derived from the same protein as one of the one or more intracellular signaling domains.

Embodiment 3: The chimeric inhibitory receptor of embodiment 2, wherein the transmembrane domain further comprises at least a portion of an extracellular domain of the same protein.

Embodiment 4: The chimeric inhibitory receptor of embodiment 1, wherein the transmembrane domain is derived from a first protein and the one or more intracellular signaling domains are derived from proteins that are distinct from the first protein.

Embodiment 5: The chimeric inhibitory receptor of any one of embodiments 1-4, wherein the one or more intracellular signaling domains are two intracellular signaling domains.

Embodiment 6: The chimeric inhibitory receptor of embodiment 5, wherein the chimeric inhibitory receptor comprises a first intracellular signaling domain derived from PECAM-1 and a second intracellular signaling domain derived from a protein selected from the group consisting of: CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

Embodiment 7: The chimeric inhibitory receptor of embodiment 5, wherein the chimeric inhibitory receptor comprises a first intracellular signaling domain derived from CD72 and a second intracellular signaling domain derived from a protein selected from the group consisting of: PECAM-1, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

Embodiment 8: The chimeric inhibitory receptor of embodiment 5, wherein the chimeric inhibitory receptor comprises a first intracellular signaling domain derived from IRTA2 and a second intracellular signaling domain derived from a protein selected from the group consisting of: PECAM-1, CD72, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

Embodiment 9: The chimeric inhibitory receptor of embodiment 5, wherein the chimeric inhibitory receptor comprises a first intracellular signaling domain derived from IRTA4 and a second intracellular signaling domain derived from a protein selected from the group consisting of: PECAM-1, CD72, IRTA2, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

Embodiment 10: The chimeric inhibitory receptor of embodiment 5, wherein the chimeric inhibitory receptor comprises a first intracellular signaling domain derived from NKIR and a second intracellular signaling domain derived from a protein selected from the group consisting of: PECAM-1, CD72, IRTA2, IRTA4. IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

Embodiment 11: The chimeric inhibitory receptor of embodiment 5, wherein the chimeric inhibitory receptor comprises a first intracellular signaling domain derived from IL1RAP and a second intracellular signaling domain derived from a protein selected from the group consisting of: PECAM-1, CD72, IRTA2, IRTA4, NKIR, PTPRO, PTPRZ1, TLT1, SLAMF1, and SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

Embodiment 12: The chimeric inhibitory receptor of embodiment 5, wherein the chimeric inhibitory receptor comprises a first intracellular signaling domain derived from PTPRO and a second intracellular signaling domain derived from a protein selected from the group consisting of: PECAM-1, CD72, IRTA2, IRTA4, NKIR, IL1RAP, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

Embodiment 13: The chimeric inhibitory receptor of embodiment 5, wherein the chimeric inhibitory receptor comprises a first intracellular signaling domain derived from PTPRZ1 and a second intracellular signaling domain derived from a protein selected from the group consisting of: PECAM-1, CD72, IRTA2, IRTA4, NKIR, IL1RAP, PTPRO, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

Embodiment 14: The chimeric inhibitory receptor of embodiment 5, wherein the chimeric inhibitory receptor comprises a first intracellular signaling domain derived from TLT1 and a second intracellular signaling domain derived from a protein selected from the group consisting of: PECAM-1, CD72, IRTA2, IRTA4, NKIR, IL1RAP, PTPRO, PTPRZ1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

Embodiment 15: The chimeric inhibitory receptor of embodiment 5, wherein the chimeric inhibitory receptor comprises a first intracellular signaling domain derived from SLAMF1 and a second intracellular signaling domain derived from a protein selected from the group consisting of: PECAM-1, CD72, IRTA2, IRTA4, NKIR, IL1RAP, PTPRO, PTPRZ1, TLT1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1. KIR2DL4, KIR2DL5, SIGLEC-7, FCRH3, PCDHGC5, CDH11, IMPG2, and DSCAM.

Embodiment 16: The chimeric inhibitory receptor of embodiment 5, wherein the chimeric inhibitory receptor comprises a first intracellular signaling domain derived from SLAMF5 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1. KIR2DL4, KIR2DL5, SIGLEC-7, FCRH3, PCDHGC5, CDH11, IMPG2, and DSCAM.

Embodiment 17: The chimeric inhibitory receptor of embodiment 5, wherein the chimeric inhibitory receptor comprises a first intracellular signaling domain derived from PCDHGC3 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, LIFR, ERMAP, IL1RAPL2, CDH5, MPZL1, MPZ, FCGR2B, SIGLEC-6, MPIG6B, VSIG4, SIGLEC-12, LIR8, IRTA1. KIR2DL4, KIR2DL5, SIGLEC-7, FCRH3, PCDHGC5, CDH11, IMPG2, and DSCAM.

Embodiment 18: The chimeric inhibitory receptor of embodiment 5, wherein the chimeric inhibitory receptor comprises a first intracellular signaling domain derived from LIFR and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

Embodiment 19: The chimeric inhibitory receptor of embodiment 5, wherein the chimeric inhibitory receptor comprises a first intracellular signaling domain derived from ERMAP and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

Embodiment 20: The chimeric inhibitory receptor of embodiment 5, wherein the chimeric inhibitory receptor comprises a first intracellular signaling domain derived from IL1RAPL2 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, CDH5, MPZL1, MPZ, FCGR2B, SIGLEC-6, MPIG6B, VSIG4, SIGLEC-12, LIR8. IRTA1, KIR2DL4, KIR2DL5, SIGLEC-7, FCRH3, PCDHGC5, CDH11, IMPG2, and DSCAM.

Embodiment 21: The chimeric inhibitory receptor of embodiment 5, wherein the chimeric inhibitory receptor comprises a first intracellular signaling domain derived from CDH5 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, MPZL1, MPZ, FCGR2B, SIGLEC-6, MPIG6B, VSIG4, SIGLEC-12, LIR8. IRTA1, KIR2DL4, KIR2DL5, SIGLEC-7, FCRH3, PCDHGC5, CDH11, IMPG2, and DSCAM.

Embodiment 22: The chimeric inhibitory receptor of embodiment 5, wherein the chimeric inhibitory receptor comprises a first intracellular signaling domain derived from MPZL1 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZ, FCGR2B, SIGLEC-6, MPIG6B, VSIG4, SIGLEC-12, LIR8. IRTA1, KIR2DL4, KIR2DL5, SIGLEC-7, FCRH3, PCDHGC5, CDH11, IMPG2, and DSCAM.

Embodiment 23: The chimeric inhibitory receptor of embodiment 5, wherein the chimeric inhibitory receptor comprises a first intracellular signaling domain derived from MPZ and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

Embodiment 24: The chimeric inhibitory receptor of embodiment 5, wherein the chimeric inhibitory receptor comprises a first intracellular signaling domain derived from FCGR2B and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, SIGLEC-6, MPIG6B, VSIG4, SIGLEC-12, LIR8. IRTA1, KIR2DL4, KIR2DL5, SIGLEC-7, FCRH3, PCDHGC5, CDH11, IMPG2, and DSCAM.

Embodiment 25: The chimeric inhibitory receptor of embodiment 5, wherein the chimeric inhibitory receptor comprises a first intracellular signaling domain derived from SIGLEC-6 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, MPIG6B, VSIG4, SIGLEC-12, LIR8. IRTA1, KIR2DL4, KIR2DL5, SIGLEC-7, FCRH3, PCDHGC5, CDH11, IMPG2, and DSCAM.

Embodiment 26: The chimeric inhibitory receptor of embodiment 5, wherein the chimeric inhibitory receptor comprises a first intracellular signaling domain derived from MPIG6B and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, VSIG4, SIGLEC-12, LIR8. IRTA1, KIR2DL4, KIR2DL5, SIGLEC-7, FCRH3, PCDHGC5, CDH11, IMPG2, and DSCAM.

Embodiment 27: The chimeric inhibitory receptor of embodiment 5, wherein the chimeric inhibitory receptor comprises a first intracellular signaling domain derived from VSIG4 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, SIGLEC-12, LIR8. IRTA1, KIR2DL4, KIR2DL5, SIGLEC-7, FCRH3, PCDHGC5, CDH11, IMPG2, and DSCAM.

Embodiment 28: The chimeric inhibitory receptor of embodiment 5, wherein the chimeric inhibitory receptor comprises a first intracellular signaling domain derived from SIGLEC-12 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, LIR8, IRTA1, KIR2DL4, KIR2DL5, SIGLEC-7, FCRH3, PCDHGC5, CDH11, IMPG2, and DSCAM.

Embodiment 29: The chimeric inhibitory receptor of embodiment 5, wherein the chimeric inhibitory receptor comprises a first intracellular signaling domain derived from LIR8 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, IRTA1, KIR2DL4, KIR2DL5, SIGLEC-7, FCRH3, PCDHGC5, CDH11, IMPG2, and DSCAM.

Embodiment 30: The chimeric inhibitory receptor of embodiment 5, wherein the chimeric inhibitory receptor comprises a first intracellular signaling domain derived from IRTA1 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, KIR2DL4, KIR2DL5, SIGLEC-7, FCRH3, PCDHGC5, CDH11, IMPG2, and DSCAM.

Embodiment 31: The chimeric inhibitory receptor of embodiment 5, wherein the chimeric inhibitory receptor comprises a first intracellular signaling domain derived from KIR2DL4 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL5, SIGLEC-7, FCRH3, PCDHGC5, CDH11, IMPG2, and DSCAM.

Embodiment 32: The chimeric inhibitory receptor of embodiment 5, wherein the chimeric inhibitory receptor comprises a first intracellular signaling domain derived from KIR2DL5 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, SIGLEC-7, FCRH3, PCDHGC5, CDH11, IMPG2, and DSCAM.

Embodiment 33: The chimeric inhibitory receptor of embodiment 5, wherein the chimeric inhibitory receptor comprises a first intracellular signaling domain derived from SIGLEC-7 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, FCRH3, PCDHGC5, CDH11, IMPG2, and DSCAM.

Embodiment 34: The chimeric inhibitory receptor of embodiment 5, wherein the chimeric inhibitory receptor comprises a first intracellular signaling domain derived from FCRH3 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, PCDHGC5, CDH11, IMPG2, and DSCAM.

Embodiment 35: The chimeric inhibitory receptor of embodiment 5, wherein the chimeric inhibitory receptor comprises a first intracellular signaling domain derived from PCDHGC5 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, CDH11, IMPG2, and DSCAM.

Embodiment 36: The chimeric inhibitory receptor of embodiment 5, wherein the chimeric inhibitory receptor comprises a first intracellular signaling domain derived from CDH11 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, IMPG2, and DSCAM.

Embodiment 37: The chimeric inhibitory receptor of embodiment 5, wherein the chimeric inhibitory receptor comprises a first intracellular signaling domain derived from IMPG2 and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, CDH11, and DSCAM.

Embodiment 38: The chimeric inhibitory receptor of embodiment 5, wherein the chimeric inhibitory receptor comprises a first intracellular signaling domain derived from DSCAM and a second intracellular signaling domain derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, CDH11, and IMPG2.

Embodiment 39: The chimeric inhibitory receptor of any one of embodiments 1-38, wherein one of the one or more intracellular signaling domains is derived from PECAM-1.

Embodiment 40: The chimeric inhibitory receptor of embodiment 39, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to KCYFLRKAKAKQMPVEMSRPAVPLLNSNNEKMSDPNMEANSHYGHNDDVRNHAMKPINDNK EPLNSDVQYTEVQVSSAESHKDLGKKDTETVYSEVRKAVPDAVESRYSRTEGSLDGT (SEQ ID NO: 1).

Embodiment 41: The chimeric inhibitory receptor of embodiment 40, wherein the intracellular signaling domain comprises the amino acid sequence of KCYFLRKAKAKQMPVEMSRPAVPLLNSNNEKMSDPNMEANSHYGHNDDVRNHAMKPINDNK EPLNSDVQYTEVQVSSAESHKDLGKKDTETVYSEVRKAVPDAVESRYSRTEGSLDGT (SEQ ID NO: 1).

Embodiment 42: The chimeric inhibitory receptor of any one of embodiments 1-38, wherein one of the one or more intracellular signaling domains is derived from CD72.

Embodiment 43: The chimeric inhibitory receptor of embodiment 42, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to MAEAITYADLRFVKAPLKKSISSRLGQDPGADDDGEITYENVQVPAVLGVPSSLASSVLGDKAA VKSEQPTASWRAVTSPAVGRILPCRTTCLRY (SEQ ID NO: 2).

Embodiment 44: The chimeric inhibitory receptor of embodiment 42, wherein the intracellular signaling domain comprises the amino acid sequence of MAEAITYADLRFVKAPLKKSISSRLGQDPGADDDGEITYENVQVPAVLGVPSSLASSVLGDKAA VKSEQPTASWRAVTSPAVGRILPCRTTCLRY (SEQ ID NO: 2).

Embodiment 45: The chimeric inhibitory receptor of any one of embodiments 1-38, wherein one of the one or more intracellular signaling domains is derived from IRTA2.

Embodiment 46: The chimeric inhibitory receptor of embodiment 45, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LSRKAGRKPASDPARSPSDSDSQEPTYHNVPAWEELQPVYTNANPRGENVVYSEVRIIQEKKKH AVASDPRHLRNKGSPIIYSEVKVASTPVSGSLFLASSAPHR (SEQ ID NO: 3).

Embodiment 47: The chimeric inhibitory receptor of embodiment 45, wherein the intracellular signaling domain comprises the amino acid sequence of LSRKAGRKPASDPARSPSDSDSQEPTYHNVPAWEELQPVYTNANPRGENVVYSEVRIIQEKKKH AVASDPRHLRNKGSPIIYSEVKVASTPVSGSLFLASSAPHR (SEQ ID NO: 3).

Embodiment 48: The chimeric inhibitory receptor of any one of embodiments 1-38, wherein one of the one or more intracellular signaling domains is derived from IRTA4.

Embodiment 49: The chimeric inhibitory receptor of embodiment 48, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to HKISGESSATNEPRGASRPNPQEFTYSSPTPDMEELQPVYVNVGSVDVDVVYSQVWSMQQPESS ANIRTLLENKDSQVIYSSVKKS (SEQ ID NO: 4).

Embodiment 50: The chimeric inhibitory receptor of embodiment 48, wherein the intracellular signaling domain comprises the amino acid sequence of HKISGESSATNEPRGASRPNPQEFTYSSPTPDMEELQPVYVNVGSVDVDVVYSQVWSMQQPESS ANIRTLLENKDSQVIYSSVKKS (SEQ ID NO: 4).

Embodiment 51: The chimeric inhibitory receptor of any one of embodiments 1-38, wherein one of the one or more intracellular signaling domains is derived from NKIR.

Embodiment 52: The chimeric inhibitory receptor of embodiment 51, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to WRMMKYQQKAAGMSPEQVLQPLEGDLCYADLTLQLAGTSPQKATTKLSSAQVDQVEVEYVT MASLPKEDISYASLTLGAEDQEPTYCNMGHLSSHLPGRGPEEPTEYSTISRP (SEQ ID NO: 5).

Embodiment 53: The chimeric inhibitory receptor of embodiment 51, wherein the intracellular signaling domain comprises the amino acid sequence of WRMMKYQQKAAGMSPEQVLQPLEGDLCYADLTLQLAGTSPQKATTKLSSAQVDQVEVEYVT MASLPKEDISYASLTLGAEDQEPTYCNMGHLSSHLPGRGPEEPTEYSTISRP (SEQ ID NO: 5).

Embodiment 54: The chimeric inhibitory receptor of any one of embodiments 1-38, wherein one of the one or more intracellular signaling domains is derived from IL1RAP.

Embodiment 55: The chimeric inhibitory receptor of embodiment 54, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to YRAHFGTDETILDGKEYDIYVSYARNAEEEEFVLLTLRGVLENEFGYKLCIFDRDSLPGGIVTDET LSFIQKSRRLLVVLSPNYVLQGTQALLELKAGLENMASRGNINVILVQYKAVKETKVKELKRAK TVLTVIKWKGEKSKYPQGRFWKQLQVAMPVKKSPRRSSSDEQGLSYSSLKNV (SEQ ID NO: 6).

Embodiment 56: The chimeric inhibitory receptor of embodiment 54, wherein the intracellular signaling domain comprises the amino acid sequence of YRAHFGTDETILDGKEYDIYVSYARNAEEEEFVLLTLRGVLENEFGYKLCIFDRDSLPGGIVTDET LSFIQKSRRLLVVLSPNYVLQGTQALLELKAGLENMASRGNINVILVQYKAVKETKVKELKRAK TVLTVIKWKGEKSKYPQGRFWKQLQVAMPVKKSPRRSSSDEQGLSYSSLKNV (SEQ ID NO: 6).

Embodiment 57: The chimeric inhibitory receptor of any one of embodiments 1-38, wherein one of the one or more intracellular signaling domains is derived from PTPRO.

Embodiment 58: The chimeric inhibitory receptor of embodiment 57, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LRKKHLQMARECGAGTFVNFASLERDGKLPYNWRRSIFAFLTLLPSCLWTDYLLAFYINPWSKN GLKKRKLTNPVQLDDFDAYIKDMAKDSDYKFSLQFEELKLIGLDIPHFAADLPLNRCKNRYTNIL PYDFSRVRLVSMNEEEGADYINANYIPGYNSPQEYIATQGPLPETRNDFWKMVLQQKSQIIVML TQCNEKRRVKCDHYWPFTEEPIAYGDITVEMISEEEQDDWACRHFRINYADEMQDVMHFNYTA WPDHGVPTANAAESILQFVHMVRQQATKSKGPMIIHCSAGVGRTGTFIALDRLLQHIRDHEFVDI LGLVSEMRSYRMSMVQTEEQYIFIHQCVQLMWMKKKQQFCISDVIYENVSKS (SEQ ID NO: 7).

Embodiment 59: The chimeric inhibitory receptor of embodiment 57, wherein the intracellular signaling domain comprises the amino acid sequence of LRKKHLQMARECGAGTFVNFASLERDGKLPYNWRRSIFAFLTLLPSCLWTDYLLAFYINPWSKN GLKKRKLTNPVQLDDFDAYIKDMAKDSDYKFSLQFEELKLIGLDIPHFAADLPLNRCKNRYTNIL PYDFSRVRLVSMNEEEGADYINANYIPGYNSPQEYIATQGPLPETRNDFWKMVLQQKSQIIVML TQCNEKRRVKCDHYWPFTEEPIAYGDITVEMISEEEQDDWACRHFRINYADEMQDVMHFNYTA WPDHGVPTANAAESILQFVHMVRQQATKSKGPMIIHCSAGVGRTGTFIALDRLLQHIRDHEFVDI LGLVSEMRSYRMSMVQTEEQYIFIHQCVQLMWMKKKQQFCISDVIYENVSKS (SEQ ID NO: 7).

Embodiment 60: The chimeric inhibitory receptor of any one of embodiments 1-38, wherein one of the one or more intracellular signaling domains is derived from PTPRZ1.

Embodiment 61: The chimeric inhibitory receptor of embodiment 60, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RKCFQTAHFYLEDSTSPRVISTPPTPIFPISDDVGAIPIKHFPKHVADLHASSGFTEEFETLKEFYQE VQSCTVDLGITADSSNHPDNKHKNRYINIVAYDHSRVKLAQLAEKDGKLTDYINANYVDGYNR PKAYIAAQGPLKSTAEDFWRMIWEHNVEVIVMITNLVEKGRRKCDQYWPADGSEEYGNFLVTQ KSVQVLAYYTVRNFTLRNTKIKKGSQKGRPSGRVVTQYHYTQWPDMGVPEYSLPVLTFVRKAA YAKRHAVGPVVVHCSAGVGRTGTYIVLDSMLQQIQHEGTVNIFGFLKHIRSQRNYLVQTEEQYV FIHDTLVEAILSKETEVLDSHIHAYVNALLIPGPAGKTKLEKQFQLLSQSNIQQSDYSAALKQCNR EKNRTSSIIPVERSRVGISSLSGEGTDYINASYIMGYYQSNEFIITQHPLLHTIKDFWRMIWDHNAQ LVVMIPDGQNMAEDEFVYWPNKDEPINCESFKVTLMAEEHKCLSNEEKLIIQDFILEATQDDYV LEVRHFQCPKWPNPDSPISKTFELISVIKEEAANRDGPMIVHDEHGGVTAGTFCALTTLMHQLEK ENSVDVYQVAKMINLMRPGVFADIEQYQFLYKVILSLVSTRQEENPSTSLDSNGAALPDGNIAES LESLV (SEQ ID NO: 8).

Embodiment 62: The chimeric inhibitory receptor of embodiment 60, wherein the intracellular signaling domain comprises the amino acid sequence of RKCFQTAHFYLEDSTSPRVISTPPTPIFPISDDVGAIPIKHFPKHVADLHASSGFTEEFETLKEFYQE VQSCTVDLGITADSSNHPDNKHKNRYINIVAYDHSRVKLAQLAEKDGKLTDYINANYVDGYNR PKAYIAAQGPLKSTAEDFWRMIWEHNVEVIVMITNLVEKGRRKCDQYWPADGSEEYGNFLVTQ KSVQVLAYYTVRNFTLRNTKIKKGSQKGRPSGRVVTQYHYTQWPDMGVPEYSLPVLTFVRKAA YAKRHAVGPVVVHCSAGVGRTGTYIVLDSMLQQIQHEGTVNIFGFLKHIRSQRNYLVQTEEQYV FIHDTLVEAILSKETEVLDSHIHAYVNALLIPGPAGKTKLEKQFQLLSQSNIQQSDYSAALKQCNR EKNRTSSIIPVERSRVGISSLSGEGTDYINASYIMGYYQSNEFIITQHPLLHTIKDFWRMIWDHNAQ LVVMIPDGQNMAEDEFVYWPNKDEPINCESFKVTLMAEEHKCLSNEEKLIIQDFILEATQDDYV LEVRHFQCPKWPNPDSPISKTFELISVIKEEAANRDGPMIVHDEHGGVTAGTFCALTTLMHQLEK ENSVDVYQVAKMINLMRPGVFADIEQYQFLYKVILSLVSTRQEENPSTSLDSNGAALPDGNIAES LESLV (SEQ ID NO: 8).

Embodiment 63: The chimeric inhibitory receptor of any one of embodiments 1-38, wherein one of the one or more intracellular signaling domains is derived from TLT1.

Embodiment 64: The chimeric inhibitory receptor of embodiment 63, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to MAKRKQGNRLGVCGRFLSSRVSGMNPSSVVHHVSDSGPAAELPLDVPHIRLDSPPSFDNTTYTS LPLDSPSGKPSLPAPSSLPPLPPKVLVCSKPVTYATVIFPGGNKGGGTSCGPAQNPPNNQTPSS (SEQ ID NO: 9).

Embodiment 65: The chimeric inhibitory receptor of embodiment 63, wherein the intracellular signaling domain comprises the amino acid sequence of MAKRKQGNRLGVCGRFLSSRVSGMNPSSVVHHVSDSGPAAELPLDVPHIRLDSPPSFDNTTYTS LPLDSPSGKPSLPAPSSLPPLPPKVLVCSKPVTYATVIFPGGNKGGGTSCGPAQNPPNNQTPSS (SEQ ID NO: 9).

Embodiment 66: The chimeric inhibitory receptor of any one of embodiments 1-38, wherein one of the one or more intracellular signaling domains is derived from SLAMF1.

Embodiment 67: The chimeric inhibitory receptor of embodiment 66, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to QLRRRGKTNHYQTTVEKKSLTIYAQVQKPGPLQKKLDSFPAQDPCTTIYVAATEPVPESVQETNS ITVYASVTLPES (SEQ ID NO: 10).

Embodiment 68: The chimeric inhibitory receptor of embodiment 66, wherein the intracellular signaling domain comprises the amino acid sequence of QLRRRGKTNHYQTTVEKKSLTIYAQVQKPGPLQKKLDSFPAQDPCTTIYVAATEPVPESVQETNS ITVYASVTLPES (SEQ ID NO: 10).

Embodiment 69: The chimeric inhibitory receptor of any one of embodiments 1-38, wherein one of the one or more intracellular signaling domains is derived from SLAMF5.

Embodiment 70: The chimeric inhibitory receptor of embodiment 69, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RLFKRRQGRIFPEGSCLNTFTKNPYAASKKTIYTYIMASRNTQPAESRIYDEILQSKVLPSKEEPVN TVYSEVQFADKMGKASTQDSKPPGTSSYEIVI (SEQ ID NO: 11).

Embodiment 71: The chimeric inhibitory receptor of embodiment 69, wherein the intracellular signaling domain comprises the amino acid sequence of RLFKRRQGRIFPEGSCLNTFTKNPYAASKKTIYTYIMASRNTQPAESRIYDEILQSKVLPSKEEPVN TVYSEVQFADKMGKASTQDSKPPGTSSYEIVI (SEQ ID NO: 11).

Embodiment 72: The chimeric inhibitory receptor of any one of embodiments 1-38, wherein one of the one or more intracellular signaling domains is derived from PCDHGC3.

Embodiment 73: The chimeric inhibitory receptor of embodiment 72, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to FKVYKWKQSRDLYRAPVSSLYRTPGPSLHADAVRGGLMSPHLYHQVYLTTDSRRSDPLLKKPG AASPLASRQNTLRSCDPVFYRQVLGAESAPPGQQAPPNTDWRFSQAQRPGTSGSQNGDDTGTW PNNQFDTEMLQAMILASASEAADGSSTLGGGAGTMGLSARYGPQFTLQHVPDYRQNVYIPGSN ATLTNAAGKRDGKAPAGGNGNKKKSGKKEKK (SEQ ID NO: 95).

Embodiment 74: The chimeric inhibitory receptor of embodiment 73, wherein one of the one or more intracellular signaling domain comprises the amino acid sequence of FKVYKWKQSRDLYRAPVSSLYRTPGPSLHADAVRGGLMSPHLYHQVYLTTDSRRSDPLLKKPG AASPLASRQNTLRSCDPVFYRQVLGAESAPPGQQAPPNTDWRFSQAQRPGTSGSQNGDDTGTW PNNQFDTEMLQAMILASASEAADGSSTLGGGAGTMGLSARYGPQFTLQHVPDYRQNVYIPGSN ATLTNAAGKRDGKAPAGGNGNKKKSGKKEKK (SEQ ID NO: 95).

Embodiment 75: The chimeric inhibitor receptor of any one of embodiments 1-38, wherein one of the one or more intracellular signaling domains is derived from LIFR.

Embodiment 76: The chimeric inhibitory receptor of embodiment 75, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to YRKREWIKETFYPDIPNPENCKALQFQKSVCEGSSALKTLEMNPCTPNNVEVLETRSAFPKIEDT EIISPVAERPEDRSDAEPENHVVVSYCPPIIEEEIPNPAADEAGGTAQVIYIDVQSMYQPQAKPEEE QENDPVGGAGYKPQMHLPINSTVEDIAAEEDLDKTAGYRPQANVNTWNLVSPDSPRSIDSNSEI VSFGSPCSINSRQFLIPPKDEDSPKSNGGGWSFTNFFQNKPND (SEQ ID NO: 96).

Embodiment 77: The chimeric inhibitory receptor of embodiment 76, wherein one of the one or more intracellular signaling domain comprises the amino acid sequence of YRKREWIKETFYPDIPNPENCKALQFQKSVCEGSSALKTLEMNPCTPNNVEVLETRSAFPKIEDT EIISPVAERPEDRSDAEPENHVVVSYCPPIIEEEIPNPAADEAGGTAQVIYIDVQSMYQPQAKPEEE QENDPVGGAGYKPQMHLPINSTVEDIAAEEDLDKTAGYRPQANVNTWNLVSPDSPRSIDSNSEI VSFGSPCSINSRQFLIPPKDEDSPKSNGGGWSFTNFFQNKPND (SEQ ID NO: 96).

Embodiment 78: The chimeric inhibitor receptor of any one of embodiments 1-38, wherein one of the one or more intracellular signaling domains is derived from ERMAP.

Embodiment 79: The chimeric inhibitory receptor of embodiment 78, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to WKQRRAKEKLLYEHVTEVDNLLSDHAKEKGKLHKAVKKLRSELKLKRAAANSGWRRARLHF VAVTLDPDTAHPKLILSEDQRCVRLGDRRQPVPDNPQRFDFVVSILGSEYFTTGCHYWEVYVGD KTKWILGVCSESVSRKGKVTASPANGHWLLRQSRGNEYEALTSPQTSFRLKEPPRCVGIFLDYE AGVISFYNVTNKSHIFTFTHNFSGPLRPFFEPCLHDGGKNTAPLVICSELHKSEESIVPRPEGKGHA NGDVSLKVNSSLLPPKAPELKDIILSLPPDLGPALQELKAPSF (SEQ ID NO: 97).

Embodiment 80: The chimeric inhibitory receptor of embodiment 79, wherein one of the one or more intracellular signaling domain comprises the amino acid sequence of WKQRRAKEKLLYEHVTEVDNLLSDHAKEKGKLHKAVKKLRSELKLKRAAANSGWRRARLHF VAVTLDPDTAHPKLILSEDQRCVRLGDRRQPVPDNPQRFDFVVSILGSEYFTTGCHYWEVYVGD KTKWILGVCSESVSRKGKVTASPANGHWLLRQSRGNEYEALTSPQTSFRLKEPPRCVGIFLDYE AGVISFYNVTNKSHIFTFTHNFSGPLRPFFEPCLHDGGKNTAPLVICSELHKSEESIVPRPEGKGHA NGDVSLKVNSSLLPPKAPELKDIILSLPPDLGPALQELKAPSF (SEQ ID NO: 97).

Embodiment 81: The chimeric inhibitory receptor of any one of embodiments 1-38, wherein one of the one or more intracellular signaling domains is derived from IL1RAPL2.

Embodiment 82: The chimeric inhibitory receptor of embodiment 81, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to KCYNIELMLFYRQHFGADETNDDNKEYDAYLSYTKVDQDTLDCDNPEEEQFALEVLPDVLEKH YGYKLFIPERDLIPSGTYMEDLTRYVEQSRRLIIVLTPDYILRRGWSIFELESRLHNMLVSGEIKVI LIECTELKGKVNCQEVESLKRSIKLLSLIKWKGSKSSKLNSKFWKHLVYEMPIKKKEMLPRCHVL DSAEQGLFGELQPIPSIAMTSTSATLVSSQADLPEFHPSDSMQIRHCCRGYKHEIPATTLPVPSLGN HHTYCNLPLTLLNGQLPLNNTLKDTQEFHRNSSLLPLSSKELSFTSDIW (SEQ ID NO: 98).

Embodiment 83: The chimeric inhibitory receptor of embodiment 82, wherein one of the one or more intracellular signaling domain comprises the amino acid sequence of KCYNIELMLFYRQHFGADETNDDNKEYDAYLSYTKVDQDTLDCDNPEEEQFALEVLPDVLEKH YGYKLFIPERDLIPSGTYMEDLTRYVEQSRRLIIVLTPDYILRRGWSIFELESRLHNMLVSGEIKVI LIECTELKGKVNCQEVESLKRSIKLLSLIKWKGSKSSKLNSKFWKHLVYEMPIKKKEMLPRCHVL DSAEQGLFGELQPIPSIAMTSTSATLVSSQADLPEFHPSDSMQIRHCCRGYKHEIPATTLPVPSLGN HHTYCNLPLTLLNGQLPLNNTLKDTQEFHRNSSLLPLSSKELSFTSDIW (SEQ ID NO: 98).

Embodiment 84: The chimeric inhibitory receptor of any one of embodiments 1-38, wherein one of the one or more intracellular signaling domains is derived from CDH5.

Embodiment 85: The chimeric inhibitory receptor of embodiment 84, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RRRLRKQARAHGKSVPEIHEQLVTYDEEGGGEMDTTSYDVSVLNSVRRGGAKPPRPALDARPS LYAQVQKPPRHAPGAHGGPGEMAAMIEVKKDEADHDGDGPPYDTLHIYGYEGSESIAESLSSLG TDSSDSDVDYDFLNDWGPRFKMLAELYGSDPREELLY (SEQ ID NO: 99).

Embodiment 86: The chimeric inhibitory receptor of embodiment 85, wherein one of the one or more intracellular signaling domain comprises the amino acid sequence of RRRLRKQARAHGKSVPEIHEQLVTYDEEGGGEMDTTSYDVSVLNSVRRGGAKPPRPALDARPS LYAQVQKPPRHAPGAHGGPGEMAAMIEVKKDEADHDGDGPPYDTLHIYGYEGSESIAESLSSLG TDSSDSDVDYDFLNDWGPRFKMLAELYGSDPREELLY (SEQ ID NO: 99).

Embodiment 87: The chimeric inhibitory receptor of any one of embodiments 1-38, wherein one of the one or more intracellular signaling domains is derived from MPZL1.

Embodiment 88: The chimeric inhibitory receptor of embodiment 87, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SMILAVLYRRKNSKRDYTGCSTSESLSPVKQAPRKSPSDTEGLVKSLPSGSHQGPVIYAQLDHSG GHHSDKINKSESVVYADIRKN (SEQ ID NO: 100).

Embodiment 89: The chimeric inhibitory receptor of embodiment 88, wherein one of the one or more intracellular signaling domain comprises the amino acid sequence of SMILAVLYRRKNSKRDYTGCSTSESLSPVKQAPRKSPSDTEGLVKSLPSGSHQGPVIYAQLDHSG GHHSDKINKSESVVYADIRKN (SEQ ID NO: 100).

Embodiment 90: The chimeric inhibitory receptor of any one of embodiments 1-38, wherein one of the one or more intracellular signaling domains is derived from MPZ.

Embodiment 91: The chimeric inhibitory receptor of embodiment 90, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RYCWLRRQAALQRRLSAMEKGKLHKPGKDASKRGRQTPVLYAMLDHSRSTKAVSEKKAKGL GESRKDKK (SEQ ID NO: 101).

Embodiment 92: The chimeric inhibitory receptor of embodiment 91, wherein one of the one or more intracellular signaling domain comprises the amino acid sequence of RYCWLRRQAALQRRLSAMEKGKLHKPGKDASKRGRQTPVLYAMLDHSRSTKAVSEKKAKGL GESRKDKK (SEQ ID NO: 101).

Embodiment 93: The chimeric inhibitory receptor of any one of embodiments 1-38, wherein one of the one or more intracellular signaling domains is derived from FCGR2B.

Embodiment 94: The chimeric inhibitory receptor of embodiment 93, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VVALIYCRKKRISALPGYPECREMGETLPEKPANPTNPDEADKVGAENTITYSLLMHPDALEEPD DQNRI (SEQ ID NO: 102).

Embodiment 95: The chimeric inhibitory receptor of embodiment 94, wherein one of the one or more intracellular signaling domain comprises the amino acid sequence of VVALIYCRKKRISALPGYPECREMGETLPEKPANPTNPDEADKVGAENTITYSLLMHPDALEEPD DQNRI (SEQ ID NO: 102).

Embodiment 96: The chimeric inhibitory receptor of any one of embodiments 1-38, wherein one of the one or more intracellular signaling domains is derived from SIGLEC-6.

Embodiment 97: The chimeric inhibitory receptor of embodiment 96, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RVKTRRKKAAQPVQNTDDVNPVMVSGSRGHQHQFQTGIVSDHPAEAGPISEDEQELHYAVLHF HKVQPQEPKVTDTEYSEIKIHK (SEQ ID NO: 103).

Embodiment 98: The chimeric inhibitory receptor of embodiment 97, wherein one of the one or more intracellular signaling domain comprises the amino acid sequence of RVKTRRKKAAQPVQNTDDVNPVMVSGSRGHQHQFQTGIVSDHPAEAGPISEDEQELHYAVLHF HKVQPQEPKVTDTEYSEIKIHK (SEQ ID NO: 103).

Embodiment 99: The chimeric inhibitory receptor of any one of embodiments 1-38, wherein one of the one or more intracellular signaling domains is derived from MPIG6B.

Embodiment 100: The chimeric inhibitory receptor of embodiment 99, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to WLHRRLPPQPIRPLPRFAPLVKTEPQRPVKEEEPKIPGDLDQEPSLLYADLDHLALSRPRRLSTAD PADASTIYAVVV (SEQ ID NO: 104).

Embodiment 101: The chimeric inhibitory receptor of embodiment 100, wherein one of the one or more intracellular signaling domain comprises the amino acid sequence of WLHRRLPPQPIRPLPRFAPLVKTEPQRPVKEEEPKIPGDLDQEPSLLYADLDHLALSRPRRLSTAD PADASTIYAVVV (SEQ ID NO: 104).

Embodiment 102: The chimeric inhibitory receptor of any one of embodiments 1-38, wherein one of the one or more intracellular signaling domains is derived from VSIG4.

Embodiment 103: The chimeric inhibitory receptor of embodiment 102, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to MLCRKTSQQEHVYEAARAHAREANDSGETMRVAIFASGCSSDEPTSQNLGNNYSDEPCIGQEY QIIAQINGNYARLLDTVPLDYEFLATEGKSVC (SEQ ID NO: 105).

Embodiment 104: The chimeric inhibitory receptor of embodiment 103, wherein one of the one or more intracellular signaling domain comprises the amino acid sequence of MLCRKTSQQEHVYEAARAHAREANDSGETMRVAIFASGCSSDEPTSQNLGNNYSDEPCIGQEY QIIAQINGNYARLLDTVPLDYEFLATEGKSVC (SEQ ID NO: 105).

Embodiment 105: The chimeric inhibitory receptor of any one of embodiments 1-38, wherein one of the one or more intracellular signaling domains is derived from SIGLEC-12.

Embodiment 106: The chimeric inhibitory receptor of embodiment 105, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RSCRKKSARPAVGVGDTGMEDANAVRGSASQGPLIESPADDSPPHHAPPALATPSPEEGEIQYAS LSFHKARPQYPQEQEAIGYEYSEINIPK (SEQ ID NO: 106).

Embodiment 107: The chimeric inhibitory receptor of embodiment 106, wherein one of the one or more intracellular signaling domain comprises the amino acid sequence of RSCRKKSARPAVGVGDTGMEDANAVRGSASQGPLIESPADDSPPHHAPPALATPSPEEGEIQYAS LSFHKARPQYPQEQEAIGYEYSEINIPK (SEQ ID NO: 106).

Embodiment 108: The chimeric inhibitory receptor of any one of embodiments 1-38, wherein one of the one or more intracellular signaling domains is derived from LIR8.

Embodiment 109: The chimeric inhibitory receptor of embodiment 108, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RHRHQSKHRTSAHFYRPAGAAGPEPKDQGLQKRASPVADIQEEILNAAVKDTQPKDGVEMDAP AAASEAPQDVTYAQLHSLTLRREATEPPPSQEREPPAEPSIYAPLAIH (SEQ ID NO: 107).

Embodiment 110: The chimeric inhibitory receptor of embodiment 109, wherein one of the one or more intracellular signaling domain comprises the amino acid sequence of RHRHQSKHRTSAHFYRPAGAAGPEPKDQGLQKRASPVADIQEEILNAAVKDTQPKDGVEMDAP AAASEAPQDVTYAQLHSLTLRREATEPPPSQEREPPAEPSIYAPLAIH (SEQ ID NO: 107).

Embodiment 111: The chimeric inhibitory receptor of any one of embodiments 1-38, wherein one of the one or more intracellular signaling domains is derived from IRTA1.

Embodiment 112: The chimeric inhibitory receptor of embodiment 111, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to HCWRRRKSGVGFLGDETRLPPAPGPGESSHSICPAQVELQSLYVDVHPKKGDLVYSEIQTTQLGE EEEANTSRTLLEDKDVSVVYSEVKTQHPDNSAGKISSKDEES (SEQ ID NO: 108).

Embodiment 113: The chimeric inhibitory receptor of embodiment 112, wherein one of the one or more intracellular signaling domain comprises the amino acid sequence of HCWRRRKSGVGFLGDETRLPPAPGPGESSHSICPAQVELQSLYVDVHPKKGDLVYSEIQTTQLGE EEEANTSRTLLEDKDVSVVYSEVKTQHPDNSAGKISSKDEES (SEQ ID NO: 108).

Embodiment 114: The chimeric inhibitory receptor of any one of embodiments 1-38, wherein one of the one or more intracellular signaling domains is derived from KIR2DL4.

Embodiment 115: The chimeric inhibitory receptor of embodiment 114, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RWCSKKKDAAVMNQEPAGHRTVNREDSDEQDPQEVTYAQLDHCIFTQRKITGPSQRSKRPSTD TSVCIELPNAEPRALSPAHEHHSQALMGSSRETTALSQTQLASSNVPAAGI (SEQ ID NO: 109).

Embodiment 116: The chimeric inhibitory receptor of embodiment 115, wherein one of the one or more intracellular signaling domain comprises the amino acid sequence of RWCSKKKDAAVMNQEPAGHRTVNREDSDEQDPQEVTYAQLDHCIFTQRKITGPSQRSKRPSTD TSVCIELPNAEPRALSPAHEHHSQALMGSSRETTALSQTQLASSNVPAAGI. (SEQ ID NO: 109).

Embodiment 117: The chimeric inhibitory receptor of any one of embodiments 1-38, wherein one of the one or more intracellular signaling domains is derived from KIR2DL5.

Embodiment 118: The chimeric inhibitory receptor of embodiment 117, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LHCCCSNKKNAAVMDQEPAGDRTVNREDSDDQDPQEVTYAQLDHCVFTQTKITSPSQRPKTPP TDTTMYMELPNAKPRSLSPAHKHHSQALRGSSRETTALSQNRVASSHVPAAGI (SEQ ID NO: 110).

Embodiment 119: The chimeric inhibitory receptor of embodiment 118, wherein one of the one or more intracellular signaling domain comprises the amino acid sequence of LHCCCSNKKNAAVMDQEPAGDRTVNREDSDDQDPQEVTYAQLDHCVFTQTKITSPSQRPKTPP TDTTMYMELPNAKPRSLSPAHKHHSQALRGSSRETTALSQNRVASSHVPAAGI (SEQ ID NO: 110).

Embodiment 120: The chimeric inhibitory receptor of any one of embodiments 1-38, wherein one of the one or more intracellular signaling domains is derived from SIGLEC-7.

Embodiment 121: The chimeric inhibitory receptor of embodiment 120, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RSCRKKSARPAADVGDIGMKDANTIRGSASQGNLTESWADDNPRHHGLAAHSSGEEREIQYAP LSFHKGEPQDLSGQEATNNEYSEIKIPK (SEQ ID NO: 111).

Embodiment 122: The chimeric inhibitory receptor of embodiment 121, wherein one of the one or more intracellular signaling domain comprises the amino acid sequence of RSCRKKSARPAADVGDIGMKDANTIRGSASQGNLTESWADDNPRHHGLAAHSSGEEREIQYAP LSFHKGEPQDLSGQEATNNEYSEIKIPK (SEQ ID NO: 111).

Embodiment 123: The chimeric inhibitory receptor of any one of embodiments 1-38, wherein one of the one or more intracellular signaling domains is derived from FCRH3.

Embodiment 124: The chimeric inhibitory receptor of embodiment 123, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to HYARARRKPGGLSATGTSSHSPSECQEPSSSRPSRIDPQEPTHSKPLAPMELEPMYSNVNPGDSNP IYSQIWSIQHTKENSANCPMMHQEHEELTVLYSELKKTHPDDSAGEASSRGRAHEEDDEENYEN VPRVLLASDH (SEQ ID NO: 112).

Embodiment 125: The chimeric inhibitory receptor of embodiment 124, wherein one of the one or more intracellular signaling domain comprises the amino acid sequence of HYARARRKPGGLSATGTSSHSPSECQEPSSSRPSRIDPQEPTHSKPLAPMELEPMYSNVNPGDSNP IYSQIWSIQHTKENSANCPMMHQEHEELTVLYSELKKTHPDDSAGEASSRGRAHEEDDEENYEN VPRVLLASDH (SEQ ID NO: 112).

Embodiment 126: The chimeric inhibitory receptor of any one of embodiments 1-38, wherein one of the one or more intracellular signaling domains is derived from PCDHGC5.

Embodiment 127: The chimeric inhibitory receptor of embodiment 126, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to KCLQGNADGDGGGGQCCRRQDSPSPDFYKQSSPNLQVSSDGTLKYMEVTLRPTDSQSHCYRTC FSPASDGSDFTFLRPLSVQQPTALALEPDAIRSRSNTLRERSQQAPPNTDWRFSQAQRPGTSGSQN GDDTGTWPNNQFDTEMLQAMILASASEAADGSSTLGGGAGTMGLSARYGPQFTLQHVPDYRQ NVYIPGSNATLTNAAGKRDGKAPAGGNGNKKKSGKKEKK (SEQ ID NO: 113).

Embodiment 128: The chimeric inhibitory receptor of embodiment 127, wherein one of the one or more intracellular signaling domain comprises the amino acid sequence of KCLQGNADGDGGGGQCCRRQDSPSPDFYKQSSPNLQVSSDGTLKYMEVTLRPTDSQSHCYRTC FSPASDGSDFTFLRPLSVQQPTALALEPDAIRSRSNTLRERSQQAPPNTDWRFSQAQRPGTSGSQN GDDTGTWPNNQFDTEMLQAMILASASEAADGSSTLGGGAGTMGLSARYGPQFTLQHVPDYRQ NVYIPGSNATLTNAAGKRDGKAPAGGNGNKKKSGKKEKK (SEQ ID NO: 113).

Embodiment 129: The chimeric inhibitory receptor of any one of embodiments 1-38, wherein one of the one or more intracellular signaling domains is derived from CDH11.

Embodiment 130: The chimeric inhibitory receptor of embodiment 129, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RRQKKEPLIVFEEEDVRENIITYDDEGGGEEDTEAFDIATLONPDGINGFIPRKDIKPEYQYMPRP GLRPAPNSVDVDDFINTRIQEADNDPTAPPYDSIQIYGYEGRGSVAGSLSSLESATTDSDLDYDYL QNWGPRFKKLADLYGSKDTFDDDS (SEQ ID NO: 114).

Embodiment 131: The chimeric inhibitory receptor of embodiment 130, wherein one of the one or more intracellular signaling domain comprises the amino acid sequence of RRQKKEPLIVFEEEDVRENIITYDDEGGGEEDTEAFDIATLONPDGINGFIPRKDIKPEYQYMPRP GLRPAPNSVDVDDFINTRIQEADNDPTAPPYDSIQIYGYEGRGSVAGSLSSLESATTDSDLDYDYL QNWGPRFKKLADLYGSKDTFDDDS (SEQ ID NO: 114).

Embodiment 132: The chimeric inhibitory receptor of any one of embodiments 1-38, wherein one of the one or more intracellular signaling domains is derived from IMPG2.

Embodiment 133: The chimeric inhibitory receptor of embodiment 132, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to YFFIRTLQAHHDRSERESPFSGSSRQPDSLSSIENAVKYNPVYESHRAGCEKYEGPYPQHPFYSSA SGDVIGGLSREEIRQMYESSELSREEIQERMRVLELYANDPEFAAFVREQQVEEV (SEQ ID NO: 115).

Embodiment 134: The chimeric inhibitory receptor of embodiment 133, wherein one of the one or more intracellular signaling domain comprises the amino acid sequence of YFFIRTLQAHHDRSERESPFSGSSRQPDSLSSIENAVKYNPVYESHRAGCEKYEGPYPQHPFYSSA SGDVIGGLSREEIRQMYESSELSREEIQERMRVLELYANDPEFAAFVREQQVEEV (SEQ ID NO: 115).

Embodiment 135: The chimeric inhibitory receptor of any one of embodiments 1-38, wherein one of the one or more intracellular signaling domains is derived from DSCAM.

Embodiment 136: The chimeric inhibitory receptor of embodiment 135, wherein one of the one or more intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RRRRREQRLKRLRDAKSLAEMLMSKNTRTSDTLSKQQQTLRMHIDIPRAQLLIEERDTMETIDD RSTVLLTDADEGEAAKQKSLTVTHTVHYQSVSQATGPLVDVSDARPGTNPTTRRNAKAGPTAR NRYASQWTLNRPHPTISAHTLTTDWRLPTPRAAGSVDKESDSYSVSPSQDTDRARSSMVSTESA SSTYEELARAYEHAKMEEQLRHAKFTITECFISDTSSEQLTAGTNEYTDSLTSSTPSESGICRFTAS PPKPQDGGRVMNMAVPKAHRPGDLIHLPPYLRMDFLLNRGGPGTSRDLSLGQACLEPQKSRTL KRPTVLEPIPMEAASSASSTREGQSWQPGAVATLPQREGAELGQAAKMSSSQESLLDSRGHLKG NNPYAKSYTLV (SEQ ID NO: 116).

Embodiment 137: The chimeric inhibitory receptor of embodiment 136, wherein one of the one or more intracellular signaling domain comprises the amino acid sequence of RRRRREQRLKRLRDAKSLAEMLMSKNTRTSDTLSKQQQTLRMHIDIPRAQLLIEERDTMETIDD RSTVLLTDADEGEAAKQKSLTVTHTVHYQSVSQATGPLVDVSDARPGTNPTTRRNAKAGPTAR NRYASQWTLNRPHPTISAHTLTTDWRLPTPRAAGSVDKESDSYSVSPSQDTDRARSSMVSTESA SSTYEELARAYEHAKMEEQLRHAKFTITECFISDTSSEQLTAGTNEYTDSLTSSTPSESGICRFTAS PPKPQDGGRVMNMAVPKAHRPGDLIHLPPYLRMDFLLNRGGPGTSRDLSLGQACLEPQKSRTL KRPTVLEPIPMEAASSASSTREGQSWQPGAVATLPQREGAELGQAAKMSSSQESLLDSRGHLKG NNPYAKSYTLV (SEQ ID NO: 116).

Embodiment 138: A chimeric inhibitory receptor comprising:
an extracellular protein binding domain;
a transmembrane domain, wherein the transmembrane domain is operably linked to the extracellular protein binding domain; and
one or more intracellular signaling domains, wherein the one or more intracellular signaling domains are operably linked to the transmembrane domain,
   wherein at least one of the one or more intracellular signaling domains comprises:
i) at least one immunoreceptor tyrosine-based switch motif (ITSM); or
ii) at least one ITIM and at least one phosphatase, and
   wherein at least one of the one or more intracellular signaling domains is capable of preventing, attenuating, or inhibiting activation of a tumor-targeting chimeric receptor expressed on an immunomodulatory cell.

Embodiment 139: The chimeric inhibitory receptor of embodiment 138, comprises at least one ITIM and at least one phosphatase.

Embodiment 140: The chimeric inhibitory receptor of embodiment 139, wherein the phosphatase is a protein tyrosine phosphatase (PTP).

Embodiment 141: The chimeric inhibitory receptor of embodiment 139 or 140, wherein the one or more intracellular signaling domains are each derived from a protein selected from the group consisting of: PTPRO and PTPRZ1.

Embodiment 142: The chimeric inhibitory receptor of any one of embodiments 138-141, wherein one of the one or more intracellular signaling domains is derived from PTPRO.

Embodiment 143: The chimeric inhibitory receptor of embodiment 142, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LRKKHLQMARECGAGTFVNFASLERDGKLPYNWRRSIFAFLTLLPSCLWTDYLLAFYINPWSKN GLKKRKLTNPVQLDDFDAYIKDMAKDSDYKFSLQFEELKLIGLDIPHFAADLPLNRCKNRYTNIL PYDFSRVRLVSMNEEEGADYINANYIPGYNSPQEYIATQGPLPETRNDFWKMVLQQKSQIIVML TQCNEKRRVKCDHYWPFTEEPIAYGDITVEMISEEEQDDWACRHFRINYADEMQDVMHFNYTA WPDHGVPTANAAESILQFVHMVRQQATKSKGPMIIHCSAGVGRTGTFIALDRLLQHIRDHEFVDI LGLVSEMRSYRMSMVQTEEQYIFIHQCVQLMWMKKKQQFCISDVIYENVSKS (SEQ ID NO: 7).

Embodiment 144: The chimeric inhibitory receptor of embodiment 143, wherein the intracellular signaling domain comprises the amino acid sequence of LRKKHLQMARECGAGTFVNFASLERDGKLPYNWRRSIFAFLTLLPSCLWTDYLLAFYINPWSKN GLKKRKLTNPVQLDDFDAYIKDMAKDSDYKFSLQFEELKLIGLDIPHFAADLPLNRCKNRYTNIL PYDFSRVRLVSMNEEEGADYINANYIPGYNSPQEYIATQGPLPETRNDFWKMVLQQKSQIIVML TQCNEKRRVKCDHYWPFTEEPIAYGDITVEMISEEEQDDWACRHFRINYADEMQDVMHFNYTA WPDHGVPTANAAESILQFVHMVRQQATKSKGPMIIHCSAGVGRTGTFIALDRLLQHIRDHEFVDI LGLVSEMRSYRMSMVQTEEQYIFIHQCVQLMWMKKKQQFCISDVIYENVSKS (SEQ ID NO: 7).

Embodiment 145: The chimeric inhibitory receptor of any one of embodiments 138-141, wherein one of the one or more intracellular signaling domains is derived from PTPRZ1.

Embodiment 146: The chimeric inhibitory receptor of embodiment 145, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RKCFQTAHFYLEDSTSPRVISTPPTPIFPISDDVGAIPIKHFPKHVADLHASSGFTEEFETLKEFYQE VQSCTVDLGITADSSNHPDNKHKNRYINIVAYDHSRVKLAQLAEKDGKLTDYINANYVDGYNR PKAYIAAQGPLKSTAEDFWRMIWEHNVEVIVMITNLVEKGRRKCDQYWPADGSEEYGNFLVTQ KSVQVLAYYTVRNFTLRNTKIKKGSQKGRPSGRVVTQYHYTQWPDMGVPEYSLPVLTFVRKAA YAKRHAVGPVVVHCSAGVGRTGTYIVLDSMLQQIQHEGTVNIFGFLKHIRSQRNYLVQTEEQYV FIHDTLVEAILSKETEVLDSHIHAYVNALLIPGPAGKTKLEKQFQLLSQSNIQQSDYSAALKQCNR EKNRTSSIIPVERSRVGISSLSGEGTDYINASYIMGYYQSNEFIITQHPLLHTIKDFWRMIWDHNAQ LVVMIPDGQNMAEDEFVYWPNKDEPINCESFKVTLMAEEHKCLSNEEKLIIQDFILEATQDDYV LEVRHFQCPKWPNPDSPISKTFELISVIKEEAANRDGPMIVHDEHGGVTAGTFCALTTLMHQLEK ENSVDVYQVAKMINLMRPGVFADIEQYQFLYKVILSLVSTRQEENPSTSLDSNGAALPDGNIAES LESLV (SEQ ID NO: 8).

Embodiment 147: The chimeric inhibitory receptor of embodiment 146, wherein the intracellular signaling domain comprises the amino acid sequence of RKCFQTAHFYLEDSTSPRVISTPPTPIFPISDDVGAIPIKHFPKHVADLHASSGFTEEFETLKEFYQE VQSCTVDLGITADSSNHPDNKHKNRYINIVAYDHSRVKLAQLAEKDGKLTDYINANYVDGYNR PKAYIAAQGPLKSTAEDFWRMIWEHNVEVIVMITNLVEKGRRKCDQYWPADGSEEYGNFLVTQ KSVQVLAYYTVRNFTLRNTKIKKGSQKGRPSGRVVTQYHYTQWPDMGVPEYSLPVLTFVRKAA YAKRHAVGPVVVHCSAGVGRTGTYIVLDSMLQQIQHEGTVNIFGFLKHIRSQRNYLVQTEEQYV FIHDTLVEAILSKETEVLDSHIHAYVNALLIPGPAGKTKLEKQFQLLSQSNIQQSDYSAALKQCNR EKNRTSSIIPVERSRVGISSLSGEGTDYINASYIMGYYQSNEFIITQHPLLHTIKDFWRMIWDHNAQ LVVMIPDGQNMAEDEFVYWPNKDEPINCESFKVTLMAEEHKCLSNEEKLIIQDFILEATQDDYV LEVRHFQCPKWPNPDSPISKTFELISVIKEEAANRDGPMIVHDEHGGVTAGTFCALTTLMHQLEK ENSVDVYQVAKMINLMRPGVFADIEQYQFLYKVILSLVSTRQEENPSTSLDSNGAALPDGNIAES LESLV (SEQ ID NO: 8).

Embodiment 148: The chimeric inhibitory receptor of embodiment 138, wherein the chimeric inhibitory receptor comprises at least one ITSM.

Embodiment 149: The chimeric inhibitory receptor of embodiment 138 or 148, wherein the one or more intracellular signaling domains are each derived from a protein selected from the group consisting of: SLAMF1 and SLAMF5.

Embodiment 150: The chimeric inhibitory receptor of any one of embodiments 138, 148, or 149, wherein one of the one or more intracellular signaling domains is derived from SLAMF1.

Embodiment 151: The chimeric inhibitory receptor of embodiment 150, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to QLRRRGKTNHYQTTVEKKSLTIYAQVQKPGPLQKKLDSFPAQDPCTTIYVAATEPVPESVQETNS ITVYASVTLPES (SEQ ID NO: 10).

Embodiment 152: The chimeric inhibitory receptor of embodiment 151, wherein the intracellular signaling domain comprises the amino acid sequence of QLRRRGKTNHYQTTVEKKSLTIYAQVQKPGPLQKKLDSFPAQDPCTTIYVAATEPVPESVQETNS ITVYASVTLPES (SEQ ID NO: 10).

Embodiment 153: The chimeric inhibitory receptor of any one of embodiments 138, 148, or 149, wherein one of the one or more intracellular signaling domains is derived from SLAMF5.

Embodiment 154: The chimeric inhibitory receptor of embodiment 153, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RLFKRRQGRIFPEGSCLNTFTKNPYAASKKTIYTYIMASRNTQPAESRIYDEILQSKVLPSKEEPVN TVYSEVQFADKMGKASTQDSKPPGTSSYEIVI (SEQ ID NO: 11).

Embodiment 155: The chimeric inhibitory receptor of embodiment 154, wherein the intracellular signaling domain comprises the amino acid sequence of RLFKRRQGRIFPEGSCLNTFTKNPYAASKKTIYTYIMASRNTQPAESRIYDEILQSKVLPSKEEPVN TVYSEVQFADKMGKASTQDSKPPGTSSYEIVI (SEQ ID NO: 11).

Embodiment 156: The chimeric inhibitory receptor of any one of embodiments 1-155, wherein the transmembrane domain is derived from a protein selected from the group consisting of: CD8, CD28, CD3, CD4, 4-IBB, OX40, ICOS, 2B4, CD25, CD7, LAX, LAT, LIR1, PCAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM.

Embodiment 157: The chimeric inhibitory receptor of any one of embodiments 1-156, wherein the chimeric inhibitory receptor comprises a transmembrane domain derived from PECAM-1.

Embodiment 158: The chimeric inhibitory receptor of embodiment 157, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to GLIAVVIIGVIIALLIIAA (SEQ ID NO: 24).

Embodiment 159: The chimeric inhibitory receptor of embodiment 157, wherein the transmembrane domain comprises the amino acid sequence of GLIAVVIIGVIIALLIIAA (SEQ ID NO: 24).

Embodiment 160: The chimeric inhibitory receptor of any one of embodiments 1-156, wherein the chimeric inhibitory receptor comprises a transmembrane domain derived from LIR1.

Embodiment 161: The chimeric inhibitory receptor of embodiment 160, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VIGILVAVILLLLLLLLLFLI (SEQ ID NO: 25).

Embodiment 162: The chimeric inhibitory receptor of embodiment 160, wherein the transmembrane domain comprises the amino acid sequence of VIGILVAVILLLLLLLLLFLI (SEQ ID NO: 25).

Embodiment 163: The chimeric inhibitory receptor of any one of embodiments 1-156, wherein the chimeric inhibitory receptor comprises a transmembrane domain derived from IRTA2.

Embodiment 164: The chimeric inhibitory receptor of embodiment 163, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VAGGLLSIAGLAAGALLLYCW (SEQ ID NO: 26).

Embodiment 165: The chimeric inhibitory receptor of embodiment 163, wherein the transmembrane domain comprises the amino acid sequence of VAGGLLSIAGLAAGALLLYCW (SEQ ID NO: 26).

Embodiment 166: The chimeric inhibitory receptor of any one of embodiments 1-156, wherein the chimeric inhibitory receptor comprises a transmembrane domain derived from IRTA4.

Embodiment 167: The chimeric inhibitory receptor of embodiment 166, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LWGLFGVLGFTGVALLLYALF (SEQ ID NO: 27).

Embodiment 168: The chimeric inhibitory receptor of embodiment 166, wherein the transmembrane domain comprises the amino acid sequence of LWGLFGVLGFTGVALLLYALF (SEQ ID NO: 27).

Embodiment 169: The chimeric inhibitory receptor of any one of embodiments 1-156, wherein the chimeric inhibitory receptor comprises a transmembrane domain derived from NKIR.

Embodiment 170: The chimeric inhibitory receptor of embodiment 169, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VLLPLIFTILLLLLVAASLLA (SEQ ID NO: 28).

Embodiment 171: The chimeric inhibitory receptor of embodiment 169, wherein the transmembrane domain comprises the amino acid sequence of VLLPLIFTILLLLLVAASLLA (SEQ ID NO: 28).

Embodiment 172: The chimeric inhibitory receptor of any one of embodiments 1-156, wherein the chimeric inhibitory receptor comprises a transmembrane domain derived from IL1RAP.

Embodiment 173: The chimeric inhibitory receptor of embodiment 172, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VLLVVILIVVYHVYWLEMVLF (SEQ ID NO: 29).

Embodiment 174: The chimeric inhibitory receptor of embodiment 172, wherein the transmembrane domain comprises the amino acid sequence of VLLVVILIVVYHVYWLEMVLF (SEQ ID NO: 29).

Embodiment 175: The chimeric inhibitory receptor of any one of embodiments 1-156, wherein the chimeric inhibitory receptor comprises a transmembrane domain derived from PTPRO.

Embodiment 176: The chimeric inhibitory receptor of embodiment 175, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to ISVLAILSTLLIGLLLVTLII (SEQ ID NO: 30).

Embodiment 177: The chimeric inhibitory receptor of embodiment 175, wherein the transmembrane domain comprises the amino acid sequence of ISVLAILSTLLIGLLLVTLII (SEQ ID NO: 30).

Embodiment 178: The chimeric inhibitory receptor of any one of embodiments 1-156, wherein the chimeric inhibitory receptor comprises a transmembrane domain derived from PTPRZ1.

Embodiment 179: The chimeric inhibitory receptor of embodiment 178, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AVIPLVIVSALTFICLVVLVGILIYW (SEQ ID NO: 31).

Embodiment 180: The chimeric inhibitory receptor of embodiment 178, wherein the transmembrane domain comprises the amino acid sequence of AVIPLVIVSALTFICLVVLVGILIYW (SEQ ID NO: 31).

Embodiment 181: The chimeric inhibitory receptor of any one of embodiments 1-156, wherein the chimeric inhibitory receptor comprises a transmembrane domain derived from TLT1.

Embodiment 182: The chimeric inhibitory receptor of embodiment 181, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LIWGAVLLVGLLVAAVVLFAV (SEQ ID NO: 32).

Embodiment 183: The chimeric inhibitory receptor of embodiment 181, wherein the transmembrane domain comprises the amino acid sequence of LIWGAVLLVGLLVAAVVLFAV (SEQ ID NO: 32).

Embodiment 184: The chimeric inhibitory receptor of any one of embodiments 1-156, wherein the chimeric inhibitory receptor comprises a transmembrane domain derived from SLAMF1.

Embodiment 185: The chimeric inhibitory receptor of embodiment 66, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to WAVYAGLLGGVIMILIMVVIL (SEQ ID NO: 33).

Embodiment 186: The chimeric inhibitory receptor of embodiment 66, wherein the transmembrane domain comprises the amino acid sequence of WAVYAGLLGGVIMILIMVVIL (SEQ ID NO: 33).

Embodiment 187: The chimeric inhibitory receptor of any one of embodiments 1-156, wherein the chimeric inhibitory receptor comprises a transmembrane domain derived from SLAMF5.

Embodiment 188: The chimeric inhibitory receptor of embodiment 187, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LLSVLAMFFLLVLILSSVFLF (SEQ ID NO: 34).

Embodiment 189: The chimeric inhibitory receptor of embodiment 187, wherein the transmembrane domain comprises the amino acid sequence of LLSVLAMFFLLVLILSSVFLF (SEQ ID NO: 34).

Embodiment 190: The chimeric inhibitory receptor of any one of embodiments 1-189, wherein the chimeric inhibitory receptor comprises a transmembrane domain is derived from PCDHGC3.

Embodiment 191: The chimeric inhibitory receptor of embodiment 190, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LLLSLILVSVGFVVTVFGVII (SEQ ID NO: 139).

Embodiment 192: The chimeric inhibitory receptor of embodiment 191, wherein the transmembrane domain comprises the amino acid sequence of LLLSLILVSVGFVVTVFGVII (SEQ ID NO: 139).

Embodiment 193: The chimeric inhibitory receptor of any one of embodiments 1-189, wherein the chimeric inhibitory receptor comprises a transmembrane domain is derived from LIFR.

Embodiment 194: The chimeric inhibitory receptor of embodiment 193, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VGLIIAILIPVAVAVIVGVVTSILC (SEQ ID NO: 140).

Embodiment 195: The chimeric inhibitory receptor of embodiment 194, wherein the transmembrane domain comprises the amino acid sequence of VGLIIAILIPVAVAVIVGVVTSILC (SEQ ID NO: 140).

Embodiment 196: The chimeric inhibitory receptor of any one of embodiments 1-189, wherein the chimeric inhibitory receptor comprises a transmembrane domain is derived from ERMAP.

Embodiment 197: The chimeric inhibitory receptor of embodiment 196, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VALAVILPVLVLLIMVCLCLI (SEQ ID NO: 141).

Embodiment 198: The chimeric inhibitory receptor of embodiment 197, wherein the transmembrane domain comprises the amino acid sequence of VALAVILPVLVLLIMVCLCLI (SEQ ID NO: 141).

Embodiment 199: The chimeric inhibitory receptor of any one of embodiments 1-189, wherein the chimeric inhibitory receptor comprises a transmembrane domain is derived from IL1RAPL2.

Embodiment 200: The chimeric inhibitory receptor of embodiment 199, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to IELAGGLGAIFLLLVLLVVIY (SEQ ID NO: 142).

Embodiment 201: The chimeric inhibitory receptor of embodiment 200, wherein the transmembrane domain comprises the amino acid sequence of IELAGGLGAIFLLLVLLVVIY (SEQ ID NO: 142).

Embodiment 202: The chimeric inhibitory receptor of any one of embodiments 1-189, wherein the chimeric inhibitory receptor comprises a transmembrane domain is derived from CDH5.

Embodiment 203: The chimeric inhibitory receptor of embodiment 202, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AVVAILLCILTITVITLLIFL (SEQ ID NO: 143).

Embodiment 204: The chimeric inhibitory receptor of embodiment 203, wherein the transmembrane domain comprises the amino acid sequence of AVVAILLCILTITVITLLIFL (SEQ ID NO: 143).

Embodiment 205: The chimeric inhibitory receptor of any one of embodiments 1-189, wherein the chimeric inhibitory receptor comprises a transmembrane domain is derived from MPZL1.

Embodiment 206: The chimeric inhibitory receptor of embodiment 205, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to FPVWVVVGIVTAVVLGLTLLI (SEQ ID NO: 144).

Embodiment 207: The chimeric inhibitory receptor of embodiment 206, wherein the transmembrane domain comprises the amino acid sequence of FPVWVVVGIVTAVVLGLTLLI (SEQ ID NO: 144).

Embodiment 208: The chimeric inhibitory receptor of any one of embodiments 1-189, wherein the chimeric inhibitory receptor comprises a transmembrane domain is derived from MPZ.

Embodiment 209: The chimeric inhibitory receptor of embodiment 208, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to YGVVLGAVIGGVLGVVLLLLLLFYVV (SEQ ID NO: 145).

Embodiment 210: The chimeric inhibitory receptor of embodiment 209, wherein the transmembrane domain comprises the amino acid sequence of YGVVLGAVIGGVLGVVLLLLLLFYVV (SEQ ID NO: 145).

Embodiment 211: The chimeric inhibitory receptor of any one of embodiments 1-189, wherein the chimeric inhibitory receptor comprises a transmembrane domain is derived from FCGR2B.

Embodiment 212: The chimeric inhibitory receptor of embodiment 211, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SSSPMGIIVAVVTGIAVAAIVAA (SEQ ID NO: 146).

Embodiment 213: The chimeric inhibitory receptor of embodiment 212, wherein the transmembrane domain comprises the amino acid sequence of SSSPMGIIVAVVTGIAVAAIVAA (SEQ ID NO: 146).

Embodiment 214: The chimeric inhibitory receptor of any one of embodiments 1-189, wherein the chimeric inhibitory receptor comprises a transmembrane domain is derived from SIGLEC-6.

Embodiment 215: The chimeric inhibitory receptor of embodiment 214, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LGAVWGASITTLVFLCVCFIF (SEQ ID NO: 147).

Embodiment 216: The chimeric inhibitory receptor of embodiment 215, wherein the transmembrane domain comprises the amino acid sequence of LGAVWGASITTLVFLCVCFIF (SEQ ID NO: 147).

Embodiment 217: The chimeric inhibitory receptor of any one of embodiments 1-189, wherein the chimeric inhibitory receptor comprises a transmembrane domain is derived from MPIG6B.

Embodiment 218: The chimeric inhibitory receptor of embodiment 217, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LLIPLLGAGLVLGLGALGLVW (SEQ ID NO: 148).

Embodiment 219: The chimeric inhibitory receptor of embodiment 218, wherein the transmembrane domain comprises the amino acid sequence of LLIPLLGAGLVLGLGALGLVW (SEQ ID NO: 148).

Embodiment 220: The chimeric inhibitory receptor of any one of embodiments 1-189, wherein the chimeric inhibitory receptor comprises a transmembrane domain is derived from VSIG4.

Embodiment 221: The chimeric inhibitory receptor of embodiment 220, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VFAIILIISLCCMVVFTMAYI (SEQ ID NO: 149).

Embodiment 222: The chimeric inhibitory receptor of embodiment 221, wherein the transmembrane domain comprises the amino acid sequence of VFAIILIISLCCMVVFTMAYI (SEQ ID NO: 149).

Embodiment 223: The chimeric inhibitory receptor of any one of embodiments 1-189, wherein the chimeric inhibitory receptor comprises a transmembrane domain is derived from SIGLEC-12.

Embodiment 224: The chimeric inhibitory receptor of embodiment 223, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to FGGAGATALVFLYFCIIFVVV (SEQ ID NO: 150).

Embodiment 225: The chimeric inhibitory receptor of embodiment 225, wherein the transmembrane domain comprises the amino acid sequence of FGGAGATALVFLYFCIIFVVV (SEQ ID NO: 150).

Embodiment 226: The chimeric inhibitory receptor of any one of embodiments 1-189, wherein the chimeric inhibitory receptor comprises a transmembrane domain is derived from LIR8.

Embodiment 227: The chimeric inhibitory receptor of embodiment 226, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VVTGVSVAFVLLLFLLLFLLL (SEQ ID NO: 151).

Embodiment 228: The chimeric inhibitory receptor of embodiment 227, wherein the transmembrane domain comprises the amino acid sequence of VVTGVSVAFVLLLFLLLFLLL (SEQ ID NO: 151).

Embodiment 229: The chimeric inhibitory receptor of any one of embodiments 1-189, wherein the chimeric inhibitory receptor comprises a transmembrane domain is derived from IRTA1.

Embodiment 230: The chimeric inhibitory receptor of embodiment 229, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VAAGATGGLLSALLLAVALLF (SEQ ID NO: 152).

Embodiment 231: The chimeric inhibitory receptor of embodiment 230, wherein the transmembrane domain comprises the amino acid sequence of VAAGATGGLLSALLLAVALLF (SEQ ID NO: 152).

Embodiment 232: The chimeric inhibitory receptor of any one of embodiments 1-189, wherein the chimeric inhibitory receptor comprises a transmembrane domain is derived from KIR2DL4.

Embodiment 233: The chimeric inhibitory receptor of embodiment 232, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AVIRYSVAIILFTILPFFLLH (SEQ ID NO: 153).

Embodiment 234: The chimeric inhibitory receptor of embodiment 233, wherein the transmembrane domain comprises the amino acid sequence of AVIRYSVAIILFTILPFFLLH (SEQ ID NO: 153).

Embodiment 235: The chimeric inhibitory receptor of any one of embodiments 1-189, wherein the chimeric inhibitory receptor comprises a transmembrane domain is derived from KIR2DL5.

Embodiment 236: The chimeric inhibitory receptor of embodiment 235, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LHILIGTSVAIILFIILFFFL (SEQ ID NO: 154). In some embodiments, the transmembrane domain comprises the amino acid sequence of LHILIGTSVAIILFIILFFFL (SEQ ID NO: 154).

Embodiment 237: The chimeric inhibitory receptor of any one of embodiments 1-189, wherein the chimeric inhibitory receptor comprises a transmembrane domain is derived from SIGLEC-7.

Embodiment 238: The chimeric inhibitory receptor of embodiment 237, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to GAVGGAGATALVFLSFCVIFIVV (SEQ ID NO: 155).

Embodiment 239: The chimeric inhibitory receptor of embodiment 238, wherein the transmembrane domain comprises the amino acid sequence of GAVGGAGATALVFLSFCVIFIVV (SEQ ID NO: 155).

Embodiment 240: The chimeric inhibitory receptor of any one of embodiments 1-189, wherein the chimeric inhibitory receptor comprises a transmembrane domain is derived from FCRH3.

Embodiment 241: The chimeric inhibitory receptor of embodiment 240, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AAGITGLVLSILVLAAAAALL (SEQ ID NO: 156).

Embodiment 242: The chimeric inhibitory receptor of embodiment 241, wherein the transmembrane domain comprises the amino acid sequence of AAGITGLVLSILVLAAAAALL (SEQ ID NO: 156).

Embodiment 243: The chimeric inhibitory receptor of any one of embodiments 1-189, wherein the chimeric inhibitory receptor comprises a transmembrane domain is derived from PCDHGC5.

Embodiment 244: The chimeric inhibitory receptor of embodiment 243, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LIVALATVSLLSLVTFTFLSA (SEQ ID NO: 157).

Embodiment 245: The chimeric inhibitory receptor of embodiment 244, wherein the transmembrane domain comprises the amino acid sequence of LIVALATVSLLSLVTFTFLSA (SEQ ID NO: 157).

Embodiment 246: The chimeric inhibitory receptor of any one of embodiments 1-189, wherein the chimeric inhibitory receptor comprises a transmembrane domain is derived from CDH11.

Embodiment 247: The chimeric inhibitory receptor of embodiment 246, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to GALIAILACIVILLVIVVLFVTL (SEQ ID NO: 158).

Embodiment 248: The chimeric inhibitory receptor of embodiment 247, wherein the transmembrane domain comprises the amino acid sequence of GALIAILACIVILLVIVVLFVTL (SEQ ID NO: 158).

Embodiment 249: The chimeric inhibitory receptor of any one of embodiments 1-189, wherein the chimeric inhibitory receptor comprises a transmembrane domain is derived from IMPG2.

Embodiment 250: The chimeric inhibitory receptor of embodiment 249, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VIIGITIASVVGLLVIFSAII (SEQ ID NO: 159).

Embodiment 251: The chimeric inhibitory receptor of embodiment 250, wherein the transmembrane domain comprises the amino acid sequence of VIIGITIASVVGLLVIFSAII (SEQ ID NO: 159).

Embodiment 252: The chimeric inhibitory receptor of any one of embodiments 1-189, wherein the chimeric inhibitory receptor comprises a transmembrane domain is derived from DSCAM.

Embodiment 253: The chimeric inhibitory receptor of embodiment 252, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LVTISCILVGVLLLFVLLLVV (SEQ ID NO: 160).

Embodiment 254: The chimeric inhibitory receptor of embodiment 253, wherein the transmembrane domain comprises the amino acid sequence of LVTISCILVGVLLLFVLLLVV (SEQ ID NO: 160).

Embodiment 255: The chimeric inhibitory receptor of any one of embodiments 1-254, wherein the protein is not expressed on the target tumor.

Embodiment 256: The chimeric inhibitory receptor of any one of embodiments 1-255, wherein the protein is expressed on a non-tumor cell.

Embodiment 257: The chimeric inhibitory receptor of embodiment 256, wherein the protein is expressed on a non-tumor cell derived from a tissue selected from the group consisting of brain, neuronal tissue, endocrine, endothelial, bone, bone marrow, immune system, muscle, lung, liver, gallbladder, pancreas, gastrointestinal tract, kidney, urinary bladder, male reproductive organs, female reproductive organs, adipose, soft tissue, and skin.

Embodiment 258: The chimeric inhibitory receptor of any one of embodiments 1-257, wherein the extracellular protein binding domain comprises a ligand-binding domain.

Embodiment 259: The chimeric inhibitory receptor of any one of embodiments 1-257, wherein the extracellular protein binding domain comprises a receptor-binding domain.

Embodiment 260: The chimeric inhibitory receptor of any one of embodiments 1-257, wherein the extracellular protein binding domain comprises an antigen-binding domain.

Embodiment 261: The chimeric inhibitory receptor of embodiment 260, wherein the antigen-binding domain comprises an antibody, an antigen-binding fragment of an antibody, a F(ab) fragment, a F(ab′) fragment, a single chain variable fragment (scFv), or a single-domain antibody (sdAb).

Embodiment 262: The chimeric inhibitory receptor of embodiment 260, wherein the antigen-binding domain comprises a single chain variable fragment (scFv).

Embodiment 263: The chimeric inhibitory receptor of embodiment 262, wherein each scFv comprises a heavy chain variable domain (VH) and a light chain variable domain (VL).

Embodiment 264: The chimeric inhibitory receptor of embodiment 263, wherein the VH and VL are separated by a peptide linker.

Embodiment 265: The chimeric inhibitory receptor of embodiment 264, wherein the peptide linker comprises an amino acid sequence selected from the group consisting of:

(SEQ ID NO: 47)
GGS,
(SEQ ID NO: 48)
GGSGGS,
(SEQ ID NO: 49)
GGSGGSGGS,
(SEQ ID NO: 50)
GGSGGSGGSGGS,
(SEQ ID NO: 51)
GGSGGSGGSGGSGGS,
(SEQ ID NO: 52)
GGGS,
(SEQ ID NO: 53)
GGGSGGGS,
(SEQ ID NO: 54)
GGGSGGGSGGGS,
(SEQ ID NO: 55)
GGGSGGGSGGGSGGGS,
(SEQ ID NO: 56)
GGGSGGGSGGGSGGGSGGGS,
(SEQ ID NO: 57)
GGGGS,
(SEQ ID NO: 58)
GGGGSGGGGS,
(SEQ ID NO: 59)
GGGGSGGGGSGGGGS,
(SEQ ID NO: 60)
GGGGSGGGGSGGGGSGGGGS,
(SEQ ID NO: 61)
GGGGSGGGGSGGGGSGGGGSGGGGS,
(SEQ ID NO: 63)
GGGGSGGGGSGGGGSQSV,
(SEQ ID NO: 64)
GSTSGSGKPGSGEGSTKG,
(SEQ ID NO: 65)
SGGGGSGGGGSGGGGSGGGGSGGGSLQ,
and
(SEQ ID NO: 62)
TTTPAPRPPTPAPTIALQPLSLRPEACRPAAGGAVHTRGLDFACDQTTPG
ERSSLPAFYPGTSGSCSGCGSLSLP.

Embodiment 266: The chimeric inhibitory receptor of any one of embodiments 263-265, wherein the scFv comprises the structure VH-L-VL or VL-L-VH, wherein VH is the heavy chain variable domain, L is the peptide linker, and VL is the light chain variable domain.

Embodiment 267: The chimeric inhibitory receptor of any one of embodiments 1-266, wherein the transmembrane domain is physically linked to the extracellular protein binding domain.

Embodiment 268: The chimeric inhibitory receptor of any one of embodiments 1-267, wherein one of the one or more intracellular signaling domains is physically linked to the transmembrane domain.

Embodiment 269: The chimeric inhibitory receptor of any one of embodiments 1-268, wherein the transmembrane domain is physically linked to the extracellular protein binding domain and one of the one or more intracellular signaling domains is physically linked to the transmembrane domain.

Embodiment 270: The chimeric inhibitory receptor of any one of embodiments 1-269, wherein the extracellular protein binding domain has a high binding affinity.

Embodiment 271: The chimeric inhibitory receptor of any one of embodiments 1-269, wherein extracellular protein binding domain has a low binding affinity.

Embodiment 272: The chimeric inhibitory receptor of any one of embodiments 1-271, wherein the chimeric inhibitory receptor is capable of suppressing cytokine production from an activated immunomodulatory cell.

Embodiment 273: The chimeric inhibitory receptor of any one of embodiments 1-272, wherein the chimeric inhibitory receptor is capable of suppressing a cell-mediated immune response to a target cell, wherein the immune response is induced by activation of the immunomodulatory cell.

Embodiment 274: The chimeric inhibitory receptor of any one of embodiments 1-273, wherein the target cell is a tumor cell.

Embodiment 275: The chimeric inhibitory receptor of any one of embodiments 1-274, wherein the one or more intracellular signaling domains comprise one or more modifications.

Embodiment 276: The chimeric inhibitory receptor of embodiment 275, wherein the one or more modifications modulate sensitivity of the chimeric inhibitory receptor relative to the otherwise identical, unmodified receptor.

Embodiment 277: The chimeric inhibitory receptor of embodiment 275, wherein the one or more modifications increase sensitivity of the chimeric inhibitory receptor relative to the otherwise identical, unmodified receptor.

Embodiment 278: The chimeric inhibitory receptor of embodiment 275, wherein the one or more modifications reduce sensitivity of the chimeric inhibitory receptor relative to the otherwise identical, unmodified receptor.

Embodiment 279: The chimeric inhibitory receptor of any one embodiments 275-278, wherein the one or more modifications modulate potency of the chimeric inhibitory receptor relative to the otherwise identical, unmodified receptor.

Embodiment 280: The chimeric inhibitory receptor of embodiment 279, wherein the one or more modifications increase potency of the chimeric inhibitory receptor relative to the otherwise identical, unmodified receptor.

Embodiment 281: The chimeric inhibitory receptor of embodiment 279, wherein the one or more modifications reduce potency of the chimeric inhibitory receptor relative to the otherwise identical, unmodified receptor.

Embodiment 282: The chimeric inhibitory receptor of any one of embodiments 275-281, wherein the one or more modifications modulate basal prevention, attenuation, or inhibition of activation of the tumor-targeting chimeric receptor when expressed on an immunomodulatory cell relative to the otherwise identical, unmodified receptor.

Embodiment 283: The chimeric inhibitory receptor of embodiment 282, wherein the one or more modifications reduce basal prevention, attenuation, or inhibition relative to the otherwise identical, unmodified receptor.

Embodiment 284: The chimeric inhibitory receptor of embodiment 282, wherein the one or more modifications increase basal prevention, attenuation, or inhibition relative to the otherwise identical, unmodified receptor.

Embodiment 285: The chimeric inhibitory receptor of any one of embodiments 1-284, wherein the chimeric inhibitory receptor further comprises a spacer region positioned between the extracellular protein binding domain and the transmembrane domain and operably linked to each of the extracellular protein binding domain and the transmembrane domain.

Embodiment 286: The chimeric inhibitory receptor of any one of embodiments 1-284, wherein the chimeric inhibitory receptor further comprises a spacer region positioned between the extracellular protein binding domain and the transmembrane domain and physically linked to each of the extracellular protein binding and the transmembrane domain.

Embodiment 287: The chimeric inhibitory receptor of embodiment 285, wherein the spacer region is derived from a protein selected from the group consisting of: CD8α, CD4, CD7, CD28, IgG1, IgG4, FcγRIIIα, LNGFR, and PDGFR.

Embodiment 288: The chimeric inhibitory receptor of embodiment 285, wherein the spacer region comprises an amino acid sequence selected from the group consisting of:

(SEQ ID NO: 73)
TTTPAPRPPTPAPTIALQPLSLRPEACRPAAGGAVHTRGLDFACD,
(SEQ ID NO: 77)
ALSNSIMYFSHFVPVFLPAKPTTTPAPRPPTPAPTIASQPLSLRPEACRP
AAGGAVHTRGLDFACD,
(SEQ ID NO: 67)
AAAIEVMYPPPYLDNEKSNGTIIHVKGKHLCPSPLFPGPSKP,
(SEQ ID NO: 68)
ESKYGPPCPSCP,
(SEQ ID NO: 69)
ESKYGPPAPSAP,
(SEQ ID NO: 70)
ESKYGPPCPPCP,
(SEQ ID NO: 71)
EPKSCDKTHTCP,
(SEQ ID NO: 72)
AAAFVPVFLPAKPTTTPAPRPPTPAPTIASQPLSLRPEACRPAAGGAVHT
RGLDFACDIYIWAPLAGTCGVLLLSLVITLYCNHRN,
(SEQ ID NO: 74)
ACPTGLYTHSGECCKACNLGEGVAQPCGANQTVCEPCLDSVTFSDVVSAT
EPCKPCTECVGLQSMSAPCVEADDAVCRCAYGYYQDETTGRCEACRVCEA
GSGLVFSCQDKQNTVCEECPDGTYSDEADAEC,
(SEQ ID NO: 75)
ACPTGLYTHSGECCKACNLGEGVAQPCGANQTVC,
(SEQ ID NO: 76)
AVGQDTQEVIVVPHSLPFKV,
(SEQ ID NO: 183)
ESKYGPPCPSCPAPPVAGPSVFLFPPKPKDTLMISRTPEVTCVVVDVSQE
DPEVQFNWYVDGVEVHNAKTKPREEQFQSTYRVVSVLTVLHQDWLNGKEY
KCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQE
GNVFSCSVMHEALHNHYTQKSLSLSLGK,
and
(SEQ ID NO: 184)
ESKYGPPCPSCPGQPREPQVYTLPPSQEEMTKNQVSLTCLVKGFYPSDIA
VEWESNGQPENNYKTTPPVLDSDGSFFLYSRLTVDKSRWQEGNVFSCSVM
HEALHNHYTQKSLSLSLGK

Embodiment 289: The chimeric inhibitory receptor of any one of embodiments 285-288, wherein the spacer region modulates sensitivity of the chimeric inhibitory receptor relative to an otherwise identical chimeric inhibitory receptor lacking the spacer region.

Embodiment 290: The chimeric inhibitory receptor of embodiment 289, wherein the spacer region increases sensitivity of the chimeric inhibitory receptor relative to an otherwise identical chimeric inhibitory receptor lacking the spacer region.

Embodiment 291: The chimeric inhibitory receptor of embodiment 289, wherein the spacer region reduces sensitivity of the chimeric inhibitory receptor relative to an otherwise identical chimeric inhibitory receptor lacking the spacer region.

Embodiment 292: The chimeric inhibitory receptor of any one of embodiments 285-291, wherein the spacer region modulates potency of the chimeric inhibitory receptor relative to an otherwise identical chimeric inhibitory receptor lacking the spacer region.

Embodiment 293: The chimeric inhibitory receptor of embodiment 292, wherein the spacer region increases potency of the chimeric inhibitory receptor relative to an otherwise identical chimeric inhibitory receptor lacking the spacer region.

Embodiment 294: The chimeric inhibitory receptor of embodiment 292, wherein the spacer region reduces potency of the chimeric inhibitory receptor relative to an otherwise identical chimeric inhibitory receptor lacking the spacer region.

Embodiment 295: The chimeric inhibitory receptor of any one of embodiments 285-294, wherein the spacer region modulates basal prevention, attenuation, or inhibition of activation of the tumor-targeting chimeric receptor when expressed on an immunomodulatory cell relative to an otherwise identical chimeric inhibitory receptor lacking the spacer region.

Embodiment 296: The chimeric inhibitory receptor of embodiment 295, wherein the spacer region reduces basal prevention, attenuation, or inhibition relative to an otherwise identical chimeric inhibitory receptor lacking the spacer region.

Embodiment 297: The chimeric inhibitory receptor of embodiment 295, wherein the spacer region increases basal prevention, attenuation, or inhibition relative to an otherwise identical chimeric inhibitory receptor lacking the spacer region.

Embodiment 298: The chimeric inhibitory receptor of any one of embodiments 1-297, wherein the chimeric inhibitory receptor further comprises an intracellular spacer region positioned between the transmembrane domain and one of the one or more intracellular signaling domains and operably linked to each of the transmembrane domain and one of the one or more intracellular signaling domains.

Embodiment 299: The chimeric inhibitory receptor of any one of embodiments 1-297, wherein the chimeric inhibitory receptor further comprises an intracellular spacer region positioned between the transmembrane domain and one of the one or more intracellular signaling domains and physically linked to each of the transmembrane domain and one of the one or more intracellular signaling domains.

Embodiment 300: The chimeric inhibitory receptor of embodiment 298, wherein the intracellular spacer region modulates sensitivity of the chimeric inhibitory receptor relative to an otherwise identical chimeric inhibitory receptor lacking the intracellular spacer region.

Embodiment 301: The chimeric inhibitory receptor of embodiment 300, wherein the intracellular spacer region increases sensitivity of the chimeric inhibitory receptor relative to an otherwise identical chimeric inhibitory receptor lacking the intracellular spacer region.

Embodiment 302: The chimeric inhibitory receptor of embodiment 300, wherein the intracellular spacer region reduces sensitivity of the chimeric inhibitory receptor relative to an otherwise identical chimeric inhibitory receptor lacking the intracellular spacer region.

Embodiment 303: The chimeric inhibitory receptor of any one of embodiments 298-302, wherein the intracellular spacer region modulates potency of the chimeric inhibitory receptor relative to an otherwise identical chimeric inhibitory receptor lacking the intracellular spacer region.

Embodiment 304: The chimeric inhibitory receptor of embodiment 303, wherein the intracellular spacer region increases potency of the chimeric inhibitory receptor relative to an otherwise identical chimeric inhibitory receptor lacking the intracellular spacer region.

Embodiment 305: The chimeric inhibitory receptor of embodiment 303, wherein the intracellular spacer region reduces potency of the chimeric inhibitory receptor relative to an otherwise identical chimeric inhibitory receptor lacking the intracellular spacer region.

Embodiment 306: The chimeric inhibitory receptor of any one of embodiments 298-305, wherein the intracellular spacer region modulates basal prevention, attenuation, or inhibition of activation of the tumor-targeting chimeric receptor when expressed on an immunomodulatory cell relative to an otherwise identical chimeric inhibitory receptor lacking the intracellular spacer region.

Embodiment 307: The chimeric inhibitory receptor of embodiment 306, wherein the intracellular spacer region reduces basal prevention, attenuation, or inhibition relative to an otherwise identical chimeric inhibitory receptor lacking the intracellular spacer region.

Embodiment 308: The chimeric inhibitory receptor of embodiment 306, wherein the intracellular spacer region increases basal prevention, attenuation, or inhibition relative to an otherwise identical chimeric inhibitory receptor lacking the intracellular spacer region.

Embodiment 309: The chimeric inhibitory receptor of any one of embodiments 1-308, wherein the inhibitory chimeric receptor further comprises an enzymatic inhibitory domain.

Embodiment 310: The chimeric inhibitory receptor of embodiment 309, wherein the enzymatic inhibitory domain is capable of preventing, attenuating, or inhibiting activation of a tumor-targeting chimeric receptor when expressed on an immunomodulatory cell relative to an otherwise identical chimeric inhibitory receptor lacking the enzymatic inhibitory domain.

Embodiment 311: The chimeric inhibitory receptor of embodiment 309 or embodiment 310, wherein the enzymatic inhibitory domain comprises an enzyme catalytic domain.

Embodiment 312: The chimeric inhibitory receptor of embodiment 311, wherein the enzyme catalytic domain is derived from an enzyme selected from the group consisting of: CSK, SHP-1, PTEN, CD45, CD148, PTP-MEG1, PTP-PEST, c-CBL, CBL-b, PTPN22, LAR, PTPH1, SHIP-1, and RasGAP.

Embodiment 313: The chimeric inhibitory receptor of any one of embodiments 309-312, wherein the enzymatic inhibitory domain comprises one or more modifications that modulate basal prevention, attenuation, or inhibition.

Embodiment 314: The chimeric inhibitory receptor of embodiment 313, wherein the one or more modifications reduce basal prevention, attenuation, or inhibition relative to an otherwise identical enzymatic inhibitory domain lacking the one or more modifications.

Embodiment 315: The chimeric inhibitory receptor of embodiment 313, wherein the one or more modifications increase basal prevention, attenuation, or inhibition relative to an otherwise identical enzymatic inhibitory domain lacking the one or more modifications.

Embodiment 316: The chimeric inhibitory receptor of any one of embodiments 1-315, wherein the tumor-targeting chimeric receptor is a chimeric antigen receptor (CAR) or an engineered T cell receptor (TCR).

Embodiment 317: The chimeric inhibitory receptor of any one of embodiments 1-316, wherein the immunomodulatory cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell.

Embodiment 318: The chimeric inhibitory receptor of any one of embodiments 1-316, wherein the immunomodulatory cell is a Natural Killer (NK) cell.

Embodiment 319: A composition comprising the chimeric inhibitory receptor of any one of embodiments 1-318 and a pharmaceutically acceptable carrier, a pharmaceutically acceptable excipient, or a combination thereof.

Embodiment 320: An engineered nucleic acid encoding the chimeric inhibitory receptor of any one of embodiments 1-318.

Embodiment 321: An expression vector comprising the engineered nucleic acid of embodiment 320.

Embodiment 322: A composition comprising the engineered nucleic acid of embodiment 320 or the expression vector of embodiment 321, and a pharmaceutically acceptable carrier

Embodiment 323: A producer cell comprising the chimeric inhibitory receptor of any one of embodiments 1-318, the engineered nucleic acid of embodiment 320, or the expression vector of embodiments 321.

Embodiment 324: An isolated immunomodulatory cell comprising the chimeric inhibitory receptor of any one of embodiments 1-318, the engineered nucleic acid of embodiment 320, or the expression vector of embodiments 321.

Embodiment 325: The isolated cell of embodiment 323, wherein the cell further comprises a tumor-targeting chimeric receptor expressed on the surface of the cell.

Embodiment 326: The isolated cell of embodiment 325, wherein upon binding of the protein to the chimeric inhibitory receptor, the chimeric inhibitory receptor prevents, attenuates, or inhibits activation of the tumor-targeting chimeric receptor relative to an otherwise identical cell lacking a chimeric inhibitory receptor.

Embodiment 327: An isolated immunomodulatory cell comprising a chimeric inhibitory receptor, wherein the chimeric inhibitory receptor comprises:
an extracellular protein binding domain,
a transmembrane domain, wherein the transmembrane domain is operably linked to the extracellular protein binding domain, and
one or more intracellular signaling domains, wherein the one or more intracellular signaling domains are operably linked to the transmembrane domain, and wherein the one or more intracellular signaling domain are each derived from a protein selected from the group consisting of: PECAM-1, CD72, IRTA2, IRTA4, NKIR, IL1RAP, PTPRO, PTPRZ1, TLT1, SLAMF1, sSLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM; and
   wherein upon binding of the protein to the chimeric inhibitory receptor, the chimeric inhibitory receptor prevents, attenuates, or inhibits activation of a tumor-targeting chimeric receptor expressed on the surface of the cell.

Embodiment 328: The isolated cell of embodiment 327, wherein the cell further comprises a tumor-targeting chimeric receptor expressed on the surface of the cell.

Embodiment 329: An isolated cell comprising:
(a) a chimeric inhibitory receptor, wherein and the chimeric inhibitory receptor comprises:
   an extracellular protein binding domain,
   a transmembrane domain, wherein the transmembrane domain is operably linked to the extracellular protein binding domain, and
   one or more intracellular signaling domains, wherein the one or more intracellular signaling domains are operably linked to the transmembrane domain, and wherein the one or more intracellular signaling domain are each derived from a protein selected from the group consisting of: PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM; and
(b) a tumor-targeting chimeric receptor expressed on the surface of the cell,
wherein upon binding of the protein to the chimeric inhibitory receptor, the chimeric inhibitory receptor prevents, attenuates, or inhibits activation of the tumor-targeting chimeric receptor.

Embodiment 330: The isolated cell of any one of embodiments 323-329, wherein the chimeric inhibitory receptor is recombinantly expressed.

Embodiment 331: The isolated cell of any one of embodiments 323-330, wherein the chimeric inhibitory receptor is expressed from a vector or a selected locus from the genome of the cell.

Embodiment 332: The isolated cell of any one of embodiments 323-331, wherein the tumor-targeting chimeric receptor is a chimeric antigen receptor (CAR) or an engineered T cell receptor.

Embodiment 333: The cell of any one of embodiments 323-332, wherein prior to binding of the protein to the chimeric inhibitory receptor, the tumor-targeting chimeric receptor is capable of activating the cell.

Embodiment 334: The cell of any one of embodiments 323-333, wherein upon binding of the protein to the chimeric inhibitory receptor, the chimeric inhibitory receptor suppresses cytokine production from the activated cell.

Embodiment 335: The cell of any one of embodiments 323-334, wherein upon binding of the protein to the chimeric inhibitory receptor, the chimeric inhibitory receptor suppresses a cell-mediated immune response to a target cell, wherein the immune response is induced by activation of the immunomodulatory cell.

Embodiment 336: The cell of any one of embodiments 323-335, wherein the transmembrane domain is physically linked to the extracellular protein binding domain.

Embodiment 337: The cell of any one of embodiments 323-335, wherein the intracellular signaling domain is physically linked to the transmembrane domain.

Embodiment 338: The cell of any one of embodiments 323-335, wherein the transmembrane domain is physically linked to the extracellular protein binding domain and one of the one or more intracellular signaling domains is physically linked to the transmembrane domain.

Embodiment 339: The isolated cell of any one of embodiments 323-335, wherein the target cell is a tumor cell.

Embodiment 340: The isolated cell of any one of embodiments 323-339, wherein the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell.

Embodiment 341: The isolated cell of any one of embodiments 323-339, wherein the cell is a Natural Killer (NK) cell.

Embodiment 342: The isolated cell of any one of embodiments 323-341, wherein the cell is autologous.

Embodiment 343: The isolated cell of any one of embodiments 323-341, wherein the cell is allogeneic.

Embodiment 344: A composition comprising the isolated cell of any one of embodiments 323-343 and a pharmaceutically acceptable carrier, a pharmaceutically acceptable excipient, or a combination thereof.

Embodiment 345: A method of preventing, attenuating, or inhibiting a cell-mediated immune response induced by a tumor-targeting chimeric receptor expressed of the surface of an immunomodulatory cell, comprising:
engineering the immunomodulatory cell to express the chimeric inhibitory receptor of any one of embodiments 1-318 on the surface of the immunomodulatory cell,
wherein upon binding of a cognate antigen to the chimeric inhibitory receptor, the intracellular signaling domain prevents, attenuates, or inhibits activation of the tumor-targeting chimeric receptor.

Embodiment 346: A method of preventing, attenuating, or inhibiting activation of a tumor-targeting chimeric receptor expressed on the surface of an immunomodulatory cell, comprising:
contacting the isolated cell of any one of embodiments 323-343 or the composition of embodiment 344 with a cognate antigen of the chimeric inhibitory receptor under conditions suitable for the chimeric inhibitory receptor to bind the cognate antigen,
wherein upon binding of the antigen to the chimeric inhibitory receptor, the intracellular signaling domain prevents, attenuates, or inhibits activation of the tumor-targeting chimeric receptor.

Embodiment 347: The method of embodiment 345 or embodiment 346, wherein the tumor-targeting chimeric receptor is a chimeric antigen receptor (CAR) or an engineered T cell receptor.

Embodiment 348: The method of embodiment 347, wherein the CAR binds one or more antigens expressed on the surface of a tumor cell.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. Sec, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Inhibitory Chimeric Receptors with Different Intracellular Signaling Domains Modulate NK-Medicated Cell Killing

### Methods and Materials

### Inhibitory Chimeric Receptor and Tumor-Targeting Chimeric Receptor Constructs

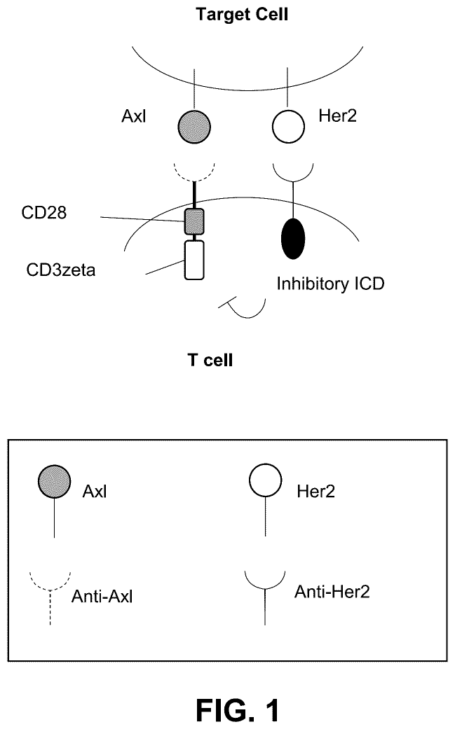

Inhibitory chimeric receptors (iCAR) were generated from different inhibitory intracellular signaling domains (ICDs). The inhibitory chimeric receptor comprised a CD8 secretion signal, an anti-Her2 (clone H3B1) scFv with a V5 tag, a CD8 hinge domain, a transmembrane domain, and an intracellular signaling domain. The V5 tag was fused to the C-terminus of the scFv in the hinge region of the iCAR. A tumor-targeting CAR (i.e., an activating CAR, aCAR) was also constructed with a Igκ secretion signal, an anti-Axl (clone 1448) scFv with a FLAG tag, a CD8 hinge domain, a CD28 transmembrane domain, and CD28 and CD3ζ intracellular signaling domains. The FLAG tag was fused to the N-terminus of the scFv. An exemplary diagram of a NK cell co-expressing an anti-Axl-CD28-CD3ζ iCAR and an anti-Her2 iCAR contacting a target cell expressing Axl and Her2 is shown in FIG. 1.

### NK Cell Transduction and Expansion

Donor-derived NK cells were obtained from PBMCs and were expanded for 10 days with mitomycin C-treated K562 feeder cells, followed by transduction with a retroviral vector encoding the aCAR and iCAR constructs. Sequences for the constructs are shown in Table 9.

### NK Cell Cytotoxicity Assay

After 6 days, cytotoxicity assays were performed by co-incubating engineered NK cells and target SEM cells engineered to overexpress Axl antigen (SA−), or both Axl and Her2 antigens (SA+). Engineered NK cells were incubated with SEM cells at an ET ratio of 1:4. After an overnight incubation (approximately 16 hours), cells were stained with viability dyes and counted via flow cytometry. The target cell reduction was quantified as 100%×(1−No. Targets treated with experimental NK cells/No. Targets treated with untransduced NK cells). CAR-mediated killing was quantified as target reduction with experimental cells minus target reduction with untransduced NK cells. Control iCAR in this context was a LIR1 iCAR with a mistargeted scFv that did not bind Her2.

### Results

The ability of an iCAR to reduce or inhibit NK cell activation in an NK cell expressing an iCAR and an aCAR that each bind different antigens was assessed.

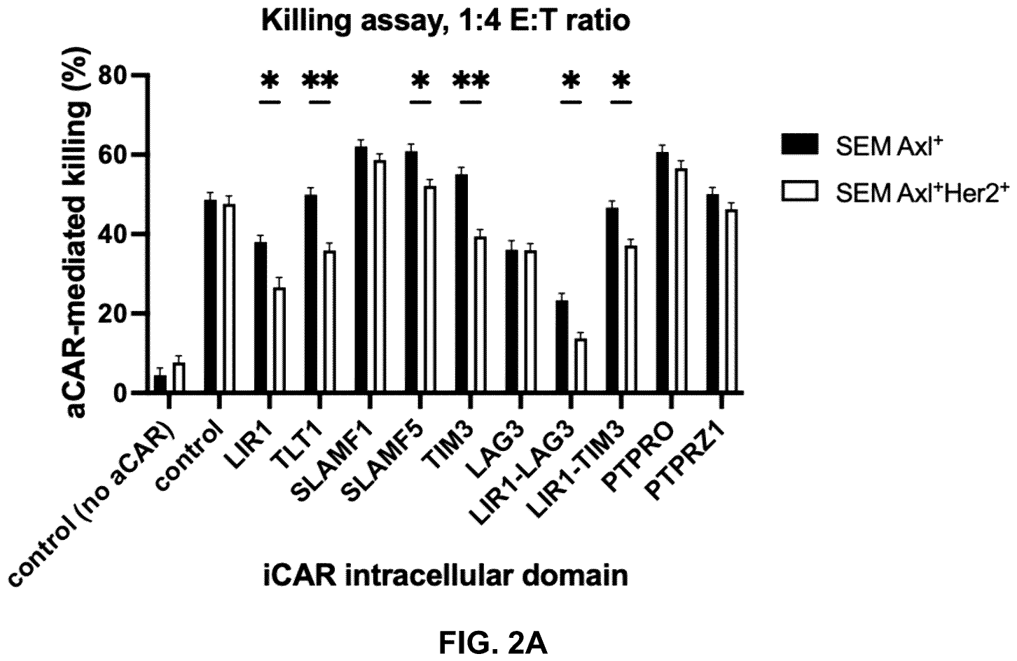

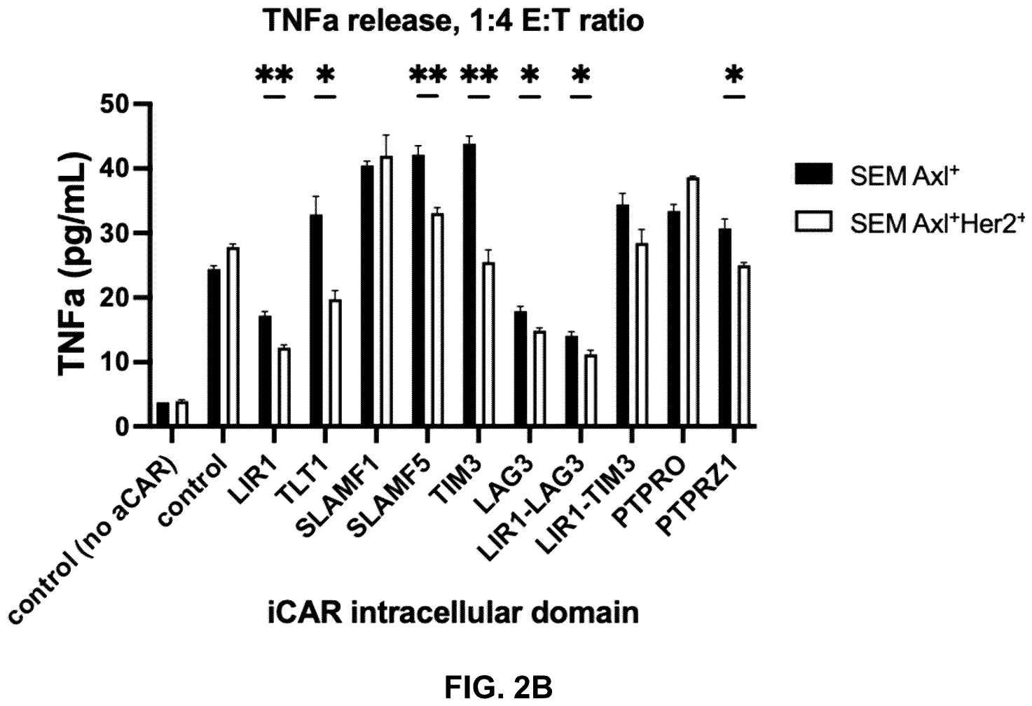

Co-culture of the aCAR (aAxl-28z) NK cells induced cytotoxicity of SEM cells expressing Axl (SA− or SA+). When NK cells were co-transduced with a vector encoding the anti-Her2 iCARs having PECAM-1, CD72, IRTA2, IRTA4, NKIR, IL1RAP, PTPRO, PTPRZ1, TLT1, SLAMF1, or SLAMF5 derived inhibitory intracellular signaling domains and transmembrane domains, these cells suppressed the NK cell-mediated cytotoxicity of the anti-Axl aCAR (aAxl-28z) after co-culture with SEM cells expressing both Axl and Her2 (SA+), but not SEM cells expressing Axl and not Her2 (SA−) (FIG. 2A). Thus, binding of the iCAR to its cognate ligand on the target cell reduces aCAR-induced cytotoxicity. The same cells were also assessed for release of TNF-alpha (TNFa) (FIG. 2B).

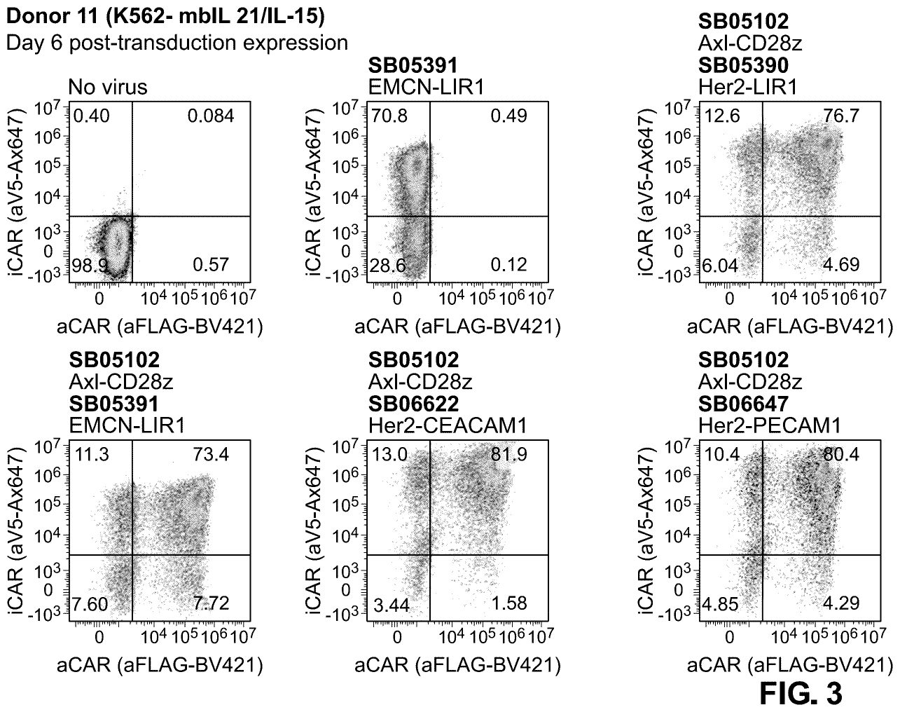

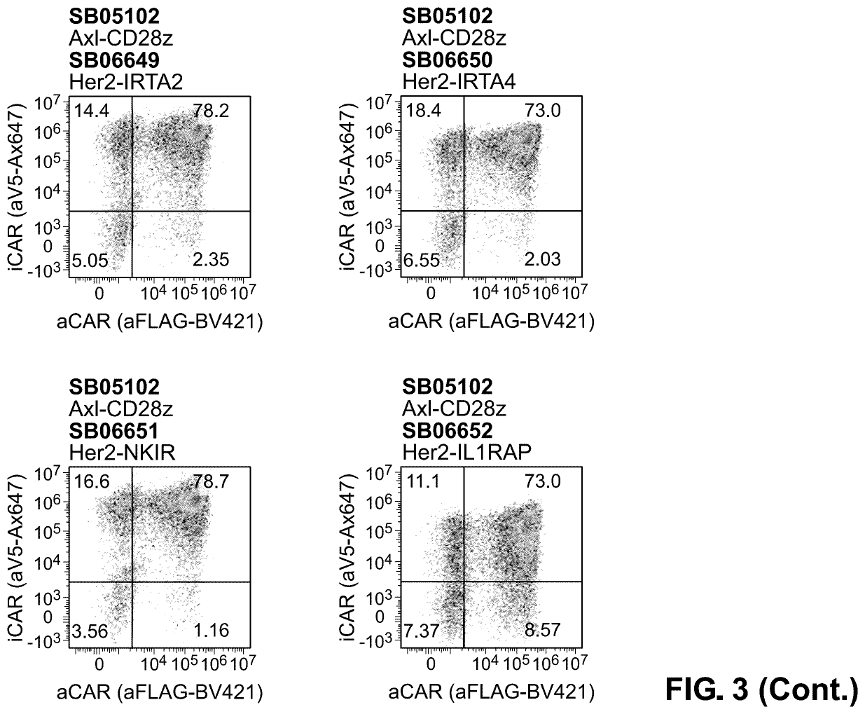

Her2 iCARs having PECAM-1, CD72, IRTA2, IRTA4, NKIR, IL1RAP, PTPRO, PTPRZ1, TLT1, SLAMF1, or SLAMF5 derived inhibitory intracellular signaling domains were expressed at high levels in retrovirus transduced NK cells without subsequent enrichment. High levels of co-expression of iCAR and aCAR were observed after co-transduction (FIG. 3).

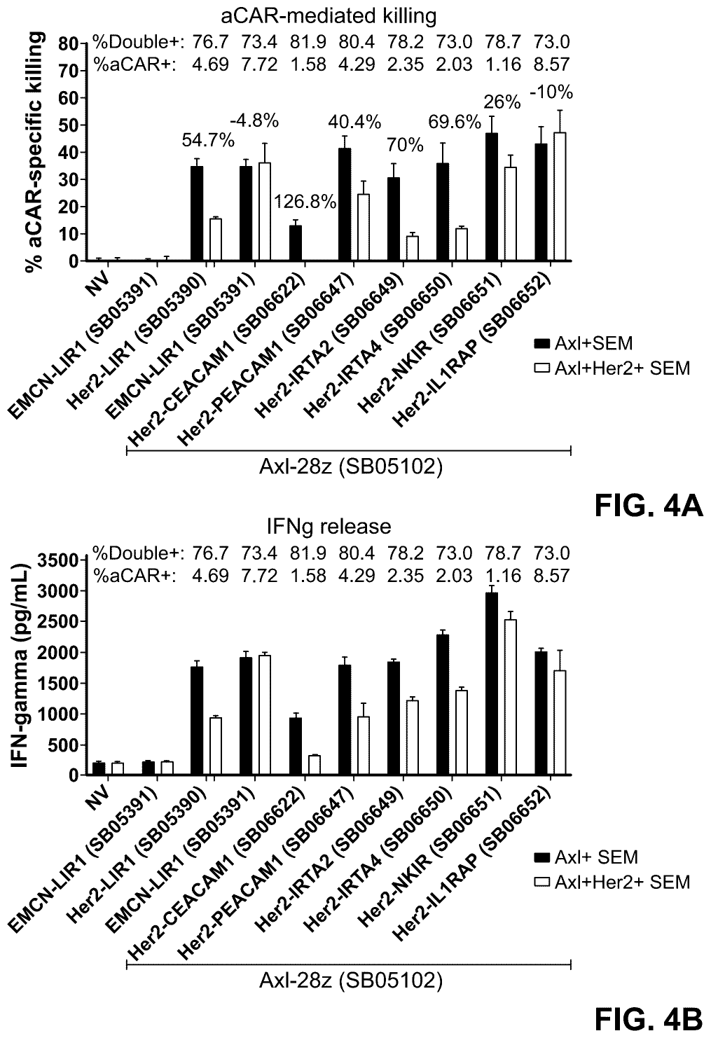

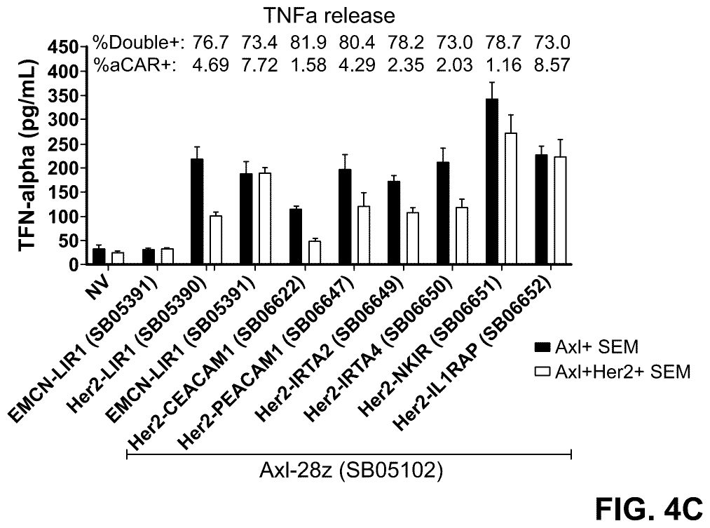

Her2 iCARs having CEACAM1, PECAM1, IRTA2, NKIR, and IL1RAP intracellular domains were transduced into donor-derived NK cells with anti-Axl aCAR (FIG. 4A). CAR specific killing was assessed by co-culturing with target cells expressing Axl (target antigen) and/or Her2 (safety antigen) as previously described. Her2 iCARs having CEACAM1, PEACAM1, IRTA4, NKIR, and IRTA2 demonstrate inhibition of cell-specific killing of target expressing both the target antigen and the safety antigen. In addition, release of interferon-gamma (FIG. 4B) and TNF-alpha (FIG. 4C) was also assessed in cells expressing the aCAR and iCARs of FIG. 4A.

### Example 2: Additional Inhibitory Chimeric Receptors with Different Intracellular Signaling Domains Modulate NK-Medicated Cell Killing

Additional inhibitory chimeric receptors were assessed in the same manner as previously described in Example 1 to assess whether these receptors can inhibit the function of an activating chimeric receptor.

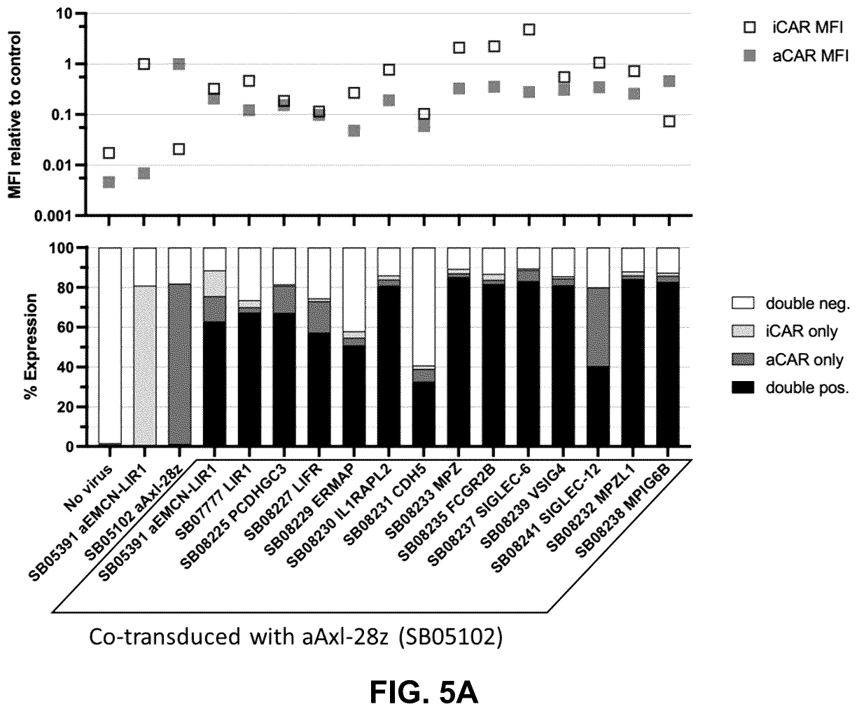

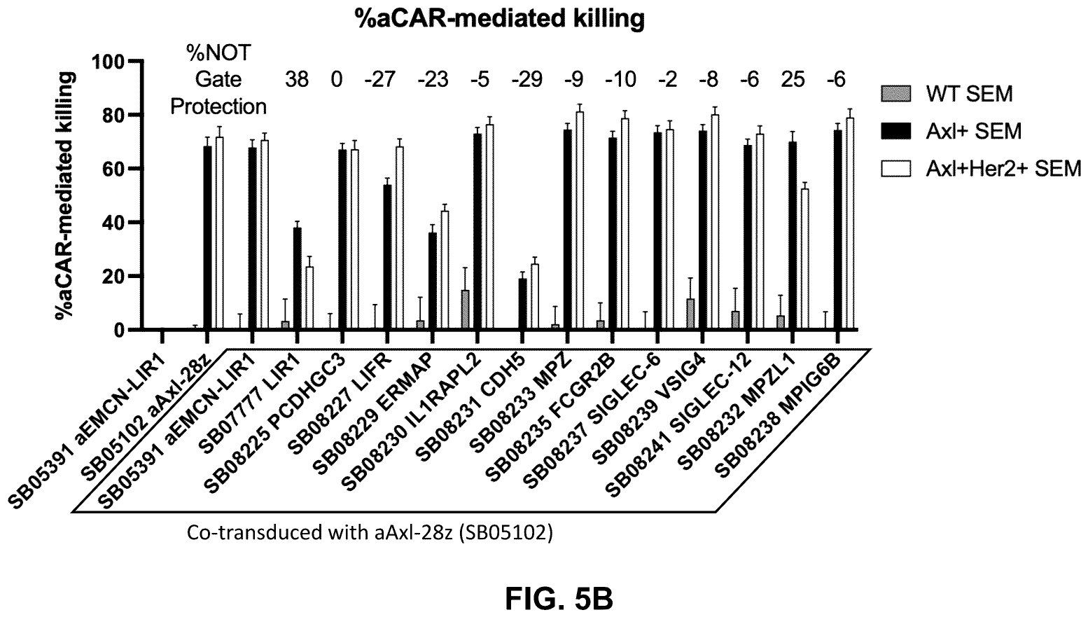

In the following study, Her2 iCARs having PCDHGC3, LIFR, ERMAP, IL1RAPL2, CDH5, MPZ, FCGR2B, SIGLEC-6, VSIG4, SIGLEC-12, MPZL1, and MPIG6B intracellular domains. The expression of the iCAR and/or aCAR were assessed (FIG. 5A). The top panel shows the mean fluorescence intensity (MFI) of the iCAR and the aCAR in each transduced cell population. The bottom panel shows the population distribution of whether cells express the iCAR, aCAR, both iCAR and aCAR, or neither.

FIG. 5B shows target cell (SEM) reduction when co-culturing SEM cells with each transduced NK cell population. Above each population, the % Protection is noted, comparing killing in Axl+ SEM cells versus Axl+/Her2+ SEM cells.

### Example 3: Additional Inhibitory Chimeric Receptors with Different Intracellular Signaling Domains Modulate NK-Medicated Cell Killing

iCARs having LIR8, IRTA1, KIR2DL4, KIR2DL5, SIGLEC-7, FCRH3, PCDHGC5, CDH11, IMPG2, and DSCAM intracellular domains are tested for their expression and NK-specific killing of target cells. iCARs are co-transduced with an aCAR to produced transduced NK cells. The transduced NK cells are co-cultured with target cells expressing the target antigen and/or the safety antigen to assess whether the iCAR can inhibit the function of the aCAR, thereby reducing killing of a target cell expressing the target antigen and the safety antigen.

Table 9 provides the full sequences of the inhibitory chimeric receptor and tumor-targeting chimeric receptor synthesized. CAR constructs were cloned into a retroviral vector. The antigen specificity and domain organization for the CAR constructs examined are described in Table 9 below, and the individual components of the chimeric antigen receptors are summarized in Table 10.

TABLE 9

| SEQ | | |
|---|---|---|
| ID | | |
| NO | Name | Sequence |
| 83 | SB06647 | MALPVTALLLPLALLLHAARPQVQLVQSGAEVKKPGESLKISCKGSG |
| | CD8ss aHer2 | YSFTSYWIAWVRQMPGKGLEYMGLIYPGDSDTKYSPSFQGQVTISVD |
| | V5 CD8h | KSVSTAYLQWSSLKPSDSAVYFCARHDVGYCTDRTCAKWPEYFQHWG |
| | PECAM-1tm | QGTLVTVSSGGGGSGGGGSGGGGSQSVLTQPPSVSAAPGQKVTISCS |
| | PECAM- | GSSSNIGNNYVSWYQQLPGTAPKLLIYDHTNRPAGVPDRFSGSKSGT |
| | 1icd P2A | SASLAISGFRSEDEADYYCASWDYTLSGWVFGGGTKLTVLGGKPIPN |
| | PuroR | PLLGLDSTNGAATTTPAPRPPTPAPTIALQPLSLRPEACRPAAGGAV |
| | | HTRGLDFACDGLIAVVIIGVIIALLIIAAKCYFLRKAKAKQMPVEMS |
| | | RPAVPLLNSNNEKMSDPNMEANSHYGHNDDVRNHAMKPINDNKEPLN |
| | | SDVQYTEVQVSSAESHKDLGKKDTETVYSEVRKAVPDAVESRYSRTE |
| | | GSLDGTGSGATNFSLLKQAGDVEENPGPMTEYKPTVRLATRDDVPRA |
| | | VRTLAAAFADYPATRHTVDPDRHIERVTELQELFLTRVGLDIGKVWV |
| | | ADDGAAVAVWTTPESVEAGAVFAEIGPRMAELSGSRLAAQQQMEGLL |
| | | APHRPKEPAWFLATVGVSPDHQGKGLGSAVVLPGVEAAERAGVPAFL |
| | | ETSAPRNLPFYERLGFTVTADVEVPEGPRTWCMTRKPGA* |
| 84 | SB06648 | MALPVTALLLPLALLLHAARPQVQLVQSGAEVKKPGESLKISCKGSG |
| | CD8ss aHer2 | YSFTSYWIAWVRQMPGKGLEYMGLIYPGDSDTKYSPSFQGQVTISVD |
| | V5 CD8h | KSVSTAYLQWSSLKPSDSAVYFCARHDVGYCTDRTCAKWPEYFQHWG |
| | LIR1tm | QGTLVTVSSGGGGSGGGGSGGGGSQSVLTQPPSVSAAPGQKVTISCS |
| | CD72icd | GSSSNIGNNYVSWYQQLPGTAPKLLIYDHTNRPAGVPDRESGSKSGT |
| | (Type II) | SASLAISGFRSEDEADYYCASWDYTLSGWVFGGGTKLTVLGGKPIPN |
| | P2A PuroR | PLLGLDSTNGAATTTPAPRPPTPAPTIALQPLSLRPEACRPAAGGAV |
| | | HTRGLDFACDVIGILVAVILLLLLLLLLFLIMAEAITYADLRFVKAP |
| | | LKKSISSRLGQDPGADDDGEITYENVQVPAVLGVPSSLASSVLGDKA |
| | | AVKSEQPTASWRAVTSPAVGRILPCRTTCLRYGSGATNFSLLKQAGD |
| | | VEENPGPMTEYKPTVRLATRDDVPRAVRTLAAAFADYPATRHTVDPD |
| | | RHIERVTELQELFLTRVGLDIGKVWVADDGAAVAVWTTPESVEAGAV |
| | | FAEIGPRMAELSGSRLAAQQQMEGLLAPHRPKEPAWFLATVGVSPDH |
| | | QGKGLGSAVVLPGVEAAERAGVPAFLETSAPRNLPFYERLGFTVTAD |
| | | VEVPEGPRTWCMTRKPGA* |
| 85 | SB06649 | MALPVTALLLPLALLLHAARPQVQLVQSGAEVKKPGESLKISCKGSG |
| | CD8ss aHer2 | YSFTSYWIAWVRQMPGKGLEYMGLIYPGDSDTKYSPSFQGQVTISVD |
| | V5 CD8h | KSVSTAYLQWSSLKPSDSAVYFCARHDVGYCTDRTCAKWPEYFQHWG |
| | IRTA2tm | QGTLVTVSSGGGGSGGGGSGGGGSQSVLTQPPSVSAAPGQKVTISCS |
| | IRTA2icd | GSSSNIGNNYVSWYQQLPGTAPKLLIYDHTNRPAGVPDRESGSKSGT |
| | P2A PuroR | SASLAISGFRSEDEADYYCASWDYTLSGWVFGGGTKLTVLGGKPIPN |
| | | PLLGLDSTNGAATTTPAPRPPTPAPTIALQPLSLRPEACRPAAGGAV |
| | | HTRGLDFACDVAGGLLSIAGLAAGALLLYCWLSRKAGRKPASDPARS |
| | | PSDSDSQEPTYHNVPAWEELQPVYTNANPRGENVVYSEVRIIQEKKK |
| | | HAVASDPRHLRNKGSPIIYSEVKVASTPVSGSLFLASSAPHRGSGAT |
| | | NFSLLKQAGDVEENPGPMTEYKPTVRLATRDDVPRAVRTLAAAFADY |
| | | PATRHTVDPDRHIERVTELQELFLTRVGLDIGKVWVADDGAAVAVWT |
| | | TPESVEAGAVFAEIGPRMAELSGSRLAAQQQMEGLLAPHRPKEPAWF |
| | | LATVGVSPDHQGKGLGSAVVLPGVEAAERAGVPAFLETSAPRNLPFY |
| | | ERLGFTVTADVEVPEGPRTWCMTRKPGA* |
| 86 | SB06650 | MALPVTALLLPLALLLHAARPQVQLVQSGAEVKKPGESLKISCKGSG |
| | CD8ss aHer2 | YSFTSYWIAWVRQMPGKGLEYMGLIYPGDSDTKYSPSFQGQVTISVD |
| | V5 CD8h | KSVSTAYLQWSSLKPSDSAVYFCARHDVGYCTDRTCAKWPEYFQHWG |
| | IRTA4tm | QGTLVTVSSGGGGSGGGGSGGGGSQSVLTQPPSVSAAPGQKVTISCS |
| | IRTA4icd | GSSSNIGNNYVSWYQQLPGTAPKLLIYDHTNRPAGVPDRESGSKSGT |
| | P2A PuroR | SASLAISGFRSEDEADYYCASWDYTLSGWVFGGGTKLTVLGGKPIPN |
| | | PLLGLDSTNGAATTTPAPRPPTPAPTIALQPLSLRPEACRPAAGGAV |
| | | HTRGLDFACDLWGLFGVLGFTGVALLLYALFHKISGESSATNEPRGA |
| | | SRPNPQEFTYSSPTPDMEELQPVYVNVGSVDVDVVYSQVWSMQQPES |
| | | SANIRTLLENKDSQVIYSSVKKSGSGATNFSLLKQAGDVEENPGPMT |
| | | EYKPTVRLATRDDVPRAVRTLAAAFADYPATRHTVDPDRHIERVTEL |
| | | QELFLTRVGLDIGKVWVADDGAAVAVWTTPESVEAGAVFAEIGPRMA |
| | | ELSGSRLAAQQQMEGLLAPHRPKEPAWFLATVGVSPDHQGKGLGSAV |
| | | VLPGVEAAERAGVPAFLETSAPRNLPFYERLGFTVTADVEVPEGPRT |
| | | WCMTRKPGA* |
| 87 | SB06651 | MALPVTALLLPLALLLHAARPQVQLVQSGAEVKKPGESLKISCKGSG |
| | CD8ss aHer2 | YSFTSYWIAWVRQMPGKGLEYMGLIYPGDSDTKYSPSFQGQVTISVD |
| | V5 CD8h | KSVSTAYLQWSSLKPSDSAVYFCARHDVGYCTDRTCAKWPEYFQHWG |
| | NKIRtm | QGTLVTVSSGGGGSGGGGSGGGGSQSVLTQPPSVSAAPGQKVTISCS |
| | NKIRicd | GSSSNIGNNYVSWYQQLPGTAPKLLIYDHTNRPAGVPDRESGSKSGT |
| | P2A PuroR | SASLAISGFRSEDEADYYCASWDYTLSGWVFGGGTKLTVLGGKPIPN |
| | | PLLGLDSTNGAATTTPAPRPPTPAPTIALQPLSLRPEACRPAAGGAV |
| | | HTRGLDFACDVLLPLIFTILLLLLVAASLLAWRMMKYQQKAAGMSPE |
| | | QVLQPLEGDLCYADLTLQLAGTSPQKATTKLSSAQVDQVEVEYVTMA |
| | | SLPKEDISYASLTLGAEDQEPTYCNMGHLSSHLPGRGPEEPTEYSTI |
| | | SRPGSGATNFSLLKQAGDVEENPGPMTEYKPTVRLATRDDVPRAVRT |
| | | LAAAFADYPATRHTVDPDRHIERVTELQELFLTRVGLDIGKVWVADD |
| | | GAAVAVWTTPESVEAGAVFAEIGPRMAELSGSRLAAQQQMEGLLAPH |
| | | RPKEPAWFLATVGVSPDHQGKGLGSAVVLPGVEAAERAGVPAFLETS |
| | | APRNLPFYERLGFTVTADVEVPEGPRTWCMTRKPGA* |
| 88 | SB06652 | MALPVTALLLPLALLLHAARPQVQLVQSGAEVKKPGESLKISCKGSG |
| | CD8ss aHer2 | YSFTSYWIAWVRQMPGKGLEYMGLIYPGDSDTKYSPSFQGQVTISVD |
| | V5 CD8h | KSVSTAYLQWSSLKPSDSAVYFCARHDVGYCTDRTCAKWPEYFQHWG |
| | IL1RAPtm | QGTLVTVSSGGGGSGGGGSGGGGSQSVLTQPPSVSAAPGQKVTISCS |
| | IL1RAPicd | GSSSNIGNNYVSWYQQLPGTAPKLLIYDHTNRPAGVPDRESGSKSGT |
| | P2A PuroR | SASLAISGFRSEDEADYYCASWDYTLSGWVFGGGTKLTVLGGKPIPN |
| | | PLLGLDSTNGAATTTPAPRPPTPAPTIALQPLSLRPEACRPAAGGAV |
| | | HTRGLDFACDVLLVVILIVVYHVYWLEMVLFYRAHFGTDETILDGKE |
| | | YDIYVSYARNAEEEEFVLLTLRGVLENEFGYKLCIFDRDSLPGGIVT |
| | | DETLSFIQKSRRLLVVLSPNYVLQGTQALLELKAGLENMASRGNINV |
| | | ILVQYKAVKETKVKELKRAKTVLTVIKWKGEKSKYPQGRFWKQLQVA |
| | | MPVKKSPRRSSSDEQGLSYSSLKNVGSGATNFSLLKQAGDVEENPGP |
| | | MTEYKPTVRLATRDDVPRAVRTLAAAFADYPATRHTVDPDRHIERVT |
| | | ELQELFLTRVGLDIGKVWVADDGAAVAVWTTPESVEAGAVFAEIGPR |
| | | MAELSGSRLAAQQQMEGLLAPHRPKEPAWFLATVGVSPDHQGKGLGS |
| | | AVVLPGVEAAERAGVPAFLETSAPRNLPFYERLGFTVTADVEVPEGP |
| | | RTWCMTRKPGA* |
| 89 | SB07298 | MALPVTALLLPLALLLHAARPQVQLVQSGAEVKKPGESLKISCKGSG |
| | CD8ss PelB | YSFTSYWIAWVRQMPGKGLEYMGLIYPGDSDTKYSPSFQGQVTISVD |
| | aHer2 V5 | KSVSTAYLQWSSLKPSDSAVYFCARHDVGYCTDRTCAKWPEYFQHWG |
| | CD8h | QGTLVTVSSGGGGSGGGGSGGGGSQSVLTQPPSVSAAPGQKVTISCS |
| | PTPRO P2A | GSSSNIGNNYVSWYQQLPGTAPKLLIYDHTNRPAGVPDRFSGSKSGT |
| | PuroR | SASLAISGFRSEDEADYYCASWDYTLSGWVFGGGTKLTVLGGKPIPN |
| | | PLLGLDSTNGAATTTPAPRPPTPAPTIALQPLSLRPEACRPAAGGAV |
| | | HTRGLDFACDISVLAILSTLLIGLLLVTLIILRKKHLQMARECGAGT |
| | | FVNFASLERDGKLPYNWRRSIFAFLTLLPSCLWTDYLLAFYINPWSK |
| | | NGLKKRKLTNPVQLDDFDAYIKDMAKDSDYKFSLQFEELKLIGLDIP |
| | | HFAADLPLNRCKNRYTNILPYDFSRVRLVSMNEEEGADYINANYIPG |
| | | YNSPQEYIATQGPLPETRNDFWKMVLQQKSQIIVMLTQCNEKRRVKC |
| | | DHYWPFTEEPIAYGDITVEMISEEEQDDWACRHFRINYADEMQDVMH |
| | | FNYTAWPDHGVPTANAAESILQFVHMVRQQATKSKGPMIIHCSAGVG |
| | | RTGTFIALDRLLQHIRDHEFVDILGLVSEMRSYRMSMVQTEEQYIFI |
| | | HQCVQLMWMKKKQQFCISDVIYENVSKSGSGATNFSLLKQAGDVEEN |
| | | PGPMTEYKPTVRLATRDDVPRAVRTLAAAFADYPATRHTVDPDRHIE |
| | | RVTELQELFLTRVGLDIGKVWVADDGAAVAVWTTPESVEAGAVFAEI |
| | | GPRMAELSGSRLAAQQQMEGLLAPHRPKEPAWFLATVGVSPDHQGKG |
| | | LGSAVVLPGVEAAERAGVPAFLETSAPRNLPFYERLGFTVTADVEVP |
| | | EGPRTWCMTRKPGA* |
| 90 | SB07299 | MALPVTALLLPLALLLHAARPQVQLVQSGAEVKKPGESLKISCKGSG |
| | CD8ss PelB | YSFTSYWIAWVRQMPGKGLEYMGLIYPGDSDTKYSPSFQGQVTISVD |
| | aHer2 V5 | KSVSTAYLQWSSLKPSDSAVYFCARHDVGYCTDRTCAKWPEYFQHWG |
| | CD8h | QGTLVTVSSGGGGSGGGGSGGGGSQSVLTQPPSVSAAPGQKVTISCS |
| | PTPRZ1 | GSSSNIGNNYVSWYQQLPGTAPKLLIYDHTNRPAGVPDRFSGSKSGT |
| | P2A PuroR | SASLAISGFRSEDEADYYCASWDYTLSGWVFGGGTKLTVLGGKPIPN |
| | | PLLGLDSTNGAATTTPAPRPPTPAPTIALQPLSLRPEACRPAAGGAV |
| | | HTRGLDFACDAVIPLVIVSALTFICLVVLVGILIYWRKCFQTAHFYL |
| | | EDSTSPRVISTPPTPIFPISDDVGAIPIKHFPKHVADLHASSGFTEE |
| | | FETLKEFYQEVQSCTVDLGITADSSNHPDNKHKNRYINIVAYDHSRV |
| | | KLAQLAEKDGKLTDYINANYVDGYNRPKAYIAAQGPLKSTAEDFWRM |
| | | IWEHNVEVIVMITNLVEKGRRKCDQYWPADGSEEYGNFLVTQKSVQV |
| | | LAYYTVRNFTLRNTKIKKGSQKGRPSGRVVTQYHYTQWPDMGVPEYS |
| | | LPVLTFVRKAAYAKRHAVGPVVVHCSAGVGRTGTYIVLDSMLQQIQH |
| | | EGTVNIFGFLKHIRSQRNYLVQTEEQYVFIHDTLVEAILSKETEVLD |
| | | SHIHAYVNALLIPGPAGKTKLEKQFQLLSQSNIQQSDYSAALKQCNR |
| | | EKNRTSSIIPVERSRVGISSLSGEGTDYINASYIMGYYQSNEFIITQ |
| | | HPLLHTIKDFWRMIWDHNAQLVVMIPDGQNMAEDEFVYWPNKDEPIN |
| | | CESFKVTLMAEEHKCLSNEEKLIIQDFILEATQDDYVLEVRHFQCPK |
| | | WPNPDSPISKTFELISVIKEEAANRDGPMIVHDEHGGVTAGTFCALT |
| | | TLMHQLEKENSVDVYQVAKMINLMRPGVFADIEQYQFLYKVILSLVS |
| | | TRQEENPSTSLDSNGAALPDGNIAESLESLVGSGATNFSLLKQAGDV |
| | | EENPGPMTEYKPTVRLATRDDVPRAVRTLAAAFADYPATRHTVDPDR |
| | | HIERVTELQELFLTRVGLDIGKVWVADDGAAVAVWTTPESVEAGAVF |
| | | AEIGPRMAELSGSRLAAQQQMEGLLAPHRPKEPAWFLATVGVSPDHQ |
| | | GKGLGSAVVLPGVEAAERAGVPAFLETSAPRNLPFYERLGFTVTADV |
| | | EVPEGPRTWCMTRKPGA* |
| 91 | SB07300 | MALPVTALLLPLALLLHAARPQVQLVQSGAEVKKPGESLKISCKGSG |
| | CD8ss PelB | YSFTSYWIAWVRQMPGKGLEYMGLIYPGDSDTKYSPSFQGQVTISVD |
| | aHer2 V5 | KSVSTAYLQWSSLKPSDSAVYFCARHDVGYCTDRTCAKWPEYFQHWG |
| | CD8h TLT1 | QGTLVTVSSGGGGSGGGGSGGGGSQSVLTQPPSVSAAPGQKVTISCS |
| | P2A PuroR | GSSSNIGNNYVSWYQQLPGTAPKLLIYDHTNRPAGVPDRESGSKSGT |
| | | SASLAISGFRSEDEADYYCASWDYTLSGWVFGGGTKLTVLGGKPIPN |
| | | PLLGLDSTNGAATTTPAPRPPTPAPTIALQPLSLRPEACRPAAGGAV |
| | | HTRGLDFACDLIWGAVLLVGLLVAAVVLFAVMAKRKQGNRLGVCGRF |
| | | LSSRVSGMNPSSVVHHVSDSGPAAELPLDVPHIRLDSPPSFDNTTYT |
| | | SLPLDSPSGKPSLPAPSSLPPLPPKVLVCSKPVTYATVIFPGGNKGG |
| | | GTSCGPAQNPPNNQTPSSGSGATNFSLLKQAGDVEENPGPMTEYKPT |
| | | VRLATRDDVPRAVRTLAAAFADYPATRHTVDPDRHIERVTELQELFL |
| | | TRVGLDIGKVWVADDGAAVAVWTTPESVEAGAVFAEIGPRMAELSGS |
| | | RLAAQQQMEGLLAPHRPKEPAWFLATVGVSPDHQGKGLGSAVVLPGV |
| | | EAAERAGVPAFLETSAPRNLPFYERLGFTVTADVEVPEGPRTWCMTR |
| | | KPGA* |
| 92 | SB07317 | MALPVTALLLPLALLLHAARPQVQLVQSGAEVKKPGESLKISCKGSG |
| | CD8ss PelB | YSFTSYWIAWVRQMPGKGLEYMGLIYPGDSDTKYSPSFQGQVTISVD |
| | aHer2 V5 | KSVSTAYLQWSSLKPSDSAVYFCARHDVGYCTDRTCAKWPEYFQHWG |
| | CD8h | QGTLVTVSSGGGGSGGGGSGGGGSQSVLTQPPSVSAAPGQKVTISCS |
| | SLAMF1 | GSSSNIGNNYVSWYQQLPGTAPKLLIYDHTNRPAGVPDRESGSKSGT |
| | P2A PuroR | SASLAISGFRSEDEADYYCASWDYTLSGWVFGGGTKLTVLGGKPIPN |
| | | PLLGLDSTNGAATTTPAPRPPTPAPTIALQPLSLRPEACRPAAGGAV |
| | | HTRGLDFACDWAVYAGLLGGVIMILIMVVILQLRRRGKTNHYQTTVE |
| | | KKSLTIYAQVQKPGPLQKKLDSFPAQDPCTTIYVAATEPVPESVQET |
| | | NSITVYASVTLPESGSGATNFSLLKQAGDVEENPGPMTEYKPTVRLA |
| | | TRDDVPRAVRTLAAAFADYPATRHTVDPDRHIERVTELQELFLTRVG |
| | | LDIGKVWVADDGAAVAVWTTPESVEAGAVFAEIGPRMAELSGSRLAA |
| | | QQQMEGLLAPHRPKEPAWFLATVGVSPDHQGKGLGSAVVLPGVEAAE |
| | | RAGVPAFLETSAPRNLPFYERLGFTVTADVEVPEGPRTWCMTRKPGA |
| 93 | SB07318 | MALPVTALLLPLALLLHAARPQVQLVQSGAEVKKPGESLKISCKGSG |
| | CD8ss PelB | YSFTSYWIAWVRQMPGKGLEYMGLIYPGDSDTKYSPSFQGQVTISVD |
| | aHer2 V5 | KSVSTAYLQWSSLKPSDSAVYFCARHDVGYCTDRTCAKWPEYFQHWG |
| | CD8h | QGTLVTVSSGGGGSGGGGSGGGGSQSVLTQPPSVSAAPGQKVTISCS |
| | SLAMF5 | GSSSNIGNNYVSWYQQLPGTAPKLLIYDHTNRPAGVPDRFSGSKSGT |
| | P2A PuroR | SASLAISGFRSEDEADYYCASWDYTLSGWVFGGGTKLTVLGGKPIPN |
| | | PLLGLDSTNGAATTTPAPRPPTPAPTIALQPLSLRPEACRPAAGGAV |
| | | HTRGLDFACDLLSVLAMFFLLVLILSSVFLFRLFKRRQGRIFPEGSC |
| | | LNTFTKNPYAASKKTIYTYIMASRNTQPAESRIYDEILQSKVLPSKE |
| | | EPVNTVYSEVQFADKMGKASTQDSKPPGTSSYEIVIGSGATNFSLLK |
| | | QAGDVEENPGPMTEYKPTVRLATRDDVPRAVRTLAAAFADYPATRHT |
| | | VDPDRHIERVTELQELFLTRVGLDIGKVWVADDGAAVAVWTTPESVE |
| | | AGAVFAEIGPRMAELSGSRLAAQQQMEGLLAPHRPKEPAWFLATVGV |
| | | SPDHQGKGLGSAVVLPGVEAAERAGVPAFLETSAPRNLPFYERLGFT |
| | | VTADVEVPEGPRTWCMTRKPGA |
| 94 | Axl-CD8z | METDTLLLWVLLLWVPGSTGAGGSDYKDDDDKGGSQVQLQESGPGLV |
| | | KPSETLSLTCTVSGYSITSNYWGWIRQPPGKGLEWMGYITYSGSTSY |
| | | NPSLKSRITISRDTSKNQFSLKLSSVTAADTAVYYCAITTFYYWGQG |
| | | TLVTVSSGGGGSGGGGSGGGGSDIQMTQSPSSLSASVGDRVTITCRA |
| | | SQDIGNYLRWFQQKPGKAPKLLISGATNLAAGVPSRFSGSGSGSDFT |
| | | LTISSLQPEDFATYYCLQSKESPWTFGQGTKVEIKRTTTTPAPRPPT |
| | | PAPTIALQPLSLRPEACRPAAGGAVHTRGLDFACDFWVLVVVGGVLA |
| | | CYSLLVTVAFIIFWVRSKRSRLLHSDYMNMTPRRPGPTRKHYQPYAP |
| | | PRDFAAYRSRVKFSRSADAPAYKQGQNQLYNELNLGRREEYDVLDKR |
| | | RGRDPEMGGKPRRKNPQEGLYNELQKDKMAEAYSEIGMKGERRRGKG |
| | | HDGLYQGLSTATKDTYDALHMQALPPR |
| 185 | SB08225 | MALPVTALLLPLALLLHAARPKYLLPTAAAGLLLLAAQPAMAQVQLV |
| | aHer2 V5 | QSGAEVKKPGESLKISCKGSGYSFTSYWIAWVRQMPGKGLEYMGLIY |
| | CD8h | PGDSDTKYSPSFQGQVTISVDKSVSTAYLQWSSLKPSDSAVYFCARH |
| | PCDHGC3 | DVGYCTDRTCAKWPEYFQHWGQGTLVTVSSGGGGSGGGGSGGGGSQS |
| | | VLTQPPSVSAAPGQKVTISCSGSSSNIGNNYVSWYQQLPGTAPKLLI |
| | | YDHTNRPAGVPDRFSGSKSGTSASLAISGFRSEDEADYYCASWDYTL |
| | | SGWVFGGGTKLTVLGGKPIPNPLLGLDSTNGAATTTPAPRPPTPAPT |
| | | IALQPLSLRPEACRPAAGGAVHTRGLDFACDLLLSLILVSVGFVVTV |
| | | FGVIIFKVYKWKQSRDLYRAPVSSLYRTPGPSLHADAVRGGLMSPHL |
| | | YHQVYLTTDSRRSDPLLKKPGAASPLASRQNTLRSCDPVFYRQVLGA |
| | | ESAPPGQQAPPNTDWRFSQAQRPGTSGSQNGDDTGTWPNNQFDTEML |
| | | QAMILASASEAADGSSTLGGGAGTMGLSARYGPQFTLQHVPDYRQNV |
| | | YIPGSNATLTNAAGKRDGKAPAGGNGNKKKSGKKEKK |
| 186 | SB08227 | MALPVTALLLPLALLLHAARPKYLLPTAAAGLLLLAAQPAMAQVQLV |
| | aHer2 V5 | QSGAEVKKPGESLKISCKGSGYSFTSYWIAWVRQMPGKGLEYMGLIY |
| | CD8h LIFR | PGDSDTKYSPSFQGQVTISVDKSVSTAYLQWSSLKPSDSAVYFCARH |
| | | DVGYCTDRTCAKWPEYFQHWGQGTLVTVSSGGGGSGGGGSGGGGSQS |
| | | VLTQPPSVSAAPGQKVTISCSGSSSNIGNNYVSWYQQLPGTAPKLLI |
| | | YDHTNRPAGVPDRESGSKSGTSASLAISGFRSEDEADYYCASWDYTL |
| | | SGWVFGGGTKLTVLGGKPIPNPLLGLDSTNGAATTTPAPRPPTPAPT |
| | | IALQPLSLRPEACRPAAGGAVHTRGLDFACDVGLIIAILIPVAVAVI |
| | | VGVVTSILCYRKREWIKETFYPDIPNPENCKALQFQKSVCEGSSALK |
| | | TLEMNPCTPNNVEVLETRSAFPKIEDTEIISPVAERPEDRSDAEPEN |
| | | HVVVSYCPPIIEEEIPNPAADEAGGTAQVIYIDVQSMYQPQAKPEEE |
| | | QENDPVGGAGYKPQMHLPINSTVEDIAAEEDLDKTAGYRPQANVNTW |
| | | NLVSPDSPRSIDSNSEIVSFGSPCSINSRQFLIPPKDEDSPKSNGGG |
| | | WSFTNFFQNKPND |
| 187 | SB08229 | MALPVTALLLPLALLLHAARPKYLLPTAAAGLLLLAAQPAMAQVQLV |
| | aHer2 V5 | QSGAEVKKPGESLKISCKGSGYSFTSYWIAWVRQMPGKGLEYMGLIY |
| | CD8h | PGDSDTKYSPSFQGQVTISVDKSVSTAYLQWSSLKPSDSAVYFCARH |
| | ERMAP | DVGYCTDRTCAKWPEYFQHWGQGTLVTVSSGGGGSGGGGSGGGGSQS |
| | | VLTQPPSVSAAPGQKVTISCSGSSSNIGNNYVSWYQQLPGTAPKLLI |
| | | YDHTNRPAGVPDRFSGSKSGTSASLAISGFRSEDEADYYCASWDYTL |
| | | SGWVFGGGTKLTVLGGKPIPNPLLGLDSTNGAATTTPAPRPPTPAPT |
| | | IALQPLSLRPEACRPAAGGAVHTRGLDFACDVALAVILPVLVLLIMV |
| | | CLCLIWKQRRAKEKLLYEHVTEVDNLLSDHAKEKGKLHKAVKKLRSE |
| | | LKLKRAAANSGWRRARLHFVAVTLDPDTAHPKLILSEDQRCVRLGDR |
| | | RQPVPDNPQRFDFVVSILGSEYFTTGCHYWEVYVGDKTKWILGVCSE |
| | | SVSRKGKVTASPANGHWLLRQSRGNEYEALTSPQTSFRLKEPPRCVG |
| | | IFLDYEAGVISFYNVTNKSHIFTFTHNFSGPLRPFFEPCLHDGGKNT |
| | | APLVICSELHKSEESIVPRPEGKGHANGDVSLKVNSSLLPPKAPELK |
| | | DIILSLPPDLGPALQELKAPSF |
| 188 | SB08230 | MALPVTALLLPLALLLHAARPKYLLPTAAAGLLLLAAQPAMAQVQLV |
| | aHer2 V5 | QSGAEVKKPGESLKISCKGSGYSFTSYWIAWVRQMPGKGLEYMGLIY |
| | CD8h | PGDSDTKYSPSFQGQVTISVDKSVSTAYLQWSSLKPSDSAVYFCARH |
| | IL1RAPL2 | DVGYCTDRTCAKWPEYFQHWGQGTLVTVSSGGGGSGGGGSGGGGSQS |
| | | VLTQPPSVSAAPGQKVTISCSGSSSNIGNNYVSWYQQLPGTAPKLLI |
| | | YDHTNRPAGVPDRFSGSKSGTSASLAISGFRSEDEADYYCASWDYTL |
| | | SGWVFGGGTKLTVLGGKPIPNPLLGLDSTNGAATTTPAPRPPTPAPT |
| | | IALQPLSLRPEACRPAAGGAVHTRGLDFACDIELAGGLGAIFLLLVL |
| | | LVVIYKCYNIELMLFYRQHFGADETNDDNKEYDAYLSYTKVDQDTLD |
| | | CDNPEEEQFALEVLPDVLEKHYGYKLFIPERDLIPSGTYMEDLTRYV |
| | | EQSRRLIIVLTPDYILRRGWSIFELESRLHNMLVSGEIKVILIECTE |
| | | LKGKVNCQEVESLKRSIKLLSLIKWKGSKSSKLNSKFWKHLVYEMPI |
| | | KKKEMLPRCHVLDSAEQGLFGELQPIPSIAMTSTSATLVSSQADLPE |
| | | FHPSDSMQIRHCCRGYKHEIPATTLPVPSLGNHHTYCNLPLTLLNGQ |
| | | LPLNNTLKDTQEFHRNSSLLPLSSKELSFTSDIW |
| 189 | SB08231 | MALPVTALLLPLALLLHAARPKYLLPTAAAGLLLLAAQPAMAQVQLV |
| | aHer2 V5 | QSGAEVKKPGESLKISCKGSGYSFTSYWIAWVRQMPGKGLEYMGLIY |
| | CD8h CDH5 | PGDSDTKYSPSFQGQVTISVDKSVSTAYLQWSSLKPSDSAVYFCARH |
| | | DVGYCTDRTCAKWPEYFQHWGQGTLVTVSSGGGGSGGGGSGGGGSQS |
| | | VLTQPPSVSAAPGQKVTISCSGSSSNIGNNYVSWYQQLPGTAPKLLI |
| | | YDHTNRPAGVPDRFSGSKSGTSASLAISGFRSEDEADYYCASWDYTL |
| | | SGWVFGGGTKLTVLGGKPIPNPLLGLDSTNGAATTTPAPRPPTPAPT |
| | | IALQPLSLRPEACRPAAGGAVHTRGLDFACDAVVAILLCILTITVIT |
| | | LLIFLRRRLRKQARAHGKSVPEIHEQLVTYDEEGGGEMDTTSYDVSV |
| | | LNSVRRGGAKPPRPALDARPSLYAQVQKPPRHAPGAHGGPGEMAAMI |
| | | EVKKDEADHDGDGPPYDTLHIYGYEGSESIAESLSSLGTDSSDSDVD |
| | | YDFLNDWGPRFKMLAELYGSDPREELLY |
| 190 | SB08232 | MALPVTALLLPLALLLHAARPKYLLPTAAAGLLLLAAQPAMAQVQLV |
| | aHer2 V5 | QSGAEVKKPGESLKISCKGSGYSFTSYWIAWVRQMPGKGLEYMGLIY |
| | CD8h | PGDSDTKYSPSFQGQVTISVDKSVSTAYLQWSSLKPSDSAVYFCARH |
| | MPZL1 | DVGYCTDRTCAKWPEYFQHWGQGTLVTVSSGGGGSGGGGSGGGGSQS |
| | | VLTQPPSVSAAPGQKVTISCSGSSSNIGNNYVSWYQQLPGTAPKLLI |
| | | YDHTNRPAGVPDRFSGSKSGTSASLAISGFRSEDEADYYCASWDYTL |
| | | SGWVFGGGTKLTVLGGKPIPNPLLGLDSTNGAATTTPAPRPPTPAPT |
| | | IALQPLSLRPEACRPAAGGAVHTRGLDFACDFPVWVVVGIVTAVVLG |
| | | LTLLISMILAVLYRRKNSKRDYTGCSTSESLSPVKQAPRKSPSDTEG |
| | | LVKSLPSGSHQGPVIYAQLDHSGGHHSDKINKSESVVYADIRKN |
| 191 | SB08233 | MALPVTALLLPLALLLHAARPKYLLPTAAAGLLLLAAQPAMAQVQLV |
| | aHer2 V5 | QSGAEVKKPGESLKISCKGSGYSFTSYWIAWVRQMPGKGLEYMGLIY |
| | CD8h MPZ | PGDSDTKYSPSFQGQVTISVDKSVSTAYLQWSSLKPSDSAVYFCARH |
| | | DVGYCTDRTCAKWPEYFQHWGQGTLVTVSSGGGGSGGGGSGGGGSQS |
| | | VLTQPPSVSAAPGQKVTISCSGSSSNIGNNYVSWYQQLPGTAPKLLI |
| | | YDHTNRPAGVPDRFSGSKSGTSASLAISGFRSEDEADYYCASWDYTL |
| | | SGWVFGGGTKLTVLGGKPIPNPLLGLDSTNGAATTTPAPRPPTPAPT |
| | | IALQPLSLRPEACRPAAGGAVHTRGLDFACDYGVVLGAVIGGVLGVV |
| | | LLLLLLFYVVRYCWLRRQAALQRRLSAMEKGKLHKPGKDASKRGRQT |
| | | PVLYAMLDHSRSTKAVSEKKAKGLGESRKDKK |
| 192 | SB08235 | MALPVTALLLPLALLLHAARPKYLLPTAAAGLLLLAAQPAMAQVQLV |
| | aHer2 V5 | QSGAEVKKPGESLKISCKGSGYSFTSYWIAWVRQMPGKGLEYMGLIY |
| | CD8h | PGDSDTKYSPSFQGQVTISVDKSVSTAYLQWSSLKPSDSAVYFCARH |
| | FCGR2B | DVGYCTDRTCAKWPEYFQHWGQGTLVTVSSGGGGSGGGGSGGGGSQS |
| | | VLTQPPSVSAAPGQKVTISCSGSSSNIGNNYVSWYQQLPGTAPKLLI |
| | | YDHTNRPAGVPDRFSGSKSGTSASLAISGFRSEDEADYYCASWDYTL |
| | | SGWVFGGGTKLTVLGGKPIPNPLLGLDSTNGAATTTPAPRPPTPAPT |
| | | IALQPLSLRPEACRPAAGGAVHTRGLDFACDSSSPMGIIVAVVTGIA |
| | | VAAIVAAVVALIYCRKKRISALPGYPECREMGETLPEKPANPTNPDE |
| | | ADKVGAENTITYSLLMHPDALEEPDDQNRI |
| 193 | SB08237 | MALPVTALLLPLALLLHAARPKYLLPTAAAGLLLLAAQPAMAQVQLV |
| | aHer2 V5 | QSGAEVKKPGESLKISCKGSGYSFTSYWIAWVRQMPGKGLEYMGLIY |
| | CD8h | PGDSDTKYSPSFQGQVTISVDKSVSTAYLQWSSLKPSDSAVYFCARH |
| | SIGLEC-6 | DVGYCTDRTCAKWPEYFQHWGQGTLVTVSSGGGGSGGGGSGGGGSQS |
| | | VLTQPPSVSAAPGQKVTISCSGSSSNIGNNYVSWYQQLPGTAPKLLI |
| | | YDHTNRPAGVPDRESGSKSGTSASLAISGFRSEDEADYYCASWDYTL |
| | | SGWVFGGGTKLTVLGGKPIPNPLLGLDSTNGAATTTPAPRPPTPAPT |
| | | IALQPLSLRPEACRPAAGGAVHTRGLDFACDLGAVWGASITTLVFLC |
| | | VCFIFRVKTRRKKAAQPVQNTDDVNPVMVSGSRGHQHQFQTGIVSDH |
| | | PAEAGPISEDEQELHYAVLHFHKVQPQEPKVTDTEYSEIKIHK |
| 194 | SB08238 | MALPVTALLLPLALLLHAARPKYLLPTAAAGLLLLAAQPAMAQVQLV |
| | aHer2 V5 | QSGAEVKKPGESLKISCKGSGYSFTSYWIAWVRQMPGKGLEYMGLIY |
| | CD8h | PGDSDTKYSPSFQGQVTISVDKSVSTAYLQWSSLKPSDSAVYFCARH |
| | MPIG6B | DVGYCTDRTCAKWPEYFQHWGQGTLVTVSSGGGGSGGGGSGGGGSQS |
| | | VLTQPPSVSAAPGQKVTISCSGSSSNIGNNYVSWYQQLPGTAPKLLI |
| | | YDHTNRPAGVPDRFSGSKSGTSASLAISGFRSEDEADYYCASWDYTL |
| | | SGWVFGGGTKLTVLGGKPIPNPLLGLDSTNGAATTTPAPRPPTPAPT |
| | | IALQPLSLRPEACRPAAGGAVHTRGLDFACDLLIPLLGAGLVLGLGA |
| | | LGLVWWLHRRLPPQPIRPLPRFAPLVKTEPQRPVKEEEPKIPGDLDQ |
| | | EPSLLYADLDHLALSRPRRLSTADPADASTIYAVVV |
| 195 | SB08239 | MALPVTALLLPLALLLHAARPKYLLPTAAAGLLLLAAQPAMAQVQLV |
| | aHer2 V5 | QSGAEVKKPGESLKISCKGSGYSFTSYWIAWVRQMPGKGLEYMGLIY |
| | CD8h | PGDSDTKYSPSFQGQVTISVDKSVSTAYLQWSSLKPSDSAVYFCARH |
| | VSIG4 | DVGYCTDRTCAKWPEYFQHWGQGTLVTVSSGGGGSGGGGSGGGGSQS |
| | | VLTQPPSVSAAPGQKVTISCSGSSSNIGNNYVSWYQQLPGTAPKLLI |
| | | YDHTNRPAGVPDRFSGSKSGTSASLAISGFRSEDEADYYCASWDYTL |
| | | SGWVFGGGTKLTVLGGKPIPNPLLGLDSTNGAATTTPAPRPPTPAPT |
| | | IALQPLSLRPEACRPAAGGAVHTRGLDFACDVFAIILIISLCCMVVF |
| | | TMAYIMLCRKTSQQEHVYEAARAHAREANDSGETMRVAIFASGCSSD |
| | | EPTSQNLGNNYSDEPCIGQEYQIIAQINGNYARLLDTVPLDYEFLAT |
| | | EGKSVC |
| 196 | SB08241 | MALPVTALLLPLALLLHAARPKYLLPTAAAGLLLLAAQPAMAQVQLV |
| | aHer2 V5 | QSGAEVKKPGESLKISCKGSGYSFTSYWIAWVRQMPGKGLEYMGLIY |
| | CD8h | PGDSDTKYSPSFQGQVTISVDKSVSTAYLQWSSLKPSDSAVYFCARH |
| | SIGLEC-12 | DVGYCTDRTCAKWPEYFQHWGQGTLVTVSSGGGGSGGGGSGGGGSQS |
| | | VLTQPPSVSAAPGQKVTISCSGSSSNIGNNYVSWYQQLPGTAPKLLI |
| | | YDHTNRPAGVPDRESGSKSGTSASLAISGFRSEDEADYYCASWDYTL |
| | | SGWVFGGGTKLTVLGGKPIPNPLLGLDSTNGAATTTPAPRPPTPAPT |
| | | IALQPLSLRPEACRPAAGGAVHTRGLDFACDFGGAGATALVFLYFCI |
| | | IFVVVRSCRKKSARPAVGVGDTGMEDANAVRGSASQGPLIESPADDS |
| | | PPHHAPPALATPSPEEGEIQYASLSFHKARPQYPQEQEAIGYEYSEI |
| | | NIPK |
| 197 | SB08738 | MALPVTALLLPLALLLHAARPQVQLVQSGAEVKKPGESLKISCKGSG |
| | aHer2 V5 | YSFTSYWIAWVRQMPGKGLEYMGLIYPGDSDTKYSPSFQGQVTISVD |
| | CD8h LIR8 | KSVSTAYLQWSSLKPSDSAVYFCARHDVGYCTDRTCAKWPEYFQHWG |
| | | QGTLVTVSSGGGGSGGGGSGGGGSQSVLTQPPSVSAAPGQKVTISCS |
| | | GSSSNIGNNYVSWYQQLPGTAPKLLIYDHTNRPAGVPDRESGSKSGT |
| | | SASLAISGFRSEDEADYYCASWDYTLSGWVFGGGTKLTVLGGKPIPN |
| | | PLLGLDSTNGAATTTPAPRPPTPAPTIALQPLSLRPEACRPAAGGAV |
| | | HTRGLDFACDVVTGVSVAFVLLLFLLLFLLLRHRHQSKHRTSAHFYR |
| | | PAGAAGPEPKDQGLQKRASPVADIQEEILNAAVKDTQPKDGVEMDAP |
| | | AAASEAPQDVTYAQLHSLTLRREATEPPPSQEREPPAEPSIYAPLAI |
| | | H |
| 198 | SB08739 | MALPVTALLLPLALLLHAARPQVQLVQSGAEVKKPGESLKISCKGSG |
| | aHer2 V5 | YSFTSYWIAWVRQMPGKGLEYMGLIYPGDSDTKYSPSFQGQVTISVD |
| | CD8h | KSVSTAYLQWSSLKPSDSAVYFCARHDVGYCTDRTCAKWPEYFQHWG |
| | IRTA1 | QGTLVTVSSGGGGSGGGGSGGGGSQSVLTQPPSVSAAPGQKVTISCS |
| | | GSSSNIGNNYVSWYQQLPGTAPKLLIYDHTNRPAGVPDRESGSKSGT |
| | | SASLAISGFRSEDEADYYCASWDYTLSGWVFGGGTKLTVLGGKPIPN |
| | | PLLGLDSTNGAATTTPAPRPPTPAPTIALQPLSLRPEACRPAAGGAV |
| | | HTRGLDFACDVAAGATGGLLSALLLAVALLFHCWRRRKSGVGFLGDE |
| | | TRLPPAPGPGESSHSICPAQVELQSLYVDVHPKKGDLVYSEIQTTQL |
| | | GEEEEANTSRTLLEDKDVSVVYSEVKTQHPDNSAGKISSKDEES |
| 199 | SB08747 | MALPVTALLLPLALLLHAARPQVQLVQSGAEVKKPGESLKISCKGSG |
| | aHer2 V5 | YSFTSYWIAWVRQMPGKGLEYMGLIYPGDSDTKYSPSFQGQVTISVD |
| | CD8h | KSVSTAYLQWSSLKPSDSAVYFCARHDVGYCTDRTCAKWPEYFQHWG |
| | KIR2DL4 | QGTLVTVSSGGGGSGGGGSGGGGSQSVLTQPPSVSAAPGQKVTISCS |
| | | GSSSNIGNNYVSWYQQLPGTAPKLLIYDHTNRPAGVPDRFSGSKSGT |
| | | SASLAISGFRSEDEADYYCASWDYTLSGWVFGGGTKLTVLGGKPIPN |
| | | PLLGLDSTNGAATTTPAPRPPTPAPTIALQPLSLRPEACRPAAGGAV |
| | | HTRGLDFACDAVIRYSVAIILFTILPFFLLHRWCSKKKDAAVMNQEP |
| | | AGHRTVNREDSDEQDPQEVTYAQLDHCIFTQRKITGPSQRSKRPSTD |
| | | TSVCIELPNAEPRALSPAHEHHSQALMGSSRETTALSQTQLASSNVP |
| | | AAGI |
| 200 | SB08748 | MALPVTALLLPLALLLHAARPQVQLVQSGAEVKKPGESLKISCKGSG |
| | aHer2 V5 | YSFTSYWIAWVRQMPGKGLEYMGLIYPGDSDTKYSPSFQGQVTISVD |
| | CD8h | KSVSTAYLQWSSLKPSDSAVYFCARHDVGYCTDRTCAKWPEYFQHWG |
| | KIR2DL5 | QGTLVTVSSGGGGSGGGGSGGGGSQSVLTQPPSVSAAPGQKVTISCS |
| | | GSSSNIGNNYVSWYQQLPGTAPKLLIYDHTNRPAGVPDRFSGSKSGT |
| | | SASLAISGFRSEDEADYYCASWDYTLSGWVFGGGTKLTVLGGKPIPN |
| | | PLLGLDSTNGAATTTPAPRPPTPAPTIALQPLSLRPEACRPAAGGAV |
| | | HTRGLDFACDLHILIGTSVAIILFIILFFFLLHCCCSNKKNAAVMDQ |
| | | EPAGDRTVNREDSDDQDPQEVTYAQLDHCVFTQTKITSPSQRPKTPP |
| | | TDTTMYMELPNAKPRSLSPAHKHHSQALRGSSRETTALSQNRVASSH |
| | | VPAAGI |
| 201 | SB08757 | MALPVTALLLPLALLLHAARPQVQLVQSGAEVKKPGESLKISCKGSG |
| | aHer2 V5 | YSFTSYWIAWVRQMPGKGLEYMGLIYPGDSDTKYSPSFQGQVTISVD |
| | CD8h | KSVSTAYLQWSSLKPSDSAVYFCARHDVGYCTDRTCAKWPEYFQHWG |
| | SIGLEC-7 | QGTLVTVSSGGGGSGGGGSGGGGSQSVLTQPPSVSAAPGQKVTISCS |
| | | GSSSNIGNNYVSWYQQLPGTAPKLLIYDHTNRPAGVPDRESGSKSGT |
| | | SASLAISGFRSEDEADYYCASWDYTLSGWVFGGGTKLTVLGGKPIPN |
| | | PLLGLDSTNGAATTTPAPRPPTPAPTIALQPLSLRPEACRPAAGGAV |
| | | HTRGLDFACDGAVGGAGATALVFLSFCVIFIVVRSCRKKSARPAADV |
| | | GDIGMKDANTIRGSASQGNLTESWADDNPRHHGLAAHSSGEEREIQY |
| | | APLSFHKGEPQDLSGQEATNNEYSEIKIPK |
| 202 | SB09038 | MALPVTALLLPLALLLHAARPQVQLVQSGAEVKKPGESLKISCKGSG |
| | aHer2 V5 | YSFTSYWIAWVRQMPGKGLEYMGLIYPGDSDTKYSPSFQGQVTISVD |
| | CD8h | KSVSTAYLQWSSLKPSDSAVYFCARHDVGYCTDRTCAKWPEYFQHWG |
| | FCRH3 | QGTLVTVSSGGGGSGGGGSGGGGSQSVLTQPPSVSAAPGQKVTISCS |
| | | GSSSNIGNNYVSWYQQLPGTAPKLLIYDHTNRPAGVPDRESGSKSGT |
| | | SASLAISGFRSEDEADYYCASWDYTLSGWVFGGGTKLTVLGGKPIPN |
| | | PLLGLDSTNGAATTTPAPRPPTPAPTIALQPLSLRPEACRPAAGGAV |
| | | HTRGLDFACDAAGITGLVLSILVLAAAAALLHYARARRKPGGLSATG |
| | | TSSHSPSECQEPSSSRPSRIDPQEPTHSKPLAPMELEPMYSNVNPGD |
| | | SNPIYSQIWSIQHTKENSANCPMMHQEHEELTVLYSELKKTHPDDSA |
| | | GEASSRGRAHEEDDEENYENVPRVLLASDH |
| 203 | SB09039 | MALPVTALLLPLALLLHAARPQVQLVQSGAEVKKPGESLKISCKGSG |
| | aHer2 V5 | YSFTSYWIAWVRQMPGKGLEYMGLIYPGDSDTKYSPSFQGQVTISVD |
| | CD8h | KSVSTAYLQWSSLKPSDSAVYFCARHDVGYCTDRTCAKWPEYFQHWG |
| | PCDHGC5 | QGTLVTVSSGGGGSGGGGSGGGGSQSVLTQPPSVSAAPGQKVTISCS |
| | | GSSSNIGNNYVSWYQQLPGTAPKLLIYDHTNRPAGVPDRESGSKSGT |
| | | SASLAISGFRSEDEADYYCASWDYTLSGWVFGGGTKLTVLGGKPIPN |
| | | PLLGLDSTNGAATTTPAPRPPTPAPTIALQPLSLRPEACRPAAGGAV |
| | | HTRGLDFACDLIVALATVSLLSLVTFTFLSAKCLQGNADGDGGGGQC |
| | | CRRQDSPSPDFYKQSSPNLQVSSDGTLKYMEVTLRPTDSQSHCYRTC |
| | | FSPASDGSDFTFLRPLSVQQPTALALEPDAIRSRSNTLRERSQQAPP |
| | | NTDWRFSQAQRPGTSGSQNGDDTGTWPNNQFDTEMLQAMILASASEA |
| | | ADGSSTLGGGAGTMGLSARYGPQFTLQHVPDYRQNVYIPGSNATLTN |
| | | AAGKRDGKAPAGGNGNKKKSGKKEKK |
| 204 | SB09040 | MALPVTALLLPLALLLHAARPQVQLVQSGAEVKKPGESLKISCKGSG |
| | aHer2 V5 | YSFTSYWIAWVRQMPGKGLEYMGLIYPGDSDTKYSPSFQGQVTISVD |
| | CD8h | KSVSTAYLQWSSLKPSDSAVYFCARHDVGYCTDRTCAKWPEYFQHWG |
| | CDH11 | QGTLVTVSSGGGGSGGGGSGGGGSQSVLTQPPSVSAAPGQKVTISCS |
| | | GSSSNIGNNYVSWYQQLPGTAPKLLIYDHTNRPAGVPDRESGSKSGT |
| | | SASLAISGFRSEDEADYYCASWDYTLSGWVFGGGTKLTVLGGKPIPN |
| | | PLLGLDSTNGAATTTPAPRPPTPAPTIALQPLSLRPEACRPAAGGAV |
| | | HTRGLDFACDGALIAILACIVILLVIVVLFVTLRRQKKEPLIVFEEE |
| | | DVRENIITYDDEGGGEEDTEAFDIATLQNPDGINGFIPRKDIKPEYQ |
| | | YMPRPGLRPAPNSVDVDDFINTRIQEADNDPTAPPYDSIQIYGYEGR |
| | | GSVAGSLSSLESATTDSDLDYDYLQNWGPRFKKLADLYGSKDTFDDD |
| | | S |
| 205 | SB09041 | MALPVTALLLPLALLLHAARPQVQLVQSGAEVKKPGESLKISCKGSG |
| | aHer2 V5 | YSFTSYWIAWVRQMPGKGLEYMGLIYPGDSDTKYSPSFQGQVTISVD |
| | CD8h | KSVSTAYLQWSSLKPSDSAVYFCARHDVGYCTDRTCAKWPEYFQHWG |
| | IMPG2 | QGTLVTVSSGGGGSGGGGSGGGGSQSVLTQPPSVSAAPGQKVTISCS |
| | | GSSSNIGNNYVSWYQQLPGTAPKLLIYDHTNRPAGVPDRFSGSKSGT |
| | | SASLAISGFRSEDEADYYCASWDYTLSGWVFGGGTKLTVLGGKPIPN |
| | | PLLGLDSTNGAATTTPAPRPPTPAPTIALQPLSLRPEACRPAAGGAV |
| | | HTRGLDFACDVIIGITIASVVGLLVIFSAIIYFFIRTLQAHHDRSER |
| | | ESPFSGSSRQPDSLSSIENAVKYNPVYESHRAGCEKYEGPYPQHPFY |
| | | SSASGDVIGGLSREEIRQMYESSELSREEIQERMRVLELYANDPEFA |
| | | AFVREQQVEEV |
| 206 | SB09042 | MALPVTALLLPLALLLHAARPQVQLVQSGAEVKKPGESLKISCKGSG |
| | aHer2 V5 | YSFTSYWIAWVRQMPGKGLEYMGLIYPGDSDTKYSPSFQGQVTISVD |
| | CD8h | KSVSTAYLQWSSLKPSDSAVYFCARHDVGYCTDRTCAKWPEYFQHWG |
| | DSCAM | QGTLVTVSSGGGGSGGGGSGGGGSQSVLTQPPSVSAAPGQKVTISCS |
| | | GSSSNIGNNYVSWYQQLPGTAPKLLIYDHTNRPAGVPDRFSGSKSGT |
| | | SASLAISGFRSEDEADYYCASWDYTLSGWVFGGGTKLTVLGGKPIPN |
| | | PLLGLDSTNGAATTTPAPRPPTPAPTIALQPLSLRPEACRPAAGGAV |
| | | HTRGLDFACDLVTISCILVGVLLLFVLLLVVRRRRREQRLKRLRDAK |
| | | SLAEMLMSKNTRTSDTLSKQQQTLRMHIDIPRAQLLIEERDTMETID |
| | | DRSTVLLTDADFGEAAKQKSLTVTHTVHYQSVSQATGPLVDVSDARP |
| | | GTNPTTRRNAKAGPTARNRYASQWTLNRPHPTISAHTLTTDWRLPTP |
| | | RAAGSVDKESDSYSVSPSQDTDRARSSMVSTESASSTYEELARAYEH |
| | | AKMEEQLRHAKFTITECFISDTSSEQLTAGTNEYTDSLTSSTPSESG |
| | | ICRFTASPPKPQDGGRVMNMAVPKAHRPGDLIHLPPYLRMDFLLNRG |
| | | GPGTSRDLSLGQACLEPQKSRTLKRPTVLEPIPMEAASSASSTREGQ |
| | | SWQPGAVATLPQREGAELGQAAKMSSSQESLLDSRGHLKGNNPYAKS |
| | | YTLV |

TABLE 10
iCAR Formats and Domains for NK Cells

| Construct | | Sequence | |
|---|---|---|---|
| (by ICD) | Domain | Type | Sequence |
| LIR1 | CD8 Signal | Amino Acid | MALPVTALLLPLALLLHAARP |
| TLT1 | Sequence | | (SEQ ID NO: 213) |
| TIM3 | | | |
| LAG3 | | | |
| LIR1-LAG3 | | | |
| LIR1-TIM3 | | | |
| SLAMF1 | | | |
| SLAMF5 | | | |
| PTPRO | | | |
| PTPRZ1 | | | |
| CEACAM-1 | | | |
| IRTA2 | | | |
| IRTA4 | | | |
| PECAM-1 | | | |
| NKIR | | | |
| IL1RAP | | | |
| LIR1 | CD8 Signal | Nucleotide | ATGGCCTTACCAGTGACCGCCTTGCTC |
| TLT1 | Sequence | | CTGCCGCTGGCCTTGCTGCTCCACGCC |
| TIM3 | | | GCCAGGCCG (SEQ ID NO: 214) |
| LAG3 | | | |
| LIR1-LAG3 | | | |
| LIR1-TIM3 | | | |
| SLAMF1 | | | |
| SLAMF5 | | | |
| PTPRO | | | |
| PTPRZ1 | | | |
| CEACAM-1 | | | |
| IRTA2 | | | |
| IRTA4 | | | |
| PECAM-1 | | | |
| NKIR | | | |
| IL1RAP | | | |
| PTPRO | PelB Signal | Amino Acid | KYLLPTAAAGLLLLAAQPAMA |
| PTPRZ1 | Sequence | | (SEQ ID NO: 215) |
| TLT1 | | | |
| SLAMF1 | | | |
| SLAMF5 | | | |
| PTPRO | PelB Signal | Nucleotide | AAATACCTATTGCCTACGGCAGCCGCT |
| PTPRZ1 | Sequence | | GGATTGTTATTACTCGCGGCCCAGCCG |
| TLT1 | | | GCCATGGCC (SEQ ID NO: 216) |
| SLAMF1 | | | |
| SLAMF5 | | | |
| LIR1 | iCAR-antigen | Amino Acid | QVQLVQSGAEVKKPGESLKISCKGSGY |
| TLT1 | specific scFv | | SFTSYWIAWVRQMPGKGLEYMGLIYPG |
| TIM3 | with (G4S)3 | | DSDTKYSPSFQGQVTISVDKSVSTAYL |
| LAG3 | linker | | QWSSLKPSDSAVYFCARHDVGYCTDRT |
| LIR1-LAG3 | | | CAKWPEYFQHWGQGTLVTVSSGGGGSG |
| LIR1-TIM3 | | | GGGSGGGGSQSVLTQPPSVSAAPGQKV |
| SLAMF1 | | | TISCSGSSSNIGNNYVSWYQQLPGTAP |
| SLAMF5 | | | KLLIYDHTNRPAGVPDRFSGSKSGTSA |
| PTPRO | | | SLAISGFRSEDEADYYCASWDYTLSGW |
| PTPRZ1 | | | VFGGGTKLTVLG (SEQ ID NO: |
| CEACAM-1 | | | 217) |
| IRTA2 | | | |
| IRTA4 | | | |
| PECAM-1 | | | |
| NKIR | | | |
| IL1RAP | | | |
| LIR1 | iCAR-antigen | Nucleotide | CAGGTGCAGCTGGTGCAGTCTGGGGCA |
| TLT1 | specific scFv | | GAGGTGAAAAAGCCCGGGGAGTCTCTG |
| TIM3 | with (G4S)3 | | AAGATCTCCTGTAAGGGTTCTGGATAC |
| LAG3 | linker | | AGCTTTACCAGCTACTGGATCGCCTGG |
| LIR1-LAG3 | | | GTGCGCCAGATGCCCGGGAAAGGCCTG |
| LIR1-TIM3 | | | GAGTACATGGGGCTCATCTATCCTGGT |
| SLAMF1 | | | GACTCTGACACCAAATACAGCCCGTCC |
| SLAMF5 | | | TTCCAAGGCCAGGTCACCATCTCAGTC |
| PTPRO | | | GACAAGTCCGTCAGCACTGCCTACTTG |
| PTPRZ1 | | | CAATGGAGCAGTCTGAAGCCCTCGGAC |
| CEACAM-1 | | | AGCGCCGTGTATTTTTGTGCGAGACAT |
| IRTA2 | | | GACGTGGGATATTGCACCGACCGGACT |
| IRTA4 | | | TGCGCAAAGTGGCCTGAATACTTCCAG |
| PECAM-1 | | | CATTGGGGCCAGGGCACCCTGGTCACC |
| NKIR | | | GTCTCCTCAGGTGGAGGCGGTTCAGGC |
| IL1RAP | | | GGAGGTGGCTCTGGCGGTGGCGGATCG |
| | | | CAGTCTGTGTTGACGCAGCCGCCCTCA |
| | | | GTGTCTGCGGCCCCAGGACAGAAGGTC |
| | | | ACCATCTCCTGCTCTGGAAGCAGCTCC |
| | | | AACATTGGGAATAATTATGTATCCTGG |
| | | | TACCAGCAGCTCCCAGGAACAGCCCCC |
| | | | AAACTCCTCATCTATGATCACACCAAT |
| | | | CGGCCCGCAGGGGTCCCTGACCGATTC |
| | | | TCTGGCTCCAAGTCTGGCACCTCAGCC |
| | | | TCCCTGGCCATCAGTGGGTTCCGGTCC |
| | | | GAGGATGAGGCTGATTATTACTGTGCC |
| | | | TCCTGGGACTACACCCTCTCGGGCTGG |
| | | | GTGTTCGGCGGAGGGACCAAGCTGACC |
| | | | GTCCTAGGT (SEQ ID NO: 218) |
| LIR1 | V5 tag | Amino Acid | GKPIPNPLLGLDSTNGAA |
| TLT1 | | | (SEQ ID NO: 219) |
| TIM3 | | | |
| LAG3 | | | |
| LIR1-LAG3 | | | |
| LIR1-TIM3 | | | |
| SLAMF1 | | | |
| SLAMF5 | | | |
| PTPRO | | | |
| PTPRZ1 | | | |
| CEACAM-1 | | | |
| IRTA2 | | | |
| IRTA4 | | | |
| PECAM-1 | | | |
| NKIR | | | |
| IL1RAP | | | |
| LIR1 | V5 tag | Nucleotide | GGGAAGCCTATCCCGAACCCTCTGTTG |
| TLT1 | | | GGTCTCGATAGTACCAATGGGGCCGCA |
| TIM3 | | | (SEQ ID NO: 220) |
| LAG3 | | | |
| LIR1-LAG3 | | | |
| LIR1-TIM3 | | | |
| SLAMF1 | | | |
| SLAMF5 | | | |
| PTPRO | | | |
| PTPRZ1 | | | |
| CEACAM-1 | | | |
| IRTA2 | | | |
| IRTA4 | | | |
| PECAM-1 | | | |
| NKIR | | | |
| IL1RAP | | | |
| LIR1 | CD8 hinge | Amino Acid | TTTPAPRPPTPAPTIALQPLSLRPEAC |
| TLT1 | | | RPAAGGAVHTRGLDFACD (SEQ ID |
| TIM3 | | | NO: 73) |
| LAG3 | | | |
| LIR1-LAG3 | | | |
| LIR1-TIM3 | | | |
| SLAMF1 | | | |
| SLAMF5 | | | |
| PTPRO | | | |
| PTPRZ1 | | | |
| CEACAM-1 | | | |
| IRTA2 | | | |
| IRTA4 | | | |
| PECAM-1 | | | |
| NKIR | | | |
| IL1RAP | | | |
| LIR1 | CD8 hinge | Nucleotide | ACCACGACGCCAGCGCCGCGACCACCA |
| TLT1 | | | ACACCGGCGCCCACCATCGCGTTGCAG |
| TIM3 | | | CCCCTGTCCCTGCGCCCAGAGGCGTGC |
| LAG3 | | | CGGCCAGCGGCGGGGGGCGCAGTGCAC |
| LIR1-LAG3 | | | ACGAGGGGGCTGGACTTCGCCTGTGAT |
| LIR1-TIM3 | | | (SEQ ID NO: 82) |
| SLAMF1 | | | |
| SLAMF5 | | | |
| PTPRO | | | |
| PTPRZ1 | | | |
| CEACAM-1 | | | |
| IRTA2 | | | |
| IRTA4 | | | |
| PECAM-1 | | | |
| NKIR | | | |
| IL1RAP | | | |
| PECAM-1 | PECAM-1 TM | Amino Acid | GLIAVVIIGVIIALLIIAA (SEQ ID |
| | Domain | | NO: 24) |
| PECAM-1 | PECAM-1 TM | Nucleotide | GGCCTGATCGCCGTGGTTATCATCGGC |
| | Domain | | GTGATCATTGCCCTGCTGATTATCGCC |
| | | | GCC (SEQ ID NO: 36) |
| LIR1 | LIR1 TM | Amino Acid | VIGILVA VILLLLLLLLLFLI (SEQ |
| | Domain | | ID NO: 25) |
| LIR1 | LIR1 TM | Nucleotide | GTTATAGGGATCCTGGTGGCTGTCATA |
| | Domain | | CTCCTCTTGCTCCTCTTGTTGCTGCTT |
| | | | TTTTTGATA (SEQ ID NO: 37) |
| IRTA2 | IRTA2 TM | Amino Acid | VAGGLLSIAGLAAGALLLYCW (SEQ |
| | Domain | | ID NO: 26) |
| IRTA2 | IRTA2 TM | Nucleotide | GTTGCTGGTGGCCTGCTGTCTATTGCT |
| | Domain | | GGACTTGCTGCTGGTGCTCTGCTGCTG |
| | | | TACTGCTGG (SEQ ID NO: 38) |
| IRTA4 | IRTA4 TM | Amino Acid | LWGLFGVLGFTGVALLLYALF (SEQ |
| | Domain | | ID NO: 27) |
| IRTA4 | IRTA4 TM | Nucleotide | CTGTGGGGACTGTTTGGCGTGTTGGGC |
| | Domain | | TTTACAGGCGTGGCCCTGCTGCTGTAC |
| | | | GCCCTGTTT (SEQ ID NO: 39) |
| NKIR | NKIR TM | Amino Acid | VLLPLIFTILLLLLVAASLLA (SEQ |
| | Domain | | ID NO: 28) |
| NKIR | NKIR TM | Nucleotide | GTTCTGCTGCCCCTGATCTTCACCATC |
| | Domain | | CTGTTGCTGCTGCTGGTGGCCGCTTCT |
| | | | CTGCTGGCT (SEQ ID NO: 40) |
| IL1RAP | IL1RAP TM | Amino Acid | VLLVVILIVVYHVYWLEMVLF (SEQ |
| | Domain | | ID NO: 29) |
| IL1RAP | IL1RAP TM | Nucleotide | GTGCTGCTGGTGGTCATCCTGATCGTG |
| | Domain | | GTGTACCACGTGTACTGGCTGGAAATG |
| | | | GTGCTGTTC (SEQ ID NO: 41) |
| PTPRO | PTPRO TM | Amino Acid | ISVLAILSTLLIGLLLVTLII (SEQ |
| | Domain | | ID NO: 30) |
| PTPRO | PTPRO TM | Nucleotide | ATCTCCGTGCTGGCCATCCTGAGCACA |
| | Domain | | CTGCTGATTGGACTGCTGCTCGTGACC |
| | | | CTGATCATC (SEQ ID NO: 42) |
| PTPRZ1 | PTPRZ1 TM | Amino Acid | AVIPLVIVSALTFICLVVLVGILIYW |
| | Domain | | (SEQ ID NO: 31) |
| PTPRZ1 | PTPRZ1TM | Nucleotide | GCCGTGATTCCTCTGGTTATCGTGTCT |
| | Domain | | GCCCTGACCTTCATCTGCCTGGTGGTG |
| | | | CTCGTGGGCATCCTGATCTATTGG |
| | | | (SEQ ID NO: 43) |
| TLT1 | TLT1 TM | Amino Acid | LIWGAVLLVGLLVAAVVLFAV (SEQ |
| | Domain | | ID NO: 32) |
| TLT1 | TLT1 TM | Nucleotide | CTGATCTGGGGAGCAGTGCTGCTTGTG |
| | Domain | | GGACTGCTGGTGGCTGCTGTGGTGCTG |
| | | | TTTGCCGTG (SEQ ID NO: 44) |
| SLAMF1 | SLAMF1 TM | Amino Acid | WAVYAGLLGGVIMILIMVVIL (SEQ |
| | Domain | | ID NO: 33) |
| SLAMF1 | SLAMF1 TM | Nucleotide | TGGGCCGTGTACGCAGGCCTGCTTGGC |
| | Domain | | GGTGTGATCATGATCCTGATTATGGTG |
| | | | GTCATCCTG (SEQ ID NO: 45) |
| SLAMF5 | SLAMF5 TM | Amino Acid | LLSVLAMFFLLVLILSSVFLF (SEQ |
| | Domain | | ID NO: 34) |
| SLAMF5 | SLAMF5 | Nucleotide | CTGCTGTCCGTCCTGGCCATGTTTTTT |
| | TM Domain | | CTGTTGGTACTCATTCTATCATCCGTG |
| | | | TTCCTGTTC (SEQ ID NO: 46) |
| PCDHGC3 | PCDHGC3 TM | Amino Acid | LLLSLILVSVGFVVTVFGVII (SEQ |
| | domain | | ID NO: 139) |
| PCDHGC3 | PCDHGC3 TM | Nucleotide | CTGCTGCTGTCTCTGATCCTGGTGTCC |
| | domain | | GTGGGCTTTGTGGTCACCGTGTTCGGC |
| | | | GTGATCATC (SEQ ID NO: 161) |
| LIFR | LIFR TM domain | Amino Acid | VGLIIAILIPVAVAVIVGVVTSILC |
| | | | (SEQ ID NO: 140) |
| LIFR | LIFR TM domain | Nucleotide | GTGGGACTGATCATTGCCATTCTGATC |
| | | | CCTGTGGCCGTGGCCGTGATTGTGGGC |
| | | | GTCGTGACATCCATCCTGTGC (SEQ |
| | | | ID NO: 162) |
| ERMAP | ERMAP TM | Amino Acid | VALAVILPVLVLLIMVCLCLI (SEQ |
| | domain | | ID NO: 141) |
| ERMAP | ERMAP TM | Nucleotide | GTGGCTCTGGCTGTGATTCTGCCTGTG |
| | domain | | CTGGTGCTGCTGATCATGGTTTGCCTG |
| | | | TGCCTGATC (SEQ ID NO: 163) |
| IL1RAPL2 | IL1RAPL2 TM | Amino Acid | IELAGGLGAIFLLLVLLVVIY (SEQ |
| | domain | | ID NO: 142) |
| IL1RAPL2 | IL1RAPL2 TM | Nucleotide | ATTGAACTGGCTGGCGGACTGGGCGCC |
| | domain | | ATCTTTCTGCTGCTGGTGCTGCTCGTG |
| | | | GTCATCTAC (SEQ ID NO: 164) |
| CDH5 | CDH5 TM | Amino Acid | AVVAILLCILTITVITLLIFL (SEQ |
| | domain | | ID NO: 143) |
| CDH5 | CDH5 TM | Nucleotide | GCCGTGGTTGCCATCCTGCTGTGTATC |
| | domain | | CTGACCATCACCGTGATTACCCTGCTG |
| | | | ATCTTCCTG (SEQ ID NO: 165) |
| MPZL1 | MPZL1 TM | Amino Acid | FPVWVVVGIVTAVVLGLTLLI (SEQ |
| | domain | | ID NO: 144) |
| MPZL1 | MPZL1 TM | Nucleotide | TTCCCTGTGTGGGTTGTCGTGGGAATC |
| | domain | | GTGACAGCTGTGGTGCTGGGACTGAC |
| | | | CCTGCTGATC (SEQ ID NO: 166) |
| MPZ | MPZ TM domain | Amino Acid | YGVVLGAVIGGVLGVVLLLLLLFYVV |
| | | | (SEQ ID NO: 145) |
| MPZ | MPZ TM domain | Nucleotide | TATGGCGTTGTGCTGGGAGCTGTGATT |
| | | | GGCGGAGTTCTGGGAGTTGTGCTGCTG |
| | | | CTCCTGCTGCTGTTTTACGTCGTG |
| | | | (SEQ ID NO: 167) |
| FCGR2B | FCGR2B TM | Amino Acid | SSSPMGIIVAVVTGIAVAAIVAA |
| | domain | | (SEQ ID NO: 146) |
| FCGR2B | FCGR2B TM | Nucleotide | AGCAGCTCTCCCATGGGCATCATTGTG |
| | domain | | GCCGTGGTCACAGGCATTGCCGTGGC |
| | | | CGCTATAGTGGCTGCT (SEQ ID |
| | | | NO: 168) |
| SIGLEC-6 | SIGLEC-6 TM | Amino Acid | LGAVWGASITTLVFLCVCFIF (SEQ |
| | domain | | ID NO: 147) |
| SIGLEC-6 | SIGLEC-6 TM | Nucleotide | CTGGGAGCTGTTTGGGGCGCCTCTATT |
| | domain | | ACAACCCTGGTGTTCCTGTGCGTGTGC |
| | | | TTCATCTTC (SEQ ID NO: 169) |
| MPIG6B | MPIG6B TM | Amino Acid | LLIPLLGAGLVLGLGALGLVW (SEQ |
| | domain | | ID NO: 148) |
| MPIG6B | MPIG6B TM | Nucleotide | CTGCTGATTCCTCTGCTTGGAGCCGGA |
| | domain | | CTGGTGCTTGGACTTGGAGCACTGGG |
| | | | ACTTGTGTGG (SEQ ID NO: 170) |
| VSIG4 | VSIG4 TM | Amino Acid | VFAIILIISLCCMVVFTMAYI (SEQ |
| | domain | | ID NO: 149) |
| VSIG4 | VSIG4 TM | Nucleotide | GTGTTCGCCATCATCCTGATCATCAGC |
| | domain | | CTGTGCTGCATGGTGGTGTTCACCATG |
| | | | GCCTACATC (SEQ ID NO: 171) |
| SIGLEC-12 | SIGLEC-12 TM | Amino Acid | FGGAGATALVFLYFCIIFVVV (SEQ |
| | domain | | ID NO: 150) |
| SIGLEC-12 | SIGLEC-12 TM | Nucleotide | TTTGGCGGAGCTGGTGCTACAGCCCTG |
| | domain | | GTGTTCCTGTACTTCTGCATCATCTTC |
| | | | GTGGTCGTG (SEQ ID NO: 172) |
| LIR8 | LIR8 TM domain | Amino Acid | VVTGVSVAFVLLLFLLLFLLL (SEQ |
| | | | ID NO: 151) |
| LIR8 | LIR8 TM domain | Nucleotide | GTTGTGACTGGGGTCTCAGTGGCCTTC |
| | | | GTCCTGCTGCTGTTCCTCCTCCTCTTC |
| | | | CTCCTCCTC (SEQ ID NO: 173) |
| IRTA1 | IRTA1 TM | Amino Acid | VAAGATGGLLSALLLAVALLF (SEQ |
| | domain | | ID NO: 152) |
| IRTA1 | IRTA1 TM | Nucleotide | GTCGCCGCGGGAGCCACTGGAGGGCTG |
| | domain | | CTCAGTGCTCTTCTCCTGGCTGTGGCC |
| | | | CTGCTGTTT (SEQ ID NO: 174) |
| KIR2DL4 | KIR2DL4 TM | Amino Acid | AVIRYSVAIILFTILPFFLLH (SEQ |
| | domain | | ID NO: 153) |
| KIR2DL4 | KIR2DL4 TM | Nucleotide | GCTGTGATACGTTATTCCGTGGCCATC |
| | domain | | ATCCTGTTCACCATCCTACCCTTCTTC |
| | | | CTGCTGCAT (SEQ ID NO: 175) |
| KIR2DL5 | KIR2DL5 TM | Amino Acid | LHILIGTSVAIILFIILFFFL (SEQ |
| | domain | | ID NO: 154) |
| KIR2DL5 | KIR2DL5 TM | Nucleotide | CTGCACATTCTGATTGGGACCTCAGTG |
| | domain | | GCTATCATCCTCTTCATCATCCTCTTC |
| | | | TTCTTTCTC (SEQ ID NO: 176) |
| SIGLEC-7 | SIGLEC-7 TM | Amino Acid | GAVGGAGATALVFLSFCVIFIVV |
| | domain | | (SEQ ID NO: 155) |
| SIGLEC-7 | SIGLEC-7 TM | Nucleotide | GGGGCGGTCGGGGGAGCTGGAGCCACA |
| | domain | | GCCCTGGTCTTCCTCTCCTTCTGTGTC |
| | | | ATCTTCATTGTAGTG (SEQ ID NO: |
| | | | 177) |
| FCRH3 | FCRH3 TM | Amino Acid | AAGITGLVLSILVLAAAAALL (SEQ |
| | domain | | ID NO: 156) |
| FCRH3 | FCRH3 TM | Nucleotide | GCCGCTGGAATTACAGGCCTGGTGCTG |
| | domain | | AGCATTCTGGTGCTGGCTGCAGCTGCT |
| | | | GCCCTGCTG (SEQ ID NO: 178) |
| PCDHGC5 | PCDHGC5 TM | Amino Acid | LIVALATVSLLSLVTFTFLSA (SEQ |
| | domain | | ID NO: 157) |
| PCDHGC5 | PCDHGC5 TM | Nucleotide | CTGATTGTGGCCCTGGCCACAGTGTCT |
| | domain | | CTGCTGAGCCTCGTGACCTTCACCTTC |
| | | | CTGAGCGCC (SEQ ID NO: 179) |
| CDH11 | CDH11 TM | Amino Acid | GALIAILACIVILLVIVVLFVTL |
| | domain | | (SEQ ID NO: 158) |
| CDH11 | CDH11 TM | Nucleotide | GGAGCCCTGATTGCCATCCTGGCCTGT |
| | domain | | ATCGTGATCCTGCTGGTCATCGTGGTG |
| | | | CTGTTCGTGACCCTG (SEQ ID NO: |
| | | | 180) |
| IMPG2 | IMPG2 TM | Amino Acid | VIIGITIASVVGLLVIFSAII (SEQ |
| | domain | | ID NO: 159) |
| IMPG2 | IMPG2 TM | Nucleotide | GTGATCATCGGCATCACAATCGCCTCT |
| | domain | | GTCGTGGGCCTGCTGGTCATCTTCAGC |
| | | | GCCATCATC (SEQ ID NO: 181) |
| DSCAM | DSCAM ICD | Amino Acid | LVTISCILVGVLLLFVLLLVV (SEQ |
| | TM domain | | ID NO: 160) |
| DSCAM | DSCAM TM | Nucleotide | CTGGTCACCATCAGCTGTATCCTCGTG |
| | domain | | GGAGTGCTGCTGCTGTTCGTCCTGCTG |
| | | | CTGGTTGTG (SEQ ID NO: 182) |
| PECAM-1 | PECAM-1 ICD | Amino Acid | KCYFLRKAKAKQMPVEMSRPAVPLLNS |
| | | | NNEKMSDPNMEANSHYGHNDDVRNHA |
| | | | MKPINDNKEPLNSDVQYTEVQVSSAES |
| | | | HKDLGKKDTETVYSEVRKAVPDAVESR |
| | | | YSRTEGSLDGT (SEQ ID NO: 1) |
| PECAM-1 | PECAM-1 ICD | Nucleotide | AAGTGCTACTTCCTGCGGAAGGCCAAG |
| | | | GCCAAGCAGATGCCCGTGGAAATGAGC |
| | | | AGACCAGCCGTGCCTCTGCTGAACAGC |
| | | | AACAACGAGAAGATGAGCGACCCCAAC |
| | | | ATGGAAGCCAACAGCCACTACGGCCAC |
| | | | AACGACGACGTGCGGAATCACGCCATG |
| | | | AAGCCCATCAACGACAACAAAGAGCCC |
| | | | CTGAACAGCGACGTGCAGTACACCGAG |
| | | | GTGCAAGTGTCTAGCGCCGAGAGCCAC |
| | | | AAGGACCTGGGCAAGAAAGACACCGAG |
| | | | ACAGTGTACAGCGAAGTGCGCAAGGCC |
| | | | GTGCCTGATGCTGTGGAAAGCAGATAC |
| | | | AGCAGAACCGAGGGCAGCCTGGATGGC |
| | | | ACAACCGTGTATTCTGAGGTGGTGCAG |
| | | | TATACAGAAGTC (SEQ ID NO: |
| | | | 12) |
| LIR1 | CD72 (Type II) | Amino Acid | MAEAITYADLRFVKAPLKKSISSRLGQ |
| | ICD | | DPGADDDGEITYENVQVPAVLGVPSSL |
| | | | ASSVLGDKAAVKSEQPTASWRAVTSPA |
| | | | VGRILPCRTTCLRY (SEQ ID NO: |
| | | | 2) |
| LIR1 | CD72 (Type II) | Nucleotide | ATGGCCGAGGCCATCACCTATGCCGAC |
| | ICD | | CTGAGATTTGTGAAGGCCCCTCTGAAG |
| | | | AAGTCCATCAGCAGCAGACTCGGCCAG |
| | | | GATCCAGGCGCTGATGATGATGGCGAG |
| | | | ATCACCTACGAGAACGTGCAGGTTCCA |
| | | | GCCGTGCTGGGAGTGCCATCTTCTCTG |
| | | | GCTAGTAGCGTGCTGGGCGATAAGGCC |
| | | | GCCGTGAAGTCTGAACAGCCTACCGCC |
| | | | TCTTGGAGAGCCGTGACATCTCCTGCC |
| | | | GTGGGCAGAATCCTGCCTTGCAGAACC |
| | | | ACCTGTCTGCGGTAC (SEQ ID NO: |
| | | | 13) |
| IRTA2 | IRTA2 ICD | Amino Acid | LSRKAGRKPASDPARSPSDSDSQEPTY |
| | | | HNVPAWEELQPVYTNANPRGENVVYSE |
| | | | VRIIQEKKKHAVASDPRHLRNKGSPII |
| | | | YSEVKVASTPVSGSLFLASSAPHR |
| | | | (SEQ ID NO: 3) |
| IRTA2 | IRTA2 ICD | Nucleotide | CTGTCTAGAAAGGCCGGCAGAAAGCCT |
| | | | GCCAGCGATCCTGCCAGATCTCCCAGC |
| | | | GACAGCGATAGCCAAGAGCCTACCTAC |
| | | | CACAATGTGCCCGCCTGGGAAGAACTG |
| | | | CAGCCCGTGTACACCAACGCCAATCCT |
| | | | AGAGGCGAGAACGTGGTGTACAGCGAA |
| | | | GTGCGGATCATCCAAGAGAAGAAAAAG |
| | | | CACGCCGTGGCCTCCGATCCTCGGCAC |
| | | | CTTAGAAACAAGGGCAGCCCCATCATC |
| | | | TACTCCGAAGTGAAGGTGGCCAGCACA |
| | | | CCTGTGTCCGGCAGTCTGTTTCTGGCC |
| | | | TCTTCTGCCCCACACCGG (SEQ ID |
| | | | NO: 14) |
| IRTA4 | IRTA4 ICD | Amino Acid | HKISGESSATNEPRGASRPNPQEFTYS |
| | | | SPTPDMEELQPVYVNVGSVDVDVVYSQ |
| | | | VWSMQQPESSANIRTLLENKDSQVIYS |
| | | | SVKKS (SEQ ID NO: 4) |
| IRTA4 | IRTA4 ICD | Nucleotide | CACAAGATCAGCGGCGAGAGCAGCGCC |
| | | | ACCAATGAACCTAGAGGCGCCAGCAGA |
| | | | CCCAATCCTCAAGAGTTCACCTACAGC |
| | | | AGCCCCACACCTGACATGGAAGAACTG |
| | | | CAGCCCGTGTACGTGAACGTGGGCTCC |
| | | | GTGGATGTGGACGTGGTGTACAGCCAA |
| | | | GTGTGGTCCATGCAGCAGCCTGAGTCC |
| | | | AGCGCCAACATCAGAACCCTGCTGGAA |
| | | | AACAAGGACTCCCAAGTGATCTACAGC |
| | | | TCCGTGAAGAAGTCC (SEQ ID NO: |
| | | | 15) |
| NKIR | NKIR ICD | Amino Acid | WRMMKYQQKAAGMSPEQVLQPLEGDLC |
| | | | YADLTLQLAGTSPQKATTKLSSAQVDQ |
| | | | VEVEYVTMASLPKEDISY ASLTLGAE |
| | | | DQEPTYCNMGHLSSHLPGRGPEEPTEY |
| | | | STISRP (SEQ ID NO: 5) |
| NKIR | NKIR ICD | Nucleotide | TGGCGGATGATGAAGTACCAGCAGAAA |
| | | | GCCGCCGGAATGAGCCCCGAACAGGTT |
| | | | CTGCAACCTCTGGAAGGCGATCTGTGC |
| | | | TACGCCGATCTGACACTGCAGCTGGCC |
| | | | GGAACATCTCCTCAGAAGGCCACCACA |
| | | | AAGCTGAGCAGCGCCCAAGTGGATCAG |
| | | | GTGGAAGTGGAATACGTGACAATGGCC |
| | | | AGCCTGCCTAAAGAGGACATCAGCTAC |
| | | | GCCAGCCTGACACTGGGAGCCGAGGAT |
| | | | CAAGAGCCCACCTACTGCAATATGGGC |
| | | | CACCTGAGCAGCCATCTGCCTGGCAGA |
| | | | GGACCTGAGGAACCTACCGAGTACAGC |
| | | | ACCATCAGCAGACCC (SEQ ID NO: |
| | | | 16) |
| IL1RAP | IL1RAP ICD | Amino Acid | YRAHFGTDETILDGKEYDIYVSYARNA |
| | | | EEEEFVLLTLRGVLENEFGYKLCIFDR |
| | | | DSLPGGIVTDETLSFIQKSRRLLVVLS |
| | | | PNYVLQGTQALLELKAGLENMASRGNI |
| | | | NVILVQYKAVKETKVKELKRAKTVLTV |
| | | | IKWKGEKSKYPQGRFWKQLQVAMPVKK |
| | | | SPRRSSSDEQGLSYSSLKNV (SEQ |
| | | | ID NO: 6) |
| IL1RAP | IL1RAP ICD | Nucleotide | TACAGAGCCCACTTCGGCACCGACGAG |
| | | | ACAATCCTGGACGGCAAAGAATACGAC |
| | | | ATCTACGTGTCCTACGCCAGAAACGCC |
| | | | GAGGAAGAGGAATTCGTCCTGCTGACC |
| | | | CTGAGAGGCGTGCTGGAAAACGAGTTC |
| | | | GGCTACAAGCTGTGCATCTTCGACCGG |
| | | | GATTCTCTGCCTGGCGGCATCGTGACA |
| | | | GATGAGACACTGAGCTTCATCCAGAAG |
| | | | TCCCGCAGACTGCTGGTCGTGCTGAGC |
| | | | CCCAATTATGTGCTGCAGGGAACACAG |
| | | | GCCCTGCTGGAACTGAAAGCCGGCCTG |
| | | | GAAAACATGGCCAGCCGGGGCAACATC |
| | | | AACGTGATCCTGGTGCAGTACAAGGCC |
| | | | GTGAAAGAGACAAAAGTCAAAGAGCTG |
| | | | AAGCGGGCCAAGACCGTGCTGACCGTG |
| | | | ATTAAGTGGAAGGGCGAGAAGTCCAAG |
| | | | TATCCCCAGGGCAGATTCTGGAAGCAG |
| | | | CTGCAGGTTGCCATGCCTGTGAAGAAG |
| | | | TCCCCAAGAAGAAGCAGCAGCGACGAG |
| | | | CAGGGACTGAGCTACAGCAGCCTGAAG |
| | | | AACGTG (SEQ ID NO: 16) |
| PTPRO | PTPRO ICD | Amino Acid | LRKKHLQMARECGAGTFVNFASLERDG |
| | | | KLPYNWRRSIFAFLTLLPSCLWTDYLL |
| | | | AFYINPWSKNGLKKRKLTNPVQLDDFD |
| | | | AYIKDMAKDSDYKFSLQFEELKLIGLD |
| | | | IPHFAADLPLNRCKNRYTNILPYDFSR |
| | | | VRLVSMNEEEGADYINANYIPGYNSPQ |
| | | | EYIATQGPLPETRNDFWKMVLQQKSQI |
| | | | IVMLTQCNEKRRVKCDHYWPFTEEPIA |
| | | | YGDITVEMISEEEQDDWACRHFRINYA |
| | | | DEMQDVMHFNYTAWPDHGVPTANAAES |
| | | | ILQFVHMVRQQATKSKGPMIIHCSAGV |
| | | | GRTGTFIALDRLLQHIRDHEFVDILGL |
| | | | VSEMRSYRMSMVQTEEQYIFIHQCVQL |
| | | | MWMKKKQQFCISDVIYENVSKS (SEQ |
| | | | ID NO: 7) |
| PTPRO | PTPRO ICD | Nucleotide | CTGCGGAAGAAACACCTCCAGATGGCC |
| | | | AGAGAATGTGGCGCCGGAACCTTCGTG |
| | | | AATTTCGCCTCTCTGGAACGCGACGGC |
| | | | AAGCTGCCTTATAACTGGCGGAGATCC |
| | | | ATCTTCGCCTTTCTGACCCTGCTGCCT |
| | | | AGCTGCCTGTGGACCGATTACCTGCTG |
| | | | GCCTTCTACATCAACCCTTGGAGCAAG |
| | | | AACGGCCTGAAGAAGCGGAAGCTGACA |
| | | | AACCCCGTGCAGCTCGACGACTTCGAC |
| | | | GCCTACATCAAGGACATGGCCAAGGAC |
| | | | TCCGACTACAAGTTCAGCCTGCAGTTC |
| | | | GAGGAACTGAAGCTGATCGGCCTGGAC |
| | | | ATCCCTCACTTCGCCGCTGACCTGCCT |
| | | | CTGAACCGGTGCAAGAACCGGTACACC |
| | | | AACATCCTGCCTTACGACTTCAGCAGA |
| | | | GTGCGGCTGGTGTCCATGAACGAGGAA |
| | | | GAGGGCGCCGACTACATCAATGCCAAC |
| | | | TACATCCCCGGCTACAACAGCCCTCAA |
| | | | GAGTATATCGCCACACAGGGCCCTCTG |
| | | | CCAGAGACAAGAAACGACTTCTGGAAG |
| | | | ATGGTCCTGCAGCAGAAAAGCCAGATC |
| | | | ATCGTGATGCTGACCCAGTGCAACGAG |
| | | | AAGCGGAGAGTGAAGTGCGACCACTAC |
| | | | TGGCCCTTCACCGAGGAACCTATCGCC |
| | | | TACGGCGACATCACAGTGGAAATGATC |
| | | | AGCGAGGAAGAACAGGACGACTGGGCC |
| | | | TGCCGGCACTTCAGAATCAACTACGCC |
| | | | GACGAGATGCAGGACGTGATGCACTTC |
| | | | AACTACACCGCCTGGCCTGATCACGGC |
| | | | GTGCCAACAGCTAATGCCGCCGAATCC |
| | | | ATCCTGCAGTTTGTGCACATGGTTCGA |
| | | | CAGCAGGCCACCAAGAGCAAGGGCCCC |
| | | | ATGATCATCCACTGTTCTGCTGGCGTG |
| | | | GGCAGAACCGGCACCTTTATCGCTCTG |
| | | | GACAGACTGCTCCAGCACATCCGGGAT |
| | | | CACGAGTTCGTGGATATCCTGGGACTC |
| | | | GTGTCCGAGATGCGGAGCTACAGAATG |
| | | | AGCATGGTGCAGACAGAGGAACAGTAC |
| | | | ATCTTCATCCACCAGTGCGTCCAGCTG |
| | | | ATGTGGATGAAGAAGAAGCAGCAGTTC |
| | | | TGCATCAGCGACGTCATCTACGAGAAC |
| | | | GTGTCCAAGAGC (SEQ ID NO: |
| | | | 18) |
| PTPRZ1 | PTPRZ1 ICD | Amino Acid | RKCFQTAHFYLEDSTSPRVISTPPTPI |
| | | | FPISDDVGAIPIKHFPKHVADLHASSG |
| | | | FTEEFETLKEFYQEVQSCTVDLGITAD |
| | | | SSNHPDNKHKNRYINIVAYDHSRVKLA |
| | | | QLAEKDGKLTDYINANYVDGYNRPKAY |
| | | | IAAQGPLKSTAEDFWRMIWEHNVEVIV |
| | | | MITNLVEKGRRKCDQYWPADGSEEYGN |
| | | | FLVTQKSVQVLAYYTVRNFTLRNTKIK |
| | | | KGSQKGRPSGRVVTQYHYTQWPDMGVP |
| | | | EYSLPVLTFVRKAAYAKRHAVGPVVVH |
| | | | CSAGVGRTGTYIVLDSMLQQIQHEGTV |
| | | | NIFGFLKHIRSQRNYLVQTEEQYVFIH |
| | | | DTLVEAILSKETEVLDSHIHAYVNALL |
| | | | IPGPAGKTKLEKQFQLLSQSNIQQSDY |
| | | | SAALKQCNREKNRTSSIIPVERSRVGI |
| | | | SSLSGEGTDYINASYIMGYYQSNEFII |
| | | | TQHPLLHTIKDFWRMIWDHNAQLVVMI |
| | | | PDGQNMAEDEFVYWPNKDEPINCESFK |
| | | | VTLMAEEHKCLSNEEKLIIQDFILEAT |
| | | | QDDYVLEVRHFQCPKWPNPDSPISKTF |
| | | | ELISVIKEEAANRDGPMIVHDEHGGVT |
| | | | AGTFCALTTLMHQLEKENSVDVYQVAK |
| | | | MINLMRPGVFADIEQYQFLYKVILSLV |
| | | | STRQEENPSTSLDSNGAALPDGNIAES |
| | | | LESLV (SEQ ID NO: 8) |
| PTPRZ1 | PTPRZ1 ICD | Nucleotide | CGGAAGTGCTTCCAGACAGCCCACTTC |
| | | | TACCTGGAAGATAGCACAAGCCCCAGA |
| | | | GTGATCAGCACCCCTCCAACACCTATC |
| | | | TTCCCCATCTCCGATGACGTGGGCGCT |
| | | | ATCCCCATCAAGCACTTTCCAAAGCAC |
| | | | GTGGCCGATCTGCACGCCAGCTCTGGC |
| | | | TTCACCGAGGAATTCGAGACACTGAAA |
| | | | GAATTCTACCAAGAGGTGCAGAGCTGC |
| | | | ACCGTGGACCTGGGAATCACAGCCGAC |
| | | | AGCAGCAATCACCCCGACAACAAGCAC |
| | | | AAGAACCGGTACATCAACATCGTGGCC |
| | | | TACGATCACAGCAGAGTGAAGCTGGCC |
| | | | CAGCTGGCCGAGAAGGATGGCAAGCTG |
| | | | ACCGACTACATCAACGCCAATTACGTG |
| | | | GACGGCTACAACAGGCCCAAGGCCTAT |
| | | | ATTGCCGCTCAGGGCCCTCTGAAGTCT |
| | | | ACCGCCGAGGACTTTTGGAGAATGATC |
| | | | TGGGAGCACAACGTGGAAGTGATCGTG |
| | | | ATGATCACCAACCTGGTGGAAAAAGGC |
| | | | AGACGGAAGTGCGATCAGTACTGGCCT |
| | | | GCCGATGGCAGCGAGGAATACGGCAAT |
| | | | TTCCTGGTCACCCAGAAAAGCGTGCAG |
| | | | GTCCTGGCCTACTACACAGTGCGGAAC |
| | | | TTCACCCTGCGGAACACCAAGATCAAG |
| | | | AAGGGCTCCCAGAAGGGCAGACCCTCT |
| | | | GGCAGAGTGGTCACACAGTACCACTAC |
| | | | ACCCAGTGGCCTGATATGGGCGTGCCA |
| | | | GAGTATAGCCTGCCAGTGCTGACCTTT |
| | | | GTGCGGAAGGCCGCCTATGCCAAGAGA |
| | | | CATGCTGTGGGACCTGTGGTGGTGCAC |
| | | | TGTTCTGCTGGCGTGGGAAGAACCGGC |
| | | | ACCTACATCGTGCTGGACAGCATGCTC |
| | | | CAGCAGATTCAGCACGAGGGCACCGTG |
| | | | AATATCTTCGGCTTCCTGAAGCACATC |
| | | | AGAAGCCAGCGGAACTACCTGGTGCAG |
| | | | ACCGAGGAACAGTACGTGTTCATCCAC |
| | | | GACACACTGGTGGAAGCCATCCTGAGC |
| | | | AAAGAAACCGAGGTGCTGGACTCCCAC |
| | | | ATCCACGCCTATGTGAACGCCCTGCTG |
| | | | ATTCCTGGACCAGCCGGCAAGACCAAG |
| | | | CTGGAAAAGCAGTTCCAGCTGCTGTCC |
| | | | CAGAGCAACATCCAGCAGAGCGACTAC |
| | | | AGCGCCGCTCTGAAGCAGTGCAACAGA |
| | | | GAGAAGAACAGAACCAGCAGCATCATC |
| | | | CCTGTGGAACGGTCCAGAGTGGGCATC |
| | | | TCTAGCTTGTCTGGCGAGGGCACAGAT |
| | | | TACATCAATGCCAGCTACATCATGGGC |
| | | | TACTACCAGTCCAACGAGTTCATCATC |
| | | | ACACAGCACCCTCTGCTGCACACCATC |
| | | | AAGGATTTCTGGCGGATGATTTGGGAC |
| | | | CACAATGCTCAGCTGGTGGTCATGATC |
| | | | CCCGACGGCCAGAATATGGCCGAGGAC |
| | | | GAGTTTGTGTACTGGCCCAACAAGGAC |
| | | | GAGCCCATCAACTGCGAGAGCTTCAAA |
| | | | GTGACCCTGATGGCCGAAGAACACAAG |
| | | | TGCCTGAGCAACGAGGAAAAGCTGATC |
| | | | ATCCAGGACTTCATCCTGGAAGCCACA |
| | | | CAGGACGACTACGTGCTGGAAGTGCGG |
| | | | CACTTTCAGTGCCCTAAGTGGCCCAAT |
| | | | CCTGACAGCCCCATCAGCAAGACCTTC |
| | | | GAGCTGATCTCCGTGATCAAAGAGGAA |
| | | | GCCGCCAACCGCGACGGCCCTATGATT |
| | | | GTGCACGATGAGCATGGCGGAGTGACC |
| | | | GCCGGAACATTTTGTGCCCTGACAACC |
| | | | CTGATGCACCAGCTCGAGAAAGAAAAC |
| | | | TCCGTGGACGTGTACCAGGTGGCCAAG |
| | | | ATGATCAACCTGATGCGGCCTGGCGTG |
| | | | TTCGCCGATATCGAGCAGTACCAGTTC |
| | | | CTGTACAAAGTGATCCTCAGCCTGGTG |
| | | | TCTACCCGGCAAGAGGAAAACCCTAGC |
| | | | ACCAGCCTGGATAGCAATGGCGCCGCA |
| | | | CTGCCCGATGGAAATATCGCCGAGTCT |
| | | | CTGGAAAGCCTCGTT (SEQ ID NO: |
| | | | 19) |
| TLT1 | TLT1 ICD | Amino Acid | MAKRKQGNRLGVCGRFLSSRVSGMNPS |
| | | | SVVHHVSDSGPAAELPLDVPHIRLDSP |
| | | | PSFDNTTYTSLPLDSPSGKPSLPAPSS |
| | | | LPPLPPKVLVCSKPVTYATVIFPGGNK |
| | | | GGGTSCGPAQNPPNNQTPSS (SEQ |
| | | | ID NO: 9) |
| TLT1 | TLT1 ICD | Nucleotide | ATGGCCAAGCGGAAGCAGGGCAATAGA |
| | | | CTGGGCGTGTGCGGCAGATTCCTGTCC |
| | | | TCTAGAGTGTCCGGCATGAACCCCAGC |
| | | | AGCGTGGTGCATCACGTGTCCGATTCT |
| | | | GGACCTGCCGCTGAGCTGCCACTGGAT |
| | | | GTGCCTCACATCAGACTGGACAGCCCA |
| | | | CCTAGCTTCGACAACACCACCTACACA |
| | | | AGCCTGCCTCTCGATAGCCCTTCCGGC |
| | | | AAGCCATCTCTGCCTGCTCCATCTTCT |
| | | | CTGCCTCCACTGCCTCCTAAGGTGCTC |
| | | | GTGTGTAGCAAGCCTGTGACCTACGCC |
| | | | ACCGTGATCTTCCCTGGCGGAAACAAA |
| | | | GGCGGCGGAACATCTTGTGGCCCCGCT |
| | | | CAGAACCCTCCTAACAATCAGACACCT |
| | | | AGCAGC (SEQ ID NO: 20) |
| SLAMF1 | SLAMF1 ICD | Amino Acid | QLRRRGKTNHYQTTVEKKSLTIYAQVQ |
| | | | KPGPLQKKLDSFPAQDPCTTIYVAATE |
| | | | PVPESVQETNSITVYASVTLPES |
| | | | (SEQ ID NO: 10) |
| SLAMF1 | SLAMF1 ICD | Nucleotide | CAGCTGCGTCGCCGTGGCAAGACGAAC |
| | | | CATTACCAGACGACCGTGGAGAAAAAA |
| | | | TCCCTGACCATTTACGCGCAGGTGCAG |
| | | | AAGCCGGGGCCCCTGCAGAAGAAGCTG |
| | | | GACTCGTTTCCTGCTCAGGACCCGTGC |
| | | | ACTACAATCTACGTGGCGGCTACGGAG |
| | | | CCCGTACCCGAATCCGTGCAGGAGACT |
| | | | AACTCCATCACCGTCTACGCTTCCGTG |
| | | | ACCCTTCCTGAGAGC (SEQ ID NO: |
| | | | 21) |
| SLAMF5 | SLAMF5 ICD | Amino Acid | RLFKRRQGRIFPEGSCLNTFTKNPYAA |
| | | | SKKTIYTYIMASRNTQPAESRIYDEIL |
| | | | QSKVLPSKEEPVNTVYSEVQFADKMGK |
| | | | ASTQDSKPPGTSSYEIVI (SEQ ID |
| | | | NO: 11) |
| SLAMF5 | SLAMF5 ICD | Nucleotide | CGCCTGTTCAAGCGCCGCCAGGGGCGC |
| | | | ATCTTCCCCGAGGGCTCTTGTCTCAAT |
| | | | ACGTTCACCAAGAACCCCTATGCTGCC |
| | | | AGCAAGAAGACCATCTACACCTACATC |
| | | | ATGGCCTCCCGCAACACCCAGCCAGCA |
| | | | GAGTCGCGCATTTACGACGAGATCCTG |
| | | | CAATCTAAGGTGCTGCCATCCAAGGAG |
| | | | GAACCCGTCAACACTGTTTACTCCGAG |
| | | | GTGCAGTTCGCGGACAAAATGGGGAAG |
| | | | GCTTCCACCCAGGACTCCAAGCCGCCT |
| | | | GGCACCTCGAGCTACGAGATCGTGATT |
| | | | (SEQ ID NO: 22) |
| PCDHGC3 | PCDHGC3 ICD | Amino Acid | FKVYKWKQSRDLYRAPVSSLYRTPGPS |
| | | | LHADAVRGGLMSPHLYHQVYLTTDSRR |
| | | | SDPLLKKPGAASPLASRQNTLRSCDPV |
| | | | FYRQVLGAESAPPGQQAPPNTDWRFSQ |
| | | | AQRPGTSGSQNGDDTGTWPNNQFDTEM |
| | | | LQAMILASASEAADGSSTLGGGAGTMG |
| | | | LSARYGPQFTLQHVPDYRQNVYIPGSN |
| | | | ATLTNAAGKRDGKAPAGGNGNKKKSGK |
| | | | KEKK (SEQ ID NO: 95) |
| PCDHGC3 | PCDHGC3 ICD | Nucleotide | TTCAAGGTGTACAAGTGGAAGCAGAGC |
| | | | CGGGACCTGTACAGAGCCCCTGTGTCC |
| | | | AGCCTGTATAGAACCCCTGGACCTAGC |
| | | | CTGCATGCCGATGCTGTTAGAGGCGGC |
| | | | CTGATGAGCCCTCACCTGTACCATCAG |
| | | | GTGTACCTGACCACCGACAGCAGAAGA |
| | | | AGCGACCCTCTGCTGAAGAAGCCTGGC |
| | | | GCTGCTTCTCCACTGGCCAGCAGACAG |
| | | | AATACCCTGAGAAGCTGCGACCCCGTG |
| | | | TTCTATAGGCAGGTTCTGGGAGCCGAA |
| | | | TCTGCCCCTCCTGGACAACAGGCCCCT |
| | | | CCTAACACCGATTGGAGATTCAGCCAG |
| | | | GCTCAGAGGCCTGGCACAAGCGGATCT |
| | | | CAGAATGGCGACGATACCGGCACCTGG |
| | | | CCTAACAACCAGTTCGACACCGAAATG |
| | | | CTGCAGGCCATGATTCTGGCCTCTGCC |
| | | | TCTGAAGCCGCCGATGGCAGTTCTACA |
| | | | CTTGGAGGCGGAGCCGGCACAATGGGA |
| | | | CTGTCTGCTAGATACGGCCCTCAGTTC |
| | | | ACCCTGCAGCACGTGCCCGATTACCGG |
| | | | CAGAACGTGTACATCCCCGGCAGCAAT |
| | | | GCCACACTGACAAACGCCGCTGGCAAG |
| | | | AGAGATGGCAAAGCTCCTGCTGGCGGC |
| | | | AACGGCAACAAGAAGAAGTCCGGCAAG |
| | | | AAAGAGAAGAAG (SEQ ID NO: |
| | | | 117) |
| LIFR | LIFR ICD | Amino Acid | YRKREWIKETFYPDIPNPENCKALQFQ |
| | | | KSVCEGSSALKTLEMNPCTPNNVEVLE |
| | | | TRSAFPKIEDTEIISPVAERPEDRSDA |
| | | | EPENHVVVSYCPPIIEEEIPNPAADEA |
| | | | GGTAQVIYIDVQSMYQPQAKPEEEQEN |
| | | | DPVGGAGYKPQMHLPINSTVEDIAAEE |
| | | | DLDKTAGYRPQANVNTWNLVSPDSPRS |
| | | | IDSNSEIVSFGSPCSINSRQFLIPPKD |
| | | | EDSPKSNGGGWSFTNFFQNKPND |
| | | | (SEQ ID NO: 96) |
| LIFR | LIFR ICD | Nucleotide | TACCGGAAGAGAGAGTGGATCAAAGAG |
| | | | ACATTCTACCCGGACATCCCCAATCCT |
| | | | GAGAACTGCAAAGCCCTGCAGTTCCAG |
| | | | AAAAGCGTGTGCGAGGGAAGCAGCGCC |
| | | | CTGAAAACCCTGGAAATGAACCCCTGC |
| | | | ACACCCAACAACGTGGAAGTGCTGGAA |
| | | | ACCAGAAGCGCTTTCCCCAAGATCGAG |
| | | | GACACCGAGATCATCAGCCCCGTGGCC |
| | | | GAAAGACCCGAGGATAGATCTGATGCC |
| | | | GAGCCTGAGAATCACGTGGTGGTGTCC |
| | | | TACTGTCCTCCTATCATCGAGGAAGAG |
| | | | ATCCCTAATCCTGCCGCCGATGAAGCC |
| | | | GGCGGAACAGCCCAAGTGATCTACATC |
| | | | GACGTGCAGAGCATGTACCAGCCTCAG |
| | | | GCCAAGCCTGAGGAAGAACAAGAGAAC |
| | | | GACCCTGTTGGCGGAGCCGGCTACAAG |
| | | | CCCCAAATGCATCTGCCCATCAACAGC |
| | | | ACCGTGGAAGATATCGCCGCCGAAGAG |
| | | | GACCTGGATAAGACCGCCGGATATAGA |
| | | | CCCCAGGCCAACGTGAACACCTGGAAC |
| | | | CTGGTGTCCCCAGACAGCCCCAGATCC |
| | | | ATCGACAGCAACAGCGAGATCGTGTCC |
| | | | TTCGGCAGCCCCTGCTCTATCAACAGC |
| | | | CGGCAGTTCCTGATTCCTCCTAAGGAC |
| | | | GAGGACAGCCCTAAGAGCAATGGCGGC |
| | | | GGATGGTCCTTCACCAATTTCTTCCAG |
| | | | AACAAGCCCAACGAC (SEQ ID NO: |
| | | | 118) |
| ERMAP | ERMAP ICD | Amino Acid | WKQRRAKEKLLYEHVTEVDNLLSDHAK |
| | | | EKGKLHKAVKKLRSELKLKRAAANSGW |
| | | | RRARLHFVAVTLDPDTAHPKLILSEDQ |
| | | | RCVRLGDRRQPVPDNPQRFDFVVSILG |
| | | | SEYFTTGCHYWEVYVGDKTKWILGVCS |
| | | | ESVSRKGKVTASPANGHWLLRQSRGNE |
| | | | YEALTSPQTSFRLKEPPRCVGIFLDYE |
| | | | AGVISFYNVTNKSHIFTFTHNFSGPLR |
| | | | PFFEPCLHDGGKNTAPLVICSELHKSE |
| | | | ESIVPRPEGKGHANGDVSLKVNSSLLP |
| | | | PKAPELKDIILSLPPDLGPALQELKAP |
| | | | SF (SEQ ID NO: 97) |
| ERMAP | ERMAP ICD | Nucleotide | TGGAAGCAGCGGAGAGCCAAAGAGAAG |
| | | | CTGCTGTACGAGCACGTGACCGAGGTG |
| | | | GACAACCTGCTGAGCGATCACGCCAAA |
| | | | GAAAAGGGCAAGCTGCACAAGGCCGTG |
| | | | AAGAAGCTGAGAAGCGAGCTGAAGCTG |
| | | | AAGCGGGCTGCCGCCAATTCTGGATGG |
| | | | CGTAGAGCCAGACTGCACTTCGTGGCC |
| | | | GTGACACTGGACCCCGATACAGCCCAT |
| | | | CCTAAGCTGATCCTGAGCGAGGACCAG |
| | | | AGATGTGTGCGGCTGGGAGATAGAAGG |
| | | | CAGCCCGTGCCTGACAACCCTCAGAGA |
| | | | TTCGACTTCGTGGTGTCCATCCTGGGC |
| | | | AGCGAGTACTTCACCACCGGCTGCCAC |
| | | | TACTGGGAAGTGTACGTGGGCGACAAG |
| | | | ACCAAGTGGATCCTGGGCGTGTGTAGC |
| | | | GAGAGCGTGTCCAGAAAGGGCAAAGTG |
| | | | ACAGCTAGCCCCGCCAATGGACACTGG |
| | | | CTGCTGAGACAGAGCAGAGGCAATGAG |
| | | | TACGAGGCCCTGACAAGCCCACAGACC |
| | | | AGCTTCCGGCTGAAAGAACCTCCTAGA |
| | | | TGCGTGGGCATCTTCCTGGATTATGAG |
| | | | GCCGGCGTGATCAGCTTCTACAACGTG |
| | | | ACCAACAAGAGCCACATCTTCACGTTC |
| | | | ACCCACAACTTCAGCGGCCCTCTGCGG |
| | | | CCATTCTTCGAGCCTTGTCTGCACGAC |
| | | | GGCGGCAAGAATACTGCCCCTCTGGTC |
| | | | ATCTGTAGCGAACTGCACAAGAGCGAG |
| | | | GAATCCATCGTGCCCAGACCTGAAGGC |
| | | | AAGGGACATGCCAATGGGGACGTGTCC |
| | | | CTGAAAGTGAACAGCAGCCTGCTGCCT |
| | | | CCTAAGGCTCCTGAGCTGAAGGACATC |
| | | | ATCCTGAGCCTGCCTCCAGACCTGGGA |
| | | | CCTGCTCTGCAAGAACTGAAGGCCCCT |
| | | | AGCTTC (SEQ ID NO: 119) |
| IL1RAPL2 | IL1RAPL2 ICD | Amino Acid | KCYNIELMLFYRQHFGADETNDDNKEY |
| | | | DAYLSYTKVDQDTLDCDNPEEEQFALE |
| | | | VLPDVLEKHYGYKLFIPERDLIPSGTY |
| | | | MEDLTRYVEQSRRLIIVLTPDYILRRG |
| | | | WSIFELESRLHNMLVSGEIKVILIECT |
| | | | ELKGKVNCQEVESLKRSIKLLSLIKWK |
| | | | GSKSSKLNSKFWKHLVYEMPIKKKEML |
| | | | PRCHVLDSAEQGLFGELQPIPSIAMTS |
| | | | TSATLVSSQADLPEFHPSDSMQIRHCC |
| | | | RGYKHEIPATTLPVPSLGNHHTYCNLP |
| | | | LTLLNGQLPLNNTLKDTQEFHRNSSLL |
| | | | PLSSKELSFTSDIW (SEQ ID NO: |
| | | | 98) |
| IL1RAPL2 | IL1RAPL2 ICD | Nucleotide | AAGTGCTACAACATCGAGCTGATGCTG |
| | | | TTCTACCGGCAGCACTTTGGCGCCGAC |
| | | | GAGACAAACGACGACAACAAAGAGTAC |
| | | | GACGCCTACCTGAGCTACACCAAGGTG |
| | | | GACCAGGACACCCTGGACTGCGACAAC |
| | | | CCTGAGGAAGAACAGTTCGCCCTCGAG |
| | | | GTGCTGCCCGACGTGCTGGAAAAGCAC |
| | | | TACGGCTACAAGCTGTTCATCCCCGAG |
| | | | CGGGATCTGATCCCTAGCGGCACCTAC |
| | | | ATGGAAGATCTGACCAGATACGTGGAA |
| | | | CAGAGCAGACGGCTGATCATCGTGCTG |
| | | | ACCCCTGACTACATCCTGCGGAGAGGC |
| | | | TGGTCCATCTTCGAGCTGGAAAGCCGG |
| | | | CTGCACAACATGCTGGTGTCCGGCGAG |
| | | | ATCAAAGTGATCCTGATCGAGTGCACC |
| | | | GAGCTGAAGGGCAAAGTGAACTGCCAA |
| | | | GAGGTGGAAAGCCTGAAGCGGAGCATC |
| | | | AAGCTGCTGAGCCTGATCAAGTGGAAG |
| | | | GGCAGCAAGAGCAGCAAGCTGAACAGC |
| | | | AAGTTCTGGAAGCACCTGGTGTACGAG |
| | | | ATGCCCATCAAAAAGAAAGAAATGCTG |
| | | | CCCCGGTGCCATGTGCTGGATTCTGCT |
| | | | GAGCAGGGCCTGTTTGGAGAGCTGCAG |
| | | | CCCATTCCTTCTATCGCCATGACCAGC |
| | | | ACCTCCGCCACACTGGTTTCTAGCCAG |
| | | | GCCGACCTGCCTGAGTTTCACCCCAGC |
| | | | GATAGCATGCAGATCCGGCACTGCTGC |
| | | | CGGGGCTATAAGCACGAGATTCCAGCC |
| | | | ACCACACTGCCCGTGCCTTCTCTGGGA |
| | | | AATCACCACACCTACTGCAACCTGCCT |
| | | | CTGACACTGCTGAACGGCCAGCTGCCA |
| | | | CTGAACAACACCCTGAAGGACACCCAA |
| | | | GAGTTCCACCGGAACAGCAGCCTGCTG |
| | | | CCTCTGTCCAGCAAAGAGCTGAGCTTC |
| | | | ACCAGCGACATCTGG (SEQ ID NO: |
| | | | 120) |
| CDH5 | CDH5 ICD | Amino Acid | RRRLRKQARAHGKSVPEIHEQLVTYDE |
| | | | EGGGEMDTTSYDVSVLNSVRRGGAKPP |
| | | | RPALDARPSLYAQVQKPPRHAPGAHGG |
| | | | PGEMAAMIEVKKDEADHDGDGPPYDTL |
| | | | HIYGYEGSESIAESLSSLGTDSSDSDV |
| | | | DYDFLNDWGPRFKMLAELYGSDPREEL |
| | | | LY (SEQ ID NO: 99) |
| CDH5 | CDH5 ICD | Nucleotide | CGGCGGAGACTGAGAAAGCAGGCTAGA |
| | | | GCCCACGGCAAGAGCGTGCCAGAGATT |
| | | | CACGAACAGCTGGTCACCTACGACGAG |
| | | | GAAGGCGGAGGCGAGATGGATACCACA |
| | | | AGCTACGATGTGTCCGTGCTGAACAGC |
| | | | GTGCGAAGAGGCGGAGCCAAACCTCCT |
| | | | AGACCTGCTCTGGATGCCAGACCTAGC |
| | | | CTGTATGCCCAGGTGCAGAAGCCTCCA |
| | | | AGACACGCTCCTGGTGCTCATGGTGGA |
| | | | CCTGGCGAAATGGCCGCCATGATCGAA |
| | | | GTGAAGAAGGACGAGGCCGACCACGAT |
| | | | GGCGACGGCCCTCCATATGATACCCTG |
| | | | CACATCTACGGCTACGAGGGCAGCGAG |
| | | | TCTATCGCCGAGTCTCTGTCTAGCCTG |
| | | | GGCACCGATAGCAGCGATAGCGACGTG |
| | | | GACTACGACTTCCTGAACGACTGGGGC |
| | | | CCTAGATTCAAGATGCTGGCCGAGCTG |
| | | | TACGGCAGCGACCCTAGAGAGGAACTG |
| | | | CTGTAC (SEQ ID NO: 121) |
| MPZL1 | MPZL1 ICD | Amino Acid | SMILAVLYRRKNSKRDYTGCSTSESLS |
| | | | PVKQAPRKSPSDTEGLVKSLPSGSHQG |
| | | | PVIYAQLDHSGGHHSDKINKSESVVYA |
| | | | DIRKN (SEQ ID NO: 100) |
| MPZL1 | MPZL1 ICD | Nucleotide | TCTATGATCCTGGCCGTGCTGTACCGG |
| | | | CGGAAGAACAGCAAGAGAGACTACACC |
| | | | GGCTGCAGCACCAGCGAGTCTCTGTCT |
| | | | CCAGTGAAGCAGGCCCCTAGAAAGAGC |
| | | | CCCTCTGATACCGAAGGCCTGGTCAAG |
| | | | TCTCTGCCCAGCGGATCTCATCAGGGC |
| | | | CCAGTGATCTATGCCCAGCTGGACCAT |
| | | | TCTGGCGGCCACCACAGCGACAAGATC |
| | | | AACAAGAGCGAGAGCGTGGTGTACGCC |
| | | | GACATCCGGAAGAAT(SEQ ID NO: |
| | | | 122) |
| MPZ | MPZ ICD | Amino Acid | RYCWLRRQAALQRRLSAMEKGKLHKPG |
| | | | KDASKRGRQTPVLYAMLDHSRSTKAVS |
| | | | EKKAKGLGESRKDKK (SEQ IDNO: |
| | | | 101) |
| MPZ | MPZ ICD | Nucleotide | CGGTACTGCTGGCTGAGAAGGCAGGCT |
| | | | GCACTGCAGAGAAGGCTGAGCGCCATG |
| | | | GAAAAGGGCAAGCTGCACAAGCCTGGC |
| | | | AAGGACGCCTCTAAGAGAGGCAGACAG |
| | | | ACCCCTGTGCTGTACGCCATGCTGGAC |
| | | | CACAGCAGAAGCACAAAGGCCGTCAGC |
| | | | GAGAAGAAGGCCAAAGGCCTGGGCGAG |
| | | | AGCCGGAAGGATAAGAAG(SEQ ID |
| | | | NO: 123) |
| FCGR2B | FCGR2B ICD | Amino Acid | VVALIYCRKKRISALPGYPECREMGET |
| | | | LPEKPANPTNPDEADKVGAENTITYSL |
| | | | LMHPDALEEPDDQNRI (SEQ ID |
| | | | NO: 102) |
| FCGR2B | FCGR2B ICD | Nucleotide | GTGGTGGCCCTGATCTACTGCCGGAAG |
| | | | AAGAGAATCTCTGCCCTGCCTGGCTAC |
| | | | CCCGAGTGTAGAGAGATGGGAGAGACA |
| | | | CTGCCCGAGAAGCCCGCCAATCCTACC |
| | | | AATCCTGACGAGGCCGACAAAGTGGGC |
| | | | GCCGAGAATACCATCACCTACAGCCTG |
| | | | CTGATGCACCCCGACGCTCTGGAAGAA |
| | | | CCCGACGACCAGAACAGAATC (SEQ |
| | | | ID NO: 124) |
| SIGLEC-6 | SIGLEC-6 ICD | Amino Acid | RVKTRRKKAAQPVQNTDDVNPVMVSGS |
| | | | RGHQHQFQTGIVSDHPAEAGPISEDEQ |
| | | | ELHYAVLHFHKVQPQEPKVTDTEYSEI |
| | | | KIHK (SEQ ID NO: 103) |
| SIGLEC-6 | SIGLEC-6 ICD | Nucleotide | AGAGTGAAAACCCGGCGGAAGAAAGCC |
| | | | GCTCAGCCCGTGCAGAATACCGACGAC |
| | | | GTGAACCCTGTGATGGTGTCCGGCTCT |
| | | | AGAGGACACCAGCACCAGTTTCAGACC |
| | | | GGCATCGTGTCTGATCACCCTGCCGAA |
| | | | GCCGGACCTATCAGCGAGGATGAGCAA |
| | | | GAGCTGCACTACGCCGTGCTGCACTTC |
| | | | CACAAGGTGCAGCCCCAAGAGCCTAAA |
| | | | GTGACCGACACCGAGTACAGCGAGATC |
| | | | AAGATCCACAAG (SEQ ID NO: |
| | | | 125) |
| MPIG6B | MPIG6B ICD | Amino Acid | WLHRRLPPQPIRPLPRFAPLVKTEPQR |
| | | | PVKEEEPKIPGDLDQEPSLLYADLDHL |
| | | | ALSRPRRLSTADPADASTIYAVVV |
| | | | (SEQ ID NO: 104) |
| MPIG6B | MPIG6B ICD | Nucleotide | TGGCTGCACAGAAGGCTGCCTCCACAG |
| | | | CCTATCAGACCCCTGCCTAGATTTGCC |
| | | | CCTCTGGTCAAGACAGAGCCCCAGCGG |
| | | | CCTGTGAAAGAGGAAGAACCTAAGATC |
| | | | CCCGGCGACCTGGACCAAGAGCCTTCT |
| | | | CTGCTGTACGCCGACCTGGATCATCTG |
| | | | GCCCTGAGCAGACCTAGACGGCTGTCT |
| | | | ACAGCCGATCCTGCCGATGCCAGCACA |
| | | | ATCTATGCTGTGGTGGTG (SEQ ID |
| | | | NO: 126) |
| VSIG4 | VSIG4 ICD | Amino Acid | MLCRKTSQQEHVYEAARAHAREANDSG |
| | | | ETMRVAIFASGCSSDEPTSQNLGNNYS |
| | | | DEPCIGQEYQIIAQINGNYARLLDTVP |
| | | | LDYEFLATEGKSVC (SEQ ID NO: |
| | | | 105) |
| VSIG4 | VSIG4 ICD | Nucleotide | ATGCTGTGCAGAAAGACCAGCCAGCAA |
| | | | GAGCACGTGTACGAGGCCGCTAGAGCC |
| | | | CATGCCAGAGAGGCTAACGATAGCGGC |
| | | | GAGACAATGAGAGTGGCCATCTTCGCC |
| | | | AGCGGCTGTAGCTCTGATGAGCCCACC |
| | | | TCTCAGAACCTGGGCAACAACTACAGC |
| | | | GACGAGCCCTGCATCGGCCAAGAGTAC |
| | | | CAGATCATTGCCCAGATCAACGGCAAC |
| | | | TACGCCCGGCTGCTGGATACCGTGCCT |
| | | | CTGGATTATGAGTTCCTGGCCACCGAG |
| | | | GGCAAGAGCGTGTGT (SEQ ID NO: |
| | | | 127) |
| SIGLEC-12 | SIGLEC-12 ICD | Amino Acid | RSCRKKSARPAVGVGDTGMEDANAVRG |
| | | | SASQGPLIESPADDSPPHHAPPALATP |
| | | | SPEEGEIQYASLSFHKARPQYPQEQEA |
| | | | IGYEYSEINIPK (SEQ ID NO: |
| | | | 106) |
| SIGLEC-12 | SIGLEC-12 ICD | Nucleotide | CGGAGCTGCCGGAAGAAGTCTGCTAGA |
| | | | CCTGCTGTTGGCGTGGGCGATACCGGA |
| | | | ATGGAAGATGCCAATGCCGTCAGAGGC |
| | | | AGCGCCTCTCAGGGACCTCTGATTGAG |
| | | | TCTCCCGCCGACGATAGCCCTCCTCAT |
| | | | CATGCTCCTCCAGCTCTGGCCACACCT |
| | | | TCTCCAGAGGAAGGCGAGATCCAGTAC |
| | | | GCCAGCCTGAGCTTCCACAAGGCCAGA |
| | | | CCTCAGTACCCTCAAGAGCAAGAGGCC |
| | | | ATCGGCTACGAGTACAGCGAGATCAAC |
| | | | ATCCCCAAG (SEQ ID NO: 128) |
| LIR8 | LIR8 ICD | Amino Acid | RHRHQSKHRTSAHFYRPAGAAGPEPKD |
| | | | QGLQKRASPVADIQEEILNAAVKDTQP |
| | | | KDGVEMDAPAAASEAPQDVTYAQLHSL |
| | | | TLRREATEPPPSQEREPPAEPSIYAPL |
| | | | AIH (SEQ ID NO: 107) |
| LIR8 | LIR8 ICD | Nucleotide | CGACATCGGCATCAGAGCAAACACAGG |
| | | | ACATCGGCCCATTTCTACCGTCCTGCA |
| | | | GGGGCTGCGGGGCCAGAGCCCAAGGAC |
| | | | CAGGGCCTGCAGAAGAGGGCCAGCCCA |
| | | | GTTGCTGACATCCAGGAGGAAATTCTC |
| | | | AATGCTGCCGTGAAGGACACACAGCCC |
| | | | AAGGACGGGGTGGAGATGGATGCTCCG |
| | | | GCTGCTGCATCTGAAGCCCCCCAGGAT |
| | | | GTGACCTACGCCCAGCTGCACAGCTTG |
| | | | ACCCTCAGACGGGAGGCAACTGAGCCT |
| | | | CCTCCATCCCAGGAAAGGGAACCTCCA |
| | | | GCTGAACCCAGCATCTACGCCCCCCTG |
| | | | GCCATCCAC (SEQ ID NO: 129) |
| IRTA1 | IRTA1 ICD | Amino Acid | HCWRRRKSGVGFLGDETRLPPAPGPGE |
| | | | SSHSICPAQVELQSLYVDVHPKKGDLV |
| | | | YSEIQTTQLGEEEEANTSRTLLEDKDV |
| | | | SVVYSEVKTQHPDNSAGKISSKDEES |
| | | | (SEQ ID NO: 108) |
| IRTA1 | IRTA1 ICD | Nucleotide | CACTGCTGGCGTCGGAGGAAGTCAGGA |
| | | | GTTGGTTTCTTGGGAGACGAAACCAGG |
| | | | CTCCCTCCCGCTCCAGGCCCAGGAGAG |
| | | | TCCTCCCATTCCATCTGCCCTGCCCAG |
| | | | GTGGAGCTTCAGTCGTTGTATGTTGAT |
| | | | GTACACCCCAAAAAGGGAGATTTGGTA |
| | | | TACTCTGAGATCCAGACTACTCAGCTG |
| | | | GGAGAAGAAGAGGAAGCTAATACCTCC |
| | | | AGGACACTTCTAGAGGATAAGGATGTC |
| | | | TCAGTTGTCTACTCTGAGGTAAAGACA |
| | | | CAACACCCAGATAACTCAGCTGGAAAG |
| | | | ATCAGCTCTAAGGATGAAGAAAGT |
| | | | (SEQ ID NO: 130) |
| KIR2DL4 | KIR2DL4 ICD | Amino Acid | RWCSKKKDAAVMNQEPAGHRTVNREDS |
| | | | DEQDPQEVTYAQLDHCIFTQRKITGPS |
| | | | QRSKRPSTDTSVCIELPNAEPRALSPA |
| | | | HEHHSQALMGSSRETTALSQTQLASSN |
| | | | VPAAGI (SEQ ID NO: 109) |
| KIR2DL4 | KIR2DL4 ICD | Nucleotide | CGCTGGTGCTCTAAAAAGAAGGATGCC |
| | | | GCGGTGATGAACCAGGAGCCCGCTGGC |
| | | | CACCGCACCGTCAACCGCGAGGACAGT |
| | | | GACGAGCAGGATCCGCAGGAGGTGACC |
| | | | TACGCGCAGTTGGATCACTGTATTTTC |
| | | | ACTCAGCGCAAGATTACCGGCCCTTCA |
| | | | CAGAGGTCCAAGCGCCCTTCGACCGAC |
| | | | ACCTCTGTCTGTATTGAGTTGCCCAAC |
| | | | GCGGAGCCAAGAGCACTGAGCCCGGCG |
| | | | CATGAGCACCACAGCCAGGCCCTGATG |
| | | | GGCTCGTCCCGCGAAACGACAGCTCTC |
| | | | TCGCAGACCCAGCTTGCTAGCTCTAAT |
| | | | GTACCAGCAGCCGGGATC (SEQ ID |
| | | | NO: 131) |
| KIR2DL5 | KIR2DL5 ICD | Amino Acid | LHCCCSNKKNAAVMDQEPAGDRTVNRE |
| | | | DSDDQDPQEVTYAQLDHCVFTQTKITS |
| | | | PSQRPKTPPTDTTMYMELPNAKPRSLS |
| | | | PAHKHHSQALRGSSRETTALSQNRVAS |
| | | | SHVPAAGI (SEQ ID NO: 110) |
| KIR2DL5 | KIR2DL5 ICD | Nucleotide | CTTCATTGCTGCTGCTCCAACAAAAAG |
| | | | AATGCTGCTGTAATGGACCAAGAGCCT |
| | | | GCCGGGGACAGAACAGTGAACAGGGAG |
| | | | GACTCTGATGATCAAGACCCTCAGGAG |
| | | | GTGACATATGCACAGTTGGATCACTGC |
| | | | GTTTTCACACAGACAAAAATCACTTCC |
| | | | CCTTCTCAGAGGCCCAAGACACCTCCA |
| | | | ACAGATACCACCATGTACATGGAACTT |
| | | | CCAAATGCTAAGCCAAGATCATTGTCT |
| | | | CCTGCCCATAAGCACCACAGTCAGGCC |
| | | | TTGAGGGGATCTTCTAGGGAGACAACA |
| | | | GCCCTGTCTCAAAACCGGGTTGCTAGC |
| | | | TCCCATGTACCAGCAGCTGGAATC |
| | | | (SEQ ID NO: 132) |
| SIGLEC-7 | SIGLEC-7 ICD | Amino Acid | RSCRKKSARPAADVGDIGMKDANTIRG |
| | | | SASQGNLTESWADDNPRHHGLAAHSSG |
| | | | EEREIQYAPLSFHKGEPQDLSGQEATN |
| | | | NEYSEIKIPK (SEQ ID NO: 111) |
| SIGLEC-7 | SIGLEC-7 ICD | Nucleotide | AGGTCCTGCAGGAAGAAATCGGCAAGG |
| | | | CCAGCAGCGGACGTGGGAGACATAGGC |
| | | | ATGAAGGATGCAAACACCATCAGGGGC |
| | | | TCAGCCTCTCAGGGTAACCTGACTGAG |
| | | | TCCTGGGCAGATGATAACCCCCGACAC |
| | | | CATGGCCTGGCTGCCCACTCCTCAGGG |
| | | | GAGGAAAGAGAGATCCAGTATGCACCC |
| | | | CTCAGCTTTCATAAGGGGGAGCCTCAG |
| | | | GACCTATCAGGACAAGAAGCCACCAAC |
| | | | AATGAGTACTCAGAGATCAAGATCCCC |
| | | | AAG (SEQ ID NO: 133) |
| FCRH3 | FCRH3 ICD | Amino Acid | HYARARRKPGGLSATGTSSHSPSECQE |
| | | | PSSSRPSRIDPQEPTHSKPLAPMELEP |
| | | | MYSNVNPGDSNPIYSQIWSIQHTKENS |
| | | | ANCPMMHQEHEELTVLYSELKKTHPDD |
| | | | SAGEASSRGRAHEEDDEENYENVPRVL |
| | | | LASDH (SEQ ID NO: 112) |
| FCRH3 | FCRH3 ICD | Nucleotide | CATTATGCCAGAGCCAGAAGAAAGCCT |
| | | | GGCGGCCTGTCTGCCACCGGCACATCT |
| | | | TCTCACAGCCCCAGCGAGTGTCAAGAG |
| | | | CCCAGCAGCAGCAGACCCAGCAGAATC |
| | | | GATCCCCAAGAGCCTACACACAGCAAG |
| | | | CCCCTGGCTCCTATGGAACTGGAACCC |
| | | | ATGTACAGCAACGTGAACCCCGGCGAC |
| | | | AGCAACCCCATCTACAGCCAGATTTGG |
| | | | AGCATCCAGCACACCAAAGAGAACAGC |
| | | | GCCAACTGTCCCATGATGCACCAAGAG |
| | | | CACGAGGAACTGACCGTGCTGTACTCC |
| | | | GAGCTGAAGAAAACACACCCCGACGAC |
| | | | TCTGCCGGCGAGGCCTCTTCTAGAGGC |
| | | | AGAGCCCATGAAGAGGACGACGAAGAG |
| | | | AACTACGAGAACGTGCCCAGAGTGCTG |
| | | | CTGGCCTCCGATCAT (SEQ ID NO: |
| | | | 134) |
| PCDHGC5 | PCDHGC5 ICD | Amino Acid | KCLQGNADGDGGGGQCCRRQDSPSPDF |
| | | | YKQSSPNLQVSSDGTLKYMEVTLRPTD |
| | | | SQSHCYRTCFSPASDGSDFTFLRPLSV |
| | | | QQPTALALEPDAIRSRSNTLRERSQQA |
| | | | PPNTDWRFSQAQRPGTSGSQNGDDTGT |
| | | | WPNNQFDTEMLQAMILASASEAADGSS |
| | | | TLGGGAGTMGLSARYGPQFTLQHVPDY |
| | | | RQNVYIPGSNATLTNAAGKRDGKAPAG |
| | | | GNGNKKKSGKKEKK (SEQ ID NO: |
| | | | 113) |
| PCDHGC5 | PCDHGC5 ICD | Nucleotide | AAGTGCCTGCAGGGAAATGCTGATGGC |
| | | | GACGGTGGCGGAGGCCAGTGTTGTAGA |
| | | | AGGCAGGATAGCCCCTCTCCAGACTTC |
| | | | TACAAGCAGAGCAGCCCCAACCTCCAG |
| | | | GTGTCCTCTGATGGCACCCTGAAGTAC |
| | | | ATGGAAGTGACCCTGAGGCCTACCGAC |
| | | | AGCCAGAGCCACTGCTACAGAACCTGC |
| | | | TTTAGCCCTGCCAGCGACGGCAGCGAC |
| | | | TTTACCTTTCTGAGGCCTCTGAGCGTG |
| | | | CAGCAGCCTACAGCTCTGGCTCTGGAA |
| | | | CCTGACGCCATCAGAAGCAGAAGCAAC |
| | | | ACCCTGAGAGAGCGGAGCCAGCAGGCC |
| | | | CCTCCTAATACCGATTGGAGATTCAGC |
| | | | CAGGCTCAGCGGCCTGGCACAAGCGGA |
| | | | TCTCAGAATGGCGACGATACCGGCACC |
| | | | TGGCCTAACAACCAGTTCGACACCGAA |
| | | | ATGCTGCAGGCCATGATTCTGGCCTCT |
| | | | GCCTCTGAAGCCGCCGATGGCAGTTCT |
| | | | ACACTTGGAGGCGGAGCCGGCACAATG |
| | | | GGACTGTCTGCTAGATACGGCCCTCAG |
| | | | TTCACCCTGCAGCACGTGCCCGACTAC |
| | | | AGACAGAACGTGTACATCCCCGGCAGC |
| | | | AACGCCACACTGACAAATGCCGCCGGA |
| | | | AAGAGAGATGGCAAAGCCCCTGCTGGC |
| | | | GGCAACGGCAACAAGAAGAAGTCCGGC |
| | | | AAGAAAGAGAAGAAG (SEQ ID NO: |
| | | | 135) |
| CDH11 | CDH11 ICD | Amino Acid | RRQKKEPLIVFEEEDVRENIITYDDEG |
| | | | GGEEDTEAFDIATLQNPDGINGFIPRK |
| | | | DIKPEYQYMPRPGLRPAPNSVDVDDFI |
| | | | NTRIQEADNDPTAPPYDSIQIYGYEGR |
| | | | GSVAGSLSSLESATTDSDLDYDYLQNW |
| | | | GPRFKKLADLYGSKDTFDDDS(SEQ |
| | | | ID NO: 114) |
| CDH11 | CDH11 ICD | Nucleotide | CGGCGGCAGAAGAAAGAACCCCTGATC |
| | | | GTGTTCGAAGAGGAAGATGTGCGCGAG |
| | | | AACATCATCACCTACGACGACGAAGGC |
| | | | GGAGGCGAAGAGGATACCGAGGCCTTC |
| | | | GATATCGCCACACTGCAGAACCCCGAC |
| | | | GGCATCAACGGCTTCATCCCCAGAAAG |
| | | | GACATCAAGCCCGAGTACCAGTACATG |
| | | | CCCAGACCTGGACTCAGACCCGCTCCT |
| | | | AATTCCGTGGACGTGGACGACTTCATC |
| | | | AACACCCGGATCCAAGAGGCCGACAAC |
| | | | GACCCTACAGCTCCTCCATACGACAGC |
| | | | ATCCAGATCTACGGCTACGAAGGCAGA |
| | | | GGAAGCGTGGCCGGATCTCTGAGCAGT |
| | | | CTGGAAAGCGCCACCACCGACAGCGAC |
| | | | CTGGATTACGACTACCTGCAGAACTGG |
| | | | GGCCCTAGATTCAAGAAGCTGGCCGAC |
| | | | CTGTACGGCAGCAAGGACACCTTCGAC |
| | | | GATGACAGC (SEQ ID NO: 136) |
| IMPG2 | IMPG2 ICD | Amino Acid | YFFIRTLQAHHDRSERESPFSGSSRQP |
| | | | DSLSSIENAVKYNPVYESHRAGCEKYE |
| | | | GPYPQHPFYSSASGDVIGGLSREEIRQ |
| | | | MYESSELSREEIQERMRVLELYANDPE |
| | | | FAAFVREQQVEEV (SEQ ID NO: |
| | | | 115) |
| IMPG2 | IMPG2 ICD | Nucleotide | TACTTTTTCATCCGGACACTGCAGGCC |
| | | | CATCACGACAGATCCGAGAGAGAGAGC |
| | | | CCTTTCAGCGGCAGCAGCAGACAGCCT |
| | | | GATAGCCTGAGCAGCATCGAGAACGCC |
| | | | GTGAAGTACAACCCCGTGTACGAGTCT |
| | | | CACAGAGCCGGCTGCGAGAAGTACGAG |
| | | | GGCCCTTATCCTCAGCACCCCTTCTAC |
| | | | AGCTCTGCCAGCGGAGATGTGATCGGC |
| | | | GGCCTGTCCAGAGAAGAGATCCGGCAG |
| | | | ATGTACGAGAGCAGCGAGCTGAGCCGG |
| | | | GAAGAGATTCAAGAGCGGATGAGAGTG |
| | | | CTGGAACTGTACGCCAACGATCCCGAG |
| | | | TTCGCCGCCTTTGTGCGAGAACAGCAG |
| | | | GTCGAGGAAGTG (SEQ ID NO: |
| | | | 137) |
| DSCAM | DSCAM ICD | Amino Acid | RRRRREQRLKRLRDAKSLAEMLMSKNT |
| | | | RTSDTLSKQQQTLRMHIDIPRAQLLIE |
| | | | ERDTMETIDDRSTVLLTDADEGEAAKQ |
| | | | KSLTVTHTVHYQSVSQATGPLVDVSDA |
| | | | RPGTNPTTRRNAKAGPTARNRYASQWT |
| | | | LNRPHPTISAHTLTTDWRLPTPRAAGS |
| | | | VDKESDSYSVSPSQDTDRARSSMVSTE |
| | | | SASSTYEELARAYEHAKMEEQLRHAKF |
| | | | TITECFISDTSSEQLTAGTNEYTDSLT |
| | | | SSTPSESGICRFTASPPKPQDGGRVMN |
| | | | MAVPKAHRPGDLIHLPPYLRMDFLLNR |
| | | | GGPGTSRDLSLGQACLEPQKSRTLKRP |
| | | | TVLEPIPMEAASSASSTREGQSWQPGA |
| | | | VATLPQREGAELGQAAKMSSSQESLLD |
| | | | SRGHLKGNNPYAKSYTLV (SEQ ID |
| | | | NO: 116) |
| DSCAM | DSCAM ICD | Nucleotide | CGGCGGAGAAGAAGAGAGCAGCGGCTG |
| | | | AAGAGACTGCGGGATGCCAAATCTCTG |
| | | | GCCGAGATGCTGATGAGCAAGAACACC |
| | | | AGAACCAGCGACACCCTGAGCAAGCAG |
| | | | CAACAGACCCTGCGGATGCACATCGAC |
| | | | ATCCCCAGAGCACAGCTGCTGATCGAG |
| | | | GAACGGGACACCATGGAAACCATCGAC |
| | | | GACAGATCCACCGTGCTGCTGACCGAT |
| | | | GCCGATTTTGGAGAGGCCGCCAAGCAG |
| | | | AAAAGCCTGACCGTGACACACACCGTG |
| | | | CACTACCAGTCTGTGTCCCAGGCTACA |
| | | | GGACCTCTGGTGGACGTGTCAGATGCC |
| | | | AGACCTGGCACAAACCCCACCACCAGA |
| | | | AGAAACGCCAAGGCCGGACCTACCGCC |
| | | | AGAAACAGATATGCCAGCCAGTGGACC |
| | | | CTGAACAGACCCCATCCTACCATCAGC |
| | | | GCCCACACACTGACCACCGATTGGAGA |
| | | | CTGCCCACACCTAGAGCCGCCGGATCT |
| | | | GTGGACAAAGAGTCCGACAGCTACAGC |
| | | | GTGTCCCCTAGCCAGGATACCGACAGA |
| | | | GCCAGATCCAGCATGGTGTCTACAGAG |
| | | | AGCGCCAGCAGCACCTACGAGGAACTG |
| | | | GCCAGAGCCTATGAGCACGCCAAGATG |
| | | | GAAGAACAGCTGCGCCACGCCAAGTTC |
| | | | ACCATCACCGAGTGCTTCATCTCCGAC |
| | | | ACCAGCAGCGAACAGCTGACCGCCGGC |
| | | | ACCAATGAGTACACCGATAGCCTGACC |
| | | | TCCAGCACACCTTCCGAGAGCGGCATC |
| | | | TGCAGGTTTACCGCCTCTCCACCTAAG |
| | | | CCTCAGGATGGCGGCAGAGTGATGAAC |
| | | | ATGGCCGTGCCTAAGGCTCACAGACCC |
| | | | GGCGACCTGATTCATCTGCCACCTTAC |
| | | | CTGAGAATGGACTTCCTGCTGAACAGA |
| | | | GGCGGCCCAGGCACAAGCAGAGATCTG |
| | | | TCTCTTGGCCAGGCCTGCCTGGAACCT |
| | | | CAGAAGTCTAGAACCCTGAAGCGGCCT |
| | | | ACCGTGCTGGAACCCATTCCTATGGAA |
| | | | GCCGCCAGCTCTGCCTCTAGCACAAGA |
| | | | GAGGGACAGTCTTGGCAGCCTGGCGCT |
| | | | GTTGCTACACTGCCTCAAAGAGAAGGC |
| | | | GCCGAACTGGGACAAGCCGCCAAAATG |
| | | | AGCAGCAGCCAAGAGAGCCTGCTGGAC |
| | | | TCTAGAGGCCACCTGAAGGGCAACAAC |
| | | | CCCTACGCCAAGAGCTACACCCTGGTG |
| | | | (SEQ ID NO: 138) |

### Example 4: Inhibitory Chimeric Receptors with Various Intracellular Signaling Domains Modulate NK-Medicated Cell Killing

### Methods

NK cells were isolated from peripheral blood and cultured and expanded for 11 days with irradiated K562 feeder cells engineered to express membrane-bound IL-21 and IL-15 in NK MACS® media with 5% human AB serum. During expansion and subsequent NK cell culture, NK cell media was supplemented with 500 U/mL IL-2 and 10 ng/mL IL-15 every 3-4 days. At day 11, NK cells were transduced with gamma-retrovirus carrying gene payloads encoding an CEA-targeting aCAR and/or VSIG2-targeting iCAR proteins with varying intracellular domains.

Constructs with the various inhibitory ICDs were in the following format (linearly linked in N-terminal to C-terminal orientation):
CD8ss—aCEA hMN14 LH—myc NGAA linker—CD8 hinge—CD28 TM—CD28 ICD—CD3z—GSG P2A—CD8SS-VSIG2 scFv—V5 NGAA linker—CD8 hinge—ICD TM-ICD Domain

A construct that included a SIRPα TM and ICD with the anti-VSIG scFv replaced with a Her2-targeting scFv was also assessed.

After 3 days post-transduction, NK cells media was removed and fresh media with 500 U/mL IL-2 and 10 ng/mL IL-15 was added. After an additional 3 days, media was replaced again with fresh media and cytokines, and expression of aCAR and iCAR was measured by antibody staining. Myc tags on aCARs and V5 tags on iCARs were stained with anti-myc Alex Fluor 488 (Cell Signaling Technology) at 1:50 dilution and anti-V5-Alexa Fluor 647 (Invitrogen) at 1:300 dilution, respectively. Cells were washed with phosphate-buffered saline supplemented with 5% fetal (FBS) bovine serum and resuspended in the viability dye SytoxBlue (Invitrogen) at 1:1000 dilution.

After an additional 2 days, cytotoxicity assays were performed using the colorectal adenocarcinoma cell line DLD-1 engineered to express CEACAM5, VSIG2, mCherry (DLD-1 CEA+VSIG2+mCherry+) as targets and RPMI supplemented with 10% FBS were used as the assay media. All cytotoxicity assays were analyzed using the Sartorius Incucyte imaging instrument. These target cells were harvested by trypsinization, washed, and counted. The two types of target cells were diluted to 1e6 cells/mL and then mixed 1:1. Then, 2e4 target cells were added in a 100 μL volume to wells of 96 well flat bottom plates. The targets were then incubated overnight (16-18 hours) prior to NK cell addition. To prepare NK cells for the assay, they were harvested, washed, and counted. Then, 1e4 NK cells were added in a 100 μL volume in triplicates to the mixed target plates.

These plates were then moved to the Incucyte® for whole-well imaging every 4 hours. The target cell reduction (also called killing) was quantified as 100%×(1−No. Targets/No. Targets (targets alone)). Suppression was quantified as 100%×(1−killing of VSIG2+ cells/killing of all VSIG2− cells) and Δ% VSIG2+ was quantified as the change in the following quantity relative to untransduced NK cells: 100%×(No. VSIG2+/No. all target cells).

### Results

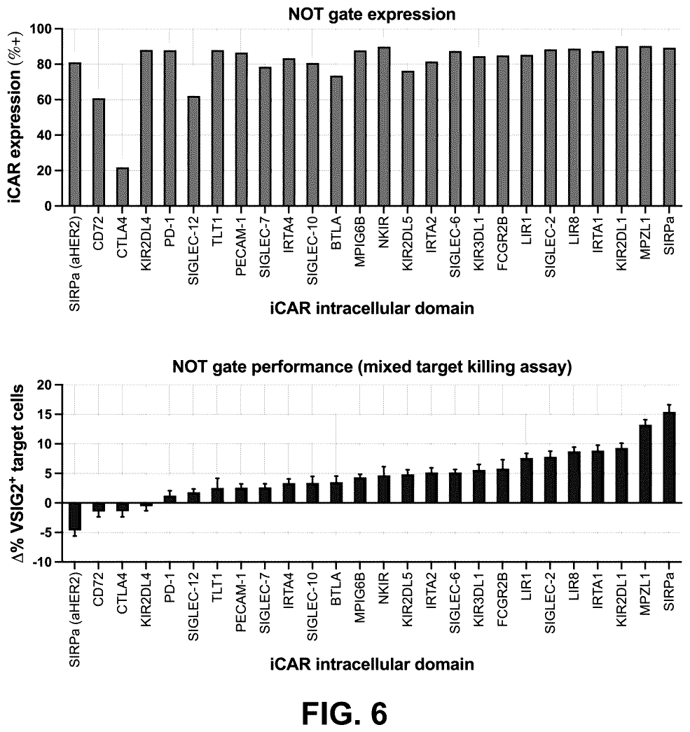

Expression of the iCARs with the various inhibitory ICDs was assessed. As shown in FIG. 6 (top panel), expression was generally equivalent across all constructs.

Anti-VSIG2 iCAR-mediated protection with the various inhibitory ICDs was assessed. As shown in FIG. 6 (bottom panel), a range of protection was shown with the majority of constructs demonstrating a protective effect, indicating the ability of iCARs with the tested ICDs provided iCAR mediated protection towards VSIG2 expressing cells. These results indicate the ability of the tested ICDs to work in iCARs having scFvs to different target antigens.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A chimeric inhibitory receptor comprising:
an extracellular protein binding domain;
a transmembrane domain, wherein the transmembrane domain is operably linked to the extracellular protein binding domain; and
one or more intracellular signaling domains, wherein the one or more intracellular signaling domains are operably linked to the transmembrane domain,
wherein the one or more intracellular signaling domains are each derived from a protein selected from the group consisting of: MPZL1, IRTA1, LIR8, PECAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZ, FCGR2B, SIGLEC-6, MPIG6B, VSIG4, SIGLEC-12, KIR2DL4, KIR2DL5, SIGLEC-7, FCRH3, PCDHGC5, CDH11, IMPG2, and DSCAM, and
wherein at least one of the one or more intracellular signaling domains is capable of preventing, attenuating, or inhibiting activation of a tumor-targeting chimeric receptor expressed on an immunomodulatory cell.

2. The chimeric inhibitory receptor of claim 1, wherein:
a. the transmembrane domain is derived from the same protein as one of the one or more intracellular signaling domains, optionally wherein the transmembrane domain further comprises at least a portion of an extracellular domain of the same protein; or
b. the transmembrane domain is derived from a first protein and the one or more intracellular signaling domains are derived from proteins that are distinct from the first protein.

3. The chimeric inhibitory receptor of claim 1, wherein:
a. one of the one or more intracellular signaling domains is derived from PECAM-1, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to KCYFLRKAKAKQMPVEMSRPAVPLLNSNNEKMSDPNMEANSHYGHNDDVRNH AMKPINDNKEPLNSDVQYTEVQVSSAESHKDLGKKDTETVYSEVRKAVPDAVE SRYSRTEGSLDGT (SEQ ID NO: 1), optionally wherein one of the one or more intracellular signaling domains comprises the amino acid sequence of KCYFLRKAKAKQMPVEMSRPAVPLLNSNNEKMSDPNMEANSHYGHNDDVRNH AMKPINDNKEPLNSDVQYTEVQVSSAESHKDLGKKDTETVYSEVRKAVPDAVE SRYSRTEGSLDGT (SEQ ID NO: 1);
b. one of the one or more intracellular signaling domains is derived from CD72, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to MAEAITYADLRFVKAPLKKSISSRLGQDPGADDDGEITYENVQVPAVLGVPSSLA SSVLGDKAAVKSEQPTASWRAVTSPAVGRILPCRTTCLRY (SEQ ID NO: 2), optionally wherein one of the one or more intracellular signaling domains comprises the amino acid sequence of MAEAITYADLRFVKAPLKKSISSRLGQDPGADDDGEITYENVQVPAVLGVPSSLA SSVLGDKAAVKSEQPTASWRAVTSPAVGRILPCRTTCLRY (SEQ ID NO: 2);
c. one of the one or more intracellular signaling domains is derived from IRTA2, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LSRKAGRKPASDPARSPSDSDSQEPTYHNVPAWEELQPVYTNANPRGENVVYSE VRIIQEKKKHAVASDPRHLRNKGSPIIYSEVKVASTPVSGSLFLASSAPHR (SEQ ID NO: 3), optionally wherein one of the one or more intracellular signaling domains comprises the amino acid sequence of LSRKAGRKPASDPARSPSDSDSQEPTYHNVPAWEELQPVYTNANPRGENVVYSE VRIIQEKKKHAVASDPRHLRNKGSPIIYSEVKVASTPVSGSLFLASSAPHR (SEQ ID NO: 3);
d. one of the one or more intracellular signaling domains is derived from IRTA4, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to HKISGESSATNEPRGASRPNPQEFTYSSPTPDMEELQPVYVNVGSVDVDVVYSQV WSMQQPESSANIRTLLENKDSQVIYSSVKKS (SEQ ID NO: 4), optionally wherein the intracellular signaling domain comprises the amino acid sequence of HKISGESSATNEPRGASRPNPQEFTYSSPTPDMEELQPVYVNVGSVDVDVVYSQV WSMQQPESSANIRTLLENKDSQVIYSSVKKS (SEQ ID NO: 4)
e. one of the one or more intracellular signaling domains is derived from NKIR, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to WRMMKYQQKAAGMSPEQVLQPLEGDLCYADLTLQLAGTSPQKATTKLSSAQV DQVEVEYVTMASLPKEDISYASLTLGAEDQEPTYCNMGHLSSHLPGRGPEEPTE YSTISRP (SEQ ID NO: 5), optionally wherein one of the one or more intracellular signaling domains comprises the amino acid sequence of WRMMKYQQKAAGMSPEQVLQPLEGDLCYADLTLQLAGTSPQKATTKLSSAQV DQVEVEYVTMASLPKEDISYASLTLGAEDQEPTYCNMGHLSSHLPGRGPEEPTE YSTISRP (SEQ ID NO: 5);
f. one of the one or more intracellular signaling domains is derived from IL1RAP, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to YRAHFGTDETILDGKEYDIYVSYARNAEEEEFVLLTLRGVLENEFGYKLCIFDRD SLPGGIVTDETLSFIQKSRRLLVVLSPNYVLQGTQALLELKAGLENMASRGNINVI LVQYKAVKETKVKELKRAKTVLTVIKWKGEKSKYPQGRFWKQLQVAMPVKKS PRRSSSDEQGLSYSSLKNV (SEQ ID NO: 6), optionally wherein one of the one or more intracellular signaling domains comprises the amino acid sequence of YRAHFGTDETILDGKEYDIYVSYARNAEEEEFVLLTLRGVLENEFGYKLCIFDRD SLPGGIVTDETLSFIQKSRRLLVVLSPNYVLQGTQALLELKAGLENMASRGNINVI LVQYKAVKETKVKELKRAKTVLTVIKWKGEKSKYPQGRFWKQLQVAMPVKKS PRRSSSDEQGLSYSSLKNV (SEQ ID NO: 6);
g. one of the one or more intracellular signaling domains is derived from PTPRO, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LRKKHLQMARECGAGTFVNFASLERDGKLPYNWRRSIFAFLTLLPSCLWTDYLL AFYINPWSKNGLKKRKLTNPVQLDDFDAYIKDMAKDSDYKFSLQFEELKLIGLDI PHFAADLPLNRCKNRYTNILPYDFSRVRLVSMNEEEGADYINANYIPGYNSPQEY IATQGPLPETRNDFWKMVLQQKSQIIVMLTQCNEKRRVKCDHYWPFTEEPIAYG DITVEMISEEEQDDWACRHFRINYADEMQDVMHFNYTAWPDHGVPTANAAESI LQFVHMVRQQATKSKGPMIIHCSAGVGRTGTFIALDRLLQHIRDHEFVDILGLVS EMRSYRMSMVQTEEQYIFIHQCVQLMWMKKKQQFCISDVIYENVSKS (SEQ ID NO: 7), optionally wherein one of the one or more intracellular signaling domains comprises the amino acid sequence of LRKKHLQMARECGAGTFVNFASLERDGKLPYNWRRSIFAFLTLLPSCLWTDYLL AFYINPWSKNGLKKRKLTNPVQLDDFDAYIKDMAKDSDYKFSLQFEELKLIGLDI PHFAADLPLNRCKNRYTNILPYDFSRVRLVSMNEEEGADYINANYIPGYNSPQEY IATQGPLPETRNDFWKMVLQQKSQIIVMLTQCNEKRRVKCDHYWPFTEEPIAYG DITVEMISEEEQDDWACRHFRINYADEMQDVMHFNYTAWPDHGVPTANAAESI LQFVHMVRQQATKSKGPMIIHCSAGVGRTGTFIALDRLLQHIRDHEFVDILGLVS EMRSYRMSMVQTEEQYIFIHQCVQLMWMKKKQQFCISDVIYENVSKS (SEQ ID NO: 7);
h. one of the one or more intracellular signaling domains is derived from PTPRZ1, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RKCFQTAHFYLEDSTSPRVISTPPTPIFPISDDVGAIPIKHFPKHVADLHASSGFTEE FETLKEFYQEVQSCTVDLGITADSSNHPDNKHKNRYINIVAYDHSRVKLAQLAEK DGKLTDYINANYVDGYNRPKAYIAAQGPLKSTAEDFWRMIWEHNVEVIVMITN LVEKGRRKCDQYWPADGSEEYGNFLVTQKSVQVLAYYTVRNFTLRNTKIKKGS QKGRPSGRVVTQYHYTQWPDMGVPEYSLPVLTFVRKAAYAKRHAVGPVVVHC SAGVGRTGTYIVLDSMLQQIQHEGTVNIFGFLKHIRSQRNYLVQTEEQYVFIHDT LVEAILSKETEVLDSHIHAYVNALLIPGPAGKTKLEKQFQLLSQSNIQQSDYSAAL KQCNREKNRTSSIIPVERSRVGISSLSGEGTDYINASYIMGYYQSNEFIITQHPLLH TIKDFWRMIWDHNAQLVVMIPDGQNMAEDEFVYWPNKDEPINCESFKVTLMAE EHKCLSNEEKLIIQDFILEATQDDYVLEVRHFQCPKWPNPDSPISKTFELISVIKEE AANRDGPMIVHDEHGGVTAGTFCALTTLMHQLEKENSVDVYQVAKMINLMRP GVFADIEQYQFLYKVILSLVSTRQEENPSTSLDSNGAALPDGNIAESLESLV (SEQ ID NO: 8), optionally wherein one of the one or more intracellular signaling domains comprises the amino acid sequence of RKCFQTAHFYLEDSTSPRVISTPPTPIFPISDDVGAIPIKHFPKHVADLHASSGFTEE FETLKEFYQEVQSCTVDLGITADSSNHPDNKHKNRYINIVAYDHSRVKLAQLAEK DGKLTDYINANYVDGYNRPKAYIAAQGPLKSTAEDFWRMIWEHNVEVIVMITN LVEKGRRKCDQYWPADGSEEYGNFLVTQKSVQVLAYYTVRNFTLRNTKIKKGS QKGRPSGRVVTQYHYTQWPDMGVPEYSLPVLTFVRKAAYAKRHAVGPVVVHC SAGVGRTGTYIVLDSMLQQIQHEGTVNIFGFLKHIRSQRNYLVQTEEQYVFIHDT LVEAILSKETEVLDSHIHAYVNALLIPGPAGKTKLEKQFQLLSQSNIQQSDYSAAL KQCNREKNRTSSIIPVERSRVGISSLSGEGTDYINASYIMGYYQSNEFIITQHPLLH TIKDFWRMIWDHNAQLVVMIPDGQNMAEDEFVYWPNKDEPINCESFKVTLMAE EHKCLSNEEKLIIQDFILEATQDDYVLEVRHFQCPKWPNPDSPISKTFELISVIKEE AANRDGPMIVHDEHGGVTAGTFCALTTLMHQLEKENSVDVYQVAKMINLMRP GVFADIEQYQFLYKVILSLVSTRQEENPSTSLDSNGAALPDGNIAESLESLV (SEQ ID NO: 8);
i. one of the one or more intracellular signaling domains is derived from TLT1, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to MAKRKQGNRLGVCGRFLSSRVSGMNPSSVVHHVSDSGPAAELPLDVPHIRLDSP PSFDNTTYTSLPLDSPSGKPSLPAPSSLPPLPPKVLVCSKPVTYATVIFPGGNKGGG TSCGPAQNPPNNQTPSS (SEQ ID NO: 9), optionally wherein one of the one or more intracellular signaling domains comprises the amino acid sequence of MAKRKQGNRLGVCGRFLSSRVSGMNPSSVVHHVSDSGPAAELPLDVPHIRLDSP PSFDNTTYTSLPLDSPSGKPSLPAPSSLPPLPPKVLVCSKPVTYATVIFPGGNKGGG TSCGPAQNPPNNQTPSS (SEQ ID NO: 9);
j. one of the one or more intracellular signaling domains is derived from SLAMF1, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to QLRRRGKTNHYQTTVEKKSLTIYAQVQKPGPLQKKLDSFPAQDPCTTIYVAATEP VPESVQETNSITVYASVTLPES (SEQ ID NO: 10), optionally wherein one of the one or more intracellular signaling domains comprises the amino acid sequence of QLRRRGKTNHYQTTVEKKSLTIYAQVQKPGPLQKKLDSFPAQDPCTTIYVAATEP VPESVQETNSITVYASVTLPES (SEQ ID NO: 10);
k. one of the one or more intracellular signaling domains is derived from SLAMF5, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RLFKRRQGRIFPEGSCLNTFTKNPYAASKKTIYTYIMASRNTQPAESRIYDEILQSK VLPSKEEPVNTVYSEVQFADKMGKASTQDSKPPGTSSYEIVI (SEQ ID NO: 11), optionally wherein one of the one or more intracellular signaling domains comprises the amino acid sequence of RLFKRRQGRIFPEGSCLNTFTKNPYAASKKTIYTYIMASRNTQPAESRIYDEILQSK VLPSKEEPVNTVYSEVQFADKMGKASTQDSKPPGTSSYEIVI (SEQ ID NO: 11);
l. one of the one or more intracellular signaling domains is derived from PCDHGC3, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to FKVYKWKQSRDLYRAPVSSLYRTPGPSLHADAVRGGLMSPHLYHQVYLTTDSR RSDPLLKKPGAASPLASRQNTLRSCDPVFYRQVLGAESAPPGQQAPPNTDWRFS QAQRPGTSGSQNGDDTGTWPNNQFDTEMLQAMILASASEAADGSSTLGGGAGT MGLSARYGPQFTLQHVPDYRQNVYIPGSNATLTNAAGKRDGKAPAGGNGNKK KSGKKEKK (SEQ ID NO: 95), optionally wherein one of the one or more intracellular signaling domains comprises the amino acid sequence of FKVYKWKQSRDLYRAPVSSLYRTPGPSLHADAVRGGLMSPHLYHQVYLTTDSR RSDPLLKKPGAASPLASRQNTLRSCDPVFYRQVLGAESAPPGQQAPPNTDWRFS QAQRPGTSGSQNGDDTGTWPNNQFDTEMLQAMILASASEAADGSSTLGGGAGT MGLSARYGPQFTLQHVPDYRQNVYIPGSNATLTNAAGKRDGKAPAGGNGNKK KSGKKEKK (SEQ ID NO: 95);
m. one of the one or more intracellular signaling domains is derived from LIFR, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to YRKREWIKETFYPDIPNPENCKALQFQKSVCEGSSALKTLEMNPCTPNNVEVLET RSAFPKIEDTEIISPVAERPEDRSDAEPENHVVVSYCPPIIEEEIPNPAADEAGGTAQ VIYIDVQSMYQPQAKPEEEQENDPVGGAGYKPQMHLPINSTVEDIAAEEDLDKT AGYRPQANVNTWNLVSPDSPRSIDSNSEIVSFGSPCSINSRQFLIPPKDEDSPKSNG GGWSFTNFFQNKPND (SEQ ID NO: 96), optionally wherein one of the one or more intracellular signaling domains comprises the amino acid sequence of YRKREWIKETFYPDIPNPENCKALQFQKSVCEGSSALKTLEMNPCTPNNVEVLET RSAFPKIEDTEIISPVAERPEDRSDAEPENHVVVSYCPPIIEEEIPNPAADEAGGTAQ VIYIDVQSMYQPQAKPEEEQENDPVGGAGYKPQMHLPINSTVEDIAAEEDLDKT AGYRPQANVNTWNLVSPDSPRSIDSNSEIVSFGSPCSINSRQFLIPPKDEDSPKSNG GGWSFTNFFQNKPND (SEQ ID NO: 96);
n. one of the one or more intracellular signaling domains is derived from ERMAP, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to WKQRRAKEKLLYEHVTEVDNLLSDHAKEKGKLHKAVKKLRSELKLKRAAANS GWRRARLHFVAVTLDPDTAHPKLILSEDQRCVRLGDRRQPVPDNPQRFDFVVSI LGSEYFTTGCHYWEVYVGDKTKWILGVCSESVSRKGKVTASPANGHWLLRQSR GNEYEALTSPOTSFRLKEPPRCVGIFLDYEAGVISFYNVTNKSHIFTFTHNFSGPLR PFFEPCLHDGGKNTAPLVICSELHKSEESIVPRPEGKGHANGDVSLKVNSSLLPPK APELKDIILSLPPDLGPALQELKAPSF (SEQ ID NO: 97), optionally wherein one of the one or more intracellular signaling domains comprises the amino acid sequence of WKQRRAKEKLLYEHVTEVDNLLSDHAKEKGKLHKAVKKLRSELKLKRAAANS GWRRARLHFVAVTLDPDTAHPKLILSEDQRCVRLGDRRQPVPDNPQRFDFVVSI LGSEYFTTGCHYWEVYVGDKTKWILGVCSESVSRKGKVTASPANGHWLLRQSR GNEYEALTSPQTSFRLKEPPRCVGIFLDYEAGVISFYNVTNKSHIFTFTHNFSGPLR PFFEPCLHDGGKNTAPLVICSELHKSEESIVPRPEGKGHANGDVSLKVNSSLLPPK APELKDIILSLPPDLGPALQELKAPSF (SEQ ID NO: 97);
o. one of the one or more intracellular signaling domains is derived from IL1RAPL2, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to KCYNIELMLFYRQHFGADETNDDNKEYDAYLSYTKVDQDTLDCDNPEEEQFAL EVLPDVLEKHYGYKLFIPERDLIPSGTYMEDLTRYVEQSRRLIIVLTPDYILRRGW SIFELESRLHNMLVSGEIKVILIECTELKGKVNCQEVESLKRSIKLLSLIKWKGSKS SKLNSKFWKHLVYEMPIKKKEMLPRCHVLDSAEQGLFGELQPIPSIAMTSTSATL VSSQADLPEFHPSDSMQIRHCCRGYKHEIPATTLPVPSLGNHHTYCNLPLTLLNG QLPLNNTLKDTQEFHRNSSLLPLSSKELSFTSDIW (SEQ ID NO: 98), optionally wherein one of the one or more intracellular signaling domains comprises the amino acid sequence of KCYNIELMLFYRQHFGADETNDDNKEYDAYLSYTKVDQDTLDCDNPEEEQFAL EVLPDVLEKHYGYKLFIPERDLIPSGTYMEDLTRYVEQSRRLIIVLTPDYILRRGW SIFELESRLHNMLVSGEIKVILIECTELKGKVNCQEVESLKRSIKLLSLIKWKGSKS SKLNSKFWKHLVYEMPIKKKEMLPRCHVLDSAEQGLFGELQPIPSIAMTSTSATL VSSQADLPEFHPSDSMQIRHCCRGYKHEIPATTLPVPSLGNHHTYCNLPLTLLNG QLPLNNTLKDTQEFHRNSSLLPLSSKELSFTSDIW (SEQ ID NO: 98);
p. one of the one or more intracellular signaling domains is derived from CDH5, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RRRLRKQARAHGKSVPEIHEQLVTYDEEGGGEMDTTSYDVSVLNSVRRGGAKP PRPALDARPSLYAQVQKPPRHAPGAHGGPGEMAAMIEVKKDEADHDGDGPPYD TLHIYGYEGSESIAESLSSLGTDSSDSDVDYDFLNDWGPRFKMLAELYGSDPREE LLY (SEQ ID NO: 99), optionally wherein one of the one or more intracellular signaling domains comprises the amino acid sequence of RRRLRKQARAHGKSVPEIHEQLVTYDEEGGGEMDTTSYDVSVLNSVRRGGAKP PRPALDARPSLYAQVQKPPRHAPGAHGGPGEMAAMIEVKKDEADHDGDGPPYD TLHIYGYEGSESIAESLSSLGTDSSDSDVDYDFLNDWGPRFKMLAELYGSDPREE LLY (SEQ ID NO: 99);
q. one of the one or more intracellular signaling domains is derived from MPZL1, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SMILAVLYRRKNSKRDYTGCSTSESLSPVKQAPRKSPSDTEGLVKSLPSGSHQGP VIYAQLDHSGGHHSDKINKSESVVYADIRKN (SEQ ID NO: 100), optionally wherein one of the one or more intracellular signaling domains comprises the amino acid sequence of SMILAVLYRRKNSKRDYTGCSTSESLSPVKQAPRKSPSDTEGLVKSLPSGSHQGP VIYAQLDHSGGHHSDKINKSESVVYADIRKN (SEQ ID NO: 100);
r. one of the one or more intracellular signaling domains is derived from MPZ, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RYCWLRRQAALQRRLSAMEKGKLHKPGKDASKRGRQTPVLYAMLDHSRSTKA VSEKKAKGLGESRKDKK (SEQ ID NO: 101), optionally wherein one of the one or more intracellular signaling domains comprises the amino acid sequence of RYCWLRRQAALQRRLSAMEKGKLHKPGKDASKRGRQTPVLYAMLDHSRSTKA VSEKKAKGLGESRKDKK (SEQ ID NO: 101);
s. one of the one or more intracellular signaling domains is derived from FCGR2B, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VVALIYCRKKRISALPGYPECREMGETLPEKPANPTNPDEADKVGAENTITYSLL MHPDALEEPDDQNRI (SEQ ID NO: 102), optionally wherein one of the one or more intracellular signaling domains comprises the amino acid sequence of VVALIYCRKKRISALPGYPECREMGETLPEKPANPTNPDEADKVGAENTITYSLL MHPDALEEPDDQNRI (SEQ ID NO: 102);
t. one of the one or more intracellular signaling domains is derived from SIGLEC-6, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RVKTRRKKAAQPVQNTDDVNPVMVSGSRGHQHQFQTGIVSDHPAEAGPISEDE QELHYAVLHFHKVQPQEPKVTDTEYSEIKIHK (SEQ ID NO: 103), optionally wherein one of the one or more intracellular signaling domains comprises the amino acid sequence of RVKTRRKKAAQPVQNTDDVNPVMVSGSRGHQHQFQTGIVSDHPAEAGPISEDE QELHYAVLHFHKVQPQEPKVTDTEYSEIKIHK (SEQ ID NO: 103);
u. one of the one or more intracellular signaling domains is derived from MPIG6B, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to WLHRRLPPQPIRPLPRFAPLVKTEPQRPVKEEEPKIPGDLDQEPSLLYADLDHLAL SRPRRLSTADPADASTIYAVVV (SEQ ID NO: 104), optionally wherein one of the one or more intracellular signaling domains comprises the amino acid sequence of WLHRRLPPQPIRPLPRFAPLVKTEPQRPVKEEEPKIPGDLDQEPSLLYADLDHLAL SRPRRLSTADPADASTIYAVVV (SEQ ID NO: 104);
v. one of the one or more intracellular signaling domains is derived from VSIG4, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to MLCRKTSQQEHVYEAARAHAREANDSGETMRVAIFASGCSSDEPTSQNLGNNY SDEPCIGQEYQIIAQINGNYARLLDTVPLDYEFLATEGKSVC (SEQ ID NO: 105), optionally wherein one of the one or more intracellular signaling domains comprises the amino acid sequence of MLCRKTSQQEHVYEAARAHAREANDSGETMRVAIFASGCSSDEPTSQNLGNNY SDEPCIGQEYQIIAQINGNYARLLDTVPLDYEFLATEGKSVC (SEQ ID NO: 105);
w. one of the one or more intracellular signaling domains is derived from SIGLEC-12, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RSCRKKSARPAVGVGDTGMEDANAVRGSASQGPLIESPADDSPPHHAPPALATP SPEEGEIQYASLSFHKARPQYPQEQEAIGYEYSEINIPK (SEQ ID NO: 106), optionally wherein one of the one or more intracellular signaling domains comprises the amino acid sequence of RSCRKKSARPAVGVGDTGMEDANAVRGSASQGPLIESPADDSPPHHAPPALATP SPEEGEIQYASLSFHKARPQYPQEQEAIGYEYSEINIPK (SEQ ID NO: 106);
x. one of the one or more intracellular signaling domains is derived from LIR8, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RHRHQSKHRTSAHFYRPAGAAGPEPKDQGLQKRASPVADIQEEILNAAVKDTQP KDGVEMDAPAAASEAPQDVTYAQLHSLTLRREATEPPPSQEREPPAEPSIYAPLAI H (SEQ ID NO: 107), optionally wherein one of the one or more intracellular signaling domains comprises the amino acid sequence of RHRHQSKHRTSAHFYRPAGAAGPEPKDQGLQKRASPVADIQEEILNAAVKDTQP KDGVEMDAPAAASEAPQDVTYAQLHSLTLRREATEPPPSQEREPPAEPSIYAPLAI H (SEQ ID NO: 107);
y. of the one or more intracellular signaling domains is derived from IRTA1, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to HCWRRRKSGVGFLGDETRLPPAPGPGESSHSICPAQVELQSLYVDVHPKKGDLV YSEIQTTQLGEEEEANTSRTLLEDKDVSVVYSEVKTQHPDNSAGKISSKDEES (SEQ ID NO: 108), optionally wherein one of the one or more intracellular signaling domains comprises the amino acid sequence of HCWRRRKSGVGFLGDETRLPPAPGPGESSHSICPAQVELQSLYVDVHPKKGDLV YSEIQTTQLGEEEEANTSRTLLEDKDVSVVYSEVKTQHPDNSAGKISSKDEES (SEQ ID NO: 108);
z. one of the one or more intracellular signaling domains is derived from KIR2DL4, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RWCSKKKDAAVMNQEPAGHRTVNREDSDEQDPQEVTYAQLDHCIFTQRKITGP SQRSKRPSTDTSVCIELPNAEPRALSPAHEHHSQALMGSSRETTALSQTQLASSNV PAAGI (SEQ ID NO: 109), optionally wherein one of the one or more intracellular signaling domains comprises the amino acid sequence of RWCSKKKDAAVMNQEPAGHRTVNREDSDEQDPQEVTYAQLDHCIFTQRKITGP SQRSKRPSTDTSVCIELPNAEPRALSPAHEHHSQALMGSSRETTALSQTQLASSNV PAAGI (SEQ ID NO: 109);
aa. one of the one or more intracellular signaling domains is derived from KIR2DL5, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LHCCCSNKKNAAVMDQEPAGDRTVNREDSDDQDPQEVTYAQLDHCVFTQTKIT SPSQRPKTPPTDTTMYMELPNAKPRSLSPAHKHHSQALRGSSRETTALSQNRVAS SHVPAAGI (SEQ ID NO: 110), optionally wherein one of the one or more intracellular signaling domains comprises the amino acid sequence of LHCCCSNKKNAAVMDQEPAGDRTVNREDSDDQDPQEVTYAQLDHCVFTQTKIT SPSQRPKTPPTDTTMYMELPNAKPRSLSPAHKHHSQALRGSSRETTALSQNRVAS SHVPAAGI (SEQ ID NO: 110);
bb. one of the one or more intracellular signaling domains is derived from SIGLEC-7, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RSCRKKSARPAADVGDIGMKDANTIRGSASQGNLTESWADDNPRHHGLAAHSS GEEREIQYAPLSFHKGEPQDLSGQEATNNEYSEIKIPK (SEQ ID NO: 111), optionally wherein one of the one or more intracellular signaling domains comprises the amino acid sequence of RSCRKKSARPAADVGDIGMKDANTIRGSASQGNLTESWADDNPRHHGLAAHSS GEEREIQYAPLSFHKGEPQDLSGQEATNNEYSEIKIPK (SEQ ID NO: 111);
cc. one of the one or more intracellular signaling domains is derived from FCRH3, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to HYARARRKPGGLSATGTSSHSPSECQEPSSSRPSRIDPQEPTHSKPLAPMELEPMY SNVNPGDSNPIYSQIWSIQHTKENSANCPMMHQEHEELTVLYSELKKTHPDDSA GEASSRGRAHEEDDEENYENVPRVLLASDH (SEQ ID NO: 112), optionally wherein one of the one or more intracellular signaling domains comprises the amino acid sequence of HYARARRKPGGLSATGTSSHSPSECQEPSSSRPSRIDPQEPTHSKPLAPMELEPMY SNVNPGDSNPIYSQIWSIQHTKENSANCPMMHQEHEELTVLYSELKKTHPDDSA GEASSRGRAHEEDDEENYENVPRVLLASDH (SEQ ID NO: 112);
dd. one of the one or more intracellular signaling domains is derived from PCDHGC5, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to KCLQGNADGDGGGGQCCRRQDSPSPDFYKQSSPNLQVSSDGTLKYMEVTLRPT DSQSHCYRTCFSPASDGSDFTFLRPLSVQQPTALALEPDAIRSRSNTLRERSQQAP PNTDWRFSQAQRPGTSGSQNGDDTGTWPNNQFDTEMLQAMILASASEAADGSS TLGGGAGTMGLSARYGPQFTLQHVPDYRQNVYIPGSNATLTNAAGKRDGKAPA GGNGNKKKSGKKEKK (SEQ ID NO: 113), optionally wherein one of the one or more intracellular signaling domains comprises the amino acid sequence of KCLQGNADGDGGGGQCCRRQDSPSPDFYKQSSPNLQVSSDGTLKYMEVTLRPT DSQSHCYRTCFSPASDGSDFTFLRPLSVQQPTALALEPDAIRSRSNTLRERSQQAP PNTDWRFSQAQRPGTSGSQNGDDTGTWPNNQFDTEMLQAMILASASEAADGSS TLGGGAGTMGLSARYGPQFTLQHVPDYRQNVYIPGSNATLTNAAGKRDGKAPA GGNGNKKKSGKKEKK (SEQ ID NO: 113);
ee. one of the one or more intracellular signaling domains is derived from CDH11, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RRQKKEPLIVFEEEDVRENIITYDDEGGGEEDTEAFDIATLQNPDGINGFIPRKDIK PEYQYMPRPGLRPAPNSVDVDDFINTRIQEADNDPTAPPYDSIQIYGYEGRGSVA GSLSSLESATTDSDLDYDYLQNWGPRFKKLADLYGSKDTFDDDS (SEQ ID NO: 114), optionally wherein one of the one or more intracellular signaling domains comprises the amino acid sequence of RRQKKEPLIVFEEEDVRENIITYDDEGGGEEDTEAFDIATLQNPDGINGFIPRKDIK PEYQYMPRPGLRPAPNSVDVDDFINTRIQEADNDPTAPPYDSIQIYGYEGRGSVA GSLSSLESATTDSDLDYDYLQNWGPRFKKLADLYGSKDTFDDDS (SEQ ID NO: 114);
ff. one of the one or more intracellular signaling domains is derived from IMPG2, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to YFFIRTLQAHHDRSERESPFSGSSRQPDSLSSIENAVKYNPVYESHRAGCEKYEGP YPQHPFYSSASGDVIGGLSREEIRQMYESSELSREEIQERMRVLELYANDPEFAAF VREQQVEEV (SEQ ID NO: 115), optionally wherein one of the one or more intracellular signaling domains comprises the amino acid sequence of YFFIRTLQAHHDRSERESPFSGSSRQPDSLSSIENAVKYNPVYESHRAGCEKYEGP YPQHPFYSSASGDVIGGLSREEIRQMYESSELSREEIQERMRVLELYANDPEFAAF VREQQVEEV (SEQ ID NO: 115);
gg. one of the one or more intracellular signaling domains is derived from DSCAM, optionally wherein one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RRRRREQRLKRLRDAKSLAEMLMSKNTRTSDTLSKQQQTLRMHIDIPRAQLLIEE RDTMETIDDRSTVLLTDADFGEAAKQKSLTVTHTVHYQSVSQATGPLVDVSDAR PGTNPTTRRNAKAGPTARNRYASQWTLNRPHPTISAHTLTTDWRLPTPRAAGSV DKESDSYSVSPSQDTDRARSSMVSTESASSTYEELARAYEHAKMEEQLRHAKFTI TECFISDTSSEQLTAGTNEYTDSLTSSTPSESGICRFTASPPKPQDGGRVMNMAVP KAHRPGDLIHLPPYLRMDFLLNRGGPGTSRDLSLGQACLEPQKSRTLKRPTVLEPI PMEAASSASSTREGQSWQPGAVATLPQREGAELGQAAKMSSSQESLLDSRGHLK GNNPYAKSYTLV (SEQ ID NO: 116), optionally wherein one of the one or more intracellular signaling domains comprises the amino acid sequence of RRRRREQRLKRLRDAKSLAEMLMSKNTRTSDTLSKQQQTLRMHIDIPRAQLLIEE RDTMETIDDRSTVLLTDADFGEAAKQKSLTVTHTVHYQSVSQATGPLVDVSDAR PGTNPTTRRNAKAGPTARNRYASQWTLNRPHPTISAHTLTTDWRLPTPRAAGSV DKESDSYSVSPSQDTDRARSSMVSTESASSTYEELARAYEHAKMEEQLRHAKFTI TECFISDTSSEQLTAGTNEYTDSLTSSTPSESGICRFTASPPKPQDGGRVMNMAVP KAHRPGDLIHLPPYLRMDFLLNRGGPGTSRDLSLGQACLEPQKSRTLKRPTVLEPI PMEAASSASSTREGQSWQPGAVATLPQREGAELGQAAKMSSSQESLLDSRGHLK GNNPYAKSYTLV (SEQ ID NO: 116).

4. (canceled)

5. (canceled)

6. (canceled)

7. The chimeric inhibitory receptor of claim 1, wherein the transmembrane domain is derived from a protein selected from the group consisting of: CD8, CD28, CD3ζ, CD4, 4-IBB, OX40, ICOS, 2B4, CD25, CD7, LAX, LAT, LIR1, PCAM-1, CD72, IRTA2, IRTA4, NKIR, IL1RAP, PTPRO, PTPRZ1, TLT1, SLAMF1, SLAMF5, PCDHGC3, LIFR, ERMAP, IL1RAPL2, CDH5, MPZL1, MPZ, FCGR2B, SIGLEC-6, MPIG6B, VSIG4, SIGLEC-12, LIR8, IRTA1, KIR2DL4, KIR2DL5, SIGLEC-7, FCRH3, PCDHGC5, CDH11, IMPG2, and DSCAM, optionally wherein:
a. the chimeric inhibitory receptor comprises a transmembrane domain derived from PECAM-1, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to GLIAVVIIGVIIALLIIAA (SEQ ID NO: 24), optionally wherein the transmembrane domain comprises the amino acid sequence of GLIAVVIIGVIIALLIIAA (SEQ ID NO: 24);
b. the chimeric inhibitory receptor comprises a transmembrane domain derived from LIR1, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VIGILVAVILLLLLLLLLFLI (SEQ ID NO: 25), optionally wherein the transmembrane domain comprises the amino acid sequence of VIGILVAVILLLLLLLLLFLI (SEQ ID NO: 25);
c. the chimeric inhibitory receptor comprises a transmembrane domain derived from IRTA2, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VAGGLLSIAGLAAGALLLYCW (SEQ ID NO: 26), optionally wherein the transmembrane domain comprises the amino acid sequence of VAGGLLSIAGLAAGALLLYCW (SEQ ID NO: 26);
d. the chimeric inhibitory receptor comprises a transmembrane domain derived from IRTA4, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LWGLFGVLGFTGVALLLYALF (SEQ ID NO: 27), optionally wherein the transmembrane domain comprises the amino acid sequence of LWGLFGVLGFTGVALLLYALF (SEQ ID NO: 27);
e. the chimeric inhibitory receptor comprises a transmembrane domain derived from NKIR, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VLLPLIFTILLLLLVAASLLA (SEQ ID NO: 28), optionally wherein the transmembrane domain comprises the amino acid sequence of VLLPLIFTILLLLLVAASLLA (SEQ ID NO: 28);
f. the chimeric inhibitory receptor comprises a transmembrane domain derived from IL1RAP, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VLLVVILIVVYHVYWLEMVLF (SEQ ID NO: 29), optionally wherein the transmembrane domain comprises the amino acid sequence of VLLVVILIVVYHVYWLEMVLF (SEQ ID NO: 29);
g. the chimeric inhibitory receptor comprises a transmembrane domain derived from PTPRO, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to ISVLAILSTLLIGLLLVTLII (SEQ ID NO: 30), optionally wherein the transmembrane domain comprises the amino acid sequence of ISVLAILSTLLIGLLLVTLII (SEQ ID NO: 30);
h. the chimeric inhibitory receptor comprises a transmembrane domain derived from PTPRZ1, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AVIPLVIVSALTFICLVVLVGILIYW (SEQ ID NO: 31), optionally wherein the transmembrane domain comprises the amino acid sequence of AVIPLVIVSALTFICLVVLVGILIYW (SEQ ID NO: 31);
i. the chimeric inhibitory receptor comprises a transmembrane domain derived from TLT1, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LIWGAVLLVGLLVAAVVLFAV (SEQ ID NO: 32), optionally wherein the transmembrane domain comprises the amino acid sequence of LIWGAVLLVGLLVAAVVLFAV (SEQ ID NO: 32);
j. the chimeric inhibitory receptor comprises a transmembrane domain derived from SLAMF1, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to WAVYAGLLGGVIMILIMVVIL (SEQ ID NO: 33), optionally wherein the transmembrane domain comprises the amino acid sequence of WAVYAGLLGGVIMILIMVVIL (SEQ ID NO: 33);
k. the chimeric inhibitory receptor comprises a transmembrane domain derived from SLAMF5, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LLSVLAMFFLLVLILSSVFLF (SEQ ID NO: 34), optionally wherein the transmembrane domain comprises the amino acid sequence of LLSVLAMFFLLVLILSSVFLF (SEQ ID NO: 34);
l. the chimeric inhibitory receptor comprises a transmembrane domain derived from PCDHGC3, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LLLSLILVSVGFVVTVFGVII (SEQ ID NO: 139), optionally wherein the transmembrane domain comprises the amino acid sequence of LLLSLILVSVGFVVTVFGVII (SEQ ID NO: 139);
m. the chimeric inhibitory receptor comprises a transmembrane domain derived from LIFR, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VGLIIAILIPVAVAVIVGVVTSILC (SEQ ID NO: 140), optionally wherein the transmembrane domain comprises the amino acid sequence of VGLIIAILIPVAVAVIVGVVTSILC (SEQ ID NO: 140);
n. the chimeric inhibitory receptor comprises a transmembrane domain derived from ERMAP, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VALAVILPVLVLLIMVCLCLI (SEQ ID NO: 141), optionally wherein the transmembrane domain comprises the amino acid sequence of VALAVILPVLVLLIMVCLCLI (SEQ ID NO: 141);
o. the chimeric inhibitory receptor comprises a transmembrane domain derived from IL1RAPL2, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to IELAGGLGAIFLLLVLLVVIY (SEQ ID NO: 142), optionally wherein the transmembrane domain comprises the amino acid sequence of IELAGGLGAIFLLLVLLVVIY (SEQ ID NO: 142);
p. the chimeric inhibitory receptor comprises a transmembrane domain derived from CDH5, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AVVAILLCILTITVITLLIFL (SEQ ID NO: 143), optionally wherein the transmembrane domain comprises the amino acid sequence of AVVAILLCILTITVITLLIFL (SEQ ID NO: 143);
q. the chimeric inhibitory receptor comprises a transmembrane domain derived from MPZL1, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to FPVWVVVGIVTAVVLGLTLLI (SEQ ID NO: 144), optionally wherein the transmembrane domain comprises the amino acid sequence of FPVWVVVGIVTAVVLGLTLLI (SEQ ID NO: 144);
r. the chimeric inhibitory receptor comprises a transmembrane domain derived from MPZ, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to YGVVLGAVIGGVLGVVLLLLLLFYVV (SEQ ID NO: 145), optionally wherein the transmembrane domain comprises the amino acid sequence of YGVVLGAVIGGVLGVVLLLLLLFYVV (SEQ ID NO: 145);
s. the chimeric inhibitory receptor comprises a transmembrane domain derived from FCGR2B, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SSSPMGIIVAVVTGIAVAAIVAA (SEQ ID NO: 146), optionally wherein the transmembrane domain comprises the amino acid sequence of SSSPMGIIVAVVTGIAVAAIVAA (SEQ ID NO: 146);
t. the chimeric inhibitory receptor comprises a transmembrane domain derived from SIGLEC-6, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LGAVWGASITTLVFLCVCFIF (SEQ ID NO: 147), optionally wherein the transmembrane domain comprises the amino acid sequence of LGAVWGASITTLVFLCVCFIF (SEQ ID NO: 147);
u. the chimeric inhibitory receptor comprises a transmembrane domain derived from MPIG6B, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LLIPLLGAGLVLGLGALGLVW (SEQ ID NO: 148), optionally wherein the transmembrane domain comprises the amino acid sequence of LLIPLLGAGLVLGLGALGLVW (SEQ ID NO: 148);
v. the chimeric inhibitory receptor comprises a transmembrane domain derived from VSIG4, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VFAIILIISLCCMVVFTMAYI (SEQ ID NO: 149), optionally wherein the transmembrane domain comprises the amino acid sequence of VFAIILIISLCCMVVFTMAYI (SEQ ID NO: 149);
w. the chimeric inhibitory receptor comprises a transmembrane domain derived from SIGLEC-12, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to FGGAGATALVFLYFCIIFVVV (SEQ ID NO: 150), optionally wherein the transmembrane domain comprises the amino acid sequence of FGGAGATALVFLYFCIIFVVV (SEQ ID NO: 150);
x. the chimeric inhibitory receptor comprises a transmembrane domain derived from LIR8, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VVTGVSVAFVLLLFLLLFLLL (SEQ ID NO: 151), optionally wherein the transmembrane domain comprises the amino acid sequence of VVTGVSVAFVLLLFLLLFLLL (SEQ ID NO: 151);
y. the chimeric inhibitory receptor comprises a transmembrane domain derived from IRTA1, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VAAGATGGLLSALLLAVALLF (SEQ ID NO: 152), optionally wherein the transmembrane domain comprises the amino acid sequence of VAAGATGGLLSALLLAVALLF (SEQ ID NO: 152);
z. the chimeric inhibitory receptor comprises a transmembrane domain derived from KIR2DL4, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AVIRYSVAIILFTILPFFLLH (SEQ ID NO: 153), optionally wherein the transmembrane domain comprises the amino acid sequence of AVIRYSVAIILFTILPFFLLH (SEQ ID NO: 153);
aa. the chimeric inhibitory receptor comprises a transmembrane domain derived from KIR2DL5, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LHILIGTSVAIILFIILFFFL (SEQ ID NO: 154), optionally wherein the transmembrane domain comprises the amino acid sequence of LHILIGTSVAIILFIILFFFL (SEQ ID NO: 154);
bb. the chimeric inhibitory receptor comprises a transmembrane domain derived from SIGLEC-7, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to GAVGGAGATALVFLSFCVIFIVV (SEQ ID NO: 155), optionally wherein the transmembrane domain comprises the amino acid sequence of GAVGGAGATALVFLSFCVIFIVV (SEQ ID NO: 155);
cc. the chimeric inhibitory receptor comprises a transmembrane domain derived from FCRH3, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AAGITGLVLSILVLAAAAALL (SEQ ID NO: 156), optionally wherein the transmembrane domain comprises the amino acid sequence of AAGITGLVLSILVLAAAAALL (SEQ ID NO: 156);
dd. the chimeric inhibitory receptor comprises a transmembrane domain derived from PCDHGC5, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LIVALATVSLLSLVTFTFLSA (SEQ ID NO: 157), optionally wherein the transmembrane domain comprises the amino acid sequence of LIVALATVSLLSLVTFTFLSA (SEQ ID NO: 157);
ee. the chimeric inhibitory receptor comprises a transmembrane domain derived from CDH11, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to GALIAILACIVILLVIVVLFVTL (SEQ ID NO: 158), optionally wherein the transmembrane domain comprises the amino acid sequence of GALIAILACIVILLVIVVLFVTL (SEQ ID NO: 158);
ff. the chimeric inhibitory receptor comprises a transmembrane domain derived from IMPG2, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VIIGITIASVVGLLVIFSAII (SEQ ID NO: 159), optionally wherein the transmembrane domain comprises the amino acid sequence of VIIGITIASVVGLLVIFSAII (SEQ ID NO: 159); or
gg. the chimeric inhibitory receptor comprises a transmembrane domain derived from DSCAM, optionally wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LVTISCILVGVLLLFVLLLVV (SEQ ID NO: 160), optionally wherein the transmembrane domain comprises the amino acid sequence of LVTISCILVGVLLLFVLLLVV (SEQ ID NO: 160).

8. The chimeric inhibitory receptor of claim 1, wherein (i) the protein is not expressed on the target tumor, optionally wherein the protein is expressed on a non-tumor cell, optionally wherein the protein is expressed on a non-tumor cell derived from a tissue selected from the group consisting of brain, neuronal tissue, endocrine, endothelial, bone, bone marrow, immune system, muscle, lung, liver, gallbladder, pancreas, gastrointestinal tract, kidney, urinary bladder, male reproductive organs, female reproductive organs, adipose, soft tissue, and skin; and/or (ii) the extracellular protein binding domain comprises a ligand-binding domain, a receptor-binding domain, and/or an antigen-binding domain, optionally wherein the antigen-binding domain comprises an antibody, an antigen-binding fragment of an antibody, a F(ab) fragment, a F(ab′) fragment, a single chain variable fragment (scFv), or a single-domain antibody (sdAb), optionally wherein the antigen-binding domain comprises a single chain variable fragment (scFv), optionally wherein the scFv comprises a heavy chain variable domain (VH) and a light chain variable domain (VL), optionally wherein the VH and VL are separated by a peptide linker, optionally wherein the peptide linker comprises an amino acid sequence selected from the group consisting of: GGS (SEQ ID NO: 47), GGSGGS (SEQ ID NO: 48), GGSGGSGGS (SEQ ID NO: 49), GGSGGSGGSGGS (SEQ ID NO: 50), GGSGGSGGSGGSGGS (SEQ ID NO: 51), GGGS (SEQ ID NO: 52), GGGSGGGS (SEQ ID NO: 53), GGGSGGGSGGGS (SEQ ID NO: 54), GGGSGGGSGGGSGGGS (SEQ ID NO: 55), GGGSGGGSGGGSGGGSGGGS (SEQ ID NO: 56), GGGGS (SEQ ID NO: 57), GGGGSGGGGS (SEQ ID NO: 58), GGGGSGGGGSGGGGS (SEQ ID NO: 59), GGGGSGGGGSGGGGSGGGGS (SEQ ID NO: 60), GGGGGGGGSGGGGSGGGGSGGGGS (SEQ ID NO: 61), GGGGGGGGSGGGGSQSV (SEQ ID NO: 63), GSTSGSGKPGSGEGSTKG (SEQ ID NO: 64), SGGGGSGGGGSGGGGSGGGGSGGGSLQ (SEQ ID NO: 65), and TTTPAPRPPTPAPTIALQPLSLRPEACRPAAGGAVHTRGLDFACDQTTPGERSSLPAFYPG TSGSCSGCGSLSLP (SEQ ID NO: 62), optionally wherein the scFv comprises the structure VH-L-VL or VL-L-VH, wherein VH is the heavy chain variable domain, L is the peptide linker, and VL is the light chain variable domain.

9. The chimeric inhibitory receptor of claim 1, wherein the chimeric inhibitory receptor further comprises a spacer region positioned between the extracellular protein binding domain and the transmembrane domain and operably linked, e.g., physically linked, to each of the extracellular protein binding domain and the transmembrane domain, optionally wherein the spacer region is derived from a protein selected from the group consisting of: CD8α, CD4, CD7, CD28, IgG1, IgG4, FcγRIIIα, LNGFR, and PDGFR, optionally wherein the spacer region comprises an amino acid sequence selected from the group consisting of:
(SEQ ID NO: 73)
TTTPAPRPPTPAPTIALQPLSLRPEACRPAAGGAVHTRGLDFACD,
(SEQ ID NO: 77)
ALSNSIMYFSHFVPVFLPAKPTTTPAPRPPTPAPTIASQPLSLRPEACRP
AAGGAVHTRGLDFACD,
(SEQ ID NO: 67)
AAAIEVMYPPPYLDNEKSNGTIIHVKGKHLCPSPLFPGPSKP,
(SEQ ID NO: 68)
ESKYGPPCPSCP,
(SEQ ID NO: 69)
ESKYGPPAPSAP,
(SEQ ID NO: 70)
ESKYGPPCPPCP,
(SEQ ID NO: 71)
EPKSCDKTHTCP,
(SEQ ID NO: 72)
AAAFVPVFLPAKPTTTPAPRPPTPAPTIASQPLSLRPEACRPAAGGAVHT
RGLDFACDIYIWAPLAGTCGVLLLSLVITLYCNHRN,
(SEQ ID NO: 74)
ACPTGLYTHSGECCKACNLGEGVAQPCGANQTVCEPCLDSVTFSDVVSAT
EPCKPCTECVGLQSMSAPCVEADDAVCRCAYGYYQDETTGRCEACRVCEA
GSGLVFSCQDKQNTVCEECPDGTYSDEADAEC,
(SEQ ID NO: 75)
ACPTGLYTHSGECCKACNLGEGVAQPCGANQTVC,
(SEQ ID NO: 76)
AVGQDTQEVIVVPHSLPFKV,
(SEQ ID NO: 183)
ESKYGPPCPSCPAPPVAGPSVFLFPPKPKDTLMISRTPEVTCVVVDVSQE
DPEVQFNWYVDGVEVHNAKTKPREEQFQSTYRVVSVLTVLHQDWLNGKEY
KCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQE
GNVFSCSVMHEALHNHYTQKSLSLSLGK,
and
(SEQ ID NO: 184)
ESKYGPPCPSCPGQPREPQVYTLPPSQEEMTKNQVSLTCLVKGFYPSDIA
VEWESNGQPENNYKTTPPVLDSDGSFFLYSRLTVDKSRWQEGNVFSCSVM
HEALHNHYTQKSLSLSLGK.

10. The chimeric inhibitory receptor of claim 1, wherein the chimeric inhibitory receptor further comprises an intracellular spacer region positioned between the transmembrane domain and one of the one or more intracellular signaling domains and operably linked, e.g., physically linked, to each of the transmembrane domain and one of the one or more intracellular signaling domains.

11. The chimeric inhibitory receptor of claim 1, wherein the inhibitory chimeric receptor further comprises an enzymatic inhibitory domain, optionally wherein the enzymatic inhibitory domain is capable of preventing, attenuating, or inhibiting activation of a tumor-targeting chimeric receptor when expressed on an immunomodulatory cell relative to an otherwise identical chimeric inhibitory receptor lacking the enzymatic inhibitory domain, optionally wherein the enzymatic inhibitory domain comprises an enzyme catalytic domain, optionally wherein the enzyme catalytic domain is derived from an enzyme selected from the group consisting of: CSK, SHP-1, PTEN, CD45, CD148, PTP-MEG1, PTP-PEST, c-CBL, CBL-b, PTPN22, LAR, PTPH1, SHIP-1, and RasGAP.

12. The chimeric inhibitory receptor of claim 1, wherein the tumor-targeting chimeric receptor is a chimeric antigen receptor (CAR) or an engineered T cell receptor (TCR).

13. The chimeric inhibitory receptor of claim 1, wherein the immunomodulatory cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell, optionally wherein the immunomodulatory cell is a Natural Killer (NK) cell.

14. A composition comprising the chimeric inhibitory receptor of claim 1 and a pharmaceutically acceptable carrier, a pharmaceutically acceptable excipient, or a combination thereof.

15. An engineered nucleic acid encoding the chimeric inhibitory receptor of claim 1.

16. An expression vector comprising the engineered nucleic acid of claim 15.

17. A composition comprising the engineered nucleic acid of claim 15 or the expression vector of claim 16, and a pharmaceutically acceptable carrier

18. A producer cell or isolated immunomodulatory cell comprising the chimeric inhibitory receptor of any claim 1 or the engineered nucleic acid of claim 15.

19. A composition comprising the isolated cell of claim 18 and a pharmaceutically acceptable carrier, a pharmaceutically acceptable excipient, or a combination thereof.

20. A method of preventing, attenuating, or inhibiting a cell-mediated immune response induced by a tumor-targeting chimeric receptor expressed of the surface of an immunomodulatory cell, comprising:
engineering the immunomodulatory cell to express the chimeric inhibitory receptor of claim 1 on the surface of the immunomodulatory cell,
wherein upon binding of a cognate antigen to the chimeric inhibitory receptor, the intracellular signaling domain prevents, attenuates, or inhibits activation of the tumor-targeting chimeric receptor.

21. A method of preventing, attenuating, or inhibiting activation of a tumor-targeting chimeric receptor expressed on the surface of an immunomodulatory cell, comprising:
contacting the isolated cell of claim 18 with a cognate antigen of the chimeric inhibitory receptor under conditions suitable for the chimeric inhibitory receptor to bind the cognate antigen,
wherein upon binding of the antigen to the chimeric inhibitory receptor, the intracellular signaling domain prevents, attenuates, or inhibits activation of the tumor-targeting chimeric receptor.
